(19) United States
(12) Patent Application Publication
(10) Pub. No.: US 2011/0243966 A1
(43) Pub. Date: Oct. 6, 2011

(54) SINGLE CHAIN Fc (ScFc) REGIONS, BINDING POLYPEPTIDES COMPRISING SAME, AND METHODS RELATED THERETO

(75) Inventors: Graham K. FARRINGTON, Acton, MA (US); Amna SAEED-KOTHE, West Roxbury, MA (US); Ellen GARBER, Cambridge, MA (US); Alexey Alexandrovich LUGOVSKOY, Woburn, MA (US)

(73) Assignee: BIOGEN IDEC MA INC., Cambridge, MA (US)

(21) Appl. No.: 13/047,534

(22) Filed: Mar. 14, 2011

Related U.S. Application Data
(62) Division of application No. 12/152,622, filed on May 14, 2008.
(60) Provisional application No. 60/930,227, filed on May 14, 2007.

(51) Int. Cl. A61K 39/395; C07H 21/00; C12N 15/63; C12N 1/00; C12N 5/10; C12P 21/00; C07K 16/00; C07K 19/00; C12N 1/19

(52) U.S. Cl. 424/178.1; 536/23.4; 435/320.1; 435/243; 435/419; 435/325; 435/69.6; 530/387.3; 530/391.7; 530/391.9; 435/254.2

(57) ABSTRACT

The present invention features inter alia polypeptides comprising an Fc region comprising genetically-fused Fc moieties. In addition, the instant invention provides, e.g., methods for treating or preventing a disease or disorder in subject by administering the binding polypeptides of the invention to said subject.

### RELATED APPLICATIONS

This application is a Divisional application of U.S. patent application Ser. No. 12/152,622 filed May 14, 2008 which claims priority to U.S. Provisional Application No. 60/930,227, filed May 14, 2007, titled “BINDING POLYPEPTIDES CONTAINING GENETICALLY-FUSED FC REGIONS AND METHODS RELATED THERETO,” which is incorporated herein by reference in its entirety. Additionally, the contents of any patents, patent applications, and references cited throughout this specification are hereby incorporated by reference in its entirety.

### BACKGROUND OF THE INVENTION

The Fc region of an immunoglobulin mediates effector functions that have been divided into two categories. In the first are functions that occur independently of antigen binding; these functions confer persistence in circulation and the ability to be transferred across cellular barriers by transcytosis (see Ward and Ghetie, Therapeutic Immunology 2:77-94, 1995, Capon et al. Nature 1989). The circulatory half-life of the IgG subclass of immunoglobulins is regulated by the affinity of the Fc region for the neonatal Fc receptor or FcRn (see Ghetie et al., Nature Biotechnol. 15:637-640, 1997; Kim et. al., Eur. J. Immunol. 24:542-548, 1994; Dall'Acqua et al. (J. Immunol. 169:5171-5180, 2002). The second general category of effector functions include those that operate after an immunoglobulin binds an antigen. In the case of IgG, these functions involve the participation of the complement cascade or Fc gamma receptor (FcγR)-bearing cells. Binding of the Fc region to an FcγR causes certain immune effects, for example, endocytosis of immune complexes, engulfment and destruction of immunoglobulin-coated particles or microorganisms (also called antibody-dependent phagocytosis, or ADCP), clearance of immune complexes, lysis of immunoglobulin-coated target cells by killer cells (called antibody-dependent cell-mediated cytotoxicity, or ADCC), release of inflammatory mediators, regulation of immune system cell activation, and regulation of immunoglobulin production.

Certain engineered binding polypeptides (e.g., antibody variants (e.g., scFvs) or antibody fragments (e.g., Fab fragments)), while benefiting from their smaller molecular size and/or monovalency, also suffer several disadvantages attributable to the absence of a functional Fc region. For example, Fab fragments have short half-lives in vivo because they lack the Fc region that is required for FcRn binding and are rapidly filtered out of the blood by the kidneys owing to their small size. While it is possible to generate monovalent, Fc-containing, binding polypeptides, current methods require either coexpression of the two heavy chain portions of a dimeric Fc region or chemical conjugation of the dimeric Fc region to a binding site (e.g., a Fab domain). These methods are inefficient since coexpression yields products that are complex mixtures representing all possible pairings of starting material in addition to aggregates and inactive protein. Consequently, yields of the desired functional binding polypeptide are low. Additionally, using prior art methods it was not possible to efficiently produce binding molecules having heteromeric Fc regions (ie., where the heavy chain portions of the dimeric Fc region differ in sequence).

Accordingly, there is a need for Fc-containing binding polypeptides which can be produced efficiently and robustly while retaining desired Fc effector function(s).

### SUMMARY OF THE INVENTION

The present invention features inter alia Fc polypeptides (e.g, Fc binding polypeptides) comprising one or more genetically-fused Fc regions. In particular, the polypeptides of the invention comprise a single chain Fc region (“scFc”) in which the component Fc moieties are genetically-fused in a single polypeptide chain such that they form a functional, dimeric Fc region. In certain embodiments, the component Fc moieties of an scFc are genetically fused in tandem via a polypeptide linker (e.g., an Fc connecting peptide) interposed between the Fc moieties. Thus, the scFc polypeptides of the invention comprise scFc region(s) formed by a single contiguous amino acid sequence which is encoded in a single open reading frame (ORF) as part of one contiguous nucleotide sequence. In contrast, the Fc regions of conventional Fc polypeptides (e.g., conventional immunoglobulins) are obligate homodimers comprising separate (i.e., unlinked) Fc domains or moieties in separate polypeptide chains that dimerize post-translationally but that are not covalently linked in tandem.

The single-chain Fc (scFc) polypeptides of the invention provide several advantages over conventional Fc polypeptides. In certain aspects, the genetically-fused Fc regions (i.e., scFc region) of a scFc polypeptide may be operably linked to the binding site of a binding polypeptide (e.g., to an antigen binding fragment (e.g., a Fab) or an scFv molecule) to form a scFc binding polypeptide, thereby imparting an effector function to the binding polypeptide or altering an existing effector function. scFc binding polypeptides of the invention may be monomeric or multimeric (e.g., dimeric). The novel scFc binding polypeptides of the invention combine the advantage of a monovalent binding polypeptide (e.g., the lack of cell-surface receptor crosslinking that can lead to improper cell signaling and/or endocytosis) with the advantage, at least in one embodiment, of Fc-mediated effector functions (e.g. an increase in half-life due to binding by FcRn, imparting FcγRI, FcγRII, and FcγRIII binding and complement activation) and, in one embodiment, of being able to fine-tune such effector functions. Moreover, the scFc binding polypeptides of the invention may be readily expressed in highly homogenous preparations that are readily scaled-up for high-yield manufacturing. For example, a binding polypeptide comprising one or more target binding sites (e.g., antigen binding sites, such as one or more scFv or Fab fragments) can be linked to either or both of the N- or C-termini of a genetically-fused Fc region (i.e., scFc region) and encoded in a single genetic construct, thereby avoiding the complex mixture of molecules that result from coexpression of two or more chains.

The scFc polypeptides of the invention also afford the opportunity to produce molecules having heteromeric scFc regions in highly homogenous preparations. It is currently very difficult to create and purify heteromeric Fc-containing molecules in which the two Fc moieties which make up a conventional Fc region are different from each other, for example in which only one of the two Fc moeities comprises an amino acid modification (e.g., a single point mutation within a single CH2 and/or CH3 domain). Given the teachings of the instant application, heteromeric scFc binding polypeptides in which fewer than all of the Fc moieties of the scFc region comprise a mutation can now be readily obtained from a single genetic construct. Such molecules are readily scaled up for manufacturing.

In one aspect, the instant invention is directed to a scFc binding polypeptide comprising (i) a first target binding site, and (ii) a first single-chain Fc (scFc) region comprising at least two genetically-fused Fc moieties, wherein the Fc moieties of the scFc region are genetically fused via a polypeptide linker sequence interposed between said Fc moieties; and wherein the scFc region imparts at least one effector function to said binding polypeptide.

In one embodiment, the invention is further directed to an scFc binding polypeptide comprising an scFc region, wherein said scFc region comprises a domain (e.g., an effector domain) selected from the group consisting of an FcRn binding portion, an FcγR binding portion, and a complement binding portion. In another embodiment, the domain is a Protein A or Protein G binding portion.

In certain embodiments, said scFc region is a heteromeric scFc region. In one embodiment, said heteromeric Fc region is hemiglycosylated.

In one embodiment, the scFc region is a heteromeric scFc region. In another embodiment, the scFc region is a homomeric scFc region.

In one embodiment, the scFc region is fully glycosylated. In another embodiment, the scFc region is aglycosylated. In yet another embodiment, said scFc region is afucosylated.

In certain embodiment, the scFc region of said polypeptide is a chimeric Fc region. For example, the scFc region may comprise CH2 domains from an IgG2 molecule and CH3 domains from an IgG4 molecule. In other embodiments, said scFc region may comprise a CH2 portion from an IgG2 molecule and a CH2 portion from an IgG4 molecule. In yet other embodiments, the scFc may comprise a modified or chimeric hinge region, e.g., a chimeric hinge comprising a middle hinge region from an IgG4 molecule and upper and lower hinge regions from an IgG1 molecule. In other embodiments, one or more cysteine residues of hinge region are substituted with a serine residue.

In one embodiment, the scFc region comprises two or more Fc domains or moieties.

In one embodiment, one or more of said Fc moieties is a domain-deleted Fc moiety selected from the group consisting of a CH2 domain-deleted Fc moiety, a CH3 domain-deleted Fc moiety, and a hinge-deleted Fc moiety.

In one embodiment, at least one of said Fc moeities comprises at least one Fc mutation at an EU convention amino acid position within said Fc moiety.

In another embodiment, two or more of said Fc moieties comprise one or more Fc mutations at EU convention amino acid positions within said Fc moieties.

In one embodiment, at least one amino acid position selected from the group consisting of 234, 236, 239, 241, 246-252, 254-256, 275, 277-288, 294, 296-298, 301, 303-307, 309, 310, 312, 313, 315, 328, 332, 334, 338, 342, 343, 350, 355, 359, 360, 361, 374, 376, 378, 381-385, 387, 389, 413, 415, 418, 422, 426, 428, 430-432, 434, 435, 438, and 441-446 (EU numbering convention) is mutated in at least one Fc moiety of a binding molecule.

In one embodiment, at least one Fc mutation is located in a hinge domain of at least one Fc moiety of a binding molecule. In another embodiment, at least one Fc mutation is located in a CH2 domain. In another embodiment, at least one Fc mutation is located in a CH3 domain.

In one embodiment, the CH3 domain comprises an engineered cysteine or thiol-containing analog thereof at one or more amino acid positions independently selected from the group consisting of 350, 355, 361, 389, 415, 441, 443, and 446b, according to the EU numbering index, of at least one Fc moiety of a binding molecule.

In one embodiment, a binding molecule of the invention has reduced glycosylation at EU position 297 of at least one Fc moiety. In another embodiment, the binding polypeptide is afucosylated at EU position 297.

In one embodiment, the polypeptide linker has a length of about 50 to about 500 amino acids. In another embodiment, the polyeptide linker has a length of about 50 to about 200 amino acids. In another embodiment, the polypeptide linker has a length of about 1 to about 50 amino acids. In yet another embodiment, the polypeptide linker has a length of about 10 to about 20 amino acids. In one embodiment, the polypeptide linker comprises a hinge region or portion thereof. In one embodiment, the hinge region is a chimeric hinge region. In one embodiment, the polypeptide linker comprises a gly/ser peptide. In one embodiment, the gly/ser peptide is of the formula (Gly₄Ser)n, wherein n is a positive integer selected from the group consisting of 1, 2, 3, 4, 5, 6, 7, 8, 9 and 10.

In one embodiment, the (Gly₄ Ser)n scFc linker is (Gly₄ Ser)4. In another embodiment, the (Gly₄ Ser)n scFc linker is (Gly₄ Ser)3.

In one embodiment, the polypeptide linker comprises said first target binding site. In one embodiment, the polypeptide linker comprises a biologically relevant peptide or portion thereof. In one embodiment, the biologically relevant polypeptide is an anti-rejection or anti-inflammatory peptide. In another embodiment, the biologically relevant polyeptide is selected from the group consisting of a cytokine inhibitory peptide, a cell adhesion inhibitory peptide, a thrombin inhibitory peptide, and a platelet inhibitory peptide. In another embodiment, the cytokine inhibitory peptide is an L-1 inhibitory peptide.

In one embodiment, the first binding site is genetically fused to the N-terminus of the scFc region. In another embodiment, the first binding site is genetically fused to the C-terminus of the scFc region.

In one embodiment, a binding molecule of the invention further comprises a second target binding site. In one embodiment, the second target binding site is operably linked to the N-terminus of the scFc region. In another embodiment, the second target binding site is operably linked to the C-terminus of the scFc region.

In another embodiment, the binding site is veneered onto an Fc moiety (e.g., 1, 2, or more CH2 domains and/or 1, 2, or more CH3 domains) of the scFc region.

In one embodiment, at least one target binding site is selected from the group consisting of an antigen binding site, a ligand binding portion of a receptor, and a receptor binding portion of a ligand.

In one embodiment, the antigen binding site is derived from an antibody. In one embodiment, the antibody is selected from the group consisting of a monoclonal antibody, a chimeric antibody, a human antibody, and a humanized antibody. In another embodiment, the antigen binding site is derived from an antibody variant selected from the group consisting of a scFv, a Fab, a minibody, a diabody, a triabody, a nanobody, a camelid, and a Dab. In another embodiment, the binding site is derived from a non-immunoglobulin binding molecule, e.g., a non-immunogloublin binding molecule is selected from the group consisting of an adnectin, an affibody, a DARPin and an anticalin.

In one embodiment, the binding polypeptide of the invention comprises at least one binding site comprising at least one CDR, variable region, or antigen binding site from an antibody selected from the group consisting of Rituximab, Daclizumab, Galiximab, CB6, Li33, 5c8, CBE11, BDA8, 14A2, B3F6, 2B8, Lym 1, Lym 2, LL2, Her2, 5E8, B1, MB1, BH3, B4, B72.3, CC49, and 5E10.

In one embodiment, the ligand binding portion of a receptor is derived from a receptor selected from the group consisting of a receptor of the Immunoglobulin (Ig) superfamily, a receptor of the TNF receptor superfamily, a receptor of the G-protein coupled receptor (GPCR) superfamily, a receptor of the Tyrosine Kinase (TK) receptor superfamily, a receptor of the Ligand-Gated (LG) superfamily, a receptor of the chemokine receptor superfamily, IL-1/Toll-like Receptor (TLR) superfamily, a receptor of the glial glial-derived neurotrophic factor (GDNF) receptor family, and a cytokine receptor superfamily. In one embodiment, said receptor of the TNF receptor superfamily is LTβR. In another embodiment, said receptor of the TNF receptor superfamily binds TNFα In yet another embodiment, said receptor of GDNF receptor family is GFRα3.

In one embodiment, the receptor binding portion of a ligand is derived from an inhibitory ligand. In one embodiment, the receptor binding portion of a ligand is derived from an activating ligand. In one embodiment, the ligand binds a receptor selected from the group consisting of a receptor of the Immunoglobulin (Ig) superfamily, a receptor of the TNF receptor superfamily, a receptor of the G-protein coupled receptor (GPCR) superfamily, a receptor of the Tyrosine Kinase (TK) receptor superfamily, a receptor of the Ligand-Gated (LG) superfamily, a receptor of the chemokine receptor superfamily, IL-1/Toll-like Receptor (TLR) superfamily, and a cytokine receptor superfamily. In one embodiment, the ligand that binds a receptor of the cytokine receptor superfamily is β-interferon.

In one embodiment, the first and second target binding sites have different binding specificities. In another embodiment, the first and second target binding sites have the same binding specificity.

In one embodiment, a binding molecule of the invention further comprises two or more scFc regions.

In one embodiment, a binding molecule of the invention is conjugated to at least one functional moiety.

In one embodiment, the functional moiety is selected from the group consisting of a blocking moiety, a detectable moiety, a diagnostic moiety, and a therapeutic moiety.

In one embodiment, the blocking moiety is selected from the group consisting of a cysteine adduct, mixed disulfide, polyethylene glycol, and polyethylene glycol maleimide.

In one embodiment, the detectable moiety is selected from the group consisting of a fluorescent moiety and isotopic moiety.

In one embodiment, the diagnostic moiety is capable of revealing the presence of a disease or disorder.

In one embodiment, the therapeutic moiety is selected from the group consisting of an anti-inflammatory agent, an anticancer agent, an anti-neurodegenerative agent, and an anti-infective agent.

In one embodiment, the functional moiety is conjugated to said polypeptide linker.

In one embodiment, the functional moiety is conjugated via a disulfide bond. In another embodiment, the functional moiety is conjugated via a heterobifunctional linker.

In one embodiment, the invention is directed to a multimeric binding polypeptide comprising a scFc binding polypeptide of the invention and second polypeptide.

In one embodiment, the second polypeptide is a binding polyeptide (e.g., a scFc binding polypeptide). In one embodiment, the second binding polypeptide comprises (i) at least a first antigen binding portion, and (ii) at least a first scFc region wherein said scFc region comprises at least two Fc moieties, and wherein said scFc region imparts at least one effector function to said binding polypeptide. In one embodiment, the scFc region of the second binding polypeptide comprises a linker polypeptide (e.g., an Fc connecting polypeptide) interposed between two Fc moieties of the scFc region.

In one embodiment, the multimeric binding polypeptide is a dimeric binding polypeptide.

In one embodiment, the first or second binding portion of a binding molecule of the invention binds to an antigen present on an immune cell or a tumor cell.

In one embodiment, at least one Fc moiety of a binding molecule of the invention is of the IgG isotype.

In one embodiment, the IgG isotype is of the IgG1 subclass.

In one embodiment, at least one Fc moiety of a binding molecule of the invention is derived from a human antibody.

In one aspect, the invention pertains to a pharmaceutical composition comprising a binding molecule of the invention.

In another aspect, the invention pertains to a nucleic acid molecule comprising a nucleotide sequence encoding the polypeptide of the invention.

In one embodiment, the nucleic acid molecule is in an expression vector.

In one embodiment, the invention pertains to a host cell comprising the expression vector comprising a nucleic acid molecule of the invention.

In one embodiment, the invention pertains to a method for producing a binding polypeptide comprising culturing a host cell.

In another aspect, the invention pertains to a method for treating or preventing a disease or disorder in a subject, comprising administering a binding molecule of the invention.

In one embodiment, the disease or disorder is selected from the group consisting of an inflammatory disorder, a neurological disorder, an autoimmune disorder, and a neoplastic disorder.

### BRIEF DESCRIPTION OF THE DRAWINGS

FIGS. 1A-D is a schematic diagram of an exemplary scFc binding polypeptide of the invention. The binding polypeptide comprises an antigen-binding site (e.g. a Fab region) linked (e.g., by a human IgG1 hinge) to a genetically-fused Fc region (i.e., single chain Fc or “scFc” region) comprised of two Fc moeities linked via a polypeptide linker (FIG. 1A). The Fab region, human IgG1 hinge, and scFc region are all encoded in a single contiguous gene or genetic construct. Expression of the construct can result in both a dimeric form (“dc”; FIG. 1B) or a monomeric (“sc”; FIG. 1C) form of the scFc binding polypeptide. The domain organization of the heavy and light chains comprising the monomeric scFc are depicted in 1D.

FIGS. 2A-D show the results of a two-step purification process for separating monomeric (“sc”) and dimeric forms (“dc”) of scFc binding polypeptides. The purification process employs affinity chromatography followed by gel filtration chromatography. FIG. 2A shows the absorbance profile of fractions eluted from a Protein A affinity column at low pH. FIG. 2B shows the corresponding SDS PAGE analysis of those eluted fractions which contain both dimeric (“dc”) and monomeric (“sc”) forms of the binding polypeptide under non-reducing conditions. Both the monomeric and dimeric forms eluted essentially as a single peak from the protein A column. FIG. 2C shows that the size-exclusion chromatography of the pooled Protein A eluant on a Superdex 200 gel filtration column resolves this mixture into two distinct peaks. FIG. 2D shows the corresponding non-reducing SDS PAGE analysis of the gel filtration fractions. The peaks represent the purified monomeric (“sc”) and dimeric (“dc”) forms, respectively.

FIG. 3 shows an SDS-PAGE of purified dimeric (“dc”) and monomeric (“sc”) forms of the scFc binding polypeptide at a preparative scale under non-reducing (Panel A) and reducing (Panel B) conditions. For each panel, Lanes 1 and 2 contain the dimeric form (“ds”; 205 kDa) and monomeric (“sc”; 105 kDa) form, respectively. Lane 3 contains a control human IgG1 antibody (Hu5c8; 150 kDa).

FIGS. 4A-B show the characterization of complexes of the monomeric (sc) or dimeric (dc) scFc polypeptide bound to the homotrimeric shCD40L antigen. FIG. 4A shows a composite of the size exclusion chromatograms obtained for the SEC-LS experiments that were performed to determine the molecular weight of each individual component and the respective complexes formed. FIG. 4B shows a schematic of the predicted complexes formed upon binding of the monomeric (i) or dimeric (ii) scFc polypeptides to shCD40L, respectively, based on the individual masses obtained by SEC-LS and the respective calculated molecular weights of the complexes.

FIG. 5 shows a composite of the elution profiles of shCD40L containing complexes formed in the presence of either the monomeric (“sc”) scFc polypeptide or the conventional human IgG1 anti-CD40L mAb, hu5C8. Molecular weights were determined by on-line LS and are denoted above each peak obtained for the complexes. The molecular weights determined for monomeric scFc (“sc”), 5C8 IgG1, and shCD40L, are 101.5 kDa, 150 kDa, and 51 kDa, respectively.

FIG. 6 shows the results of an ELISA binding assay comparing the apparent binding affinities of the monomeric scFc (sc), dimeric scFc (dc) and a conventional human IgG1 anti-CD40L antibody (Hu 5C8) for the antigen, shCD40L coated on the plate. The monovalent scFc has an approximately 2-fold weaker EC50 vs. the WT mAb that has a significant avidity advantage due to it's ability to bind ligand bivalently.

FIG. 7A shows the results of an ELISA binding assay comparing the apparent FcRn binding affinity of the dimeric (“dc”) and monomeric (“sc”) forms of the scFc binding polyeptide, with that of the conventional IgG1 antibody (Hu 5C8). FcRn binding was determined using biotinylated forms of both a human and a rat FcRn-Fc fusion construct. In this assay each Fc containing construct was coated on the plate and binding of a biotinylated rat or human FcRn-Fc construct was detected with streptavidin HRP. The determined c-value for the binding of human FcRn-Fc (but not rat FcRn-Fc) to the monomeric scFc polypeptide (“sc”) was three- to four-fold lower then that of the Hu5C8 or dimeric (“dc”) scFc polypeptide. FIG. 7B shows the analytical SEC elution profiles for rat FcRn-Fc (“rFcRn-Fc”), the hemiglycosylated (“Hemigly scFc”) and fully glycosylated (“Fully gly scFc”) 5c8 scFcs and complexes formed upon the mixing of the rat FcRn-Fc with the scFc. Light scattering analysis was used to determine the respective molecular masses of the individual components and the complexes contained within the peaks. The masses determined for the complexes obtained indicate that both FcRn binding sites on each scFc are functional and are predicted to form a complex comprising 2 FcRnFc:2 scFc as depicted.

FIG. 8 depicts a molecular model of an exemplary monomeric form (sc) of a scFc binding polypeptide comprising two Fc moieties linked in tandem by a linker region. The model provides an example of a heteromeric scFc region. The scFc binding polypeptide contains a single site-specific Fc mutation which results in deglycosylation in one Fc moiety (“Fc moiety #2”) and glycosylation (“Sugar #1”) in the CH2 domain of the second Fc moiety (“Fc moiety #1”).

FIG. 9 depicts a rod diagram of the front and side views of the crystal structure of a scFc solved to a 3 Å resolution. Crystals were obtained for the scFc region in the absence of the F(ab) domains. The scFc is shown in grey superimposed on a fucosylated IgG1 Fc (pdb code 2DTQ; black). The superposition indicates a deviation of 0.489 A rmsd over 417 alpha-carbon atoms, which is essentially an identical backbone conformation. The only significant difference is that the scFc includes a partially ordered hinge region and an additional Galactose on one half of the scFc which is not present in the fucosylated IgG1 Fc structure. The scFc structure was solved to 3.0 Å resolution with an Rfree of 35% and an R-factor of 25%.

FIG. 10 depicts the advantage of using a scFc polypeptide of the invention in screening for bispecific antibody function. The scFc region prevents unwanted heterogeneous combinations of the binding domains. Such heterogeneity would result in complicating assays designed to screen for activities unique to bispecific antibodies. “Path 1” is an example of the heterogeneous binding domain combinations that would typically occur when three genes are coexpressed in a eukaryotic system to form a bispecific antibody: (A) a single chain F(ac), scF(ab), fused to the N-terminus of an Fc domain; (B) a F(ab) fragment fused to the C-terminus of the CH3 of an Fc; (C) the light chain comprising the CL and VL domains. “Path 2” depicts an example of how fusing (A) and (B) into a single, contiguous, genetic construct by means of an interposed linker sequence results in the two Fc moieties being genetically fused to form an scFc polypeptide (D). Coexpression of (C) and (D) results in the homogeonous expression of a single bispecific mAb.

FIGS. 11A-I are schematics of exemplary scFc binding polypeptides of the invention. FIG. 11A is a schematic of a scFc binding polypeptide comprising a binding site at the N-terminus. FIG. 11B is a schematic of a scFc binding polypeptide comprising a binding site at the C-terminus. FIG. 11C is a schematic of scFc binding polypeptide comprising a binding site in the N-terminal CH2-CH3 interdomain region. FIG. 11D is a schematic of a scFc binding polypeptide comprising a binding site in the C-terminal CH2-CH3 interdomain region. FIG. 11E is a schematic of a scFc binding polypeptide comprising a binding site in the linker polypeptide. FIG. 11F is a schematic of a scFc binding polypeptide comprising a binding site veneered onto an N-terminal CH2 domain. FIG. 11G is a schematic of a scFc binding polypeptide comprising a binding site veneered onto an N-terminal CH3 domain. FIG. 10H is a schematic of a scFc binding polypeptide comprising a binding site veneered onto a C-terminal CH2 domain. FIG. 11 is a schematic of a scFc binding polypeptide comprising a binding site veneered onto a C-terminal CH3 domain. It is recognized by those skilled in the art that a scFc binding polypeptide of the invention may comprise any combination of the features depicted in FIGS. 11A-I.

FIG. 12 shows a comparison of the protein expression profiles obtained for scFcs containing G4S linkers of 2 different lengths (1×G4S vs. 3×G4S, i.e., 5 vs. 15 amino acids). Linker length was found to correlate directly with scFc yield such that protein expressed from constructs comprising the longer linker yielded significantly greater amounts of scFc vs. dcFc.

FIG. 13 depicts serum concentrations of a 1×G4S and 3×G4S linked scFc polypeptides relative to a wild-type human IgG1 antibody (hu5c8) measured in rats over a 2 week time period. The 3×G4S scFc has a long β-phase half-life (12 days) that is similar to WT IgG1 (14 days).

FIGS. 14A and B depict the results of an Alphascreen assay performed to evaluate Fcγ-binding activity of scFc polypeptides. Binding of hemiglycosylated and fully glycosylated 5c8 scFcs to human and cynomolgus Fcγ receptors indicated was compared with WT and aglycosylated 5c8 IgG1. The binding of scFc and WT IgG1 to these receptors in vitro appears to be very similar suggesting that scFcs should similarly be able to engage Fcg receptors in vivo.

FIG. 15 shows the deconvoluted mass spectra obtained for the 3×G4S linked, hemiglycosylated scFc pre- and post PNGaseF treatment for deglycosylation of the protein. Spectra were generated for the scFc light chain before (FIG. 15A) and following (FIG. 15B) deglycosylation. The determined mass of the deglycosylated light chain is 23,854 Da. FIGS. 15C and D depect spectra of the scFc heavy chain before (FIG. 15C) and following (FIG. 15D) deglycosylation. The determined mass of the deglycosylated scFc heavy chain is 75,703 Da.

FIG. 16 shows the comparative thermal stabilities of the scFc molecules compared to WT huIgG1 mAb and Fc as measured by differential scanning calorimetry (DSC). The stability of the CH2 domain of the fully glycosylated scFc (ASK048) is similar to WT IgG1. The hemiglycosylated scFc has somewhat lower stability most likely due to increased domain flexibility being contributed by the aglycosylated CH2 of the second Fc moiety.

FIG. 17 depicts the heavy chain amino acid sequence of an (G4S) 1-linked hemiglycosylated, 5C8 scFc IgG1 antibody construct (pEAG2066; SEQ ID NO: 1). The construct has the general structure VH-CH1-Hinge Domain-CH2(1)-CH3(1)-G4S linker-Hinge Domain-CH2(2)-CH3(2), Whereas the first, more N-terminal Fc moiety (residues 222-447, SEQ ID NO:2) is wild-type with respect to its glycosylation pattern, the second, more C-terminal, Fc moiety (residues 458-683, SEQ ID NO:3) contains an amino acid substitution (T299A, EU numbering) that produces an aglycosylated Fc. In addition, the scFc contains a C220S substitution (EU numbering) in a hinge domain. The location of the T299A and C220S mutations are indicated in bold. The hinge domains are italicized and the CH1 and CH3 constant domains are underlined in the sequence.

FIG. 18 depicts the nucleotide sequence (SEQ ID NO:4) corresponding to the heavy chain sequence of pEAG2066 in FIG. 17. The antibody signal sequence is underlined.

FIG. 19A depicts the light chain amino acid sequence of an exemplary 5C8 antibody construct (pEAG2027; SEQ ID NO:5). FIG. 19B depicts the corresponding nucleotide sequence (SEQ ID NO:6). The antibody signal sequence is underlined.

FIG. 20 depicts the heavy chain amino acid sequence (pEAG2146; SEQ ID NO:7) of an exemplary fully glycosylated, 1×G4S-linked, 5C8 IgG1 scFc antibody construct comprising a homomeric, scFc region in which both the N-terminal (residues 22-447) and C-terminal (residues 458-683) Fc moieties are glycosylated. The component Fc moieties of the construct are annotated as in FIG. 17.

FIG. 21 depicts the nucleotide sequence (SEQ ID NO:8) corresponding to the heavy chain sequence of pEAG2146 in FIG. 17. The antibody signal sequence is underlined.

FIG. 22 depicts the heavy chain amino acid sequence (pEAG2147; SEQ ID NO:9) of an exemplary scFc hu5C8 IgG1 antibody construct comprising a hemiglycosylated, 1×G4S-linked, scFc region wherein the second, more C-terminal, Fc moiety (residues 458-683; SEQ ID NO: 10) comprises an altered hinge domain (GSEPKSSDKTHTSPPSPAPELLGGPSVFLF, SEQ ID NO:11), wherein the hinge cysteine residues have been substituted by serines. The sequence is annotated as in FIG. 17.

FIG. 23 depicts the nucleotide sequence (SEQ ID NO: 12) corresponding to the heavy chain sequence of pEAG2147 in FIG. 22. The antibody signal sequence is underlined.

FIG. 24 depicts the heavy chain amino acid sequence (pASK043; SEQ ID NO: 13) of an exemplary scFc antibody construct comprising a hemiglycosyated, (G4S)₃ linked scFc region in the context of the human IgG1 mAb, 5C8. The component domains of the construct are annotated in the figure by separate sequence identifiers.

FIG. 25 depicts the nucleotide sequence (SEQ ID NO: 14) corresponding to the heavy chain sequence of pASK043 in FIG. 24. The antibody signal sequence is underlined.

FIG. 26 depicts the heavy chain amino acid sequence (ASK048; SEQ ID NO: 15) of an exemplary scFc 5C8 IgG1 antibody construct comprising a fully glycosyated, (G4S)₃ linked scFc region in which both Fc moieties are glycosylated. The component domains of the construct are annotated in the figure by separate sequence identifiers.

FIG. 27 depicts the nucleotide sequence (SEQ ID NO: 16) corresponding to the heavy chain sequence of ASK048 in FIG. 26. The antibody signal sequence is underlined.

FIG. 28 depicts the heavy chain amino acid sequence (ASK052; SEQ ID NO:17) of an exemplary scFc 5C8 IgG1 antibody construct comprising an aglycosylated, (G4S)₃ linked scFc region. The component domains of the construct are annotated in the figure by separate sequence identifiers.

FIG. 29 depicts the nucleotide sequence (SEQ ID NO: 18) corresponding to the heavy chain sequence of ASK052 in FIG. 29. The antibody signal sequence is underlined.

FIG. 30 depicts the heavy chain amino acid sequence (pASK053; SEQ ID NO: 19) of an exemplary scFc 5C8 IgG1 antibody construct comprising an aglycosylated, (G4S)₁ linked scFc region. The component domains of the construct are annotated in the figure by separate sequence identifiers.

FIG. 31 depicts the nucleotide sequence (SEQ ID NO:20) corresponding to the heavy chain sequence of pASK053 in FIG. 30. The antibody signal sequence is underlined.

FIG. 32 depicts the heavy chain amino acid sequence of an exemplary anti-LINGO scFc antibody construct (pEAG2148; SEQ ID NO:21) comprising a hemiglycosylated, 1×G4S-linked, scFc region in the context of the human, anti-LINGO IgG1 mAb, Li33. Whereas the first, more N-terminal Fc moiety (residues 223-448) is wild-type with respect to its glycosylation pattern, the second, more C-terminal Fc moiety (residues 549-684) contains an amino acid substitution that produces an aglycosylated Fc. The component domains of the construct are annotated as in FIG. 17.

FIG. 33 depicts the nucleotide sequence (SEQ ID NO:22) corresponding to the heavy chain sequence of pEAG2148 in FIG. 32. The antibody signal sequence is underlined.

FIG. 34A depicts the light chain amino acid sequence of the exemplary Li33 scFc antibody construct (pXW435; SEQ ID NO:23). FIG. 34B depicts the corresponding nucleotide sequence (SEQ ID NO:24). The antibody signal sequence is underlined.

FIG. 35 depicts the heavy chain amino acid sequence of an exemplary anti-LINGO scFc antibody construct (ASK050; SEQ ID NO:25) comprising an aglycosylated, 3×G4S-linked, scFc region in the context of the human, anti-LINGO IgG1 mAb, Li33.

FIG. 36 depicts the nucleotide sequence (SEQ ID NO:26) corresponding to the heavy chain sequence of ASK050 in FIG. 35. The antibody signal sequence is underlined.

FIG. 37 depicts the heavy chain amino acid sequence of an exemplary anti-LINGO scFc antibody construct (ASK051; SEQ ID NO:27) comprising an aglycosylated, 1×G4S-linked, scFc region in the context of the human, anti-LINGO IgG1 mAb, Li33.

FIG. 38 depicts the nucleotide sequence (SEQ ID NO:28) corresponding to the heavy chain sequence of ASK051 in FIG. 37. The antibody signal sequence is underlined.

FIG. 39 shows the improved protein concentration dependent solubility characteristics of the anti-LINGO, scFc antibody molecule (EAG2148).

FIG. 40A depicts the heavy chain amino acid sequence of an exemplary anti-CD2, chimeric CB6 scFc IgG1 antibody construct (ASK058; SEQ ID NO:29) comprising a fully glycosylated, 3×G4S-linked, scFc region in the context of the anti-CD2, chimeric IgG1 mAb, CB6. FIG. 40B depicts the light chain amino acid sequence of the CB6 scFc IgG1 antibody construct (EAG2276; SEQ ID NO:56).

FIG. 41 depicts the nucleotide sequence (SEQ ID NO:30) corresponding to the heavy chain sequence of ASK058 in FIG. 40. The antibody signal sequence is underlined. FIG. 41b depicts the nucleotide sequence (SEQ ID NO:) corresponding to the heavy chain sequence of EAG2276 in FIG. 40B.

FIG. 42 depicts the heavy chain amino acid sequence of an exemplary anti-CD2, chimeric CB6 scFc IgG1 antibody construct (ASK062; SEQ ID NO:31) comprising a fully glycosylated, 4×G4S-linked, scFc region in the context of the anti-CD2, chimeric IgG1 mAb, CB6.

FIG. 43 depicts the nucleotide sequence (SEQ ID NO:32) corresponding to the heavy chain sequence of ASK062 in FIG. 42. The antibody signal sequence is underlined.

FIG. 44 depicts the heavy chain amino acid sequence of an exemplary anti-CD2, chimeric CB6 scFc IgG1 antibody construct (ASK063; SEQ ID NO:33) comprising a fully glycosylated, 5×G4S-linked, scFc region in the context of the anti-CD2, chimeric IgG1 mAb, CB6.

FIG. 45 depicts the nucleotide sequence (SEQ ID NO:34) corresponding to the heavy chain sequence of ASK063 in FIG. 44. The antibody signal sequence is underlined.

FIG. 46 depicts the heavy chain amino acid sequence of an exemplary anti-CD2, chimeric CB6 scFc IgG1 antibody construct (ASK064; SEQ ID NO:35) comprising a fully glycosylated, 6×G4S-linked, scFc region in the context of the anti-CD2, chimeric IgG1 mAb, CB6.

FIG. 47 depicts the nucleotide sequence (SEQ ID NO:36) corresponding to the heavy chain sequence of ASK064 in FIG. 46. The antibody signal sequence is underlined.

FIG. 48 depicts the amino acid sequence an exemplary GFRα3 immunoadhesin protein (ASK-057; SEQ ID NO:37) comprising a (G4S)₃-linked, fully glycosylated, scFc region fused to the extracellular domain of the neublastin receptor GFRα3.

FIG. 49 depicts the nucleotide sequence (SEQ ID NO:38) of corresponding to the amino acid sequence of ASK-057 in FIG. 48. The signal sequence is underlined.

FIG. 50 shows the non-reducing SDS-PAGE and analytical SEC-LS characterization of the GFRα3:scFc fusion protein obtained after 2-step purification.

FIG. 51 depicts the amino acid sequence of an exemplary Interferon-β immunoadhesin construct (pEAG2149; SEQ ID NO:39) comprising a hemiglycosylated, 1×G4S-linked, scFc region fused to Interferon-β (residues 1-67). Whereas the first, more N-terminal, Fc moiety (residues 168-393) is wild-type with respect to its glycosylation pattern, the second, more C-terminal, Fc moiety (residues 404-629) contains an amino acid substitution that produces an aglycosylated Fc. The component domains of the construct are annotated as in FIG. 17.

FIG. 52 depicts the nucleotide sequence (SEQ ID NO:40) corresponding to sequence of pEAG2149 in FIG. 52. The signal sequence is underlined.

FIG. 53 depicts the amino acid sequence of an exemplary LTβR immunoadhesin construct (EAG2190; SEQ ID NO:41) comprising a hemiglycosylated, 3×G4S-linked, scFc region fused to LTβR.

FIG. 54 depicts the nucleotide sequence (SEQ ID NO:42) corresponding to sequence of EAG2190 in FIG. 54. The signal sequence is underlined.

FIG. 55 depicts the amino acid sequence of an exemplary LTβR immunoadhesin construct (EAG2191; SEQ ID NO:43) comprising a hemiglycosylated, 3×G4S-linked, scFc region fused to LTβR.

FIG. 56 depicts the nucleotide sequence (SEQ ID NO:44) corresponding to sequence of EAG2191 in FIG. 56. The signal sequence is underlined.

FIG. 57 depicts the characterization of LTβR:scFc fusion polypeptide (EAG2190) by SDS-PAGE (FIG. 58A) and analytical gel filtration (FIG. 58B). In Lanes 1 and 2 in FIG. 58A are nonreducing and contain 1 and 2 ug protein, while lanes 4 and 5 contain reductant and 2 and 1 ugs of the LI33 scFc respectively. Lane 3 contains the molecular weight standards with the mass of the relevant standards are indicated.

FIG. 58 depicts mass spectrometry (MS) of N-deglycosylated reduced LTβR:scFc.

FIG. 59 depicts the results of an ELISA (FIG. 59A) and FACS analysis (FIG. 59B) evaluating the binding affinity of the monomeric LTβR:scFc to LTa1β2.

FIG. 60 depicts the amino acid (FIG. 60A; SEQ ID NO:45) and nucleotide (FIG. 60B; SEQ ID NO:46) sequences of an exemplary hemiglycosylated, 1×G4S-linked, scFc region (EAG2181) of the invention. The N and/or C-terminus of said scFc region may be fused to any art-recognized binding site.

FIG. 61 depicts the amino acid (FIG. 61A; SEQ ID NO:47) and nucleotide (FIG. 61B; SEQ ID NO:48) sequences of an exemplary fully glycosylated, 3×G4S-linked, scFc region (ASK054) of the invention. The N and/or C-terminus of said scFc region may be fused to any art-recognized binding site.

FIG. 62 depicts the amino acid (FIG. 62A; SEQ ID NO:49) and nucleotide (FIG. 62B; SEQ ID NO:50) sequences of an exemplary fully glycosylated, 1×G4S-linked, scFc region (ASK055) of the invention. The N and/or C-terminus of said scFc region may be fused to any art-recognized binding site.

FIG. 63 depicts the amino acid (FIG. 63A; SEQ ID NO:51) and nucleotide (FIG. 63B; SEQ ID NO:52) sequences of the heavy chain (ASK016) of an exemplary anti-LTβR antibody (BDA8). In certain embodiments, a scFc binding polypeptide of the invention comprises a binding site of BDA8.

FIG. 64 depicts a list of FDA-approved antibodies or other antibodies. In certain embodiments, the scFc binding polypeptides of the invention may comprise an antigen binding site derived from one the depicted antibodies.

### DETAILED DESCRIPTION OF THE INVENTION

The present invention advances the art by providing, e.g., binding polypeptides comprising, e.g., (i) at least one binding site or binding domain; and (ii) at least one genetically fused Fc region (i.e., single-chain Fc (“scFc”) region). In preferred embodiments, the scFc region comprises at least two Fc moieties which are genetically fused via a linker polypeptide (e.g., an Fc connecting peptide) interposed between said Fc moieties). In one embodiment, the binding site may comprise a antigen binding fragment of an antibody molecule (e.g., F(ab) or scFv) which is fused (e.g., via either the VH or VL of the Fab or scFv) to either or both N- and C-termini of the scFc region. In another embodiment, the binding domain may comprise a receptor fusion protein fused to either or both N- and C-termini of the genetically fused Fc region. Such fusions can be made either C-terminally or N-terminally to the desired binding site. Expression of the binding polypeptides of the invention from a single contiguous genetic construct has numerous advantages over conventional protein expression methods which involve the co-expression of two genes, one expressing a first Fc domain and a separate second gene consisting of a binding site fused to a second Fc domain with disulfide bonds linking the two polypeptide chains. The problems of such conventional constructs include significant heterogeneity within the population of resulting molecules, such that the desired molecule must be purified away from undesired molecules, thereby resulting in a decline in total yield of the desired molecule. The advantages of the polypeptides of the invention compared to other molecules which employ traditional Fc regions or which lack such regions are discussed below using antibody molecules or exemplary fragments thereof to illustrate:

Lack of scrambling upon expression of a binding domain fused to a scFc: It is difficult to construct antibodies with only one F(ab) arm, or Fc fusion proteins with only one fused functional polypeptide. Current methods require the coexpression of two genes: one encoding one heavy chain of an Ab including a first Fc moiety (Fc1 e.g., VH, CH1, hinge, CH2, and CH3 domains) and a second gene encoding a second Fc moiety (Fc2 e.g., hinge, CH2, and CH3 domains) to obtain the desired molecule. Co-expression leads to an undesirably complex mixture of molecules predicted to be in a 1:2:1 mixture of Fc1+Fc1: Fc1+Fc2: Fc2+Fc2, but the ratios can vary greatly from this theoretical prediction resulting in suboptimal yields of the desired protein. Expression of monovalent fusion proteins with a single fusion molecule linked to a conventional, dimeric, Fc can be especially important in preventing inappropriate folding events that can occur when two identical molecules are folding in close proximity. These misfolded Fc fusion proteins can be difficult to separate from the properly folded, bivalent, Fc protein since the only difference between the two is often a heterogenous misfolding event. The subject scFc fusion proteins cannot undergo scrambling of the protein domains because this construction does not fix the molecules in close proximity to each other during the folding process.

Enhancement of antibody fragment (e.g., F(ab)) half-life via addition of FcRn binding: The therapeutic application of antibody fragments (e.g., F(ab)s) is often desirable because it enables blocking of cell surface receptors without target receptor crosslinking and, thus, without subsequent undesirable signaling such as can occur upon receptor engagement by a bivalent antibody. Such crosslinking of surface receptors can cause clustering of receptors and down regulation of the target receptor from the surface of the cell. A F(ab) construct is inherently monovalent and thus cannot cause receptor cross-linking or clustering.

One of the significant drawbacks to the application of antibody fragments such as F(ab)s in vivo is their poor serum persistence or half-life. The addition of an Fc region to a F(ab) fragment results in pharmacokinetic half-life similar to an intact mAb. Typically the half-life of F(ab)s is elongated by the chemical addition of a PEG moiety to a specific thiol after preparation and purification of the F(ab). The PEGylation reaction adds a significant complication to the preparation of the product. The PEGylation chemistry has to be optimized for each F(ab) and decreases the product yield. The pegylation also complicates the final product analysis since PEGylated materials are of heterogenous molecular weight.

Addition of FcγRI, FcγRII and FcγRIII functionalities to an antibody fragment (e.g., an F(ab)): Antibody fragments such as F(ab)s and pegylated F(ab)s lack the ability to interact with FcγRI, FcγII and FcγII. Engagement of Fc receptors is desirable in certain circumstances. For example the anti-CD20 antibody depends on the Fc functionality for the ADCC dependent depletion of unwanted cancerous B-cells. In addition, monovalent F(ab)s are preferred over bivalent mAbs in cases where the crosslinking of receptors by an antibody would lead to receptor internalization. Such internalization may well be undesirable if the efficacy of the drug is dependent upon an Fc dependent ADCC depletion mechanism. A F(ab) fragment linked to a scFc polypeptide of the invention therefore represents an optimal construct because it embodies the desired characteristics of monovalency and Fcγ receptor engagement.

ScFc molecules allow for production of heteromeric Fc regions. Site-specific mutations within Fc regions have been useful in creating Fc-variant mAbs with improved Fc functionality. Examples are mutations that enhance the affinity to, e.g., various FcγRI, FcγRII and/or FcγRIII. The single chain Fc (scFc) molecules of the invention can be used to create a heteromeric scFc region containing a specific point mutation in only one Fc moiety or different combinations of point mutations in both moieties of the scFc region. An example would be the expression of scFc construct containing Asn 297 glycosylation in only one of two Fc moieties. This molecule shows somewhat decreased FcR affinity, but is not inactive in FcγRIII binding.

In order to provide a clear understanding of the specification and claims, the following definitions are conveniently provided below.

### I. DEFINITIONS

As used herein, the term “polypeptide” refers to a polymer of two or more of the natural amino acids or non-natural amino acids.

The term “amino acid” includes alanine (Ala or A); arginine (Arg or R); aspar-agine (Asn or N); aspartic acid (Asp or D); cysteine (Cys or C); glutamine (Gln or Q); glutamic acid (Glu or E); glycine (Gly or G); histidine (H is or H); isoleucine (Ile or I): leucine (Leu or L); lysine (Lys or K); methionine (Met or M); phenylalanine (Phe or F); proline (Pro or P); serine (Ser or S); threonine (Thr or T); tryptophan (Trp or W); tyrosine (Tyr or Y); and valine (Val or V). Non-traditional amino acids are also within the scope of the invention and include norleucine, ornithine, norvaline, homoserine, and other amino acid residue analogues such as those described in Ellman et al. Meth. Enzym. 202:301-336 (1991). To generate such non-naturally occurring amino acid residues, the procedures of Noren et al. Science 244:182 (1989) and Ellman et al., supra, can be used. Briefly, these procedures involve chemically activating a suppressor tRNA with a non-naturally occurring amino acid residue followed by in vitro transcription and translation of the RNA. Introduction of the non-traditional amino acid can also be achieved using peptide chemistries known in the art. As used herein, the term “polar amino acid” includes amino acids that have net zero charge, but have non-zero partial charges in different portions of their side chains (e.g. M, F, W, S, Y, N, Q, C). These amino acids can participate in hydrophobic interactions and electrostatic interactions.

As used herein, the term “charged amino acid” include amino acids that can have non-zero net charge on their side chains (e.g. R, K, H, E, D). These amino acids can participate in hydrophobic interactions and electrostatic interactions. As used herein the term “amino acids with sufficient steric bulk” includes those amino acids having side chains which occupy larger 3 dimensional space. Exemplary amino acids having side chain chemistries of sufficient steric bulk include tyrosine, tryptophan, arginine, lysine, histidine, glutamic acid, glutamine, and methionine, or analogs or mimetics thereof.

An “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, the present larger “peptide insertions”, can be made, e.g. insertion of about three to about five or even up to about ten, fifteen, or twenty amino acid residues. The inserted residue(s) may be naturally occurring or non-naturally occurring as disclosed above. An “amino acid deletion” refers to the removal of at least one amino acid residue from a predetermined amino acid sequence.

As used herein, the term “protein” refers to a polypeptide or a composition comprising more than one polypeptide. Accordingly, proteins may be either monomers or multimers. For example, in one embodiment, a binding protein of the invention is a dimer. In one embodiment, the dimers of the invention are homodimers, comprising two identical monomeric subunits or polypeptides (e.g., two identical scFc polypeptides). In another embodiment, the dimers of the invention are heterodimers, comprising two non-identical monomeric subunits or polypeptides (e.g., two non-identical scFc polypeptides). The subunits of the dimer may comprise one or more polypeptide chains (e.g., target binding chains comprising an scFc molecule). For example, in one embodiment, the dimers comprise at least two polypeptide chains (e.g, at least two scFc polypeptide chains). In one embodiment, the dimers comprise two polypeptide chains.

In another embodiment, the dimers comprise three polypeptide chains. In another embodiment, the dimers comprise four polypeptide chains.

In certain preferred embodiments, the polypeptides of the invention are scFc polypeptides. As used herein, the term scFc polypeptide refers to a polypeptide comprising a single-chain Fc (scFc) region.

In other preferred embodiments, the polypeptides of the invention are binding polypeptides. As used herein, the term “binding polypeptide” refers to polypeptides that comprise at least one target binding site or binding domain that specifically binds to a target molecule (such as an antigen or binding partner). For example, in one embodiment, a binding polypeptide of the invention comprises an immunoglobulin antigen binding site or the portion of a receptor molecule responsible for ligand binding or the portion of a ligand molecule that is responsible for receptor binding. The binding polypeptides of the invention preferably also comprise at least two Fc moieties derived from one or more immunoglobulin (Ig) molecules. For example, in preferred embodiments, the binding polypeptide is a scFc polypeptide comprising at least two Fc moieties that are genetically fused. In one embodiment a binding polypeptide of the invention comprises additional modifications. Exemplary modifications are described in more detail below. For example, in certain preferred embodiments, a polypeptide of the invention may optionally comprise a flexible polypeptide linker interposed between at least two Fc moieties of a genetically fused Fc region (i.e., a scFc region). In another embodiment, a binding polypeptide may be modified to add a functional moiety (e.g., PEG, a drug, or a label).

The binding polypeptides of the invention comprise at least one binding site. In one embodiment, the binding polypeptides of the invention comprise at least two binding sites. In one embodiment, the binding polypeptides comprise two binding sites.

In another embodiment, the binding polypeptides comprise three binding sites. In another embodiment, the binding polypeptides comprise four binding sites. In one embodiment, the binding sites are linked to each other in tandem. In other embodiments, the binding sites are located at different positions of the binding polypeptide. For example, one or more binding sites may be linked to either one or both ends of a genetically fused Fc region (i.e., a single-chain Fc (scFc) region).

The terms “binding domain” or “binding site”, as used herein, shall refer to the portion, region, or site of binding polypeptide that mediates specific binding with a target molecule (e.g. an antigen, ligand, receptor, substrate or inhibitor). Exemplary binding domains include an antigen binding site, a receptor binding domain of a ligand, a ligand binding domain of a receptor or an enzymatic domain. The term “ligand binding domain” as used herein refers to a native receptor (e.g., cell surface receptor) or a region or derivative thereof retaining at least a qualitative ligand binding ability, and preferably the biological activity of the corresponding native receptor. The term “receptor binding domain” as used herein refers to a native ligand or region or derivative thereof retaining at least a qualitative receptor binding ability, and preferably the biological activity of the corresponding native ligand. In one embodiment, the binding polypeptides of the invention have at least one binding domain specific for a molecule targeted for reduction or elimination, e.g., a cell surface antigen or a soluble antigen. In preferred embodiments, the binding domain comprises or consists of an antigen binding site (e.g., comprising a variable heavy chain sequence and variable light chain sequence or six CDRs from an antibody placed into alternative framework regions (e.g., human framework regions optionally comprising one or more amino acid substitutions).

The term “binding affinity”, as used herein, includes the strength of a binding interaction and therefore includes both the actual binding affinity as well as the apparent binding affinity. The actual binding affinity is a ratio of the association rate over the disassociation rate. Therefore, conferring or optimizing binding affinity includes altering either or both of these components to achieve the desired level of binding affinity. The apparent affinity can include, for example, the avidity of the interaction.

The term “binding free energy” or “free energy of binding”, as used herein, includes its art-recognized meaning, and, in particular, as applied to binding site-ligand or Fc-FcR interactions in a solvent. Reductions in binding free energy enhance affinities, whereas increases in binding free energy reduce affinities.

The term “specificity” includes the number of potential binding sites which specifically bind (e.g., immunoreact with) a given target. A binding polypeptide may be monospecific and contain one or more binding sites which specifically bind the same target (e.g., the same epitope) or the binding polypeptide may be multispecific and contain two or more binding sites which specifically bind different regions of the same target (e.g., different epitopes) or different targets. In one embodiment, multispecific binding polypeptide (e.g., a bispecific polypeptide) having binding specificity for more than one target molecule (e.g., more than one antigen or more than one epitope on the same antigen) can be made. In another embodiment, the multispecific binding polypeptide has at least one binding domain specific for a molecule targeted for reduction or elimination and at least one binding domain specific for a target molecule on a cell. In another embodiment, the multispecific binding polypeptide has at least one binding domain specific for a molecule targeted for reduction or elimination and at least one binding domain specific for a drug. In yet another embodiment, the multispecific binding polypeptide has at least one binding domain specific for a molecule targeted for reduction or elimination and at least one binding domain specific for a prodrug. In yet another embodiment, the multispecific binding polypeptides are tetravalent antibodies that have two binding domains specific for one target molecule and two binding sites specific for the second target molecule.

As used herein the term “valency” refers to the number of potential binding domains in a binding polypeptide or protein. Each binding domain specifically binds one target molecule. When a binding polypeptide comprises more than one binding domain, each binding domain may specifically bind the same or different molecules (e.g., may bind to different ligands or different antigens, or different epitopes on the same antigen). In one embodiment, the binding polypeptides of the invention are monovalent. In another embodiment, the binding polypeptides of the invention are multivalent. In another embodiment, the binding polypeptides of the invention are bivalent. In another embodiment, the binding polyeptides of the invention are trivalent. In yet another embodiment, the binding polypeptides of the invention are tetravalent.

In certain aspects, the binding polypeptides of invention employ polypeptide linkers. As used herein, the term “polypeptide linkers” refers to a peptide or polypeptide sequence (e.g., a synthetic peptide or polypeptide sequence) which connects two domains in a linear amino acid sequence of a polypeptide chain. For example, polypeptide linkers may be used to connect a binding site to a genetically fused Fc region. Preferably, such polypeptide linkers provide flexibility to the polypeptide molecule. For example, in one embodiment, a VH domain or VL domain is fused or linked to a genetically fused Fc region (i.e., scFc region) via a polypeptide linker (the N- or C-terminus of the polypeptide linker is attached to the C- or N-terminus of the genetically fused Fc region and the N-terminus of the polypeptide linker is attached to the N- or C-terminus of the VH or VL domain).

In certain embodiments the polypeptide linker is used to connect (e.g., genetically fuse) two Fc moieties or domains. Such polypeptide linkers are also referred to herein as Fc connecting polypeptides. As used herein, the term “Fc connecting polypeptide” refers specifically to a linking polypeptide which connects (e.g., genetically fuses) two Fc moieties or domains.

A binding molecule of the invention may comprise more than one peptide linker.

As used herein the term “properly folded polypeptide” includes polypeptides (e.g., binding polypeptides of the invention) in which all of the functional domains comprising the polypeptide are distinctly active. As used herein, the term “improperly folded polypeptide” includes polypeptides in which at least one of the functional domains of the polypeptide is not active. As used herein, a “properly folded Fc polypeptide” or “properly folded Fc region” comprises a genetically-fused Fc region (i.e., scFc region) in which at least two component Fc moieties are properly folded such that the resulting scFc region comprises at least one effector function.

A polypeptide or amino acid sequence “derived from” a designated polypeptide or protein refers to the origin of the polypeptide. Preferably, the polypeptide or amino acid sequence which is derived from a particular sequence has an amino acid sequence that is essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the sequence.

Polypeptides derived from another peptide may have one or more mutations relative to the starting polypeptide, e.g., one or more amino acid residues which have been substituted with another amino acid residue or which has one or more amino acid residue insertions or deletions. Preferably, the polypeptide comprises an amino acid sequence which is not naturally occurring. Such variants necessarily have less than 100% sequence identity or similarity with the starting antibody. In a preferred embodiment, the variant will have an amino acid sequence from about 75% to less than 100% amino acid sequence identity or similarity with the amino acid sequence of the starting polypeptide, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and most preferably from about 95% to less than 100%, e.g., over the length of the variant molecule.

In one embodiment, there is one amino acid difference between a starting polypeptide sequence and the sequence derived therefrom. Identity or similarity with respect to this sequence is defined herein as the percentage of amino acid residues in the candidate sequence that are identical (i.e. same residue) with the starting amino acid residues, after aligning the sequences and introducing gaps, if necessary, to achieve the maximum percent sequence identity.

Preferred binding polypeptides of the invention comprise an amino acid sequence (e.g., at least one Fc moiety or domain) derived from a human immunoglobulin sequence. However, polypeptides may comprise one or more amino acids from another mammalian species. For example, a primate Fc domain or binding site may be included in the subject polypeptides. Alternatively, one or more murine amino acids may be present in a polypeptide. Preferred polypeptides of the invention are not immunogenic.

It will also be understood by one of ordinary skill in the art that the binding polypeptides of the invention may be altered such that they vary in amino acid sequence from the naturally occurring or native polypeptides from which they were derived, while retaining the desirable activity of the native polypeptides. For example, nucleotide or amino acid substitutions leading to conservative substitutions or changes at “non-essential” amino acid residues may be made. An isolated nucleic acid molecule encoding a non-natural variant of a binding polypeptide derived from an immunoglobulin (e.g., an Fc domain, moiety, or antigen binding site) can be created by introducing one or more nucleotide substitutions, additions or deletions into the nucleotide sequence of the immunoglobulin such that one or more amino acid substitutions, additions or deletions are introduced into the encoded protein. Mutations may be introduced by standard techniques, such as site-directed mutagenesis and PCR-mediated mutagenesis.

The binding polypeptides of the invention may comprise conservative amino acid substitutions at one or more amino acid residues, e.g., at essential or non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in a binding polypeptide is preferably replaced with another amino acid residue from the same side chain family. In another embodiment, a string of amino acids can be replaced with a structurally similar string that differs in order and/or composition of side chain family members. Alternatively, in another embodiment, mutations may be introduced randomly along all or part of a coding sequence, such as by saturation mutagenesis, and the resultant mutants can be incorporated into binding polypeptides of the invention and screened for their ability to bind to the desired target.

In the context of polypeptides, a “linear sequence” or a “sequence” is the order of amino acids in a polypeptide in an amino to carboxyl terminal direction in which residues that neighbor each other in the sequence are contiguous in the primary structure of the polypeptide.

As used herein, the terms “linked,” “fused”, or “fusion”, are used interchangeably. These terms refer to the joining together of two more elements or components, by whatever means including chemical conjugation or recombinant means. Methods of chemical conjugation (e.g., using heterobifunctional crosslinking agents) are known in the art.

As used herein, the term “genetically fused” or “genetic fusion” refers to the co-linear, covalent linkage or attachment of two or more proteins, polypeptides, or fragments thereof via their individual peptide backbones, through genetic expression of a single polynucleotide molecule encoding those proteins, polypeptides, or fragments. Such genetic fusion results in the expression of a single contiguous genetic sequence. Preferred genetic fusions are in frame, i.e., two or more open reading frames (ORFs) are fused to form a continuous longer ORF, in a manner that maintains the correct reading frame of the original ORFs. Thus, the resulting recombinant fusion protein is a single polypeptide containing two or more protein segments that correspond to polypeptides encoded by the original ORFs (which segments are not normally so joined in nature). Although the reading frame is thus made continuous throughout the fused genetic segments, the protein segments may be physically or spatially separated by, for example, an in-frame polypeptide linker.

As used herein, the term “Fc region” shall be defined as the portion of a native immunoglobulin formed by the respective Fc domains (or Fc moieties) of its two heavy chains. A native Fc region is homodimeric. In contrast, the term “genetically-fused Fc region” or “single-chain Fc region” (scFc region), as used herein, refers to a synthetic Fc region comprised of Fc domains (or Fc moieties) genetically linked within a single polypeptide chain (i.e., encoded in a single contiguous genetic sequence). Accordingly, a genetically-fused Fc region (i.e., a scFc region) is monomeric.

As used herein, the term “Fc domain” refers to the portion of a single immunoglobulin heavy chain beginning in the hinge region just upstream of the papain cleavage site (i.e. residue 216 in IgG, taking the first residue of heavy chain constant region to be 114) and ending at the C-terminus of the antibody. Accordingly, a complete Fc domain comprises at least a hinge domain, a CH2 domain, and a CH3 domain.

As used herein, the term “Fc domain portion” or “Fc moiety” includes an amino acid sequence of an Fc domain or derived from an Fc domain. In certain embodiments, an Fc moiety comprises at least one of: a hinge (e.g., upper, middle, and/or lower hinge region) domain, a CH2 domain, a CH3 domain, a CH4 domain, or a variant, portion, or fragment thereof. In other embodiments, an Fc moiety comprises a complete Fc domain (i.e., a hinge domain, a CH2 domain, and a CH3 domain). In one embodiment, a Fc moiety comprises a hinge domain (or portion thereof) fused to a CH3 domain (or portion thereof). In another embodiment, a Fc moiety comprises a CH2 domain (or portion thereof) fused to a CH3 domain (or portion thereof). In another embodiment, a Fc moiety consists of a CH3 domain or portion thereof. In another embodiment, a Fc moiety consists of a hinge domain (or portion thereof) and a CH3 domain (or portion thereof). In another embodiment, a Fc moiety consists of a CH2 domain (or portion thereof) and a CH3 domain. In another embodiment, a Fc moiety consists of a hinge domain (or portion thereof) and a CH2 domain (or portion thereof). In one embodiment, an Fc moiety lacks at least a portion of a CH2 domain (e.g., all or part of a CH2 domain). In one embodiment, an Fc region of the invention (an scFc region) comprises at least the portion of an Fc molecule known in the art to be required for FcRn binding. In another embodiment, an Fc region of the invention (an scFc region) comprises at least the portion of an Fc molecule known in the art to be required for FcγR binding. In one embodiment, an Fc region of the invention (an scFc region) comprises at least the portion of an Fc molecule known in the art to be required for Protein A binding. In one embodiment, an Fc region of the invention (an scFc region) comprises at least the portion of an Fc molecule known in the art to be required for protein G binding.

As set forth herein, it will be understood by one of ordinary skill in the art that any Fc domain may be modified such that it varies in amino acid sequence from the native Fc domain of a naturally occurring immunoglobulin molecule. In certain exemplary embodiments, the Fc moiety retains an effector function (e.g., FcγR binding).

The Fc domains or moeities of a polypeptide of the invention may be derived from different immunoglobulin molecules. For example, an Fc domain or moiety of a polypeptide may comprise a CH2 and/or CH3 domain derived from an IgG1 molecule and a hinge region derived from an IgG3 molecule. In another example, an Fc domain or moiety can comprise a chimeric hinge region derived, in part, from an IgG1 molecule and, in part, from an IgG3 molecule. In another example, an Fc domain or moiety can comprise a chimeric hinge derived, in part, from an IgG1 molecule and, in part, from an IgG4 molecule.

As used herein, the term “immunoglobulin” includes a polypeptide having a combination of two heavy and two light chains whether or not it possesses any relevant specific immunoreactivity. As used herein, the term “antibody” refers to such assemblies (e.g., intact antibody molecules, antibody fragments, or variants thereof) which have significant known specific immunoreactive activity to an antigen of interest (e.g. a tumor associated antigen). Antibodies and immunoglobulins comprise light and heavy chains, with or without an interchain covalent linkage between them. Basic immunoglobulin structures in vertebrate systems are relatively well understood.

As will be discussed in more detail below, the generic term “antibody” includes five distinct classes of antibody that can be distinguished biochemically. Fc moieties from each class of antibodies are clearly within the scope of the present invention, the following discussion will generally be directed to the IgG class of immunoglobulin molecules. With regard to IgG, immunoglobulins comprise two identical light polypeptide chains of molecular weight approximately 23,000 Daltons, and two identical heavy chains of molecular weight 53,000-70,000. The four chains are joined by disulfide bonds in a “Y” configuration wherein the light chains bracket the heavy chains starting at the mouth of the “Y” and continuing through the variable domain.

Light chains of an immunoglobulin are classified as either kappa or lambda (κ, μ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgA₁, etc. are well characterized and are known to confer functional specialization. Modified versions of each of these classes and isotypes are readily discernable to the skilled artisan in view of the instant disclosure and, accordingly, are within the scope of the instant invention.

Both the light and heavy chains are divided into regions of structural and functional homology. The term “region” refers to a part or portion of a single immunoglobulin (as is the case with the term “Fc region”) or a single antibody chain and includes constant regions or variable regions, as well as more discrete parts or portions of said domains. For example, light chain variable domains include “complementarity determining regions” or “CDRs” interspersed among “framework regions” or “FRs”, as defined herein.

Certain regions of an immunoglobulin may be defined as “constant” (C) regions or “variable” (V) regions, based on the relative lack of sequence variation within the regions of various class members in the case of a “constant region”, or the significant variation within the regions of various class members in the case of a “variable regions”. The terms “constant region” and “variable region” may also be used functionally. In this regard, it will be appreciated that the variable regions of an immunoglobulin or antibody determine antigen recognition and specificity. Conversely, the constant regions of an immunoglobulin or antibody confer important effector functions such as secretion, transplacental mobility, Fc receptor binding, complement binding, and the like. The subunit structures and three dimensional configuration of the constant regions of the various immunoglobulin classes are well known.

The constant and variable regions of immunoglobulin heavy and light chains are folded into domains. The term “domain” refers to an independently folding, globular region of a heavy or light chain polypeptide comprising peptide loops (e.g., comprising 3 to 4 peptide loops) stabilized, for example, by β-pleated sheet and/or intrachain disulfide bond. Constant region domains on the light chain of an immunoglobulin are referred to interchangeably as “light chain constant region domains”, “CL regions” or “CL domains”. Constant domains on the heavy chain (e.g. hinge, CH1, CH2 or CH3 domains) are referred to interchangeably as “heavy chain constant region domains”, “CH” region domains or “CH domains”. Variable domains on the light chain are referred to interchangeably as “light chain variable region domains”, “VL region domains or “VL domains”. Variable domains on the heavy chain are referred to interchangeably as “heavy chain variable region domains”, “VH region domains” or “VH domains”.

By convention the numbering of the variable and constant region domains increases as they become more distal from the antigen binding site or amino-terminus of the immunoglobulin or antibody. The N-terminus of each heavy and light immunoglobulin chain is a variable region and at the C-terminus is a constant region; the CH3 and CL domains actually comprise the carboxy-terminus of the heavy and light chain, respectively. Accordingly, the domains of a light chain immunoglobulin are arranged in a VL-CL orientation, while the domains of the heavy chain are arranged in the VH-CH1-hinge-CH2-CH3 orientation.

Amino acid positions in a heavy chain constant region, including amino acid positions in the CH1, hinge, CH2, and CH3 domains, are numbered herein according to the EU index numbering system (see Kabat et al., in “Sequences of Proteins of Immunological Interest”, U.S. Dept. Health and Human Services, 5th edition, 1991). In contrast, amino acid positions in a light chain constant region (e.g. CL domains) are numbered herein according to the Kabat index numbering system (see Kabat et al., ibid).

As used herein, the term “V_{H} domain” includes the amino terminal variable domain of an immunoglobulin heavy chain, and the term “V_{L} domain” includes the amino terminal variable domain of an immunoglobulin light chain according to the Kabat index numbering system.

As used herein, the term “CH1 domain” includes the first (most amino terminal) constant region domain of an immunoglobulin heavy chain that extends, e.g., from about EU positions 118-215. The CH1 domain is adjacent to the VH domain and amino terminal to the hinge region of an immunoglobulin heavy chain molecule, and does not form a part of the Fc region of an immunoglobulin heavy chain. In one embodiment, a binding polypeptide of the invention comprises a CH1 domain derived from an immunoglobulin heavy chain molecule (e.g., a human IgG1 or IgG4 molecule).

As used herein, the term “hinge region” includes the portion of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al. J. Immunol. 1998, 161:4083).

As used herein, the term “CH2 domain” includes the portion of a heavy chain immunoglobulin molecule that extends, e.g., from about EU positions 231-340. The CH2 domain is unique in that it is not closely paired with another domain. Rather, two N-linked branched carbohydrate chains are interposed between the two CH2 domains of an intact native IgG molecule. In one embodiment, an binding polypeptide of the invention comprises a CH2 domain derived from an IgG1 molecule (e.g. a human IgG1 molecule). In another embodiment, an binding polypeptide of the invention comprises a CH2 domain derived from an IgG4 molecule (e.g., a human IgG4 molecule). In an exemplary embodiment, a polypeptide of the invention comprises a CH2 domain (EU positions 231-340), or a portion thereof.

As used herein, the term “CH3 domain” includes the portion of a heavy chain immunoglobulin molecule that extends approximately 110 residues from N-terminus of the CH2 domain, e.g., from about position 341-446b (EU numbering system). The CH3 domain typically forms the C-terminal portion of the antibody. In some immunoglobulins, however, additional domains may extend from CH3 domain to form the C-terminal portion of the molecule (e.g. the CH4 domain in the μ chain of IgM and the ε chain of IgE). In one embodiment, an binding polypeptide of the invention comprises a CH3 domain derived from an IgG1 molecule (e.g., a human IgG1 molecule). In another embodiment, an binding polypeptide of the invention comprises a CH3 domain derived from an IgG4 molecule (e.g., a human IgG4 molecule).

As used herein, the term “CL domain” includes the first (most amino terminal) constant region domain of an immunoglobulin light chain that extends, e.g. from about Kabat position 107A-216. The CL domain is adjacent to the V_{L} domain. In one embodiment, an binding polypeptide of the invention comprises a CL domain derived from a kappa light chain (e.g., a human kappa light chain).

As used herein, the term “effector function” refers to the functional ability of the Fc region or portion thereof to bind proteins and/or cells of the immune system and mediate various biological effects. Effector functions may be antigen-dependent or antigen-independent. A decrease in effector function refers to a decrease in one or more effector functions, while maintaining the antigen binding activity of the variable region of the antibody (or fragment thereof). Increase or decreases in effector function, e.g., Fc binding to an Fc receptor or complement protein, can be expressed in terms of fold change (e.g., changed by 1-fold, 2-fold, and the like) and can be calculated based on, e.g., the percent changes in binding activity determined using assays the are well-known in the art.

As used herein, the term “antigen-dependent effector function” refers to an effector function which is normally induced following the binding of an antibody to a corresponding antigen. Typical antigen-dependent effector functions include the ability to bind a complement protein (e.g. C1q). For example, binding of the C1 component of complement to the Fc region can activate the classical complement system leading to the opsonisation and lysis of cell pathogens, a process referred to as complement-dependent cytotoxicity (CDCC). The activation of complement also stimulates the inflammatory response and may also be involved in autoimmune hypersensitivity.

Other antigen-dependent effector functions are mediated by the binding of antibodies, via their Fc region, to certain Fc receptors (“FcRs”) on cells. There are a number of Fc receptors which are specific for different classes of antibody, including IgG (gamma receptors, or IgγRs), IgE (epsilon receptors, or IgεRs), IgA (alpha receptors, or IgαRs) and IgM (mu receptors, or IgμRs). Binding of antibody to Fc receptors on cell surfaces triggers a number of important and diverse biological responses including endocytosis of immune complexes, engulfment and destruction of antibody-coated particles or microorganisms (also called antibody-dependent phagocytosis, or ADCP), clearance of immune complexes, lysis of antibody-coated target cells by killer cells (called antibody-dependent cell-mediated cytotoxicity, or ADCC), release of inflammatory mediators, regulation of immune system cell activation, placental transfer and control of immunoglobulin production.

Certain Fc receptors, the Fc gamma receptors (FcγRs), play a critical role in either abrogating or enhancing immune recruitment. FcγRs are expressed on leukocytes and are composed of three distinct classes: FcγRI, FcγRII, and FcγRIII (Gessner et al., Ann. Hematol., (1998), 76: 231-48). Structurally, the FcγRs are all members of the immunoglobulin superfamily, having an IgG-binding α-chain with an extracellular portion composed of either two or three Ig-like domains. Human FcγRI (CD64) is expressed on human monocytes, exhibits high affinity binding (Ka=10⁸-10⁹ M⁻¹) to monomeric IgG1, IgG3, and IgG4. Human FcγRII (CD32) and FcγRIII (CD16) have low affinity for IgG1 and IgG3 (Ka<10⁷ M⁻¹), and can bind only complexed or polymeric forms of these IgG isotypes. Furthermore, the FcγRII and FcγIII classes comprise both “A” and “B” forms. FcγIIa (CD32a) and FcγRIIIa (CD16a) are bound to the surface of macrophages, NK cells and some T cells by a transmembrane domain while FcγIIb (CD32b) and FcγRIIIb (CD16b) are selectively bound to cell surface of granulocytes (e.g. neutrophils) via a phosphatidyl inositol glycan (GPI) anchor. The respective murine homologs of human FcγRI, FcγRII, and FcγRIII are FcγRIIa, FcγRIIb/1, and Fcγ10.

As used herein, the term “antigen-independent effector function” refers to an effector function which may be induced by an antibody, regardless of whether it has bound its corresponding antigen. Typical antigen-independent effector functions include cellular transport, circulating half-life and clearance rates of immunoglobulins, and facilitation of purification. A structurally unique Fc receptor, the “neonatal Fc receptor” or “FcRn”, also known as the salvage receptor, plays a critical role in regulating half-life and cellular transport. Other Fc receptors purified from microbial cells (e.g. Staphylococcal Protein A or G) are capable of binding to the Fc region with high affinity and can be used to facilitate the purification of the Fc-containing polypeptide.

Unlike Fcγs which belong to the Immunoglobulin superfamily, human FcRns structurally resemble polypeptides of Major Histoincompatibility Complex (MHC) Class I (Ghetie and Ward, Immunology Today, (1997), 18(12): 592-8). FcRn is typically expressed as a heterodimer consisting of a transmembrane α or heavy chain in complex with a soluble β or light chain (β2 microglobulin). FcRn shares 22-29% sequence identity with Class I MHC molecules and has a non-functional version of the MHC peptide binding groove (Simister and Mostov, Nature, (1989), 337: 184-7. Like MHC, the α chain of FcRn consists of three extracellular domains (α1, α2, α3) and a short cytoplasmic tail anchors the protein to the cell surface. The α1 and α2 domains interact with FcR binding sites in the Fc region of antibodies (Raghavan et al., Immunity, (1994), 1: 303-15). FcRn is expressed in the maternal placenta or yolk sac of mammals and it is involved in transfer of IgGs from mother to fetus. FcRn is also expressed in the small intestine of rodent neonates, where it is involved in the transfer across the brush border epithelia of maternal IgG from ingested colostrum or milk. FcRn is also expressed in numerous other tissues across numerous species, as well as in various endothelial cell lines. It is also expressed in human adult vascular endothelium, muscle vasculature, and hepatic sinusoids. FcRn is thought to play an additional role in maintaining the circulatory half-life or serum levels of IgG by binding it and recycling it to the serum. The binding of FcRn to IgG molecules is strictly pH-dependent with an optimum binding at a pH of less than 7.0.

As used herein, the term “half-life” refers to a biological half-life of a particular binding polypeptide in vivo. Half-life may be represented by the time required for half the quantity administered to a subject to be cleared from the circulation and/or other tissues in the animal. When a clearance curve of a given binding polypeptide is constructed as a function of time, the curve is usually biphasic with a rapid α-phase and longer β-phase. The β-phase typically represents an equilibration of the administered Fc polypeptide between the intra- and extra-vascular space and is, in part, determined by the size of the polypeptide. The β-phase typically represents the catabolism of the binding polypeptide in the intravascular space. Therefore, in a preferred embodiment, the term half-life as used herein refers to the half-life of the binding polypeptide in the β-phase. The typical β phase half-life of a human antibody in humans is 21 days.

As indicated above, the variable regions of an antibody allow it to selectively recognize and specifically bind epitopes on antigens. That is, the V_{L} domain and V_{H} domain of an antibody combine to form the variable region (Fv) that defines a three dimensional antigen binding site. This quaternary antibody structure forms the antigen binding site present at the end of each arm of the Y. More specifically, the antigen binding site is defined by three complementary determining regions (CDRs) on each of the heavy and light chain variable regions.

As used herein, the term “antigen binding site” includes a site that specifically binds (immunoreacts with) an antigen such as a cell surface or soluble antigen). In one embodiment, the binding site includes an immunoglobulin heavy chain and light chain variable region and the binding site formed by these variable regions determines the specificity of the antibody. An antigen binding site is formed by variable regions that vary from one polypeptide to another. In one embodiment, a binding polypeptide of the invention comprises an antigen binding site comprising at least one heavy or light chain CDR of an antibody molecule (e.g., the sequence of which is known in the art or described herein). In another embodiment, a binding polypeptide of the invention comprises an antigen binding site comprising at least two CDRs from one or more antibody molecules. In another embodiment, a binding polypeptide of the invention comprises an antigen binding site comprising at least three CDRs from one or more antibody molecules. In another embodiment, a binding polypeptide of the invention comprises an antigen binding site comprising at least four CDRs from one or more antibody molecules. In another embodiment, a binding polypeptide of the invention comprises an antigen binding site comprising at least five CDRs from one or more antibody molecules. In another embodiment, a binding polypeptide of the invention comprises an antigen binding site comprising six CDRs from an antibody molecule. Exemplary antibody molecules comprising at least one CDR that can be included in the subject binding polypeptides are known in the art and exemplary molecules are described herein.

As used herein, the term “CDR” or “complementarity determining region” means the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of amino acid residues when compared against each other. The amino acid residues which encompass the CDRs as defined by each of the above cited references are set forth for comparison. Preferably, the term “CDR” is a CDR as defined by Kabat based on sequence comparisons.

CDR Definitions

| | Kabat¹ | Chothia² | MacCallum³ |
|---|---|---|---|
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

The term “framework region” or “FR region” as used herein, includes the amino acid residues that are part of the variable region, but are not part of the CDRs (e.g., using the Kabat definition of CDRs). Therefore, a variable region framework is between about 100-120 amino acids in length but includes only those amino acids outside of the CDRs. For the specific example of a heavy chain variable region and for the CDRs as defined by Kabat et al., framework region 1 corresponds to the domain of the variable region encompassing amino acids 1-30; framework region 2 corresponds to the domain of the variable region encompassing amino acids 36-49; framework region 3 corresponds to the domain of the variable region encompassing amino acids 66-94, and framework region 4 corresponds to the domain of the variable region from amino acids 103 to the end of the variable region. The framework regions for the light chain are similarly separated by each of the light chain variable region CDRs. Similarly, using the definition of CDRs by Chothia et al. or McCallum et al. the framework region boundaries are separated by the respective CDR termini as described above. In preferred embodiments, the CDRs are as defined by Kabat.

In naturally occurring antibodies, the six CDRs present on each monomeric antibody are short, non-contiguous sequences of amino acids that are specifically positioned to form the antigen binding site as the antibody assumes its three dimensional configuration in an aqueous environment. The remainder of the heavy and light variable domains show less inter-molecular variability in amino acid sequence and are termed the framework regions. The framework regions largely adopt a β-sheet conformation and the CDRs form loops which connect, and in some cases form part of, the β-sheet structure. Thus, these framework regions act to form a scaffold that provides for positioning the six CDRs in correct orientation by inter-chain, non-covalent interactions. The antigen binding site formed by the positioned CDRs defines a surface complementary to the epitope on the immunoreactive antigen. This complementary surface promotes the non-covalent binding of the antibody to the immunoreactive antigen epitope. The position of CDRs can be readily identified by one of ordinary skill in the art.

In certain embodiments, the binding polypeptides of the invention comprise at least two antigen binding domains (e.g., within the same binding polypeptide (e.g, at both the N- and C-terminus of a single polypeptide) or linked to each component binding polypeptide of a mutimeric binding protein of the invention) that provide for the association of the binding polypeptide with the selected antigen. The antigen binding domains need not be derived from the same immunoglobulin molecule. In this regard, the variable region may or may not be derived from any type of animal that can be induced to mount a humoral response and generate immunoglobulins against the desired antigen. As such, the variable region may be, for example, of mammalian origin e.g., may be human, murine, non-human primate (such as cynomolgus monkeys, macaques, etc.), lupine, camelid (e.g., from camels, llamas and related species).

The term “antibody variant” or “modified antibody” includes an antibody which does not occur in nature and which has an amino acid sequence or amino acid side chain chemistry which differs from that of a naturally-derived antibody by at least one amino acid or amino acid modification as described herein. As used herein, the term “antibody variant” includes synthetic forms of antibodies which are altered such that they are not naturally occurring, e.g., antibodies that comprise at least two heavy chain portions but not two complete heavy chains (such as, domain deleted antibodies or minibodies); multispecific forms of antibodies (e.g., bispecific, trispecific, etc.) altered to bind to two or more different antigens or to different epitopes on a single antigen); heavy chain molecules joined to scFv molecules; single-chain antibodies; diabodies; triabodies; and antibodies with altered effector function and the like.

As used herein the term “scFv molecule” includes binding molecules which consist of one light chain variable domain (VL) or portion thereof, and one heavy chain variable domain (VH) or portion thereof, wherein each variable domain (or portion thereof) is derived from the same or different antibodies. scFv molecules preferably comprise an scFv linker interposed between the VH domain and the VL domain. ScFv molecules are known in the art and are described, e.g., in U.S. Pat. No. 5,892,019, Ho et al. 1989. Gene 77:51; Bird et al. 1988 Science 242:423; Pantoliano et al. 1991. Biochemistry 30:10117; Milenic et al. 1991. Cancer Research 51:6363; Takkinen et al. 1991. Protein Engineering 4:837.

A “scFv linker” as used herein refers to a moiety interposed between the VL and VH domains of the scFv. scFv linkers preferably maintain the scFv molecule in a antigen binding conformation. In one embodiment, a scFv linker comprises or consists of an scFv linker peptide. In certain embodiments, a scFv linker peptide comprises or consists of a gly-ser polypeptide linker. In other embodiments, a scFv linker comprises a disulfide bond.

As used herein, the term “gly-ser polypeptide linker” refers to a peptide that consists of glycine and serine residues. An exemplary gly/ser polypeptide linker comprises the amino acid sequence (Gly₄ Ser)ₙ. In one embodiment, n=1. In one embodiment, n=2. In another embodiment, n=3, i.e., (Gly₄ Ser)₃. In another embodiment, n=4, i.e., (Gly₄ Ser)₄. In another embodiment, n=5. In yet another embodiment, n=6. In another embodiment, n=7. In yet another embodiment, n=8. In another embodiment, n=9. In yet another embodiment, n=10. Another exemplary gly/ser polypeptide linker comprises the amino acid sequence Ser(Gly₄Ser)ₙ. In one embodiment, n=1. In one embodiment, n=2. In a preferred embodiment, n=3. In another embodiment, n=4. In another embodiment, n=5. In yet another embodiment, n=6.

As used herein the term “protein stability” refers to an art-recognized measure of the maintenance of one or more physical properties of a protein in response to an environmental condition (e.g. an elevated or lowered temperature). In one embodiment, the physical property is the maintenance of the covalent structure of the protein (e.g. the absence of proteolytic cleavage, unwanted oxidation or deamidation). In another embodiment, the physical property is the presence of the protein in a properly folded state (e.g. the absence of soluble or insoluble aggregates or precipitates).

The term “glycosylation” refers to the covalent linking of one or more carbohydrates to a polypeptide. Typically, glycosylation is a posttranslational event which can occur within the intracellular milieu of a cell or extract therefrom. The term glycosylation includes, for example, N-linked glycosylation (where one or more sugars are linked to an asparagine residue) and/or O-linked glycosylation (where one or more sugars are linked to an amino acid residue having a hydroxyl group (e.g., serine or threonine).

A used herein, the term “native cysteine” shall refer to a cysteine amino acid that occurs naturally at a particular amino acid position of a polypeptide and which has not been modified, introduced, or altered by the hand of man. The term “engineered cysteine residue or analog thereof” or “engineered cysteine or analog thereof” shall refer to a non-native cysteine residue or a cysteine analog (e.g. thiol-containing analogs such as thiazoline-4-carboxylic acid and thiazolidine-4 carboxylic acid (thioproline, Th)), which is introduced by synthetic means (e.g. by recombinant techniques, in vitro peptide synthesis, by enzymatic or chemical coupling of peptides or some combination of these techniques) into an amino acid position of a polypeptide that does not naturally contain a cysteine residue or analog thereof at that position.

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the CH1 and CL regions are linked by native disulfide bonds and the two heavy chains are linked by two native disulfide bonds at positions corresponding to 239 and 242 using the Kabat numbering system (position 226 or 229, EU numbering system).

As used herein, the term “bonded cysteine” shall refer to a native or engineered cysteine residue within a polypeptide which forms a disulfide bond or other covalent bond with a second native or engineered cysteine or other residue present within the same or different polypeptide. An “intrachain bonded cysteine” shall refer to a bonded cysteine that is covalently bonded to a second cysteine present within the same polypeptide (ie. an intrachain disulfide bond). An “interchain bonded cysteine” shall refer to a bonded cysteine that is covalently bonded to a second cysteine present within a different polypeptide (ie. an interchain disulfide bond).

As used herein, the term “free cysteine” refers to a native or engineered cysteine amino acid residues within a polypeptide sequence (and analogs or mimetics thereof, e.g. thiazoline-4-carboxylic acid and thiazolidine-4 carboxylic acid (thioproline, Th)) that exists in a substantially reduced form. Free cysteines are preferably capable of being modified with an effector moiety of the invention.

The term “thiol modification reagent” shall refer to a chemical agent that is capable of selectively reacting with the thiol group of an engineered cysteine residue or analog thereof in a binding polypeptide (e.g., within an polypeptide linker of a binding polypeptide), and thereby providing means for site-specific chemical addition or crosslinking of effector moieties to the binding polypeptide, thereby forming a modified binding polypeptide. Preferably the thiol modification reagent exploits the thiol or sulfhydryl functional group which is present in a free cysteine residue. Exemplary thiol modification reagents include maleimides, alkyl and aryl halides, α-haloacyls, and pyridyl disulfides.

The term “functional moiety” includes moieties which, preferably, add a desirable function to the binding polypeptide. Preferably, the function is added without significantly altering an intrinsic desirable activity of the polypeptide, e.g., the antigen-binding activity of the molecule. A binding polypeptide of the invention may comprise one or more functional moieties, which may be the same or different. Examples of useful functional moieties include, but are not limited to, an effector moiety, an affinity moiety, and a blocking moiety.

Exemplary blocking moieties include moieties of sufficient steric bulk and/or charge such that reduced glycosylation occurs, for example, by blocking the ability of a glycosidase to glycosylate the polypeptide. The blocking moiety may additionally or alternatively, reduce effector function, for example, by inhibiting the ability of the Fc region to bind a receptor or complement protein. Preferred blocking moieties include cysteine adducts, cystine, mixed disulfide adducts, and PEG moieties. Exemplary detectable moieties include fluorescent moieties, radioisotopic moieties, radiopaque moieties, and the like.

With respect to conjugation of chemical moieties, the term “linking moiety” includes moieties which are capable of linking a functional moiety to the remainder of the binding polypeptide. The linking moiety may be selected such that it is cleavable or non-cleavable. Uncleavable linking moieties generally have high systemic stability, but may also have unfavorable pharmacokinetics.

The term “spacer moiety” is a nonprotein moiety designed to introduce space into a molecule. In one embodiment a spacer moiety may be an optionally substituted chain of 0 to 100 atoms, selected from carbon, oxygen, nitrogen, sulfur, etc. In one embodiment, the spacer moiety is selected such that it is water soluble. In another embodiment, the spacer moiety is polyalkylene glycol, e.g., polyethylene glycol or polypropylene glycol.

The terms “PEGylation moiety” or “PEG moiety” includes a polyalkylene glycol compound or a derivative thereof, with or without coupling agents or derivitization with coupling or activating moieties (e.g., with thiol, triflate, tresylate, azirdine, oxirane, or preferably with a maleimide moiety, e.g., PEG-maleimide). Other appropriate polyalkylene glycol compounds include, maleimido monomethoxy PEG, activated PEG polypropylene glycol, but also charged or neutral polymers of the following types: dextran, colominic acids, or other carbohydrate based polymers, polymers of amino acids, and biotin derivatives.

As used herein, the term “effector moiety” (E) may comprise diagnostic and therapeutic agents (e.g. proteins, nucleic acids, lipids, drug moieties, and fragments thereof) with biological or other functional activity. For example, a binding polypeptide comprising an effector moiety conjugated to a binding polypeptide has at least one additional function or property as compared to the unconjugated polypeptide. For example, the conjugation of a cytotoxic drug moiety (e.g., an effector moiety) to a binding polypeptide (e.g., via its polypeptide linker) results in the formation of a modified polypeptide with drug cytotoxicity as second function (i.e. in addition to antigen binding). In another example, the conjugation of a second binding polypeptide to the first binding polypeptide may confer additional binding properties.

In one aspect, wherein the effector moiety is a genetically encoded therapeutic or diagnostic protein or nucleic acid, the effector moiety may be synthesized or expressed by either peptide synthesis or recombinant DNA methods that are well known in the art.

In another aspect, wherein the effector is a non-genetically encoded peptide or a drug moiety, the effector moiety may be synthesized artificially or purified from a natural source.

As used herein, the term “drug moiety” includes anti-inflammatory, anticancer, anti-infective (e.g., anti-fungal, antibacterial, anti-parasitic, anti-viral, etc.), and anesthetic therapeutic agents. In a further embodiment, the drug moiety is an anticancer or cytotoxic agent. Compatible drug moieties may also comprise prodrugs.

As used herein, the term “prodrug” refers to a precursor or derivative form of a pharmaceutically active agent that is less active, reactive or prone to side effects as compared to the parent drug and is capable of being enzymatically activated or otherwise converted into a more active form in vivo. Prodrugs compatible with the invention include, but are not limited to, phosphate-containing prodrugs, amino acid-containing prodrugs, thiophosphate-containing prodrugs, sulfate containing prodrugs, peptide containing prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs that can be converted to the more active cytotoxic free drug. One skilled in the art may make chemical modifications to the desired drug moiety or its prodrug in order to make reactions of that compound more convenient for purposes of preparing modified binding proteins of the invention. The drug moieties also include derivatives, pharmaceutically acceptable salts, esters, amides, and ethers of the drug moieties described herein. Derivatives include modifications to drugs identified herein which may improve or not significantly reduce a particular drug's desired therapeutic activity.

As used herein, the term “anticancer agent” includes agents which are detrimental to the growth and/or proliferation of neoplastic or tumor cells and may act to reduce, inhibit or destroy malignancy. Examples of such agents include, but are not limited to, cytostatic agents, alkylating agents, antibiotics, cytotoxic nucleosides, tubulin binding agents, hormones and hormone antagonists, and the like. Any agent that acts to retard or slow the growth of immunoreactive cells or malignant cells is within the scope of the present invention.

An “affinity tag” or an “affinity moiety” is a chemical moiety that is attached to one or more of the binding polypeptide, polypeptide linker, or effector moiety in order to facilitate its separation from other components during a purification procedure. Exemplary affinity domains include the His tag, chitin binding domain, maltose binding domain, biotin, and the like.

An “affinity resin” is a chemical surface capable of binding the affinity domain with high affinity to facilitate separation of the protein bound to the affinity domain from the other components of a reaction mixture. Affinity resins can be coated on the surface of a solid support or a portion thereof. Alternatively, the affinity resin can comprise the solid support. Such solid supports can include a suitably modified chromatography column, microtiter plate, bead, or biochip (e.g. glass wafer). Exemplary affinity resins are comprised of nickel, chitin, amylase, and the like.

The term “vector” or “expression vector” is used herein to mean vectors used in accordance with the present invention as a vehicle for introducing into and expressing a desired polynucleotide in a cell. As known to those skilled in the art, such vectors may easily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

For the purposes of this invention, numerous expression vector systems may be employed. For example, one class of vector utilizes DNA elements which are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Exemplary vectors include those described in U.S. Pat. Nos. 6,159,730 and 6,413,777, and U.S. Patent Application No. 2003 0157641 A1. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. In one embodiment, an inducible expression system can be employed. Additional elements may also be needed for optimal synthesis of mRNA. These elements may include signal sequences, splice signals, as well as transcriptional promoters, enhancers, and termination signals. In one embodiment, a secretion signal, e.g., any one of several well characterized bacterial leader peptides (e.g., pelB, phoA, or ompA), can be fused in-frame to the N terminus of a polypeptide of the invention to obtain optimal secretion of the polypeptide. (Lei et al. (1988), Nature, 331:543; Better et al. (1988) Science, 240:1041; Mullinax et al., (1990). PNAS, 87:8095).

The term “host cell” refers to a cell that has been transformed with a vector constructed using recombinant DNA techniques and encoding at least one heterologous gene. In descriptions of processes for isolation of proteins from recombinant hosts, the terms “cell” and “cell culture” are used interchangeably to denote the source of protein unless it is clearly specified otherwise. In other words, recovery of protein from the “cells” may mean either from spun down whole cells, or from the cell culture containing both the medium and the suspended cells. The host cell line used for protein expression is most preferably of mammalian origin; those skilled in the art are credited with ability to preferentially determine particular host cell lines which are best suited for the desired gene product to be expressed therein. Exemplary host cell lines include, but are not limited to, DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/O (mouse myeloma), P3×63-Ag3.653 (mouse myeloma), BFA-1c1BPT (bovine endothelial cells), RAJI (human lymphocyte) and 293 (human kidney). CHO cells are particularly preferred. Host cell lines are typically available from commercial services, the American Tissue Culture Collection or from published literature. The polypeptides of the invention can also be expressed in non-mammalian cells such as bacteria or yeast or plant cells. In this regard it will be appreciated that various unicellular non-mammalian microorganisms such as bacteria can also be transformed; i.e. those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the polypeptides typically become part of inclusion bodies. The polypeptides must be isolated, purified and then assembled into functional molecules.

In addition to prokaryotes, eukaryotic microbes may also be used. Saccharomyces cerevisiae, or common baker's yeast, is the most commonly used among eukaryotic microorganisms although a number of other strains are commonly available including Pichia pastoris. For expression in Saccharomyces, the plasmid YRp7, for example, (Stinchcomb et al., (1979), Nature, 282:39; Kingsman et al., (1979), Gene, 7:141; Tschemper et al., (1980), Gene, 10:157) is commonly used. This plasmid already contains the TRP1 gene which provides a selection marker for a mutant strain of yeast lacking the ability to grow in tryptophan, for example ATCC No. 44076 or PEP4-1 (Jones, (1977), Genetics, 85:12). The presence of the trpl lesion as a characteristic of the yeast host cell genome then provides an effective environment for detecting transformation by growth in the absence of tryptophan.

In vitro production allows scale-up to give large amounts of the desired altered binding polypeptides of the invention. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, hydrophobic interaction chromatography (HIC, chromatography over DEAE-cellulose or affinity chromatography.

As used herein, “tumor-associated antigens” means any antigen which is generally associated with tumor cells, i.e., occurring at the same or to a greater extent as compared with normal cells. More generally, tumor associated antigens comprise any antigen that provides for the localization of immunoreactive antibodies at a neoplastic cell irrespective of its expression on non-malignant cells. Such antigens may be relatively tumor specific and limited in their expression to the surface of malignant cells. Alternatively, such antigens may be found on both malignant and non-malignant cells. In certain embodiments, the binding polypeptides of the present invention preferably bind to tumor-associated antigens. Accordingly, the binding polypeptide of the invention may be derived, generated or fabricated from any one of a number of antibodies that react with tumor associated molecules.

As used herein, the term “malignancy” refers to a non-benign tumor or a cancer. As used herein, the term “cancer” includes a malignancy characterized by deregulated or uncontrolled cell growth. Exemplary cancers include: carcinomas, sarcomas, leukemias, and lymphomas. The term “cancer” includes primary malignant tumors (e.g., those whose cells have not migrated to sites in the subject's body other than the site of the original tumor) and secondary malignant tumors (e.g., those arising from metastasis, the migration of tumor cells to secondary sites that are different from the site of the original tumor).

As used herein, the phrase “subject that would benefit from administration of a binding polypeptide” includes subjects, such as mammalian subjects, that would benefit from administration of binding polypeptides used, e.g., for detection of an antigen recognized by a binding polypeptide of the invention (e.g., for a diagnostic procedure) and/or from treatment with a binding polypeptide to reduce or eliminate the target recognized by the binding polypeptide. For example, in one embodiment, the subject may benefit from reduction or elimination of a soluble or particulate molecule from the circulation or serum (e.g., a toxin or pathogen) or from reduction or elimination of a population of cells expressing the target (e.g., tumor cells). As discussed above, the binding polypeptide can be used in unconjugated form or can be conjugated, e.g., to a drug, prodrug, or an isotope, to form a modified binding polypeptide for administering to said subject.

### II. BINDING POLYPEPTIDES COMPRISING SINGLE-CHAIN FC (“SCFC”) REGIONS

In certain aspects, the invention provides binding polypeptides comprising at least one genetically fused Fc region or portion thereof within a single polypeptide chain (i.e., binding polypeptides comprising a single-chain Fc (scFc) region). Preferred polypeptides of the invention comprise at least two Fc moieties (e.g., 2, 3, 4, 5, 6, or more Fc moieties) or Fc moieties within the same linear polypeptide chain. Preferably, at least two (more preferably all) of the Fc moieties are capable of folding (e.g., intramolecularly or intermolecularly folding) to form at least one functional scFc region which imparts an effector function to the polypeptide. For example, in one preferred embodiment, a binding polypeptide of the invention is capable of binding, via its scFc region, to an Fc receptor (e.g. an FcRn, an FcγR receptor (e.g., FcγRIII), or a complement protein (e.g. C1q)) in order to trigger an immune effector function (e.g., antibody-dependent cytotoxicity (ADCC), phagocytosis, or complement-dependent cytotoxicity (CDCC)).

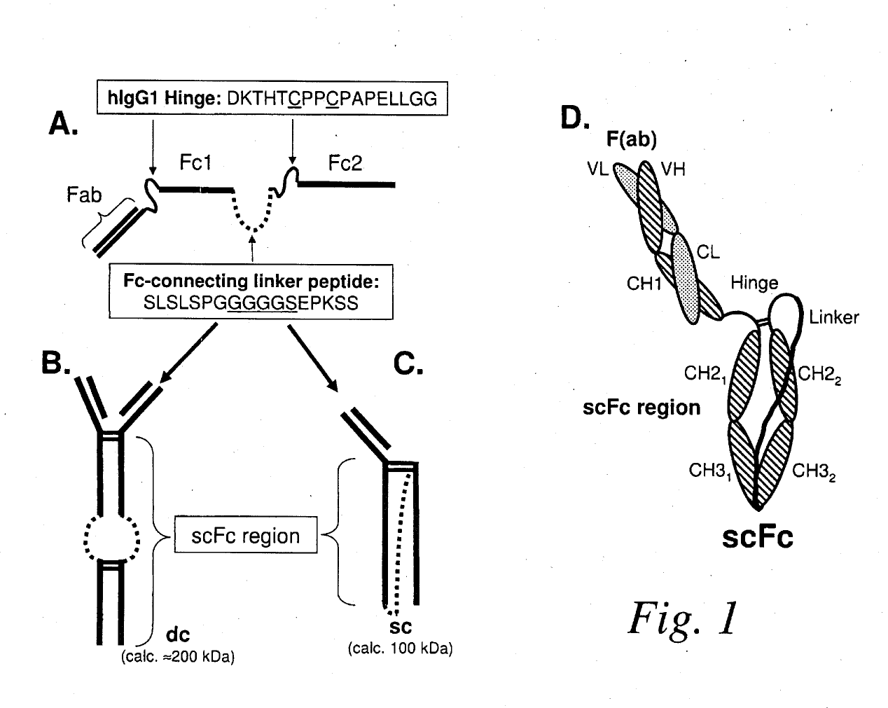

In certain embodiments, at least two of the Fc moieties of the genetically fused Fc region (i.e., scFc region) are directly fused to each other in a contiguous linear sequence of amino acids such that there is no intervening amino acid or peptide between the C-terminus of the first Fc moiety and the N-terminus of the second Fc moiety. In more preferred embodiments, however, at least two of the Fc moieties (more preferably all) of the genetically-fused Fc region (i.e., scFc region) are genetically fused via a polypeptide linker (e.g., a synthetic linker) interposed between the at least two Fc moieties. The polypeptide linker ensures optimal folding, alignment, and/or juxtaposition of the at least two Fc moieties such that the scFc region is capable of binding with suitable affinity to an Fc receptor, thereby triggering an effector function. For example, in certain embodiments the genetically-fused Fc region (i.e., scFc region) is capable of folding intramolecularly (see, e.g., the monomeric (“sc”) scFc construct in FIG. 1), whereas in other embodiments, the genetically-fused Fc region (i.e., scFc region) is capable of forming a dimeric scFc construct. In certain embodiments, the genetically-fused Fc region (i.e., scFc region) is capable of binding to an Fc receptor with a binding affinity of at least 10⁻⁷ M (e.g., at least 10⁻⁸ M, at least 10⁻⁹ M, at least 10⁻¹⁰ M, at least 10⁻¹¹M, or at least 10⁻¹² M).

In certain embodiments, the polypeptides of the invention may comprise a scFc region comprising Fc moieties of the same, or substantially the same, sequence composition (herein termed a “homomeric scFc region”). In other embodiments, the polypeptides of the invention may comprise a scFc region comprising at least two Fc moieties which are of different sequence composition (i.e., herein termed a “heteromeric scFc region”). In certain embodiments, the binding polypeptides of the invention comprise a scFc region comprising at least one insertion or amino acid substitution. In one exemplary embodiment, the heteromeric scFc region comprises an amino acid substitution in a first Fc moiety (e.g., an amino acid substitution of Asparagine at EU position 297), but not in a second Fc moiety.

In certain embodiments, the scFc region is hemi-glycosylated. For example, the heteromeric scFc region may comprise a first, glycosylated, Fc moiety (e.g., a glycosylated CH2 region) and a second, aglycosylated, Fc moiety (e.g., an aglycosylated CH2 region), wherein a linker is interposed between the glycosylated and aglycosylated Fc moieties. In other embodiments, the scFc region is fully glycosylated, i.e., all of the Fc moieties are glycosylated. In still further embodiments, the scFc region may be aglycosylated, i.e., none of the Fc moieties are glycosylated.

The binding polypeptides of the invention may be assembled together or with other polypeptides to form multimeric binding polypeptides or proteins (also, referred to herein as “multimers”). The multimeric binding polypeptide or proteins of the invention comprise at least one binding polypeptide of the invention. Accordingly, the invention is directed without limitation to monomeric as well as multimeric (e.g., dimeric, trimeric, tetrameric, and hexameric) binding polyeptides or proteins and the like. In certain embodiments, the constituent binding polypeptides of said multimers are the same (ie. homomeric multimers, e.g. homodimers, homotrimers, homotetramers). In other embodiments, at least two constituent polypeptides of the multimeric proteins of the invention are different (ie. heteromeric multimers, e.g. heterodimers, heterotrimers, heterotetramers).

In certain embodiments, at least two binding polypeptides of the invention are capable of forming a dimer. For example, in certain embodiments the genetically-fused Fc region (i.e., scFc region) of a binding polypeptide remains unfolded, such that its constituent Fc moieties associate not with each other, but with corresponding Fc moieties in another binding polyeptide (see, e.g., the dimeric (“dc”) scFc construct in FIG. 1B).

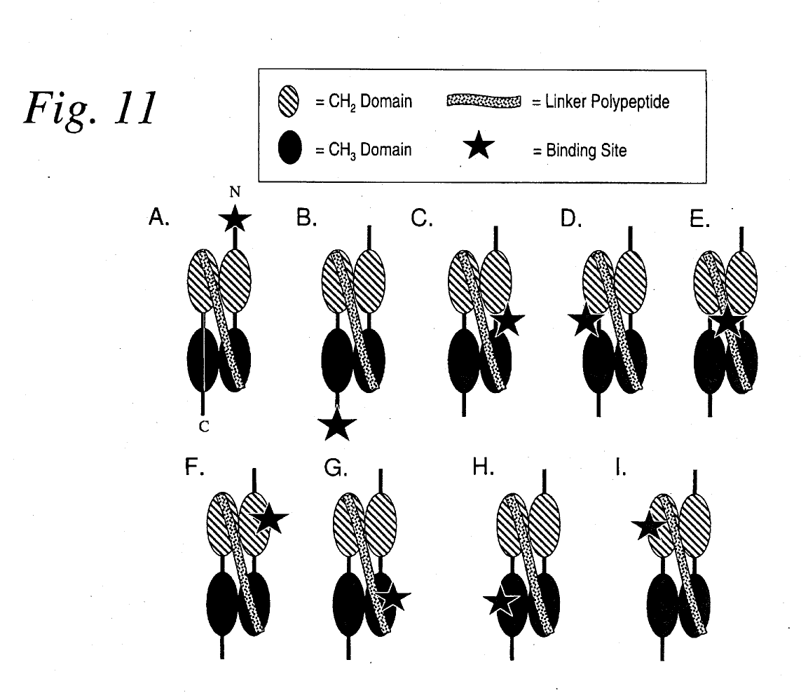

A variety of binding polypeptides of alternative designs are also within the scope of the invention. For example, one or more binding sites can be fused to, linked with, or incorporated within (e.g., veneered onto) a scFc region of the invention in a multiple orientations. FIG. 11 depicts a variety of non-limiting examples of such scFc binding polypeptides. In one exemplary embodiment, a binding polypeptide of the invention comprises a binding site fused to the N-terminus of a scFc region (FIG. 11A). In another exemplary embodiment, a binding polypeptide comprises a binding site at the C-terminus of a scFc region (FIG. 11B). The binding polypeptide of the invention may comprise binding sites at both the C-terminus and the N-terminus of a scFc region. In yet other embodiments, a binding polypeptide of the invention may comprise a binding site in an N-terminal and/or C-terminal interdomain region of a scFc region (e.g., between the CH2 and CH3 domains of a first, N-terminal, Fc moiety (FIG. 11C) or a second, C-terminal, Fc moiety (FIG. 11D)). Alternatively, the binding site may be incorporated in an interdomain region between the hinge and CH2 domains of an Fc moiety. In other embodiments, a binding polyeptide may comprise one or more binding sites within a linker polypeptide of a scFc region (FIG. 11E).

In still further embodiments, the binding polyeptide of the invention comprises a binding site which is introduced into an Fc moiety of a scFc region. For example, a binding site may be veneered into an N-terminal CH2 domain (FIG. 1F), an N-terminal CH3 domain (FIG. 1G), a C-terminal CH2 domain (FIG. 1H), and/or a C-terminal CH3 domain (FIG. 11). In one embodiment, the CDR loops of an antibody are veneered into one or both CH3 domains scFc region. Methods for veneering CDR loops and other binding moeties into the CH2 and/or CH3 domains of an Fc region are disclosed, for example, in International PCT Publication No. WO 08/003,116, which is incorporated by reference herein.

It is recognized by those skilled in the art that a scFc binding polypeptide of the invention may comprise two or more binding sites (e.g., 2, 3, 4, or more binding sites) which are linked, fused, or integrated (e.g., veneered) into a scFc region of the invention using any combination of the orientations depicted in FIGS. 11A-I.

### A. Fc Moieties

Fc moieties useful for producing the binding polypeptides of the present invention may be obtained from a number of different sources. In preferred embodiments, a Fc moiety of the binding polypeptide is derived from a human immunoglobulin. It is understood, however, that the Fc moiety may be derived from an immunoglobulin of another mammalian species, including for example, a rodent (e.g. a mouse, rat, rabbit, guinea pig) or non-human primate (e.g. chimpanzee, macaque) species. Moreover, the binding polypeptide Fc domain or portion thereof may be derived from any immunoglobulin class, including IgM, IgG, IgD, IgA and IgE, and any immunoglobulin isotype, including IgG1, IgG2, IgG3 and IgG4. In a preferred embodiment, the human isotype IgG1 is used.

A variety of Fc moiety gene sequences (e.g. human constant region gene sequences) are available in the form of publicly accessible deposits. Constant region domains comprising an Fc moiety sequence can be selected having a particular effector function (or lacking a particular effector function) or with a particular modification to reduce immunogenicity. Many sequences of antibodies and antibody-encoding genes have been published and suitable Fc moiety sequences (e.g. hinge, CH2, and/or CH3 sequences, or portions thereof) can be derived from these sequences using art recognized techniques. The genetic material obtained using any of the foregoing methods may then be altered or synthesized to obtain polypeptides of the present invention. It will further be appreciated that the scope of this invention encompasses alleles, variants and mutations of constant region DNA sequences.

Fc moiety sequences can be cloned, e.g., using the polymerase chain reaction and primers which are selected to amplify the domain of interest. To clone an Fc moiety sequence from an antibody, mRNA can be isolated from hybridoma, spleen, or lymph cells, reverse transcribed into DNA, and antibody genes amplified by PCR. PCR amplification methods are described in detail in U.S. Pat. Nos. 4,683,195; 4,683,202; 4,800,159; 4,965,188; and in, e.g., “PCR Protocols: A Guide to Methods and Applications” Innis et al. eds., Academic Press, San Diego, Calif. (1990); Ho et al. 1989. Gene 77:51; Horton et al. 1993. Methods Enzymol. 217:270). PCR may be initiated by consensus constant region primers or by more specific primers based on the published heavy and light chain DNA and amino acid sequences. As discussed above, PCR also may be used to isolate DNA clones encoding the antibody light and heavy chains. In this case the libraries may be screened by consensus primers or larger homologous probes, such as mouse constant region probes. Numerous primer sets suitable for amplification of antibody genes are known in the art (e.g., 5′ primers based on the N-terminal sequence of purified antibodies (Benhar and Pastan. 1994. Protein Engineering 7:1509); rapid amplification of cDNA ends (Ruberti, F. et al. 1994. J. Immunol. Methods 173:33); antibody leader sequences (Larrick et al. 1989 Biochem. Biophys. Res. Commun. 160:1250). The cloning of antibody sequences is further described in Newman et al., U.S. Pat. No. 5,658,570, filed Jan. 25, 1995, which is incorporated by reference herein.

The binding polypeptides of the invention may comprise two or more Fc moieties (e.g., 2, 3, 4, 5, 6, 7, 8, 9, 10, or more Fc moieties). These two or more Fc moieties can form a Fc region. In one embodiment, the Fc moieties may be of different types. In one embodiment, at least one Fc moiety present in the binding polypeptide comprises a hinge domain or portion thereof. In another embodiment, the binding polypeptide of the invention comprises at least one Fc moiety which comprises at least one CH2 domain or portion thereof. In another embodiment, the binding polypeptide of the invention comprises at least one Fc moiety which comprises at least one CH3 domain or portion thereof. In another embodiment, the binding polypeptide of the invention comprises at least one Fc moiety which comprises at least one CH4 domain or portion thereof. In another embodiment, the binding polypeptide of the invention comprises at least one Fc moiety which comprises at least one hinge domain or portion thereof and at least one CH2 domain or portion thereof (e.g, in the hinge-CH2 orientation). In another embodiment, the binding polypeptide of the invention comprises at least one Fc moiety which comprises at least one CH2 domain or portion thereof and at least one CH3 domain or portion thereof (e.g, in the CH2-CH3 orientation). In another embodiment, the binding polypeptide of the invention comprises at least one Fc moiety comprising at least one hinge domain or portion thereof, at least one CH2 domain or portion thereof, and least one CH3 domain or portion thereof, for example in the orientation hinge-CH2-CH3, hinge-CH3-CH2, or CH2-CH3-hinge.

In certain embodiments, the binding polypeptide comprises at least one complete Fc region derived from one or more immunoglobulin heavy chains (e.g., an Fc domain including hinge, CH2, and CH3 domains, although these need not be derived from the same antibody). In other embodiments, the binding polypeptide comprises at least two complete Fc regions derived from one or more immunoglobulin heavy chains. In preferred embodiments, the complete Fc moiety is derived from a human IgG immunoglobulin heavy chain (e.g., human IgG1).

In another embodiment, a binding polypeptide of the invention comprises at least one Fc moiety comprising a complete CH3 domain (about amino acids 341-438 of an antibody Fc region according to EU numbering). In another embodiment, a binding polypeptide of the invention comprises at least one Fc moiety comprising a complete CH2 domain (about amino acids 231-340 of an antibody Fc region according to EU numbering). In another embodiment, a binding polypeptide of the invention comprises at least one Fc moiety comprising at least a CH3 domain, and at least one of a hinge region (about amino acids 216-230 of an antibody Fc region according to EU numbering), and a CH2 domain. In one embodiment, a binding polypeptide of the invention comprises at least one Fc moiety comprising a hinge and a CH3 domain. In another embodiment, a binding polypeptide of the invention comprises at least one Fc moiety comprising a hinge, a CH2, and a CH3 domain. In preferred embodiments, the Fc moiety is derived from a human IgG immunoglobulin heavy chain (e.g., human IgG1).

The constant region domains or portions thereof making up an Fc moiety of a binding polypeptide of the invention may be derived from different immunoglobulin molecules. For example, a polypeptide of the invention may comprise a CH2 domain or portion thereof derived from an IgG1 molecule and a CH3 region or portion thereof derived from an IgG3 molecule. In another example, a binding polypeptide can comprise an Fc moiety comprising a hinge domain derived, in part, from an IgG1 molecule and, in part, from an IgG3 molecule. As set forth herein, it will be understood by one of ordinary skill in the art that an Fc moiety may be altered such that it varies in amino acid sequence from a naturally occurring antibody molecule.

In another embodiment, a binding polypeptide of the invention comprises an scFc region comprising one or more truncated Fc moieties that are nonetheless sufficient to confer Fc receptor (FcR) binding properties to the Fc region. For example, the portion of an Fc domain that binds to FcRn (i.e., the FcRn binding portion) comprises from about amino acids 282-438 of IgG1, EU numbering. Thus, an Fc moiety of a binding polypeptide of the invention may comprise or consist of an FcRn binding portion. FcRn binding portions may be derived from heavy chains of any isotype, including IgG1, IgG2, IgG3 and IgG4. In one embodiment, an FcRn binding portion from an antibody of the human isotype IgG1 is used. In another embodiment, an FcRn binding portion from an antibody of the human isotype IgG4 is used.

In one embodiment, a binding polypeptide of the invention lacks one or more constant region domains of a complete Fc region, i.e., they are partially or entirely deleted. In a certain embodiments binding polypeptides of the invention will lack an entire CH2 domain (ΔCH2 constructs). Those skilled in the art will appreciate that such constructs may be preferred due to the regulatory properties of the CH2 domain on the catabolic rate of the antibody. In certain embodiments, binding polypeptides of the invention comprise CH2 domain-deleted Fc regions derived from a vector (e.g., from IDEC Pharmaceuticals, San Diego) encoding an IgG1 human constant region domain (see, e.g., WO 02/060955A2 and WO02/096948A2). This exemplary vector is engineered to delete the CH2 domain and provide a synthetic vector expressing a domain-deleted IgG₁ constant region. It will be noted that these exemplary constructs are preferably engineered to fuse a binding CH3 domain directly to a hinge region of the respective Fc domain.

In other constructs it may be desirable to provide a peptide spacer between one or more constituent Fc moieties. For example, a peptide spacer may be placed between a hinge region and a CH2 domain and/or between a CH2 and a CH3 domains. For example, compatible constructs could be expressed wherein the CH2 domain has been deleted and the remaining CH3 domain (synthetic or unsynthetic) is joined to the hinge region with a 5-20 amino acid peptide spacer. Such a peptide spacer may be added, for instance, to ensure that the regulatory elements of the constant region domain remain free and accessible or that the hinge region remains flexible. Preferably, any linker peptide compatible with the instant invention will be relatively non-immunogenic and not prevent proper folding of the scFc region.

i) Changes to Fc Amino Acids

In certain embodiments, an Fc moiety employed in a binding polypeptide of the invention is altered, e.g., by amino acid mutation (e.g., addition, deletion, or substitution). As used herein, the term “Fc moiety variant” refers to an Fc moiety having at least one amino acid substitution as compared to the wild-type Fc from which the Fc moiety is derived. For example, wherein the Fc moiety is derived from a human IgG1 antibody, a variant comprises at least one amino acid mutation (e.g., substitution) as compared to a wild type amino acid at the corresponding position of the human IgG1 Fc region.

The amino acid substitution(s) of an Fc variant may be located at a position within the Fc moiety referred to as corresponding to the portion number that that residue would be given in an Fc region in an antibody (as set forth using the EU numbering convention). One of skill in the art can readily generate alignments to determine what the EU number corresponding to a position in an Fc moiety would be.

In one embodiment, the Fc variant comprises a substitution at an amino acid position located in a hinge domain or portion thereof. In another embodiment, the Fc variant comprises a substitution at an amino acid position located in a CH2 domain or portion thereof. In another embodiment, the Fc variant comprises a substitution at an amino acid position located in a CH3 domain or portion thereof. In another embodiment, the Fc variant comprises a substitution at an amino acid position located in a CH4 domain or portion thereof.

In certain embodiments, the binding polypeptides of the invention comprise an Fc variant comprising more than one amino acid substitution. The binding polypeptides of the invention may comprise, for example, 2, 3, 4, 5, 6, 7, 8, 9, 10 or more amino acid substitutions. Preferably, the amino acid substitutions are spatially positioned from each other by an interval of at least 1 amino acid position or more, for example, at least 2, 3, 4, 5, 6, 7, 8, 9, or 10 amino acid positions or more. More preferably, the engineered amino acids are spatially positioned apart from each other by an interval of at least 5, 10, 15, 20, or 25 amino acid positions or more.

In certain embodiments, the Fc variant confers an improvement in at least one effector function imparted by an Fc region comprising said wild-type Fc domain (e.g., an improvement in the ability of the Fc region to bind to Fc receptors (e.g. FcγRI, FcγRII, or FcγRIII) or complement proteins (e.g. C1q), or to trigger antibody-dependent cytotoxicity (ADCC), phagocytosis, or complement-dependent cytotoxicity (CDCC)). In other embodiments, the Fc variant provides an engineered cysteine residue

The binding polypeptides of the invention may employ art-recognized Fc variants which is known to impart an improvement in effector function and/or FcR binding. Specifically, a binding molecule of the invention may include, for example, a change (e.g., a substitution) at one or more of the amino acid positions disclosed in International PCT Publications WO88/07089A1, WO96/14339A1, WO98/05787A1, WO98/23289A1, WO99/51642A1, WO99/58572A1, WO00/09560A2, WO00/32767A1, WO00/42072A2, WO02/44215A2, WO02/060919A2, WO03/074569A2, WO04/016750A2, WO04/029207A2, WO04/035752A2, WO04/063351 A2, WO04/074455A2, WO04/099249A2, WO05/040217A2, WO04/044859, WO05/070963A1, WO05/077981A2, WO05/092925A2, WO05/123780A2, WO06/019447A1, WO06/047350A2, and WO06/085967A2; US Patent Publication Nos. US2007/0231329, US2007/0231329, US2007/0237765, US2007/0237766, US2007/0237767, US2007/0243188, US20070248603, US20070286859, US20080057056; or U.S. Pat. Nos. 5,648,260; 5,739,277; 5,834,250; 5,869,046; 6,096,871; 6,121,022; 6,194,551; 6,242,195; 6,277,375; 6,528,624; 6,538,124; 6,737,056; 6,821,505; 6,998,253; 7,083,784; and 7,317,091, each of which is incorporated by reference herein. In one embodiment, the specific change (e.g., the specific substitution of one or more amino acids disclosed in the art) may be made at one or more of the disclosed amino acid positions. In another embodiment, a different change at one or more of the disclosed amino acid positions (e.g., the different substitution of one or more amino acid position disclosed in the art) may be made.

In preferred embodiments, a binding polypeptide of the invention may comprise an Fc moiety variant comprising an amino acid substitution at an amino acid position corresponding to EU amino acid position that is within the “15 Angstrom Contact Zone” of an Fc moiety. The 15 Angstrom Zone includes residues located at EU positions 243 to 261, 275 to 280, 282-293, 302 to 319, 336 to 348, 367, 369, 372 to 389, 391, 393, 408, and 424-440 of a full-length, wild-type Fc moiety.

In certain embodiments, a binding polypeptide of the invention comprises an amino acid substitution to an Fc moiety which alters the antigen-independent effector functions of the antibody, in particular the circulating half-life of the antibody. Such binding polypeptides exhibit either increased or decreased binding to FcRn when compared to binding polypeptides lacking these substitutions and, therefore, have an increased or decreased half-life in serum, respectively. Fc variants with improved affinity for FcRn are anticipated to have longer serum half-lives, and such molecules have useful applications in methods of treating mammals where long half-life of the administered polypeptide is desired, e.g., to treat a chronic disease or disorder. In contrast, Fc variants with decreased FcRn binding affinity are expected to have shorter half-lives, and such molecules are also useful, for example, for administration to a mammal where a shortened circulation time may be advantageous, e.g. for in vivo diagnostic imaging or in situations where the starting polypeptide has toxic side effects when present in the circulation for prolonged periods. Fc variants with decreased FcRn binding affinity are also less likely to cross the placenta and, thus, are also useful in the treatment of diseases or disorders in pregnant women. In addition, other applications in which reduced FcRn binding affinity may be desired include those applications in which localization the brain, kidney, and/or liver is desired. In one exemplary embodiment, the binding polypeptides of the invention exhibit reduced transport across the epithelium of kidney glomeruli from the vasculature. In another embodiment, the binding polypeptides of the invention exhibit reduced transport across the blood brain barrier (BBB) from the brain, into the vascular space. In one embodiment, a binding polypeptide with altered FcRn binding comprises at least one Fc moiety (e.g, one or two Fc moieties) having one or more amino acid substitutions within the “FcRn binding loop” of an Fc moiety. The FcRn binding loop is comprised of amino acid residues 280-299 (according to EU numbering) of a wild-type, full-length, Fc moiety. In other embodiments, a binding polypeptide of the invention having altered FcRn binding affinity comprises at least one Fc moiety (e.g, one or two Fc moieties) having one or more amino acid substitutions within the 15 \acute{Å} FcRn “contact zone.” As used herein, the term 15 \acute{Å} FcRn “contact zone” includes residues at the following positions of a wild-type, full-length Fc moiety: 243-261, 275-280, 282-293, 302-319, 336-348, 367, 369, 372-389, 391, 393, 408, 424, 425-440 (EU numbering). In preferred embodiments, a binding polypeptide of the invention having altered FcRn binding affinity comprises at least one Fc moiety (e.g, one or two Fc moieties) having one or more amino acid substitutions at an amino acid position corresponding to any one of the following EU positions: 256, 277-281, 283-288, 303-309, 313, 338, 342, 376, 381, 384, 385, 387, 434 (e.g., N434A or N434K), and 438. Exemplary amino acid substitutions which altered FcRn binding activity are disclosed in International PCT Publication No. WO05/047327 which is incorporated by reference herein.

In other embodiments, a binding polypeptide of the invention comprises an Fc variant comprising an amino acid substitution which alters the antigen-dependent effector functions of the polypeptide, in particular ADCC or complement activation, e.g., as compared to a wild type Fc region. In exemplary embodiment, said binding polypeptides exhibit altered binding to an Fc gamma receptor (e.g., CD16). Such binding polypeptides exhibit either increased or decreased binding to FcR gamma when compared to wild-type polypeptides and, therefore, mediate enhanced or reduced effector function, respectively. Fc variants with improved affinity for FcγRs are anticipated to enhance effector function, and such molecules have useful applications in methods of treating mammals where target molecule destruction is desired, e.g., in tumor therapy. In contrast, Fc variants with decreased FcγR binding affinity are expected to reduce effector function, and such molecules are also useful, for example, for treatment of conditions in which target cell destruction is undesirable, e.g., where normal cells may express target molecules, or where chronic administration of the polypeptide might result in unwanted immune system activation. In one embodiment, the polypeptide comprising an scFc exhibits at least one altered antigen-dependent effector function selected from the group consisting of opsonization, phagocytosis, complement dependent cytotoxicity, antigen-dependent cellular cytotoxicity (ADCC), or effector cell modulation as compared to a polypeptide comprising a wild type Fc region.

In one embodiment the binding polypeptide exhibits altered binding to an activating FcγR (e.g. FcγI, FcγIIa, or FcγRIIIa). In another embodiment, the binding polypeptide exhibits altered binding affinity to an inhibitory FcγR (e.g. FcγRIIb). In other embodiments, a binding polypeptide of the invention having increased FcγR binding affinity (e.g. increased FcγRIIIa binding affinity) comprises at least one Fc moiety (e.g, one or two Fc moieties) having an amino acid substitution at an amino acid position corresponding to one or more of the following positions: 239, 268, 298, 332, 334, and 378 (EU numbering). In other embodiments, a binding polypeptide of the invention having decreased FcγR binding affinity (e.g. decreased FcγRI, FcγRII, or FcγRIIIa binding affinity) comprises at least one Fc moiety (e.g, one or two Fc moieties) having an amino acid substitution at an amino acid position corresponding to one or more of the following positions: 234, 236, 239, 241, 251, 252, 261, 265, 268, 293, 294, 296, 298, 299, 301, 326, 328, 332, 334, 338, 376, 378, and 435 (EU numbering). In other embodiments, a binding polypeptide of the invention having increased complement binding affinity (e.g. increased C1q binding affinity) comprises an Fc moiety (e.g, one or two Fc moieties) having an amino acid substitution at an amino acid position corresponding to one or more of the following positions: 251, 334, 378, and 435 (EU numbering). In other embodiments, a binding polypeptide of the invention having decreased complement binding affinity (e.g. decreased C1q binding affinity) comprises an Fc moiety (e.g, one or two Fc moieties) having an amino acid substitution at an amino acid position corresponding to one or more of the following positions: 239, 294, 296, 301, 328, 333, and 376 (EU numbering). Exemplary amino acid substitutions which altered FcγR or complement binding activity are disclosed in International PCT Publication No. WO05/063815 which is incorporated by reference herein. In certain preferred embodiments, binding polypeptide of the invention may comprise one or more of the following specific substitutions: S239D, S239E, M252T, H268D, H268E, I332D, I332E, N434A, and N434K (i.e., one or more of these substitutions at an amino acid position corresponding to one or more of these EU numbered position in an antibody Fc region).

A binding polypeptide of the invention may also comprise an an amino acid substitution which alters the glycosylation of the binding polypeptide. For example, the scFc region of the binding polypeptide may comprise an Fc moiety having a mutation leading to reduced glycosylation (e.g., N- or O-linked glycosylation) or may comprise an altered glycoform of the wild-type Fc moiety (e.g., a low fucose or fucose-free glycan). In exemplary embodiments, the Fc moiety comprises reduced glycosylation of the N-linked glycan normally found at amino acid position 297 (EU numbering). In another exemplary embodiment, the Fc moiety comprises a low fucose or fucose free glycan at amino acid position 297 (EU numbering). In another embodiment, the binding polypeptide has an amino acid substitution near or within a glycosylation motif, for example, an N-linked glycosylation motif that contains the amino acid sequence NXT or NXS. In a particular embodiment, the binding polypeptide comprises an amino acid substitution at an amino acid position corresponding to 299 of Fc (EU numbering). Exemplary amino acid substitutions which reduce or alter glycosylation are disclosed in International PCT Publication No. WO05/018572 and US Patent Publication No. 2007/0111281, which are incorporated by reference herein.

In other embodiments, a binding polypeptide of the invention comprises at least one Fc moiety having engineered cysteine residue or analog thereof which is located at the solvent-exposed surface. Preferably the engineered cysteine residue or analog thereof does not interfere with an effector function conferred by the scFc region. More preferably, the alteration does not interfere with the ability of the scFc region to bind to Fc receptors (e.g. FcγRI, FcγRII, or FcγRIII) or complement proteins (e.g. C1q), or to trigger immune effector function (e.g., antibody-dependent cytotoxicity (ADCC), phagocytosis, or complement-dependent cytotoxicity (CDCC)). In preferred embodiments, the binding polypeptides of the invention comprise an Fc moiety comprising at least one engineered free cysteine residue or analog thereof that is substantially free of disulfide bonding with a second cysteine residue. In preferred embodiments, the binding polypeptides of the invention may comprise an Fc moiety having engineered cysteine residues or analogs thereof at one or more of the following positions in the CH3 domain: 349-371, 390, 392, 394-423, 441-446, and 446b (EU numbering). In more preferred embodiments, the binding polypeptides of the invention comprise an Fc variant having engineered cysteine residues or analogs thereof at any one of the following positions: 350, 355, 359, 360, 361, 389, 413, 415, 418, 422, 441, 443, and EU position 446b (EU numbering). Any of the above engineered cysteine residues or analogs thereof may subsequently be conjugated to a functional moiety using art-recognized techniques (e.g., conjugated with a thiol-reactive heterobifunctional linker).

In one embodiment, the binding polypeptide of the invention may comprise a genetically fused Fc region (i.e., scFc region) having two or more of its constituent Fc moieties independently selected from the Fc moieties described herein. In one embodiment, the Fc moieties are the same. In another embodiment, at least two of the Fc moieties are different. For example, the Fc moieties of the binding polypeptides of the invention comprise the same number of amino acid residues or they may differ in length by one or more amino acid residues (e.g., by about 5 amino acid residues (e.g., 1, 2, 3, 4, or 5 amino acid residues), about 10 residues, about 15 residues, about 20 residues, about 30 residues, about 40 residues, or about 50 residues). In yet other embodiments, the Fc moieties of the binding polypeptides of the invention may differ in sequence at one more amino acid positions. For example, at least two of the Fc moieties may differ at about 5 amino acid positions (e.g., 1, 2, 3, 4, or 5 amino acid positions), about 10 positions, about 15 positions, about 20 positions, about 30 positions, about 40 positions, or about 50 positions).

### B. Polypeptide Linkers

In certain aspects, it is desirable to employ a polypeptide linker to genetically fuse two or more Fc domains or moieties of an scFc region of a binding polypeptide of the invention. Such polypeptide linkers are referred to herein as “Fc connecting polypeptides”. In one embodiment, the polypeptide linker is synthetic. As used herein the term “synthetic” with respect to a polypeptide linker includes peptides (or polypeptides) which comprise an amino acid sequence (which may or may not be naturally occurring) that is linked in a linear sequence of amino acids to a sequence (which may or may not be naturally occurring) (e.g., an Fc moiety sequence) to which it is not naturally linked in nature. For example, said polypeptide linker may comprise non-naturally occurring polypeptides which are modified forms of naturally occurring polypeptides (e.g., comprising a mutation such as an addition, substitution or deletion) or which comprise a first amino acid sequence (which may or may not be naturally occurring). The polypeptide linkers of the invention may be employed, for instance, to ensure that Fc moieties or domains of the genetically-fused Fc region (i.e., scFc region) are juxtaposed to ensure proper folding and formation of a functional scFc region. Preferably, a polypeptide linker compatible with the instant invention will be relatively non-immunogenic and not inhibit any non-covalent association among monomer subunits of a binding protein.

In certain embodiments, the binding polypeptides of the invention employ a polypeptide linker to join any two or more Fc moieties or domains in frame in a single polypeptide chain. In one embodiment, the two or more Fc moieties or domains may be independently selected from any of the Fc moieties discussed in section A supra. For example, in certain embodiments, a polypeptide linker can be used to fuse identical Fc moeities, thereby forming a homomeric scFc region. In other embodiments, a polypeptide linker can be used to fuse different Fc moieties (e.g. a wild-type Fc moiety and a Fc moiety variant), thereby forming a heteromeric scFc region. In other embodiments, a polypeptide linker of the invention can be used to genetically fuse the C-terminus of a first Fc moiety (e.g. a hinge domain or portion thereof, a CH2 domain or portion thereof, a complete CH3 domain or portion thereof, a FcRn binding portion, an FcγR binding portion, a complement binding portion, or portion thereof) to the N-terminus of a second Fc moiety (e.g., a complete Fc domain).

In one embodiment, a synthetic polypeptide linker comprises a portion of an Fc moiety. For example, in one embodiment, a polypeptide linker can comprise an immunoglobulin hinge domain of an IgG1, IgG2, IgG3, and/or IgG4 antibody. In another embodiment, a polypeptide linker can comprise a CH2 domain of an IgG1, IgG2, IgG3, and/or IgG4 antibody. In other embodiments, a polypeptide linker can comprise a CH3 domain of an IgG1, IgG2, IgG3, and/or IgG4 antibody. Other portions of an immunoglobulin (e.g. a human immunoglobulin) can be used as well. For example, a polypeptide linker can comprise a CH1 domain or portion thereof, a CL domain or portion thereof, a VH domain or portion thereof, or a VL domain or portion thereof. Said portions can be derived from any immunoglobulin, including, for example, an IgG1, IgG2, IgG3, and/or IgG4 antibody.

In exemplary embodiments, a polypeptide linker can comprise at least a portion of an immunoglobulin hinge region. In one embodiment, a polypeptide linker comprises an upper hinge domain (e.g., an IgG1, an IgG2, an IgG3, or IgG4 upper hinge domain).

In another embodiment, a polypeptide linker comprises a middle hinge domain (e.g., an IgG1, an IgG2, an IgG3, or an IgG4 middle hinge domain). In another embodiment, a polypeptide linker comprises a lower hinge domain (e.g., an IgG1, an IgG2, an IgG3, or an IgG4 lower hinge domain). Exemplary hinge domain portions are listed in Table 1 below. In addition, any sub-portion of these exemplary hinges may be employed (e.g, the repeat portion of the IgG3 middle region (i.e., EPKSCDTPPPCPRCP).

TABLE 1
IgG1, IgG2, IgG3 and IgG4 Hinge Domains

| IgG | Upper Hinge | Middle Hinge | Lower Hinge |
|---|---|---|---|
| IgG1 | EPKSCDKTHT | CPPCP | APELLGGP |
| | (SEQ ID NO: 15) | (SEQ ID NO: 16) | (SEQ ID NO: 17) |
| IgG2 | ERKCCVE | CPPCP | APPVAGP |
| | (SEQ ID NO: 82) | (SEQ ID NO: 16) | (SEQ ID NO: 83) |
| IgG3 | ELKTPLGDTTHT | CPRCP (EPKSCDTPPPCPRCP)₃ | APELLGGP |
| | (SEQ ID NO: 18) | (SEQ ID NO: 19) | (SEQ ID NO: 20) |
| IgG4 | ESKYGPP | CPSCP | APEFLGGP |
| | (SEQ ID NO: 21) | (SEQ ID NO: 22) | (SEQ ID NO: 23) |

In other embodiments, polypeptide linkers can be constructed which combine hinge elements derived from the same or different antibody isotypes. In one embodiment, the polypeptide linker comprises a chimeric hinge comprising at least a portion of an IgG1 hinge region and at least a portion of an IgG2 hinge region. In one embodiment, the polypeptide linker comprises a chimeric hinge comprising at least a portion of an IgG1 hinge region and at least a portion of an IgG3 hinge region. In another embodiment, a polypeptide linker comprises a chimeric hinge comprising at least a portion of an IgG1 hinge region and at least a portion of an IgG4 hinge region. In one embodiment, the polypeptide linker comprises a chimeric hinge comprising at least a portion of an IgG2 hinge region and at least a portion of an IgG3 hinge region. In one embodiment, the polypeptide linker comprises a chimeric hinge comprising at least a portion of an IgG2 hinge region and at least a portion of an IgG4 hinge region. In one embodiment, the polypeptide linker comprises a chimeric hinge comprising at least a portion of an IgG1 hinge region, at least a portion of an IgG2 hinge region, and at least a portion of an IgG4 hinge region. In another embodiment, a polypeptide linker can comprise an IgG1 upper and middle hinge and a single IgG3 middle hinge repeat motif.

In another embodiment, a polypeptide linker can comprise an IgG4 upper hinge, an IgG1 middle hinge and a IgG2 lower hinge.

In another embodiment, a polypeptide linker comprises or consists of a gly-ser linker. As used herein, the term “gly-ser linker” refers to a peptide that consists of glycine and serine residues. An exemplary gly/ser linker comprises an amino acid sequence of the formula (Gly₄Ser)n, wherein is a positive integer (e.g., 1, 2, 3, 4, or 5). A preferred gly/ser linker is (Gly₄Ser)4. Another preferred gly/ser linker is (Gly₄Ser)3. Another exemplary gly-ser linker is GGGSSGGGSG (SEQ ID NO:24). In certain embodiments, said gly-ser linker may be inserted between two other sequences of the polypeptide linker (e.g., any of the polypeptide linker sequences described herein). In other embodiments, a gly-ser linker is attached at one or both ends of another sequence of the polypeptide linker (e.g., any of the polypeptide linker sequences described herein). In yet other embodiments, two or more gly-ser linker are incorporated in series in a polypeptide linker. In one embodiment, a polypeptide linker of the invention comprises at least a portion of an upper hinge region (e.g., derived from an IgG1, IgG2, IgG3, or IgG4 molecule), at least a portion of a middle hinge region (e.g., derived from an IgG1, IgG2, IgG3, or IgG4 molecule) and a series of gly/ser amino acid residues (e.g., a gly/ser linker such as (Gly₄Ser)n).

In another embodiment, a polypeptide linker comprises an amino acid sequence such as described in WO 02/060955. In another embodiment, a polypeptide linker comprises the amino acid sequence IGKTISKKAK. Another exemplary polypeptide linker comprises the sequence (G4S)₄GGGAS.

A particularly preferred polypeptide linker comprises the amino acid sequence SLSLSPGGGGGSEPKSS. Another preferred polypeptide linker comprises a human IgG1 hinge sequence, e.g., DKTHTCPPCPAPELLGG. Yet another preferred polypeptide linker comprises both sequences.

In one embodiment, a polypeptide linker of the invention comprises a non-naturally occurring immunoglobulin hinge region domain, e.g., a hinge region domain that is not naturally found in the polypeptide comprising the hinge region domain and/or a hinge region domain that has been altered so that it differs in amino acid sequence from a naturally occurring immunoglobulin hinge region domain. In one embodiment, mutations can be made to hinge region domains to make a polypeptide linker of the invention. In one embodiment, a polypeptide linker of the invention comprises a hinge domain which does not comprise a naturally occurring number of cysteines, i.e., the polypeptide linker comprises either fewer cysteines or a greater number of cysteines than a naturally occurring hinge molecule. In one embodiment of the invention, a polypeptide linker comprises hinge region domain comprising a proline residue at an amino acid position corresponding to amino acid position 230 (EU numbering system). In one embodiment, a polypeptide linker comprises an alanine residue at an amino acid position corresponding to position 231 (EU numbering system). In another embodiment, a polypeptide linker of the invention comprises a proline residue at an amino acid position corresponding to position 232 (EU numbering system)). In one embodiment, a polypeptide linker comprises a cysteine residue at an amino acid position corresponding to position 226 (EU numbering system). In one embodiment, a polypeptide linker comprises a serine residue at an amino acid position corresponding to position 226 (EU numbering system). In one embodiment, a polypeptide linker comprises a cysteine residue at an amino acid position corresponding to position 229 (EU numbering system). In one embodiment, a polypeptide linker comprises a serine residue at an amino acid position corresponding to position 229 (EU numbering system).

In other embodiments, a polypeptide linker of the invention comprises a biologically relevant peptide sequence or a sequence portion thereof. For example, a biologically relevant peptide sequence may include, but is not limited to, sequences derived from an anti-rejection or anti-inflammatory peptide. Said anti-rejection or anti-inflammatory peptides may be selected from the group consisting of a cytokine inhibitory peptide, a cell adhesion inhibitory peptide, a thrombin inhibitory peptide, and a platelet inhibitory peptide. In a one preferred embodiment, a polypeptide linker comprises a peptide sequence selected from the group consisting of an IL-1 inhibitory or antagonist peptide sequence, an erythropoietin (EPO)-mimetic peptide sequence, a thrombopoietin (TPO)-mimetic peptide sequence, G-CSF mimetic peptide sequence, a TNF-antagonist peptide sequence, an integrin-binding peptide sequence, a selectin antagonist peptide sequence, an anti-pathogenic peptide sequence, a vasoactive intestinal peptide (VIP) mimetic peptide sequence, a calmodulin antagonist peptide sequence, a mast cell antagonist, a SH3 antagonist peptide sequence, an urokinase receptor (UKR) antagonist peptide sequence, a somatostatin or cortistatin mimetic peptide sequence, and a macrophage and/or T-cell inhibiting peptide sequence. Exemplary peptide sequences, any one of which may be employed as a polypeptide linker, are disclosed in U.S. Pat. No. 6,660,843, which is incorporated by reference herein.

In other embodiments, a polypeptide linker comprises one or more of any one of the binding sites described infra (e.g., a Fab, an scFv molecule, a receptor binding portion of ligand, a ligand binding portion of a receptor, etc.).

It will be understood that variant forms of these exemplary polypeptide linkers can be created by introducing one or more nucleotide substitutions, additions or deletions into the nucleotide sequence encoding a polypeptide linker such that one or more amino acid substitutions, additions or deletions are introduced into the polypeptide linker. For example, mutations may be introduced by standard techniques, such as site-directed mutagenesis and PCR-mediated mutagenesis. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

Polypeptide linkers of the invention are at least one amino acid in length and can be of varying lengths. In one embodiment, a polypeptide linker of the invention is from about 1 to about 50 amino acids in length. As used in this context, the term about indicates +/−two amino acid residues. Since linker length must be a positive interger, the length of from about 1 to about 50 amino acids in length, means a length of from 1 to 48-52 amino acids in length. In another embodiment, a polypeptide linker of the invention is from about 10-20 amino acids in length. In another embodiment, a polypeptide linker of the invention is from about 15 to about 50 amino acids in length.

In another embodiment, a polypeptide linker of the invention is from about 20 to about 45 amino acids in length. In another embodiment, a polypeptide linker of the invention is from about 15 to about 25 amino acids in length. In another embodiment, a polypeptide linker of the invention is from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, or 60 amino acids in length.

Polypeptide linkers can be introduced into polypeptide sequences using techniques known in the art. Modifications can be confirmed by DNA sequence analysis. Plasmid DNA can be used to transform host cells for stable production of the polypeptides produced.

### C. Target Binding Sites

In certain aspects, the binding polypeptides of the invention comprise at least one target binding site. Accordingly, the binding polypeptides of the invention typically comprise at least one binding site and at least one genetically-fused Fc region (i.e., scFc region).

In one embodiment, the binding site is operably linked (e.g., chemically conjugated or genetically fused (e.g., either directly or via a polypeptide linker)) to the N-terminus of a genetically-fused Fc region. In another embodiment, the binding site is operably linked (e.g., chemically conjugated or genetically fused (e.g., either directly or via a polypeptide linker)) to the C-terminus of a genetically-fused Fc region. In other embodiments, a binding site is operably linked (e.g., chemically conjugated or genetically fused (e.g., either directly or via a polypeptide linker)) via an amino acid side chain of the genetically-fused Fc region. In certain exemplary embodiments, the binding site is fused to a genetically-fused Fc region (i.e., scFc region) via a human immunoglobulin hinge domain or portion thereof (e.g., a human IgG1 sequence, e.g., DKTHTCPPCPAPELLGG (SEQ ID NO: 81)).

In certain embodiments, the binding polypeptides of the invention comprise two binding sites and at least one genetically-fused Fc region. For example, binding sites may be operably linked to both the N-terminus and C-terminus of a single genetically-fused Fc region. In other exemplary embodiments, binding sites may be operably linked to both the N- and C-terminal ends of multiple genetically-fused Fc regions (e.g., two, three, four, five, or more scFc regions) which are linked together in series to form a tandem array of genetically-fused Fc regions.

In other embodiments, two or more binding sites are linked to each other (e.g., via a polypeptide linker) in series, and the tandem array of binding sites is operably linked (e.g., chemically conjugated or genetically fused (e.g., either directly or via a polypeptide linker)) to either the C-terminus or the N-terminus of a single genetically-fused Fc region (i.e., a single scFc region) or a tandem array of genetically-fused Fc regions (i.e., tandem scFc regions). In other embodiments, the tandem array of binding sites is operably linked to both the C-terminus and the N-terminus of a single genetically-fused Fc region or a tandem array of genetically-fused Fc regions.

In other embodiments, a binding polypeptide of the invention is a trivalent binding polypeptide comprising three binding sites. An exemplary trivalent binding polypeptide of the invention is bispecific or trispecific. For example, a trivalent binding polypeptide may be bivalent (i.e., have two binding sites) for one specificity and monovalent for a second specificity.

In yet other embodiments, a binding polypeptide of the invention is a tetravalent binding polypeptide comprising four binding sites. An exemplary tetravalent binding polypeptide of the invention is bispecific. For example, a tetravalent binding polypeptide may be bivalent (i.e., have two binding sites) for each specificity.

As mentioned above, in other embodiments, one or more binding sites may be inserted between two Fc moieties of a genetically-fused Fc region (i.e., scFc region). For example, one or more binding sites may form all or part of a polypeptide linker of a binding polypeptide of the invention.

Preferred binding polypeptides of the invention comprise at least one of an antigen binding site (e.g., an antigen binding site of an antibody, antibody variant, or antibody fragment), a receptor binding portion of ligand, or a ligand binding portion of a receptor.

In other embodiments, the binding polypeptides of the invention comprise at least one binding site comprising one or more of any one of the biologically-relevant peptides discussed supra.

In certain embodiments, the binding polypeptides of the invention have at least one binding site specific for a target molecule which mediates a biological effect. In one embodiment, the binding site modulates cellular activation or inhibition (e.g., by binding to a cell surface receptor and resulting in transmission of an activating or inhibitory signal). In one embodiment, the binding site is capable of initiating transduction of a signal which results in death of the cell (e.g., by a cell signal induced pathway, by complement fixation or exposure to a payload (e.g., a toxic payload) present on the binding molecule), or which modulates a disease or disorder in a subject (e.g., by mediating or promoting cell killing, by promoting lysis of a fibrin clot or promoting clot formation, or by modulating the amount of a substance which is bioavailable (e.g., by enhancing or reducing the amount of a ligand such as TNFα in the subject)). In another embodiment, the binding polypeptides of the invention have at least one binding site specific for an antigen targeted for reduction or elimination, e.g., a cell surface antigen or a soluble antigen, together with at least one genetically-fused Fc region (i.e., scFc region).

In another embodiment, binding of the binding polypeptides of the invention to a target molecule (e.g. antigen) results in the reduction or elimination of the target molecule, e.g., from a tissue or from circulation. In another embodiment, the binding polypeptide has at least one binding site specific for a target molecule that can be used to detect the presence of the target molecule (e.g., to detect a contaminant or diagnose a condition or disorder). In yet another embodiment, a binding polypeptide of the invention comprises at least one binding site that targets the molecule to a specific site in a subject (e.g., to a tumor cell, an immune cell, or blood clot).

In certain embodiments, the binding polypeptides of the invention may comprise two or more binding sites. In one embodiment, the binding sites are identical. In another embodiment, the binding sites are different.

In other embodiments, the binding polypeptides of the invention may be assembled together or with other polypeptides to form binding proteins having two or more polypeptides (“binding proteins” or “multimers”), wherein at least one polypeptide of the multimer is a binding polypeptide of the invention. Exemplary multimeric forms include dimeric, trimeric, tetrameric, and hexameric altered binding proteins and the like. In one embodiment, the polypeptides of the binding protein are the same (ie. homomeric altered binding proteins, e.g. homodimers, homotetramers). In another embodiment, the polypeptides of the binding protein are different (e.g. heteromeric).

i. Antigen Binding Sites

### (a) Antibodies

In certain embodiments, a binding polypeptide of the invention comprises at least one antigen binding site of an antibody. Binding polypeptides of the invention may comprise a variable region or portion thereof (e.g. a VL and/or VH domain) derived from an antibody using art recognized protocols. For example, the variable domain may be derived from antibody produced in a non-human mammal, e.g., murine, guinea pig, primate, rabbit or rat, by immunizing the mammal with the antigen or a fragment thereof. See Harlow & Lane, supra, incorporated by reference for all purposes. The immunoglobulin may be generated by multiple subcutaneous or intraperitoneal injections of the relevant antigen (e.g., purified tumor associated antigens or cells or cellular extracts comprising such antigens) and an adjuvant. This immunization typically elicits an immune response that comprises production of antigen-reactive antibodies from activated splenocytes or lymphocytes.

While the variable region may be derived from polyclonal antibodies harvested from the serum of an immunized mammal, it is often desirable to isolate individual lymphocytes from the spleen, lymph nodes or peripheral blood to provide homogenous preparations of monoclonal antibodies (MAbs) from which the desired variable region is derived. Rabbits or guinea pigs are typically used for making polyclonal antibodies. Mice are typically used for making monoclonal antibodies. Monoclonal antibodies can be prepared against a fragment by injecting an antigen fragment into a mouse, preparing “hybridomas” and screening the hybridomas for an antibody that specifically binds to the antigen. In this well known process (Kohler et al., (1975), Nature, 256:495) the relatively short-lived, or mortal, lymphocytes from the mouse which has been injected with the antigen are fused with an immortal tumor cell line (e.g. a myeloma cell line), thus, producing hybrid cells or “hybridomas” which are both immortal and capable of producing the antibody genetically encoded by the B cell. The resulting hybrids are segregated into single genetic strains by selection, dilution, and regrowth with each individual strain comprising specific genes for the formation of a single antibody. They produce antibodies which are homogeneous against a desired antigen and, in reference to their pure genetic parentage, are termed “monoclonal”.

Hybridoma cells thus prepared are seeded and grown in a suitable culture medium that preferably contains one or more substances that inhibit the growth or survival of the unfused, parental myeloma cells. Those skilled in the art will appreciate that reagents, cell lines and media for the formation, selection and growth of hybridomas are commercially available from a number of sources and standardized protocols are well established. Generally, culture medium in which the hybridoma cells are growing is assayed for production of monoclonal antibodies against the desired antigen. Preferably, the binding specificity of the monoclonal antibodies produced by hybridoma cells is determined by immunoprecipitation or by an in vitro assay, such as a radioimmunoassay (RIA) or enzyme-linked immunosorbent assay (ELISA). After hybridoma cells are identified that produce antibodies of the desired specificity, affinity and/or activity, the clones may be subcloned by limiting dilution procedures and grown by standard methods (Goding, Monoclonal Antibodies: Principles and Practice, pp 59-103 (Academic Press, 1986)). It will further be appreciated that the monoclonal antibodies secreted by the subclones may be separated from culture medium, ascites fluid or serum by conventional purification procedures such as, for example, affinity chromatography (e.g., protein-A, protein-G, or protein-L affinity chromatography), hydroxylapatite chromatography, gel electrophoresis, or dialysis.

Optionally, antibodies may be screened for binding to a specific region or desired fragment of the antigen without binding to other nonoverlapping fragments of the antigen. The latter screening can be accomplished by determining binding of an antibody to a collection of deletion mutants of the antigen and determining which deletion mutants bind to the antibody. Binding can be assessed, for example, by Western blot or ELISA. The smallest fragment to show specific binding to the antibody defines the epitope of the antibody. Alternatively, epitope specificity can be determined by a competition assay is which a test and reference antibody compete for binding to the antigen. If the test and reference antibodies compete, then they bind to the same epitope or epitopes sufficiently proximal such that binding of one antibody interferes with binding of the other.

DNA encoding the desired monoclonal antibody may be readily isolated and sequenced using any of the conventional procedures described supra for the isolation of constant region domain sequences (e.g., by using oligonucleotide probes that are capable of binding specifically to genes encoding the heavy and light chains of murine antibodies). The isolated and subcloned hybridoma cells serve as a preferred source of such DNA. More particularly, the isolated DNA (which may be synthetic as described herein) may be used to clone the desired variable region sequences for incorporation in the binding polypeptides of the invention.

In other embodiments, the binding site is derived from a fully human antibody. Human or substantially human antibodies may be generated in transgenic animals (e.g., mice) that are incapable of endogenous immunoglobulin production (see e.g., U.S. Pat. Nos. 6,075,181, 5,939,598, 5,591,669 and 5,589,369, each of which is incorporated herein by reference). For example, it has been described that the homozygous deletion of the antibody heavy-chain joining region in chimeric and germ-line mutant mice results in complete inhibition of endogenous antibody production. Transfer of a human immunoglobulin gene array to such germ line mutant mice will result in the production of human antibodies upon antigen challenge. Another preferred means of generating human antibodies using SCID mice is disclosed in U.S. Pat. No. 5,811,524 which is incorporated herein by reference. It will be appreciated that the genetic material associated with these human antibodies may also be isolated and manipulated as described herein.

Yet another highly efficient means for generating recombinant antibodies is disclosed by Newman, Biotechnology, 10: 1455-1460 (1992). Specifically, this technique results in the generation of primatized antibodies that contain monkey variable domains and human constant sequences. This reference is incorporated by reference in its entirety herein. Moreover, this technique is also described in commonly assigned U.S. Pat. Nos. 5,658,570, 5,693,780 and 5,756,096 each of which is incorporated herein by reference.

In another embodiment, lymphocytes can be selected by micromanipulation and the variable genes isolated. For example, peripheral blood mononuclear cells can be isolated from an immunized mammal and cultured for about 7 days in vitro. The cultures can be screened for specific IgGs that meet the screening criteria. Cells from positive wells can be isolated. Individual Ig-producing B cells can be isolated by FACS or by identifying them in a complement-mediated hemolytic plaque assay. Ig-producing B cells can be micromanipulated into a tube and the VH and VL genes can be amplified using, e.g., RT-PCR. The VH and VL genes can be cloned into an antibody expression vector and transfected into cells (e.g., eukaryotic or prokaryotic cells) for expression.

Alternatively, variable (V) domains can be obtained from libraries of variable gene sequences from an animal of choice. Libraries expressing random combinations of domains, e.g., V_{H} and V_{L} domains, can be screened with a desired antigen to identify elements which have desired binding characteristics. Methods of such screening are well known in the art. For example, antibody gene repertoires can be cloned into a X bacteriophage expression vector (Huse, W D et al. (1989). Science, 2476:1275). In addition, cells (Francisco et al. (1994), PNAS, 90:10444; Georgiou et al. (1997), Nat. Biotech., 15:29; Boder and Wittrup (1997) Nat. Biotechnol. 15:553; Boder et al. (2000), PNAS, 97:10701; Daugtherty, P. et al. (2000) J. Immunol. Methods. 243:211) or viruses (e.g., Hoogenboom, H R. (1998), Immunotechnology 4:1; Winter et al. (1994). Annu. Rev. Immunol. 12:433; Griffiths, A D. (1998). Curr. Opin. Biotechnol. 9:102) expressing antibodies on their surface can be screened.

Those skilled in the art will also appreciate that DNA encoding antibody variable domains may also be derived from antibody libraries expressed in phage, yeast, or bacteria using methods known in the art. Exemplary methods are set forth, for example, in EP 368 684B1;U.S. Pat. No. 5,969,108; Hoogenboom et al., (2000)Immunol. Today 21:371; Nagy et al. (2002) Nat. Med. 8:801; Huie et al. (2001), PNAS, 98:2682; Lui et al. (2002), J. Mol. Biol. 315:1063, each of which is incorporated herein by reference. Several publications (e.g., Marks et al. (1992), Bio/Technology 10:779-783) have described the production of high affinity human antibodies by chain shuffling, as well as combinatorial infection and in vivo recombination as a strategy for constructing large phage libraries. In another embodiment, ribosomal display can be used to replace bacteriophage as the display platform (see, e.g., Hanes, et al. (1998), PNAS 95:14130; Hanes and Pluckthun. (1999), Curr. Top. Microbiol. Immunol. 243:107; He and Taussig. (1997), Nuc. Acids Res., 25:5132; Hanes et al. (2000), Nat. Biotechnol. 18:1287; Wilson et al. (2001), PNAS, 98:3750; or Irving et al. (2001) J. Immunol. Methods 248:31).

Preferred libraries for screening are human variable gene libraries. V_{L} and V_{H} domains from a non-human source may also be used. Libraries can be naïve, from immunized subjects, or semi-synthetic (Hoogenboom and Winter. (1992). J. Mol. Biol. 227:381; Griffiths et al. (1995) EMBO J. 13:3245; de Kruif et al. (1995). J. Mol. Biol. 248:97; Barbas et al. (1992), PNAS, 89:4457). In one embodiment, mutations can be made to immunoglobulin domains to create a library of nucleic acid molecules having greater heterogeneity (Thompson et al. (1996), J. Mol. Biol. 256:77; Lamminmaki et al. (1999), J. Mol. Biol. 291:589; Caldwell and Joyce. (1992), PCR Methods Appl. 2:28; Caldwell and Joyce. (1994), PCR Methods Appl. 3:S136). Standard screening procedures can be used to select high affinity variants. In another embodiment, changes to VH and VL sequences can be made to increase antibody avidity, e.g., using information obtained from crystal structures using techniques known in the art.

Moreover, variable region sequences useful for producing the binding polypeptides of the present invention may be obtained from a number of different sources. For example, as discussed above, a variety of human gene sequences are available in the form of publicly accessible deposits. Many sequences of antibodies and antibody-encoding genes have been published and suitable variable region sequences (e.g. VL and VH sequences) can be chemically synthesized from these sequences using art recognized techniques.

In another embodiment, at least one variable region domain present in a binding polypeptide of the invention is catalytic (Shokat and Schultz. (1990). Annu. Rev. Immunol. 8:335). Variable region domains with catalytic binding specificities can be made using art recognized techniques (see, e.g., U.S. Pat. No. 6,590,080, U.S. Pat. No. 5,658,753). Catalytic binding specificities can work by a number of basic mechanisms similar to those identified for enzymes to stabilize the transition state, thereby reducing the free energy of activation. For example, general acid and base residues can be optimally positioned for participation in catalysis within catalytic active sites; covalent enzyme-substrate intermediates can be formed; catalytic antibodies can also be in proper orientation for reaction and increase the effective concentration of reactants by at least seven orders of magnitude (Fersht et al., (1968), J. Am. Chem. Soc. 90:5833) and thereby greatly reduce the entropy of a chemical reaction. Finally, catalytic antibodies can convert the energy obtained upon substrate binding and/or subsequent stabilization of the transition state intermediate to drive the reaction.

Acid or base residues can be brought into the antigen binding site by using a complementary charged molecule as an immunogen. This technique has proved successful for elicitation of antibodies with a hapten containing a positively-charged ammonium ion (Shokat, et al., (1988), Chem. Int. Ed. Engl. 27:269-271). In another approach, antibodies can be elicited to stable compounds that resemble the size, shape, and charge of the transition state intermediate of a desired reaction (i.e., transition state analogs). See U.S. Pat. No. 4,792,446 and U.S. Pat. No. 4,963,355 which describe the use of transition state analogs to immunize animals and the production of catalytic antibodies. Both of these patents are hereby incorporated by reference. Such molecules can be administered as part of an immunoconjugate, e.g., with an immunogenic carrier molecule, such as KLH.

In another embodiment, a variable region domain of an altered antibody of the invention consists of a V_{H} domain, e.g., derived from camelids, which is stable in the absence of a V_{L} chain (Hamers-Casterman et al. (1993). Nature, 363:446; Desmyter et al. (1996). Nat. Struct. Biol. 3: 803; Decanniere et al. (1999). Structure, 7:361; Davies et al. (1996). Protein Eng., 9:531; Kortt et al. (1995). J Protein Chem., 14:167).

Further, a binding polypeptide of the invention may comprise a variable domain or CDR derived from a fully murine, fully human, chimeric, humanized, non-human primate or primatized antibody. Non-human antibodies, or fragments or domains thereof, can be altered to reduce their immunogenicity using art recognized techniques. Humanized antibodies are antibodies derived from non-human antibodies, that have been modified to retain or substantially retain the binding properties of the parent antibody, but which are less immunogenic in humans that the parent, non-human antibodies. In the case of humanized target antibodies, this may be achieved by various methods, including (a) grafting the entire non-human variable domains onto human constant regions to generate chimeric target antibodies; (b) grafting at least a part of one or more of the non-human complementarity determining regions (CDRs) into a human framework and constant regions with or without retention of critical framework residues; (c) transplanting the entire non-human variable domains, but “cloaking” them with a human-like section by replacement of surface residues. Such methods are disclosed in Morrison et al., (1984), PNAS. 81: 6851-5; Morrison et al., (1988), Adv. Immunol. 44: 65-92; Verhoeyen et al., (1988), Science 239: 1534-1536; Padlan, (1991), Molec. Immun. 28: 489-498; Padlan, (1994), Molec. Immun. 31: 169-217; and U.S. Pat. Nos. 5,585,089, 5,693,761 and 5,693,762 all of which are hereby incorporated by reference in their entirety.

De-immunization can also be used to decrease the immunogenicity of a binding polypeptide of the invention. As used herein, the term “de-immunization” includes modification of T cell epitopes (see, e.g., WO9852976A1, WO0034317A2). For example, VH and VL sequences are analyzed and a human T cell epitope “map” from each V region showing the location of epitopes in relation to complementarity-determining regions (CDRs) and other key residues within the sequence is generated. Individual T cell epitopes from the T cell epitope map are analyzed in order to identify alternative amino acid substitutions with a low risk of altering the activity of the final antibody. A range of alternative VH and VL sequences are designed comprising combinations of amino acid substitutions and these sequences are subsequently incorporated into a range of polypeptides of the invention that are tested for function. Typically, between 12 and 24 variant antibodies are generated and tested. Complete heavy and light chain genes comprising modified V and human C regions are then cloned into expression vectors and the subsequent plasmids introduced into cell lines for the production of whole antibody. The antibodies are then compared in appropriate biochemical and biological assays, and the optimal variant is identified.

In one embodiment, the variable domains employed in a binding polypeptide of the invention are altered by at least partial replacement of one or more CDRs. In another embodiment, variable domains can optionally be altered, e.g., by partial framework region replacement and sequence changing. In making a humanized variable region the CDRs may be derived from an antibody of the same class or even subclass as the antibody from which the framework regions are derived, however, it is envisaged that the CDRs will be derived from an antibody of different class and preferably from an antibody from a different species. It may not be necessary to replace all of the CDRs with the complete CDRs from the donor variable region to transfer the antigen binding capacity of one variable domain to another. Rather, it may only be necessary to transfer those residues that are necessary to maintain the activity of the binding domain. Given the explanations set forth in U.S. Pat. Nos. 5,585,089, 5,693,761 and 5,693,762, it will be well within the competence of those skilled in the art, either by carrying out routine experimentation or by trial and error testing to obtain a functional antigen binding site with reduced immunogenicity.

In one embodiment, a binding polypeptide of the invention comprises at least one CDR from an antibody that recognizes a desired target. In another embodiment, an altered antibody of the present invention comprises at least two CDRs from an antibody that recognizes a desired target. In another embodiment, an altered antibody of the present invention comprises at least three CDRs from an antibody that recognizes a desired target. In another embodiment, an altered antibody of the present invention comprises at least four CDRs from an antibody that recognizes a desired target. In another embodiment, an altered antibody of the present invention comprises at least five CDRs from an antibody that recognizes a desired target. In another embodiment, an altered antibody of the present invention comprises all six CDRs from an antibody that recognizes a desired target.

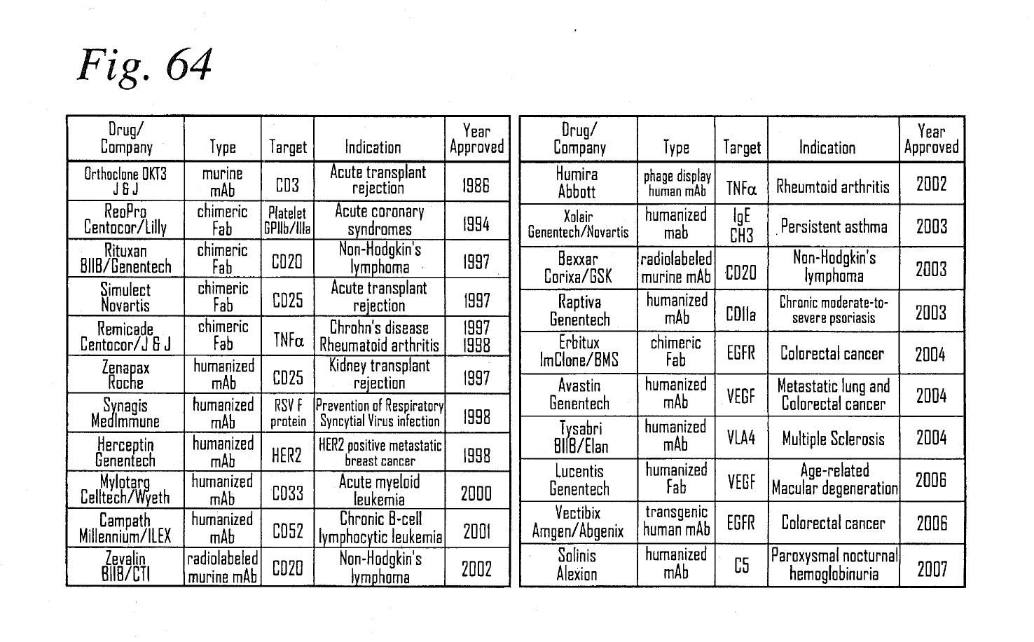

Exemplary antibodies from which binding sites can be derived for use in the binding molecules of the invention are known in the art. For example, antibodies currently approved by the FDA can be used to derive binding sites. Exemplary such antibodies are set forth in FIG. 64.

In one embodiment, antigen binding sites employed in the binding polypeptides of the present invention may be immunoreactive with one or more tumor-associated antigens. For example, for treating a cancer or neoplasia an antigen binding domain of a binding polypeptide preferably binds to a selected tumor associated antigen. Given the number of reported antigens associated with neoplasias, and the number of related antibodies, those skilled in the art will appreciate that a binding polypeptide of the invention may comprise a variable region sequence or portion thereof derived from any one of a number of whole antibodies. More generally, such a variable region sequence may be obtained or derived from any antibody (including those previously reported in the literature) that reacts with an antigen or marker associated with the selected condition. Exemplary tumor-associated antigens bound by the binding polypeptides of the invention include for example, pan B antigens (e.g. CD20 found on the surface of both malignant and non-malignant B cells such as those in non-Hodgkin's lymphoma) and pan T cell antigens (e.g. CD2, CD3, CD5, CD6, CD7). Other exemplary tumor associated antigens comprise but are not limited to MAGE-1, MAGE-3, MUC-1, HPV 16, HPV E6 & E7, TAG-72, CEA, α-Lewis^{y}, L6-Antigen, CD19, CD22, CD23, CD25, CD30, CD33, CD37, CD44, CD52, CD56, CD80, mesothelin, PSMA, HLA-DR, EGF Receptor, VEGF, VEGF Receptor, Cripto antigen, and HER2 Receptor.

In other embodiments, the binding polypeptide of the invention may comprise the complete antigen binding site (or variable regions or CDR sequences thereof) from antibodies that have previously been reported to react with tumor-associated antigens. Exemplary antibodies capable of reacting with tumor-associated antigens include: 2B8, Lym 1, Lym 2, LL2, Her2, B1, BR96, MB1, BH3, B4, B72.3, 5E8, B3F6, 5E10, α-CD33, α-CanAg, α-CD56, α-CD44v6, α-Lewis, and α-CD30. More specifically, these exemplary antibodies include, but are not limited to 2B8 and C2B8 (Zevalin® and Rituxan®, Biogen Idec, Cambridge), Lym 1 and Lym 2 (Techniclone), LL2 (Immunomedics Corp., New Jersey), Trastuzumab (Herceptin®, Genentech Inc., South San Francisco), Tositumomab (Bexxar®, Coulter Pharm., San Francisco), Alemtzumab (Campath®, Millennium Pharmaceuticals, Cambridge), Gemtuzumab ozogamicin (Mylotarg®, Wyeth-Ayerst, Philadelphia), Abagovomab (Menarini, Italy), CEA-Scamm (Immunomedics, Morris Plains, N.J.), Capromab (Prostascint®, Cytogen Corp.), Edrecolomab (Panorex®, Johnson & Johnson, New Brunswick, N.J.), Igovomab (CIS Bio Intl., France), Mitumomab (BEC2, Imclone Systems, Somerville, N.J.), Nofetumomab (Verluma®, Boehringer Ingleheim, Ridgefield, Conn.), OvaRex (Altarex Corp., Waltham, Mass.), Satumomab (Onoscint®, Cytogen Corp.), Apolizumab (REMITOGEN™, Protein Design Labs, Fremont, Calif.), Labetuzumab (CEACIDE™, Immunomedics Inc., Morris Plains, N.J.), Pertuzumab (OMNITARG™, Genentech Inc., S. San Francisco, Calif.), Panitumumab (Vectibix®, Amgen, Thousand Oaks, Calif.), Cetuximab (Erbitux®, Imclone Systems, New York), Bevacizumab (Avastin®, Genentech Inc., South San Francisco), BR96, BL22, LMB9, LMB2, MB1, BH3, B4, B72.3 (Cytogen Corp.), SS1 (NeoPharm), CC49 (National Cancer Institute), Cantuzumab mertansine (ImmunoGen, Cambridge), MNL 2704 (Milleneum Pharmaceuticals, Cambridge), Bivatuzumab mertansine (Boehringer Ingelheim, Germany), Trastuzumab-DM1 (Genentech, South San Francisco), My9-6-DM1 (ImmunoGen, Cabridge), SGN-10, -15, -25, and -35 (Seattle Genetics, Seattle), and 5E10 (University of Iowa). In yet other embodiments, the binding polyeptides may comprise the binding site of an anti-CD23 antibody (e.g., Lumiliximab), an anti-CD80 antibody (e.g., Galiximab), or an anti-VL5/α5β1-integrin antibody (e.g., Volociximab). In other embodiments, the binding polypeptides of the present invention will bind to the same tumor-associated antigens as the antibodies enumerated immediately above. In particularly preferred embodiments, the polypeptides will be derived from or bind the same antigens as Y2B8, C2B8, CC49 and C5E10.

Other binding sites that can be incorporated into the subject binding molecules include those found in: Orthoclone OKT3 (anti-CD3) (Johnson&Johnson, Brunswick, N.J.), ReoPro® (anti-GpIIb/gIIa)(Centocor, Horsham, Pa.), Zenapax® (anti-CD25)(Roche, Basel, Switzerland), Remicade® (anti-TNFα)(Centocor, Horsham, Pa.), Simulect® (anti-CD25)(Novartis, Basel, Switzerland), Synagis® (anti-RSV)(Medimmune, Gaithersburg, Md.), Humira® (anti-TNFα)(Abbott, Abbott Park, Ill.), Xolair® (anti-IgE)(Genentech, South San Francisco, Calif.), Raptiva® (anti-CD11a)(Genentech), Tysabri® (BiogenIdec, Cambridge, Mass.), Lucentis® (anti-VEGF)(Genentech), and Soliris® (Alexion Pharmaceuticals, Cheshire, Conn.).

In one embodiment, a binding molecule of the invention may have one or more binding sites derived from one or more of the following antibodies. tositumomab (BEXXAR®), muromonab (ORTHOCLONE®) and ibritumomab (ZEVALIN®), cetuximab (ERBITUX™), rituximab (MABTHERA®/RITUXAN®), infliximab (REMICADE®), abciximab (REOPRO®) and basiliximab (SIMULECT®), efalizumab (RAPTIVA®, bevacizumab (AVASTIN®), alemtuzumab (CAMPATH®), trastuzumab (HERCEPTIN®), gemtuzumab (MYLOTARG®), palivizumab (SYNAGIS®), omalizumab (XOLAIR®), daclizumab (ZENAPAX®), natalizumab (TYSABRI®) and ranibizumab (LUVENTIS®), adalimumab (HUMIRA®) and panitumumab (VECTIBIX®).

In one embodiment, the binding polypeptide will bind to the same tumor-associated antigen as Rituxan®. Rituxan® (also known as, rituximab, IDEC-C2B8 and C2B8) was the first FDA-approved monoclonal antibody for treatment of human B-cell lymphoma (see U.S. Pat. Nos. 5,843,439; 5,776,456 and 5,736,137 each of which is incorporated herein by reference). Y2B8 (90Y labeled 2B8; Zevalin®; ibritumomab tiuxetan) is the murine, parent antibody of C2B8. Rituxan® is a chimeric, anti-CD20 monoclonal antibody which is growth inhibitory and reportedly sensitizes certain lymphoma cell lines for apoptosis by chemotherapeutic agents in vitro. The antibody efficiently binds human complement, has strong FcR binding, and can effectively kill human lymphocytes in vitro via both complement dependent (CDC) and antibody-dependent (ADCC) mechanisms (Reff et al., Blood 83: 435-445 (1994)). Those skilled in the art will appreciate that binding polypeptide of the invention may comprises variable regions or CDRs of C2B8 or 2B8, in order to provide binding polypeptide that are even more effective in treating patients presenting with CD20+ malignancies.

In other embodiments of the present invention, the binding polypeptide of the invention will bind to the same tumor-associated antigen as CC49. CC49 binds human tumor-associated antigen TAG-72 which is associated with the surface of certain tumor cells of human origin, specifically the LS174T tumor cell line. LS174T is a variant of the LS180 colon adenocarcinoma line.

Binding polypeptides of the invention may comprise antigen binding sites derived from numerous murine monoclonal antibodies that have been developed and which have binding specificity for TAG-72. One of these monoclonal antibodies, designated B72.3, is a murine IgG1 produced by hybridoma B72.3. B72.3 is a first generation monoclonal antibody developed using a human breast carcinoma extract as the immunogen (see Colcher et al., Proc. Natl. Acad. Sci. (USA), 78:3199-3203 (1981); and U.S. Pat. Nos. 4,522,918 and 4,612,282, each of which is incorporated herein by reference). Other monoclonal antibodies directed against TAG-72 are designated “CC” (for colon cancer). As described by Schlom et al. (U.S. Pat. No. 5,512,443 which is incorporated herein by reference) CC monoclonal antibodies are a family of second generation murine monoclonal antibodies that were prepared using TAG-72 purified with B72.3. Because of their relatively good binding affinities to TAG-72, the following CC antibodies are preferred: CC49, CC 83, CC46, CC92, CC30, CC11, and CC15. Schlom et al. have also produced variants of a humanized CC49 antibody as disclosed in PCT/US99/25552 and single chain Fv (scFv) constructs as disclosed in U.S. Pat. No. 5,892,019, each of which is also incorporated herein by reference. Those skilled in the art will appreciate that each of the foregoing antibodies, constructs or recombinants, and variations thereof, may be synthetic and used to provide binding sites for the production of binding polypeptides in accordance with the present invention.

In addition to the anti-TAG-72 antibodies discussed above, various groups have also reported the construction and partial characterization of domain-deleted CC49 and B72.3 antibodies (e.g., Calvo et al. Cancer Biotherapy, 8(1):95-109 (1993), Slavin-Chiorini et al. Int. J. Cancer 53:97-103 (1993) and Slavin-Chiorini et al. Cancer. Res. 55:5957-5967 (1995). Accordingly, binding polypeptides may comprise antigen binding sites, variable region, or CDRs derived from these antibodies as well.

In one embodiment, a binding polypeptide of the invention comprises an antigen binding site that binds to the CD23 antigen (U.S. Pat. No. 6,011,138). In a preferred embodiment, a binding polypeptide of the invention binds to the same epitope as the 5E8 antibody. In another embodiment, a binding polypeptide of the invention comprises at least one CDR (e.g., 1, 2, 3, 4, 5, or 6 CDRs) from an anti-CD23 antibody, e.g., the 5E8 antibody (e.g., Lumiliximab).

In one embodiment, a binding polypeptide of the invention binds to the CRIPTO-I antigen (WO02/088170A2 or WO03/083041A2). In a more preferred embodiment, a binding polypeptide of the invention binds to the same epitope as the B3F6 antibody. In still another embodiment, an altered antibody of the invention comprises at least one CDR (e.g., 1, 2, 3, 4, 5, or 6 CDRs) or variable region from an anti-CRIPTO-I antibody, e.g., the B3F6 antibody.

In another embodiment, a binding polypeptide of the invention binds to antigen which is a member of the TNF superfamily of receptors (“TNFRs”). In another embodiment, the binding molecules of the invention bind at least one target that transduces a signal to a cell, e.g., by binding to a cell surface receptor, such as a TNF family receptor. By “transduces a signal” it is meant that by binding to the cell, the binding molecule converts the extracellular influence on the cell surface receptor into a cellular response, e.g., by modulating a signal transduction pathway. The term “TNF receptor” or “TNF receptor family member” refers to any receptor belonging to the Tumor Necrosis Factor (“TNF”) superfamily of receptors. Members of the TNF Receptor Superfamily (“TNFRSF”) are characterized by an extracellular region with two or more cysteine-rich domains (˜40 amino acids each) arranged as cysteine knots (see Dempsey et al., Cytokine Growth Factor Rev. (2003). 14(3-4):193-209). Upon binding their cognate TNF ligands, TNF receptors transduce signals by interacting directly or indirectly with cytoplasmic adapter proteins known as TRAFs (TNF receptor associate factors). TRAFs can induce the activation of several kinase cascades that ultimately lead to the activation of signal transduction pathways such as NF-KappaB, JNK, ERK, p38 and PI3K, which in turn regulate cellular processes ranging from immune function and tissue differentiation to apoptosis. The nucleotide and amino acid sequences of several TNF receptors family members are known in the art and include at least 29 human genes: TNFRSF1A (TNFR1, also known as DR1, CD120a, TNF-R-I p55, TNF-R, TNFRI, TNFAR, TNF-R55, p55TNFR, p55R, or TNFR60, GenBank GI No. 4507575; see also U.S. Pat. No. 5,395,760)), TNFRSF1B (CD120b, also known as p75, TNF-R, TNF-R-II, TNFR80, TNFR2, TNF-R75, TNFBR, or p75TNFR; GenBank GI No. 4507577), TNFRSF3 (Lymphotoxin Beta Receptor (LTβR), also known as TNFR2-RP, CD18, TNFR-RP, TNFCR, or TNF-R-III; GI Nos. 4505038 and 20072212), TNFRSF4 (OX40, also known as ACT35, TXGP1L, or CD134 antigen; GI Nos. 4507579 and 8926702), TNFRSF5 (CD40, also known as p50 or Bp50; GI Nos. 4507581 and 23312371), TNFRSF6 (FAS, also known as FAS-R, DcR-2, DR2, CD95, APO-1, or APT1; GenBank GI Nos. 4507583, 23510421, 23510423, 23510425, 23510427, 23510429, 23510431, and 23510434)), TNFRSF6B (DcR3, DR3; GenBank GI Nos. 4507569, 23200021, 23200023, 23200025, 23200027, 23200029, 23200031, 23200033, 23200035, 23200037, and 23200039), TNFRSF7 (CD27, also known as Tp55 or S152; GenBank GI No. 4507587), TNFRSF8 (CD30, also known as Ki-1, or D1S166E; GenBank GI Nos. 4507589 and 23510437), TNFRSF9 (4-1-BB, also known as CD137 or ILA; GI Nos. 5730095 and 728738), TNFRSF10A (TRAIL-R1, also known as DR4 or Apo2; GenBank GI No. 21361086), TNFRSF10B (TRAIL-R2, also known as DR5, KILLER, TRICK2A, or TRICKB; GenBank GI Nos. 22547116 and 22547119), TNFRSF10C (TRAIL-R3, also known as DcRI, LIT, or TRID; GenBank GI No. 22547121), TNFRSF10D (TRAIL-R4, also known as DcR2 or TRUNDD), TNFRSF11A (RANK; GenBank GI No. 4507565; see U.S. Pat. Nos. 6,562,948; 6,537,763; 6,528,482; 6,479,635; 6,271,349; 6,017,729), TNFRSF11B (Osteoprotegerin (OPG), also known as OCIF or TRI; GI Nos. 38530116, 22547122 and 33878056), TNFRSF12 (Translocating chain-Association Membrane Protein (TRAMP), also known as DR3, WSL-1, LARD, WSL-LR, DDR3, TR3, APO-3, Fn14, or TWEAKR; GenBank GI No. 7706186; US Patent Application Publication No. 2004/0033225A1), TNFRSF12L (DR3L), TNFRSF13B (TAC1; GI No. 6912694), TNFRSF13C (BAFFR; GI No. 16445027), TNFRSF14 (Herpes Virus Entry Mediator (HVEM), also known as ATAR, TR2, LIGHTR, or HVEA; GenBank GI Nos. 23200041, 12803895, and 3878821), TNFRSF16 (Low-Affinity Nerve Growth Factor Receptor (LNGFR), also known as Neurotrophin Receptor or p75(NTR); GenBank GI Nos. 128156 and 4505393), TNFRSF17 (BCM, also known as BCMA; GI No. 23238192), TNFRSF18 (AITR, also known as GITR; GenBank GI Nos. 4759246, 23238194 and 23238197), TNFRSF19 (Troy/Trade, also known as TAJ; GenBank GI Nos. 23238202 and 23238204), TNFRSF20 (RELT, also known as FLJ14993; GI Nos. 21361873 and 23238200), TNFRSF21 (DR6), TNFRSF22 (SOBa, also known as Tnfrh2 or 2810028K06Rik), and TNFRSF23 (mSOB, also known as Tnfrh1). Other TNF family members include EDAR1 (Ectodysplasin A Receptor, also known as Downless (DL), ED3, ED5, ED1R, EDA3, EDA1R, EDA-A1R; GenBank GI No. 11641231; U.S. Pat. No. 6,355,782), XEDAR (also known as EDA-A2R; GenBank GI No. 11140823); and CD39 (GI Nos. 2135580 and 765256). In another embodiment, an altered antibody of the invention binds to a TNF receptor family member lacking a death domain. In one embodiment, the TNF receptor lacking a death domain is involved in tissue differentiation. In a more specific embodiment, the TNF receptor involved in tissue differentiation is selected from the group consisting of LTPR, RANK, EDARI, XEDAR, Fn14, Troy/Trade, and NGFR. In another embodiment, the TNF receptor lacking a death domain is involved in immune regulation. In a more specific embodiment, TNF receptor family member involved in immune regulation is selected from the group consisting of TNFR2, HVEM, CD27, CD30, CD40, 4-1BB, OX40, and GITR.

In another embodiment, a binding polypeptide of the invention binds to a TNF ligand belonging to the TNF ligand superfamily. TNF ligands bind to distinct receptors of the TNF receptor superfamily and exhibit 15-25% amino acid sequence homology with each other (Gaur et al., Biochem. Pharmacol. (2003), 66(8):1403-8). The nucleotide and amino acid sequences of several TNF Receptor (Ligand) Superfamily (“TNFSF”) members are known in the art and include at least 16 human genes: TNFSF1 (also known as Lymphotoxin-α (LTA), TNFβ or LT, GI No.:34444 and 6806893), TNFSF2 (also known as TNF, TNFα; or DIF; GI No. 25952111), TNFSF3 (also known as Lymphotoxin-β (LTB), TNFC, or p33), TNFSF4 (also known as OX-40L, gp34, CD134L, or tax-transcriptionally activated glycoprotein 1, 34 kD (TXGP1); GI No. 4507603), TNFSF5 (also known as CD40LG, IMD3, HIGM1, CD40L, hCD40L, TRAP, CD154, or gp39; GI No. 4557433), TNFSF6 (also known as FasL or APT1LG1; GenBank GI No. 4557329), TNFSF7 (also known as CD70, CD27L, or CD27LG; GI No. 4507605), TNFSF8 (also known as CD30LG, CD30L, or CD153; GI No. 4507607), TNFSF9 (also known as 4-IBB-L or ILA ligand; GI No. 4507609), TNFSF10 (also known as TRAIL, Apo-2L, or TL2; GI No. 4507593), TNFSF11 (also known as TRANCE, RANKL, OPGL, or ODF; GI Nos. 4507595 and 14790152), TNFSF12 (also known as Fn14L, TWEAK, DR3LG, or APO3L; GI Nos. 4507597 and 23510441), TNFSF13 (also known as APRIL), TNFSF14 (also known as LIGHT, LTg, or HVEM-L; GI Nos. 25952144 and 25952147), TNFSF15 (also known as TL1 or VEGI), or TNFSF16 (also known as AITRL, TL6, hGITRL, or GITRL; GI No. 4827034). Other TNF ligand family members include EDAR1 & XEDAR ligand (ED1; GI No. 4503449; Monreal et al. (1998) Am J Hum Genet. 63:380), Troy/Trade ligand, BAFF (also known as TALL1; GI No. 5730097), and NGF ligands (e.g. NGF-β (GI No. 4505391), NGF-2/NTF3; GI No. 4505469), NTF5 (GI No. 5453808)), BDNF (GI Nos. 25306267, 25306235, 25306253, 25306257, 25306261, 25306264; IFRD1 (GI No. 450-4607)).

In one embodiment, a binding polypeptide of the invention binds to LTβR antibody (e.g. to the same epitope as (i.e., competes with) a CBE11 or BDA8 antibody). Exemplary anti-LTβR antibodies are set forth in WO 98/017313 and WO 02/30986, which are incorporated herein by reference. In still another embodiment, an altered antibody of the invention comprises at least one CDR (e.g., 1, 2, 3, 4, 5, or 6 CDRs) from an anti-LTβR antibody, e.g., the CBE11 antibody or the BDA8 antibody.

In another preferred embodiment, a binding polypetide of the invention binds TRAIL-R2 (e.g. to the same epitope as (i.e., competes with) a 14A2 antibody). In still another embodiment, a polypeptide of the invention comprises at least one CDR (e.g., 1, 2, 3, 4, 5, or 6 CDRs) from an anti-TRAIL-R2 antibody, e.g., the 14A2 antibody.

In yet another preferred embodiment, a binding polypetide of the invention binds to the same epitope as an anti-CD2 antibody (e.g., a chimeric CB6 (“chB6”) antibody). Exemplary anti-CD2 antibodies from which the binding polypeptides of the invention may be derived include the mouse antibody CB6 as well as chimeric versions thereof, e.g., the IgG1 chCB6 antibody disclosed in the Examples. In particular embodiments, an anti-CD2 binding polypeptide of the invention comprises a heavy chain sequence selected from the group consisting of SEQ ID NO:29 (ASK058), SEQ ID NO:31 (ASK062), SEQ ID NO:33 (ASK063), and SEQ ID NO:35 (ASK064).

Still other embodiments of the present invention comprise altered antibodies that are derived from or bind to the same tumor associated antigen as C5E10. As set forth in co-pending application Ser. No. 09/104,717, C5E10 is an antibody that recognizes a glycoprotein determinant of approximately 115 kDa that appears to be specific to prostate tumor cell lines (e.g. DU145, PC3, or ND1). Thus, in conjunction with the present invention, polypeptides that specifically bind to the same tumor-associated antigen recognized by C5E10 antibodies could be used alone or conjugated with an effector moiety by the methods of the invention, thereby providing a modified polypeptide that is useful for the improved treatment of neoplastic disorders. In particularly preferred embodiments, the starting polypeptide will be derived or comprise all or part of the antigen binding region of the C5E10 antibody as secreted from the hybridoma cell line having ATCC accession No. PTA-865. The resulting polypeptide could then be conjugated to a therapeutic effector moiety as described below and administered to a patient suffering from prostate cancer in accordance with the methods herein.

In still other embodiments, a binding polypeptide of the invention binds to a molecule which is useful in treating an autoimmune or inflammatory disease or disorder. For example, a binding polypeptide may bind to an antigen present on an immune cell (e.g., a B or T cell) or an autoantigen responsible for an autoimmune disease or disorder. The antigen associated with an autoimmune or inflammatory disorder may be a tumor-associated antigen described supra. Thus, a tumor associated antigen may also be an autoimmune or inflammatory associated disorder. As used herein, the term “autoimmune disease or disorder” refers to disorders or conditions in a subject wherein the immune system attacks the body's own cells, causing tissue destruction. Autoimmune diseases include general autoimmune diseases, i.e., in which the autoimmune reaction takes place simultaneously in a number of tissues, or organ specific autoimmune diseases, i.e., in which the autoimmune reaction targets a single organ. Examples of autoimmune diseases that can be diagnosed, prevented or treated by the methods and compositions of the present invention include, but are not limited to, Crohn's disease; Inflammatory bowel disease (IBD); systemic lupus erythematosus; ulcerative colitis; rheumatoid arthritis; Goodpasture's syndrome; Grave's disease; Hashimoto's thyroiditis; pemphigus vulgaris; myasthenia gravis; scleroderma; autoimmune hemolytic anemia; autoimmune thrombocytopenic purpura; polymyositis and dermatomyositis; pernicious anemia; Sjögren's syndrome; ankylosing spondylitis; vasculitis; type I diabetes mellitus; neurological disorders, multiple sclerosis, and secondary diseases caused as a result of autoimmune diseases.

In other embodiments, the binding polypeptides of the invention bind to a target molecule associated with an inflammatory disease or disorder. As used herein the term “inflammatory disease or disorder” includes diseases or disorders which are caused, at least in part, or exacerbated by inflammation, e.g., increased blood flow, edema, activation of immune cells (e.g., proliferation, cytokine production, or enhanced phagocytosis). For example, a binding polyeptide of the invention may bind to an inflammatory factor (e.g., a matrix metalloproteinase (MMP), TNFα, an interleukin, a plasma protein, a cytokine, a lipid metabolite, a protease, a toxic radical, a mitochondrial protein, an apoptotic protein, an adhesion molecule, etc.) involved or present in an area in aberrant amounts, e.g., in amounts which may be advantageous to alter, e.g., to benefit the subject. The inflammatory process is the response of living tissue to damage. The cause of inflammation may be due to physical damage, chemical substances, micro-organisms, tissue necrosis, cancer or other agents. Acute inflammation is short-lasting, e.g., lasting only a few days. If it is longer lasting however, then it may be referred to as chronic inflammation.

Inflammatory disorders include acute inflammatory disorders, chronic inflammatory disorders, and recurrent inflammatory disorders. Acute inflammatory disorders are generally of relatively short duration, and last for from about a few minutes to about one to two days, although they may last several weeks. The main characteristics of acute inflammatory disorders include increased blood flow, exudation of fluid and plasma proteins (edema) and emigration of leukocytes, such as neutrophils. Chronic inflammatory disorders, generally, are of longer duration, e.g., weeks to months to years or even longer, and are associated histologically with the presence of lymphocytes and macrophages and with proliferation of blood vessels and connective tissue. Recurrent inflammatory disorders include disorders which recur after a period of time or which have periodic episodes. Examples of recurrent inflammatory disorders include asthma and multiple sclerosis. Some disorders may fall within one or more categories. Inflammatory disorders are generally characterized by heat, redness, swelling, pain and loss of function. Examples of causes of inflammatory disorders include, but are not limited to, microbial infections (e.g., bacterial, viral and fungal infections), physical agents (e.g., burns, radiation, and trauma), chemical agents (e.g., toxins and caustic substances), tissue necrosis and various types of immunologic reactions. Examples of inflammatory disorders include, but are not limited to, osteoarthritis, rheumatoid arthritis, acute and chronic infections (bacterial, viral and fungal); acute and chronic bronchitis, sinusitis, and other respiratory infections, including the common cold; acute and chronic gastroenteritis and colitis; acute and chronic cystitis and urethritis; acute respiratory distress syndrome; cystic fibrosis; acute and chronic dermatitis; acute and chronic conjunctivitis; acute and chronic serositis (pericarditis, peritonitis, synovitis, pleuritis and tendinitis); uremic pericarditis; acute and chronic cholecystis; acute and chronic vaginitis; acute and chronic uveitis; drug reactions; and burns (thermal, chemical, and electrical).

In one preferred embodiment, a binding polypeptide of the invention binds to CD40L antibody (e.g., to the same epitope as (i.e., competes with) a 5C8 antibody). In still another embodiment, a polypeptide of the invention comprises at least one antigen binding site, one or more CDRs (e.g., 1, 2, 3, 4, 5, or 6 CDRs), or one or more variable regions (VH or VL) from an anti-CD40L antibody (e.g. a 5C8 antibody). CD40L (CD154, gp39), a transmembrane protein, is expressed on activated CD4+ T cells, mast cells, basophils, eosinophils, natural killer (NK) cells, and activated platelets. CD40L is important for T-cell-dependent B-cell responses. A prominent function of CD40L, isotype switching, is demonstrated by the hyper-immunoglobulin M (IgM) syndrome in which CD40L is congenitally deficient. The interaction of CD40L-CD40 (on antigen-presenting cells such as dendritic cells) is essential for T-cell priming and the T-cell-dependent humoral immune response. Therefore, interruption of the CD40-CD40L interaction with an anti-CD40L monoclonal antibody (mAb) has been considered to be a possible therapeutic strategy in human autoimmune disease, based upon the above information and on studies in animals. Exemplary anti-CD40L antibodies from which the binding polypeptides of the invention may be derived include the mouse antibody 5C8, disclosed in U.S. Pat. No. 5,474,771, which is incorporated by reference herein, as well as humanized versions thereof, e.g., the IgG1 Hu5C8 antibody disclosed in the Examples. Other anti-CD40L antibodies are known in the art (see e.g., U.S. Pat. No. 5,961,974 and International Publication No. WO 96/23071). In particular embodiments, an anti-CD40L binding polypeptide of the invention comprises a heavy chain sequence selected from the group consisting of SEQ ID NO: 1 (EAG2066), SEQ ID NO:7 (EAG2146), SEQ ID NO:9 (EAG2147), SEQ ID NO:13 (ASK043), SEQ ID NO:15 (ASK048), SEQ ID NO:17 (ASK052), and SEQ ID NO:19 (ASK053).

In yet other embodiments, a binding polypeptide of the invention binds to a molecule which is useful in treating a neurological disease or disorder. For example, a binding polypeptide may bind to an antigen present on a neural cell (e.g., a neuron, a glial cell, or a). In certain embodiments, the antigen associated with a neurological disorder may be an autoimmune or inflammatory disorder described supra. As used herein, the term “neurological disease or disorder” includes disorders or conditions in a subject wherein the nervous system either degenerates (e.g., neurodegenerative disorders, as well as disorders where the nervous system fails to develop properly or fails to regenerate following injury, e.g., spinal cord injury. Examples of neurological disorders that can be diagnosed, prevented or treated by the methods and compositions of the present invention include, but are not limited to, Multiple Sclerosis, Huntington's Disease, Alzheimer's Disease, Parkinson's Disease, neuropathic pain, traumatic brain injury, Guillain-Barré syndrome and chronic inflammatory demyelinating polyneuropathy (CIDP).

In one preferred embodiment, a binding polypeptide of the invention binds to the same epitope as an anti-LINGO antibody (e.g., a Li33 antibody). In still another embodiment, a polypeptide of the invention comprises at least one antigen binding site, one or more CDRs, or one or more variable regions (VH or VL) from an anti-LINGO antibody (e.g. a Li33 antibody). In particular embodiments, an anti-LINGO binding polypeptide of the invention comprises a heavy chain sequence selected from SEQ ID NO 21 (EAG2148), SEQ ID NO:22 (ASK050) and SEQ ID NO:23 (ASK051).

### (b) Antigen Binding Fragments

In other embodiments, a binding site of a binding polypeptide of the invention may comprise an antigen binding fragment. The term “antigen-binding fragment” refers to a polypeptide fragment of an immunoglobulin, antibody, or antibody variant which binds antigen or competes with intact antibody (i.e., with the intact antibody from which they were derived) for antigen binding (i.e., specific binding). For example, said antigen binding fragments can be derived from any of the antibodies or antibody variants described supra. Antigen binding fragments can be produced by recombinant or biochemical methods that are well known in the art. Exemplary antigen-binding fragments include Fv, Fab, Fab′, and (Fab′)₂.

In exemplary embodiments, a binding polypeptide of the invention comprises at least one antigen binding fragment that is operably linked (e.g., chemically conjugated or genetically-fused (e.g., directly fused or fused via a polypeptide linker)) to the C-terminus and/or N-terminus of a genetically-fused Fc region (i.e., a scFc region). In one exemplary embodiment, a binding polypeptide of the invention comprises an antigen binding fragment (e.g, a Fab) which is operably linked to the N-terminus (or C-terminus) of at least one genetically-fused Fc region via a hinge domain or portion thereof (e.g., an IgG1 hinge or portion thereof, e.g., a human IgG1 hinge). An exemplary hinge domain portion comprises the sequence DKTHTCPPCPAPELLGG.

### (c) Single Chain Binding Molecules

In other embodiments, a binding molecule of the invention may comprise a binding site from single chain binding molecule (e.g., a singe chain variable region or scFv). Techniques described for the production of single chain antibodies (U.S. Pat. No. 4,694,778; Bird, Science 242:423-442 (1988); Huston et al., Proc. Natl. Acad. Sci. USA 85:5879-5883 (1988); and Ward et al., Nature 334:544-554 (1989)) can be adapted to produce single chain binding molecules. Single chain antibodies are formed by linking the heavy and light chain fragments of the Fv region via an amino acid bridge, resulting in a single chain antibody. Techniques for the assembly of functional Fv fragments in E coli may also be used (Skerra et al., Science 242:1038-1041 (1988)).

In certain embodiments, a binding polypeptide of the invention comprises one or more binding sites or regions comprising or consisting of a single chain variable region sequence (scFv). Single chain variable region sequences comprise a single polypeptide having one or more antigen binding sites, e.g., a V_{L} domain linked by a flexible linker to a V_{H} domain. The V_{L} and/or V_{H} domains may be derived from any of the antibodies or antibody variants described supra. ScFv molecules can be constructed in a V_{H}-linker-V_{L} orientation or V_{L}-linker-V_{H} orientation. The flexible linker that links the V_{L} and V_{H} domains that make up the antigen binding site preferably comprises from about 10 to about 50 amino acid residues. In one embodiment, the polypeptide linker is a gly-ser polypeptide linker. An exemplary gly/ser polypeptide linker is of the formula (Gly4Ser)n, wherein n is a positive integer (e.g., 1, 2, 3, 4, 5, or 6). Other polypeptide linkers are known in the art. Antibodies having single chain variable region sequences (e.g. single chain Fv antibodies) and methods of making said single chain antibodies are well-known in the art (see e.g., Ho et al. 1989. Gene 77:51; Bird et al. 1988 Science 242:423; Pantoliano et al. 1991. Biochemistry 30:10117; Milenic et al. 1991. Cancer Research 51:6363; Takkinen et al. 1991. Protein Engineering 4:837).

In certain embodiments, a scFv molecule employed in a binding polypeptide of the invention is a stabilized scFv molecule. In one embodiment, the stabilized cFv molecule may comprise a scFv linker interposed between a V_{H} domain and a V_{L} domain, wherein the V_{H} and V_{L} domains are linked by a disulfide bond between an amino acid in the V_{H} and an amino acid in the V_{L} domain. In other embodiments, the stabilized scFv molecule may comprise a scFv linker having an optimized length or composition. In yet other embodiments, the stabilized scFv molecule may comprise a V_{H} or V_{L} domain having at least one stabilizing amino acid substitution(s). In yet another embodiment, a stabilized scFv molecule may have at least two of the above listed stabilizing features. Stabilized scFv molecules have improved protein stability or impart improved protein stability to the binding polypeptide to which it is operably linked. Preferred scFv linkers of the invention improve the thermal stability of a binding polypeptide of the invention by at least about 2° C. or 3° C. as compared to a conventional binding polypeptide Comparisons can be made, for example, between the scFv molecules of the invention. In certain preferred embodiments, the stabilized scFv molecule comprises a (Gly₄Ser)₄ scFv linker and a disulfide bond which links V_{H} amino acid 44 and V_{L} amino acid 100. Other exemplary stabilized scFv molecules which may be employed in the binding polypeptides of the invention are described in U.S. Provisional Patent Application No. 60/873,996, filed on Dec. 8, 2006 or U.S. patent application Ser. No. 11/725,970, filed on Mar. 19, 2007, each of which is incorporated herein by reference in its entirety.

In certain exemplary embodiments, the binding polypeptides of the invention comprise at least one scFv molecule that is operably linked (e.g., chemically conjugated or genetically-fused (e.g., directly fused or fused via a polypeptide linker) to the C-terminus and/or N-terminus of a genetically-fused Fc region (i.e., a scFc region). In one exemplary embodiment, a binding polypeptide of the invention comprises at least one scFv molecule (e.g, one or more stabilized scFv molecules) which are operably linked to the N-terminus (or C-terminus) of at least one genetically-fused Fc region via a hinge domain or portion thereof (e.g., an IgG1 hinge or portion thereof, e.g., a human IgG1 hinge). An exemplary hinge domain portion comprises the sequence DKTHTCPPCPAPELLGG.

In certain embodiments, a binding polypeptide of the invention comprises a tetravalent binding site or region formed by fusing two or more scFv molecules in series. For example, in one embodiment, scFv molecules are combined such that a first scFv molecule is operably linked at its N-terminus (e.g., via a polypeptide linker (e.g., a gly/ser polypeptide linker)) to at least one additional scFv molecule having the same or different binding specificity. Tandem arrays of scFv molecules are operably linked to the N-terminus and/or C-terminus of at least one genetically-fused Fc region (i.e., a scFc region) to form a binding polypeptide of the invention.

In another embodiment, a binding polypeptide of the invention comprises a tetravalent binding site or region which is formed by operably linking a scFv molecule (e.g. via a polypeptide linker) to an antigen biding fragment (e.g., a Fab fragment). Said tetravalent binding site or region is operably linked to the N-terminus and/or C-terminus of at least one genetically-fused Fc region (i.e., a scFc region) to form a binding polypeptide of the invention.

### (d) Modified Antibodies

In other aspects, the binding polypeptides of the invention may comprise antigen binding sites, or portions thereof, derived from modified forms of antibodies. Exemplary such forms include, e.g., minibodies, diabodies, triabodies, nanobodies, camelids, Dabs, tetravalent antibodies, intradiabodies (e.g., Jendreyko et al. 2003. J. Biol. Chem. 278:47813), fusion proteins (e.g., antibody cytokine fusion proteins, proteins fused to at least a portion of an Fc receptor), and bispecific antibodies. Other modified antibodies are described, for example in U.S. Pat. No. 4,745,055; EP 256,654; Faulkner et al., Nature 298:286 (1982); EP 120,694; EP 125,023; Morrison, J. Immun. 123:793 (1979); Kohler et al., Proc. Natl. Acad. Sci. USA 77:2197 (1980); Raso et al., Cancer Res. 41:2073 (1981); Morrison et al., Ann. Rev. Immunol. 2:239 (1984); Morrison, Science 229:1202 (1985); Morrison et al., Proc. Natl. Acad. Sci. USA 81:6851 (1984); EP 255,694; EP 266,663; and WO 88/03559. Reassorted immunoglobulin chains also are known. See, for example, U.S. Pat. No. 4,444,878; WO 88/03565; and EP 68,763 and references cited therein.

In one embodiment, a binding polyeptide of the invention comprises an antigen binding site or region which is a minibody or an antigen binding site derived therefrom. Minibodies are dimeric molecules made up of two polypeptide chains each comprising a scFv molecule which is fused to a CH3 domain or portion thereof via a polypeptide linker. Minibodies can be made by linking a scFv component and polypeptide linker-CH3 component using methods described in the art (see, e.g., U.S. Pat. No. 5,837,821 or WO 94/09817A1). These components can be isolated from separate plasmids as restriction fragments and then ligated and recloned into an appropriate vector (e.g., an expression vector). Appropriate assembly (e.g., of the open reading frame (ORF) encoding the monomeric minibody polypeptide chain) can be verified by restriction digestion and DNA sequence analysis. In one embodiment, a binding polypeptide of the invention comprises the scFv component of a minibody which is operably linked to at least one genetically-fused Fc region (i.e., scFc region). In another embodiment, a binding polyeptide of the invention comprises a tetravalent minibody as a binding site or region. Tetravalent minibodies can be constructed in the same manner as minibodies, except that two scFv molecules are linked using a polypeptide linker. The linked scFv-scFv construct is then operably linked to a genetically-fused Fc region (i.e., to a scFc region) to form a binding polypeptide of the invention.

In another embodiment, a binding polyeptide of the invention comprises an antigen binding site or region which is a diabody or an antigen binding site derived therefrom. Diabodies are dimeric, tetravalent molecules each having a polypeptide similar to scFv molecules, but usually having a short (e.g., less than 10 and preferably 1-5) amino acid residue linker connecting both variable domains, such that the V_{L} and V_{H} domains on the same polypeptide chain cannot interact. Instead, the V_{L} and V_{H} domain of one polypeptide chain interact with the V_{H} and V_{L} domain (respectively) on a second polypeptide chain (see, for example, WO 02/02781). In one embodiment, a binding polypeptide of the invention comprises a diabody which is operably linked to the N-terminus and/or C-terminus of at least one genetically-fused Fc region (i.e., scFc region).

In certain embodiments, the binding molecule comprises a single domain binding molecule (e.g. a single domain antibody) linked to an scFc. Exemplary single domain molecules include an isolated heavy chain variable domain (V_{H}) of an antibody, i.e., a heavy chain variable domain, without a light chain variable domain, and an isolated light chain variable domain (V_{L}) of an antibody, i.e., a light chain variable domain, without a heavy chain variable domain. Exemplary single-domain antibodies employed in the binding molecules of the invention include, for example, the Camelid heavy chain variable domain (about 118 to 136 amino acid residues) as described in Hamers-Casterman, et al., Nature 363:446-448 (1993), and Dumoulin, et al., Protein Science 11:500-515 (2002). Other exemplary single domain antibodies include single VH or VL domains, also known as Dabs® (Domantis Ltd., Cambridge, UK). Yet other single domain antibodies include shark antibodies (e.g., shark Ig-NARs). Shark Ig-NARs comprise a homodimer of one variable domain (V-NAR) and five C-like constant domains (C-NAR), wherein diversity is concentrated in an elongated CDR3 region varying from 5 to 23 residues in length. In camelid species (e.g., llamas), the heavy chain variable region, referred to as VHH, forms the entire antigen-binding domain. The main differences between camelid VHH variable regions and those derived from conventional antibodies (VH) include (a) more hydrophobic amino acids in the light chain contact surface of VH as compared to the corresponding region in VHH, (b) a longer CDR3 in VHH, and (c) the frequent occurrence of a disulfide bond between CDR1 and CDR3 in VHH. Methods for making single domain binding molecules are described in U.S. Pat. Nos. 6,005,079 and 6,765,087, both of which are incorporated herein by reference. Exemplary single domain antibodies comprising VHH domains include Nanobodies® (Ablynx NV, Ghent, Belgium).

### (e) Non-Immunoglobulin Binding Molecules

In certain other embodiments, the binding polypeptides of the invention comprise one or more binding sites derived from a non-immunoglobulin binding molecule. As used herein, the term “non-immunoglobulin binding molecules” are binding molecules whose binding sites comprise a portion (e.g., a scaffold or framework) which is derived from a polypeptide other than an immunoglobulin, but which may be engineered (e.g., mutagenized) to confer a desired binding specificity.

Other examples of binding molecules comprising binding sites not derived from antibody molecules include receptor binding sites and ligand binding sites which are discussed in more detail infra.

Non-immunoglobulin binding molecules can comprise binding site portions that are derived from a member of the immunoglobulin superfamily that is not an immunoglobulin (e.g. a T-cell receptor or a cell-adhesion protein (e.g., CTLA-4, N-CAM, telokin)). Such binding molecules comprise a binding site portion which retains the conformation of an immunoglobulin fold and is capable of specifically binding an IGF1-R eptitope. In other embodiments, non-immunoglobulin binding molecules of the invention also comprise a binding site with a protein topology that is not based on the immunoglobulin fold (e.g. such as ankyrin repeat proteins or fibronectins) but which nonetheless are capable of specifically binding to a target (e.g. an IGF-1R epitope).

Non-immunoglobulin binding molecules may be identified by selection or isolation of a target-binding variant from a library of binding molecules having artificially diversified binding sites. Diversified libraries can be generated using completely random approaches (e.g., error-prone PCR, exon shuffling, or directed evolution) or aided by art-recognized design strategies. For example, amino acid positions that are usually involved when the binding site interacts with its cognate target molecule can be randomized by insertion of degenerate codons, trinucleotides, random peptides, or entire loops at corresponding positions within the nucleic acid which encodes the binding site (see e.g., U.S. Pub. No. 20040132028). The location of the amino acid positions can be identified by investigation of the crystal structure of the binding site in complex with the target molecule. Candidate positions for randomization include loops, flat surfaces, helices, and binding cavities of the binding site. In certain embodiments, amino acids within the binding site that are likely candidates for diversification can be identified by their homology with the immunoglobulin fold. For example, residues within the CDR-like loops of fibronectin may be randomized to generate a library of fibronectin binding molecules (see, e.g., Koide et al., J. Mol. Biol., 284: 1141-1151 (1998)). Other portions of the binding site which may be randomized include flat surfaces. Following randomization, the diversified library may then be subjected to a selection or screening procedure to obtain binding molecules with the desired binding characteristics, e.g. specific binding to an IGF-1R epitope described supra. For example, selection can be achieved by art-recognized methods such as phage display, yeast display, or ribosome display.

In one embodiment, a binding molecule of the invention comprises a binding site from a fibronectin binding molecule. Fibronectin binding molecules (e.g., molecules comprising the Fibronectin type I, II, or III domains) display CDR-like loops which, in contrast to immunoglobulins, do not rely on intra-chain disulfide bonds. Methods for making fibronectin binding polypeptides are described, for example, in WO 01/64942 and in U.S. Pat. Nos. 6,673,901, 6,703,199, 7,078,490, and 7,119,171, which are incorporated herein by reference. In one exemplary embodiment, the fibronectin binding polypeptide is as AdNectin® (Adnexus Therpaeutics, Waltham, Mass.).

In another embodiment, a binding molecule of the invention comprises a binding site from an Affibody® (Abcam, Cambridge, Mass.). Affibodies are derived from the immunoglobulin binding domains of staphylococcal Protein A (SPA) (see e.g., Nord et al., Nat. Biotechnol., 15: 772-777 (1997)). Affibody binding sites employed in the invention may be synthesized by mutagenizing an SPA-related protein (e.g., Protein Z) derived from a domain of SPA (e.g., domain B) and selecting for mutant SPA-related polypeptides having binding affinity for an IGF-1R epitope. Other methods for making affibody binding sites are described in U.S. Pat. Nos. 6,740,734 and 6,602,977 and in WO 00/63243, each of which is incorporated herein by reference.

In another embodiment, a binding molecule of the invention comprises a binding site from an Anticalin® (Pieris A G, Friesing, Germany). Anticalins (also known as lipocalins) are members of a diverse β-barrel protein family whose function is to bind target molecules in their barrel/loop region. Lipocalin binding sites may be engineered to bind an IGF-1R epitope by randomizing loop sequences connecting the strands of the barrel (see e.g., Schlehuber et al., Drug Discov. Today, 10: 23-33 (2005); Beste et al., PNAS, 96: 1898-1903 (1999). Anticalin binding sites employed in the binding molecules of the invention may be obtainable starting from polypeptides of the lipocalin family which are mutated in four segments that correspond to the sequence positions of the linear polypeptide sequence comprising amino acid positions 28 to 45, 58 to 69, 86 to 99 and 114 to 129 of the Bilin-binding protein (BBP) of Pieris brassica. Other methods for making anticalin binding sites are described in WO99/16873 and WO 05/019254, each of which is incorporated herein by reference.

In another embodiment, a binding molecule of the invention comprises a binding site from a cysteine-rich polypeptide. Cysteine-rich domains employed in the practice of the present invention typically do not form a α-helix, a β sheet, or a β-barrel structure. Typically, the disulfide bonds promote folding of the domain into a three-dimensional structure. Usually, cysteine-rich domains have at least two disulfide bonds, more typically at least three disulfide bonds. An exemplary cysteine-rich polypeptide is an A domain protein. A-domains (sometimes called “complement-type repeats”) contain about 30-50 or 30-65 amino acids. In some embodiments, the domains comprise about 35-45 amino acids and in some cases about 40 amino acids. Within the 30-50 amino acids, there are about 6 cysteine residues. Of the six cysteines, disulfide bonds typically are found between the following cysteines: C1 and C3, C2 and C5, C4 and C6. The A domain constitutes a ligand binding moiety. The cysteine residues of the domain are disulfide linked to form a compact, stable, functionally independent moiety. Clusters of these repeats make up a ligand binding domain, and differential clustering can impart specificity with respect to the ligand binding. Exemplary proteins containing A-domains include, e.g., complement components (e.g., C6, C7, C8, C9, and Factor I), serine proteases (e.g., enteropeptidase, matriptase, and corin), transmembrane proteins (e.g., ST7, LRP3, LRP5 and LRP6) and endocytic receptors (e.g., Sortilin-related receptor, LDL-receptor, VLDLR, LRP1, LRP2, and ApoER2). Methods for making A domain proteins of a desired binding specificity are disclosed, for example, in WO 02/088171 and WO 04/044011, each of which is incorporated herein by reference.

In other embodiments, a binding molecule of the invention comprises a binding site from a repeat protein. Repeat proteins are proteins that contain consecutive copies of small (e.g., about 20 to about 40 amino acid residues) structural units or repeats that stack together to form contiguous domains. Repeat proteins can be modified to suit a particular target binding site by adjusting the number of repeats in the protein. Exemplary repeat proteins include Designed Ankyrin Repeat Proteins (i.e., a DARPins®, Molecular Partners, Zurich, Switzerland) (see e.g., Binz et al., Nat. Biotechnol., 22: 575-582 (2004)) or leucine-rich repeat proteins (ie., LRRPs) (see e.g., Pancer et al., Nature, 430: 174-180 (2004)). All so far determined tertiary structures of ankyrin repeat units share a characteristic composed of a β-hairpin followed by two antiparallel α-helices and ending with a loop connecting the repeat unit with the next one. Domains built of ankyrin repeat units are formed by stacking the repeat units to an extended and curved structure. LRRP binding sites from part of the adaptive immune system of sea lampreys and other jawless fishes and resemble antibodies in that they are formed by recombination of a suite of leucine-rich repeat genes during lymphocyte maturation. Methods for making DARpin or LRRP binding sites are described in WO 02/20565 and WO 06/083275, each of which is incorporated herein by reference.

Other non-immunoglobulin binding sites which may be employed in binding molecules of the invention include binding sites derived from Src homology domains (e.g. SH2 or SH3 domains), PDZ domains, beta-lactamase, high affinity protease inhibitors, or small disulfide binding protein scaffolds such as scorpion toxins. Methods for making binding sites derived from these molecules have been disclosed in the art, see e.g., Silverman et al., Nat. Biotechnol., 23(12): 1493-4 (2005); Panni et al, J. Biol. Chem., 277: 21666-21674 (2002), Schneider et al., Nat. Biotechnol., 17: 170-175 (1999); Legendre et al., Protein Sci., 11:1506-1518 (2002); Stoop et al., Nat. Biotechnol., 21: 1063-1068 (2003); and Vita et al., PNAS, 92: 6404-6408 (1995). Yet other binding sites may be derived from a binding domain selected from the group consisting of an EGF-like domain, a Kringle-domain, a PAN domain, a G1a domain, a SRCR domain, a Kunitz/Bovine pancreatic trypsin Inhibitor domain, a Kazal-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain, a Link domain, a Thrombospondin type I domain, an Immunoglobulin-like domain, a C-type lectin domain, a MAM domain, a von Willebrand factor type A domain, a Somatomedin B domain, a WAP-type four disulfide core domain, a F5/8 type C domain, a Hemopexin domain, a Laminin-type EGF-like domain, a C2 domain, a CTLA-4 domain, and other such domains known to those of ordinary skill in the art, as well as derivatives and/or variants thereof. Additional non-immunoglobulin binding polypeptides include Avimers® (Avidia, Inc., Mountain View, Calif. see International PCT Publication No. WO 06/055689 and US Patent Pub 2006/0234299), Telobodies® (Biotech Studio, Cambridge, Mass.), Evibodies® (Evogenix, Sydney, Australia see U.S. Pat. No. 7,166,697), and Microbodies® (Nascacell Technologies, Munich, Germany).

ii. Binding Portions of Receptors and Ligands

In other aspects, the binding polypeptides of the invention comprise a ligand binding site of a receptor and/or a receptor binding portion of a ligand which is operably linked to at least one genetically-fused Fc region.

In certain embodiments, the binding polypeptide is a fusion of a ligand binding portion of a receptor and/or a receptor binding portion of a ligand with a genetically-fused Fc region (i.e., scFc region). Any transmembrane regions or lipid or phospholipid anchor recognition sequences of the ligand binding receptor are preferably inactivated or deleted prior to fusion. DNA encoding the ligand or ligand binding partner is cleaved by a restriction enzyme at or proximal to the 5′ and 3′ ends of the DNA encoding the desired ORF segment. The resultant DNA fragment is then readily inserted (e.g., ligated in-frame) into DNA encoding a genetically-fused Fc region. The precise site at which the fusion is made may be selected empirically to optimize the secretion or binding characteristics of the soluble fusion protein. DNA encoding the fusion protein is then subcloned into an appropriate expression vector than can be transfected into a host cell for expression.

In one embodiment, a binding polypeptide of the invention combines the binding site(s) of the ligand or receptor (e.g. the extracellular domain (ECD) of a receptor) with at least one genetically-fused Fc region (i.e., scFc region). In one embodiment, the binding domain of the ligand or receptor domain will be operably linked (e.g. fused via a polypeptide linker) to the C-terminus of a genetically-fused Fc region. N-terminal fusions are also possible. In exemplary embodiments, fusions are made to the C-terminus of the genetically-fused Fc region, or immediately N-terminal to the hinge domain a genetically-fused Fc region.

In certain embodiments, the binding site or domain of the ligand-binding portion of a receptor may be derived from a receptor bound by an antibody or antibody variant described supra. In other embodiments, the ligand binding portion of a receptor is derived from a receptor selected from the group consisting of a receptor of the Immunoglobulin (Ig) superfamily (e.g., a soluble T-cell receptor, e.g., mTCR® (Medigene AG, Munich, Germany), a receptor of the TNF receptor superfamily described supra (e.g., a soluble TNF® receptor of an immunoadhesin, e.g., Enbrel® (Wyeth, Madison, N.J.)), a receptor of the Glial Cell-Derived Neurotrophic Factor (GDNF) receptor family (e.g., GFRα3), a receptor of the G-protein coupled receptor (GPCR) superfamily, a receptor of the Tyrosine Kinase (TK) receptor superfamily, a receptor of the Ligand-Gated (LG) superfamily, a receptor of the chemokine receptor superfamily, IL-1/Toll-like Receptor (TLR) superfamily, and a cytokine receptor superfamily.

In other embodiments, the binding site or domain of the receptor-binding portion of a ligand may be derived from a ligand bound by an antibody or antibody variant described supra. For example, the ligand may bind a receptor selected from the group consisting of a receptor of the Immunoglobulin (Ig) superfamily, a receptor of the TNF receptor superfamily, a receptor of the G-protein coupled receptor (GPCR) superfamily, a receptor of the Tyrosine Kinase (TK) receptor superfamily, a receptor of the Ligand-Gated (LG) superfamily, a receptor of the chemokine receptor superfamily, IL-1/Toll-like Receptor (TLR) superfamily, and a cytokine receptor superfamily. In one exemplary embodiment, the binding site of the receptor-binding portion of a ligand is derived from a ligand belonging to the TNF ligand superfamily described supra (e.g., CD40L).

In other exemplary embodiments, a binding polypeptide of the invention may comprise one or more ligand binding domains or receptor binding domains derived from one or more of the following proteins:

1. Cytokines and Cytokine Receptors

Cytokines have pleiotropic effects on the proliferation, differentiation, and functional activation of lymphocytes. Various cytokines, or receptor binding portions thereof, can be utilized in the fusion proteins of the invention. Exemplary cytokines include the interleukins (e.g. IL-1, IL-2, IL-3, IL-4, IL-5, IL-6, IL-7, IL-8, IL-10, IL-1, IL-12, IL-13, and IL-18), the colony stimulating factors (CSFs) (e.g. granulocyte CSF (G-CSF), granulocyte-macrophage CSF (GM-CSF), and monocyte macrophage CSF (M-CSF)), tumor necrosis factor (TNF) alpha and beta, cytotoxic T lymphocyte antigen 4 (CTLA-4), and interferons such as interferon-α, β, or γ (U.S. Pat. Nos. 4,925,793 and 4,929,554).

Cytokine receptors typically consist of a ligand-specific alpha chain and a common beta chain. Exemplary cytokine receptors include those for GM-CSF, IL-3 (U.S. Pat. No. 5,639,605), IL-4 (U.S. Pat. No. 5,599,905), IL-5 (U.S. Pat. No. 5,453,491), IL10 receptor, IFN-γ (EP0240975), and the TNF family of receptors (e.g., TNFα (e.g. TNFR-1 (EP 417, 563), TNFR-2 (EP 417,014) lymphotoxin beta receptor).

2. Adhesion Proteins

Adhesion molecules are membrane-bound proteins that allow cells to interact with one another. Various adhesion proteins, including leukocyte homing receptors and cellular adhesion molecules, or receptor binding portions thereof, can be incorporated in a fusion protein of the invention. Leucocyte homing receptors are expressed on leucocyte cell surfaces during inflammation and include the β-1 integrins (e.g. VLA-1, 2, 3, 4, 5, and 6) which mediate binding to extracellular matrix components, and the β2-integrins (e.g. LFA-1, LPAM-1, CR3, and CR4) which bind cellular adhesion molecules (CAMs) on vascular endothelium. Exemplary CAMs include ICAM-1, ICAM-2, VCAM-1, and MAdCAM-1. Other CAMs include those of the selectin family including E-selectin, L-selectin, and P-selectin.

3. Chemokines

Chemokines, chemotactic proteins which stimulate the migration of leucocytes towards a site of infection, can also be incorporated into a fusion protein of the invention. Exemplary chemokines include Macrophage inflammatory proteins (MIP-1-α and MIP-1-β), neutrophil chemotactic factor, and RANTES (regulated on activation normally T-cell expressed and secreted).

4. Growth Factors and Growth Factor Receptors

Growth factors or their receptors (or receptor binding or ligand binding portions thereof) may be incorporated in the fusion proteins of the invention. Exemplary growth factors include Vascular Endothelial Growth Factor (VEGF) and its isoforms (U.S. Pat. No. 5,194,596); Fibroblastic Growth Factors (FGF), including aFGF and bFGF; atrial natriuretic factor (ANF); hepatic growth factors (HGFs; U.S. Pat. Nos. 5,227,158 and 6,099,841), neurotrophic factors such as bone-derived neurotrophic factor (BDNF), glial cell derived neurotrophic factor ligands (e.g., GDNF, neuturin, artemin, and persephin), neurotrophin-3, -4, -5, or -6 (NT-3, NT-4, NT-5, or NT-6), or a nerve growth factor such as NGF-β platelet-derived growth factor (PDGF) (U.S. Pat. Nos. 4,889,919, 4,845,075, 5,910,574, and 5,877,016); transforming growth factors (TGF) such as TGF-alpha and TGF-beta (WO 90/14359), osteoinductive factors including bone morphogenetic protein (BMP); insulin-like growth factors-I and -II (IGF-I and IGF-II; U.S. Pat. Nos. 6,403,764 and 6,506,874); Erythropoietin (EPO); Thrombopoeitin (TPO; stem-cell factor (SCF), thrombopoietin (TPO, c-Mpl ligand), and the Wnt polypeptides (U.S. Pat. No. 6,159,462).

Exemplary growth factor receptors which may be used as targeting receptor domains of the invention include EGF receptors; VEGF receptors (e.g. Flt1 or Flk1/KDR), PDGF receptors (WO 90/14425); HGF receptors (U.S. Pat. Nos. 5,648,273, and 5,686,292), and neurotrophic receptors including the low affinity receptor (LNGFR), also termed as p75^{NTR} or p75, which binds NGF, BDNF, and NT-3, and high affinity receptors that are members of the trk family of the receptor tyrosine kinases (e.g. trkA, trkB (EP 455,460), trkC (EP 522,530)).

5. Hormones

Exemplary growth hormones for use as targeting agents in the fusion proteins of the invention include renin, human growth hormone (HGH; U.S. Pat. No. 5,834,598), N-methionyl human growth hormone; bovine growth hormone; growth hormone releasing factor; parathyroid hormone (PTH); thyroid stimulating hormone (TSH); thyroxine; proinsulin and insulin (U.S. Pat. Nos. 5,157,021 and 6,576,608); follicle stimulating hormone (FSH); calcitonin, luteinizing hormone (LH), leptin, glucagons; bombesin; somatropin; mullerian-inhibiting substance; relaxin and prorelaxin; gonadotropin-associated peptide; prolactin; placental lactogen; OB protein; or mullerian-inhibiting substance.

6. Clotting Factors

Exemplary blood coagulation factors for use as targeting agents in the fusion proteins of the invention include the clotting factors (e.g., factors V, VII, VIII, IX, X, XI, XII and XIII, von Willebrand factor); tissue factor (U.S. Pat. Nos. 5,346,991, 5,349,991, 5,726,147, and 6,596,84); thrombin and prothrombin; fibrin and fibrinogen; plasmin and plasminogen; plasminogen activators, such as urokinase or human urine or tissue-type plasminogen activator (t-PA).

In one exemplary embodiments, a binding polypeptide of the invention is a fusion protein or immunoadhesin comprising a soluble LTβR receptor and a scFc region. For example, the binding polypeptide may comprise a heavy chain sequence of SEQ ID NO: 37 (ASK057).

In another exemplary embodiments, a binding polypeptide of the invention is a fusion protein or immunoadhesin comprising an interferon (e.g., β-interferon) and a scFc region. For example, the binding polypeptide may comprise a heavy chain sequence of SEQ ID NO: 39 (EAG2149).

In another exemplary embodiments, a binding polypeptide of the invention is a fusion protein or immunoadhesin comprising a soluble LTβR receptor and a scFc region. For example, the binding polypeptide may comprise a heavy chain sequence of SEQ ID NO: 41 (EAG2190) or SEQ ID NO:43 (EAG2191).

### III. MULTISPECIFIC BINDING POLYPEPTIDES

In certain particular aspects, a binding polypeptide of the invention is multispecific, i.e., has at least one binding site that binds to a first molecule or epitope of a molecule and at least one second binding site that binds to a second molecule or to a second epitope of the first molecule. Multispecific binding molecules of the invention may comprise at least two binding sites, wherein at least one of the binding sites is derived from or comprises a binding site from one of binding molecules described supra. In certain embodiments, at least one binding site of a multispecific binding molecule of the invention is an antigen binding region of an antibody or an antigen binding fragment thereof (e.g. an antibody or antigen binding fragment described supra).

### (a) Bispecific Molecules

In one embodiment, a binding polypeptide of the invention is bispecific. Bispecific binding polypeptides can bind to two different target sites, e.g., on the same target molecule or on different target molecules. For example, in the case of the binding polypeptides of the invention, a bispecific variant thereof can bind to two different epitopes, e.g., on the same antigen or on two different antigens. Bispecific binding polypeptides can be used, e.g., in diagnostic and therapeutic applications. For example, they can be used to immobilize enzymes for use in immunoassays. They can also be used in diagnosis and treatment of cancer, e.g., by binding both to a tumor associated molecule and a detectable marker (e.g., a chelator which tightly binds a radionuclide). Bispecific binding polypeptide can also be used for human therapy, e.g., by directing cytotoxicity to a specific target (for example by binding to a pathogen or tumor cell and to a cytotoxic trigger molecule, such as the T cell receptor or the Fcγ receptor). Bispecific binding polypeptides can also be used, e.g., as fibrinolytic agents or vaccine adjuvants.

Examples of bispecific binding polypeptides include those with at least two arms directed against different tumor cell antigens; bispecific altered binding proteins with at least one arm directed against a tumor cell antigen and at least one arm directed against a cytotoxic trigger molecule (such as anti-Fc.gamma.RI/anti-CD15, anti-p185.sup.HER2/Fc.gamma.RIII (CD16), anti-CD3/anti-malignant B-cell (1D10), anti-CD3/anti-p185.sup.HER2, anti-CD3/anti-p97, anti-CD 3/anti-renal cell carcinoma, anti-CD3/anti-OVCAR-3, anti-CD3/L-D1 (anti-colon carcinoma), anti-CD3/anti-melanocyte stimulating hormone analog, anti-EGF receptor/anti-CD3, anti-CD3/anti-CAMA1, anti-CD3/anti-CD 19, anti-CD3/MoV18, anti-neural cell adhesion molecule (NCAM)/anti-CD3, anti-folate binding protein (FBP)/anti-CD3, anti-pan carcinoma associated antigen (AMOC-31)/anti-CD3); bispecific binding polypeptides with at least one arm which binds specifically to a tumor antigen and at least one arm which binds to a toxin (such as anti-saporin/anti-Id-1, anti-CD22/anti-saporin, anti-CD7/anti-saporin, anti-CD38/anti-saporin, anti-CEA/anti-ricin A chain, anti-interferon-.alpha.(IFN-.alpha.)/anti-hybridoma idiotype, anti-CEA/anti-vinca alkaloid); bispecific binding polypeptides for converting enzyme activated prodrugs (such as anti-CD30/anti-alkaline phosphatase (which catalyzes conversion of mitomycin phosphate prodrug to mitomycin alcohol)); bispecific binding polypeptides which can be used as fibrinolytic agents (such as anti-fibrin/anti-tissue plasminogen activator (tPA), anti-fibrin/anti-urokinase-type plasminogen activator (uPA)); bispecific binding polypeptides for targeting immune complexes to cell surface receptors (such as anti-low density lipoprotein (LDL)/anti-Fc receptor (e.g. Fc.gamma.RI, Fc.gamma.RII or Fc.gamma.RIII)); bispecific binding polypeptides for use in therapy of infectious diseases (such as anti-CD3/anti-herpes simplex virus (HSV), anti-T-cell receptor:CD3 complex/anti-influenza, anti-Fc.gamma.R/anti-HIV; bispecific binding polypeptides for tumor detection in vitro or in vivo such as anti-CEA/anti-EOTUBE, anti-CEA/anti-DPTA, anti-p185HER2/anti-hapten); bispecific binding polypeptides as vaccine adjuvants (see Fanger et al., supra); and bispecific binding polypeptides as diagnostic tools (such as anti-rabbit IgG/anti-ferritin, anti-horse radish peroxidase (HRP)/anti-hormone, anti-somatostatin/anti-substance P, anti-HRP/anti-FITC, anti-CEA/anti-.beta.-galactosidase (see Nolan et al., supra)). Examples of trispecific polypeptides include anti-CD3/anti-CD4/anti-CD37, anti-CD3/anti-CD5/anti-CD37 and anti-CD3/anti-CD8/anti-CD37.

In a preferred embodiment, a bispecific binding polypeptide of the invention has one arm which binds to CRIPTO-I. In another preferred embodiment, a bispecific binding polypeptide of the invention has one arm which binds to LTβR. In another preferred embodiment, a bispecific binding polypeptide of the invention has one arm which binds to TRAIL-R2. In another preferred embodiment, a bispecific binding polypeptide of the invention has one arm which binds to LTβR and one arm which binds to TRAIL-R2.

Multispecific binding polypeptide of the invention may be monovalent for each specificity or be multivalent for each specificity. For example, binding polypeptides of the invention may comprise one binding site that reacts with a first target molecule and one binding site that reacts with a second target molecule or it may comprise two binding sites that react with a first target molecule and two binding sites that react with a second target molecule.

Binding polypeptides of the invention may have at least two binding specificities from two or more binding domains of a ligand or receptor). They can be assembled as heterodimers, heterotrimers or heterotetramers, essentially as disclosed in WO 89/02922 (published Apr. 6, 1989), in EP 314, 317 (published May 3, 1989), and in U.S. Pat. No. 5,116,964 issued May 2, 1992. Examples include CD4-IgG/TNFreceptor-IgG and CD4-IgG/L-selectin-IgG. The last mentioned molecule combines the lymph node binding function of the lymphocyte homing receptor (LHR, L-selectin), and the HIV binding function of CD4, and finds potential application in the prevention or treatment of HIV infection, related conditions, or as a diagnostic.

(b) scFv-Containing Multispecific Binding Molecules

In one embodiment, the multispecific binding molecules of the invention are multispecific binding molecules comprising at least one scFv molecule, e.g. an scFv molecule described supra. In other embodiments, the multispecific binding molecules of the invention comprise two scFv molecules, e.g. a bispecific scFv (Bis-scFv). In certain embodiments, the scFv molecule is a conventional scFv molecule. In other embodiments, the scFv molecule is a stabilized scFv molecule described supra. In certain embodiments, a multispecific binding molecule may be created by linking a scFv molecule (e.g., a stabilized scFv molecule) with a binding molecule scaffold comprising an scFc molecule. In one embodiment, the starting molecule is selected from the binding molecules described supra, and the scFv molecule and the starting binding molecule have different binding sites. For example, a binding molecule of the invention may comprise a scFv molecule with a first binding specificity linked to a second scFv molecule or a non-scFv binding molecule, that imparts second binding specificity. In one embodiment, a binding molecule of the invention is a naturally occurring antibody to which a stabilized scFv molecule has been fused.

When a stabilized scFv is linked to a parent binding molecule, linkage of the stabilized scFv molecule preferably improves the thermal stability of the binding molecule by at least about 2° C. or 3° C. In one embodiment, the scFv-containing binding molecule of the invention has a 1° C. improved thermal stability as compared to a conventional binding molecule. In another embodiment, a binding molecule of the invention has a 2° C. improved thermal stability as compared to a conventional binding molecule. In another embodiment, a binding molecule of the invention has a 4, 5, 6° C. improved thermal stability as compared to a conventional binding molecule.

In one embodiment, the multispecific-binding molecules of the invention comprise at least one scFv (e.g. 2, 3, or 4 scFvs, e.g., stabilized scFvs). Further details regarding scFv molecules can be found in U.S. Ser. No. 11/725,970, incorporated by reference herein.

In one embodiment, the binding molecules of the invention are multispecific multivalent binding molecules having at least one scFv fragment with a first binding specificity and at least one scFv with a second binding specificity. In preferred embodiments, at least one of the scFv molecules is stabilized.

In another embodiment, the binding molecules of the invention are scFv tetravalent binding molecules. In preferred embodiments at least one of the scFv molecules is stabilized.

### (c) Multispecific Binding Molecule Fragments

In certain embodiments, binding polypeptide of the invention may comprise a binding site from a multispecific binding molecule fragment. Multispecific binding molecule fragments include bispecific Fab2 or multispecific (e.g. trispecific) Fab3 molecules. For example, a multispecific binding molecule fragment may comprise chemically conjugated multimers (e.g. dimers, trimers, or tetramers) of Fab or scFv molecules having different specificities.

### (d) Tandem Variable Domain Binding Molecules

In other embodiments, the multispecific binding molecule of the invention may comprise a binding molecule comprising tandem antigen binding sites. For example, a variable domain may comprise an antibody heavy chain that is engineered to include at least two (e.g., two, three, four, or more) variable heavy domains (VH domains) that are directly fused or linked in series, and an antibody light chain that is engineered to include at least two (e.g., two, three, four, or more) variable light domains (VL domains) that are direct fused or linked in series. The VH domains interact with corresponding VL domains to forms a series of antigen binding sites wherein at least two of the binding sites bind different epitopes. Tandem variable domain binding molecules may comprise two or more of heavy or light chains and are of higher order valency (e.g., bivalent or tetravalent). Methods for making tandem variable domain binding molecules are known in the art, see e.g. WO 2007/024715.

### (e) Dual Specificity Binding Molecules

In other embodiments, the multispecific binding molecule of the invention may comprise a single binding site having dual binding specificity. For example, a dual specificity binding molecule of the invention may comprise a binding site which cross-reacts with two epitopes. Art-recognized methods for producing dual specificity binding molecules are known in the art. For example, dual specificity binding molecules can be isolated by screening for binding molecules which bind both a first epitope and counter-screening the isolated binding molecules for the ability to bind to a second epitope.

### (f) Multispecific Fusion Proteins

In another embodiment, a multispecific binding molecule of the invention is a multispecific fusion protein. As used herein the phrase “multispecific fusion protein” designates fusion proteins (as hereinabove defined) having at least two binding specificities and further comprising an scFc. Multispecific fusion proteins can be assembled, e.g., as heterodimers, heterotrimers or heterotetramers, essentially as disclosed in WO 89/02922 (published Apr. 6, 1989), in EP 314, 317 (published May 3, 1989), and in U.S. Pat. No. 5,116,964 issued May 2, 1992. Preferred multispecific fusion proteins are bispecific. In certain embodiments, at least of the binding specificities of the multispecific fusion protein comprises an scFv, e.g., a stabilized scFv.

A variety of other multivalent antibody constructs may be developed by one of skill in the art using routine recombinant DNA techniques, for example as described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison (1985) Science 229:1202-1207; Oi et al. (1986) BioTechniques 4:214; U.S. Pat. No. 5,225,539; Jones et al. (1986) Nature 321:552-525; Verhoeyan et al. (1988) Science 239:1534; Beidler et al. (1988) J. Immunol. 141:4053-4060; and Winter and Milstein, Nature, 349, pp. 293-99 (1991)). Preferably non-human antibodies are “humanized” by linking the non-human antigen binding domain with a human constant domain (e.g. Cabilly et al., U.S. Pat. No. 4,816,567; Morrison et al., Proc. Natl. Acad. Sci. U.S.A., 81, pp. 6851-55 (1984)).

Other methods which may be used to prepare multivalent antibody constructs are described in the following publications: Ghetie, Maria-Ana et al. (2001) Blood 97:1392-1398; Wolff, Edith A. et al. (1993) Cancer Research 53:2560-2565; Ghetie, Maria-Ana et al. (1997) Proc. Natl. Acad. Sci. 94:7509-7514; Kim, J C. et al. (2002) Int. J. Cancer 97(4):542-547; Todorovska, Aneta et al. (2001) Journal of Immunological Methods 248:47-66; Coloma M. J. et al. (1997) Nature Biotechnology 15:159-163; Zuo, Zhuang et al. (2000) Protein Engineering (Suppl.) 13(5):361-367; Santos A. D., et al. (1999) Clinical Cancer Research 5:3118s-3123s; Presta, Leonard G. (2002) Current Pharmaceutical Biotechnology 3:237-256; van Spriel, Annemiek et al., (2000) Review Immunology Today 21(8) 391-397.

### IV. PREPARATION OF BINDING POLYPEPTIDES

Having selected a binding site for incorporation into an scFc scaffold, a variety of methods are available for producing a binding molecule of the invention. Methods for linking desired target binding sites, whether derived from antibodies or other molecules, to scFc scaffolds are known in the art.

It will be understood that because of the degeneracy of the code, a variety of nucleic acid sequences will encode the amino acid sequence of the binding polypeptide. The desired polynucleotide can be produced by de novo solid-phase DNA synthesis or by PCR mutagenesis of an earlier prepared polynucleotide encoding the target polypeptide.

Oligonucleotide-mediated mutagenesis is one method for preparing a substitution, in-frame insertion, or alteration (e.g., altered codon) to introduce a codon encoding an amino acid substitution (e.g., into an Fc variant moiety). For example, the starting polypeptide DNA is altered by hybridizing an oligonucleotide encoding the desired mutation to a single-stranded DNA template. After hybridization, a DNA polymerase is used to synthesize an entire second complementary strand of the template that incorporates the oligonucleotide primer. In one embodiment, genetic engineering, e.g., primer-based PCR mutagenesis, is sufficient to incorporate an alteration, as defined herein, for producing a polynucleotide encoding an binding polypeptide of the invention.

Polynucleotide sequence encoding the binding polypeptide can then be inserted in a suitable expression vector and transfected into prokaryotic or eukaryotic host cells such as E. coli cells, simian COS cells, Chinese Hamster Ovary (CHO) cells or myeloma cells that do not otherwise produce said proteins, for recombinant expression.

For the purposes of this invention, numerous expression vector systems may be employed. These expression vectors are typically replicable in the host organisms either as episomes or as an integral part of the host chromosomal DNA. Expression vectors may include expression control sequences including, but not limited to, promoters (e.g., naturally-associated or heterologous promoters), enhancers, signal sequences, splice signals, enhancer elements, and transcription termination sequences. Preferably, the expression control sequences are eukaryotic promoter systems in vectors capable of transforming or transfecting eukaryotic host cells. Expression vectors may also utilize DNA elements which are derived from animal viruses such as bovine papilloma virus, polyoma virus, adenovirus, vaccinia virus, baculovirus, retroviruses (RSV, MMTV or MOMLV), cytomegalovirus (CMV), or SV40 virus. Others involve the use of polycistronic systems with internal ribosome binding sites.

Commonly, expression vectors contain selection markers (e.g., ampicillin-resistance, hygromycin-resistance, tetracycline resistance or neomycin resistance) to permit detection of those cells transformed with the desired DNA sequences (see, e.g., Itakura et al., U.S. Pat. No. 4,704,362). Cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation.

A preferred expression vector is NEOSPLA (U.S. Pat. No. 6,159,730). This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. This vector has been found to result in very high level expression of antibodies upon incorporation of variable and constant region genes, transfection in CHO cells, followed by selection in G418 containing medium and methotrexate amplification. Vector systems are also taught in U.S. Pat. Nos. 5,736,137 and 5,658,570, each of which is incorporated by reference in its entirety herein. This system provides for high expression levels, e.g., >30 pg/cell/day. Other exemplary vector systems are disclosed e.g., in U.S. Pat. No. 6,413,777.

Where the binding polypeptide of the invention comprises the antigen binding site of an antibody, polynucleotides encoding additional light and heavy chain variable regions, optionally linked to a genetically-fused Fc region (i.e., scFc region), may be inserted into the same or different expression vector. The DNA segments encoding immunoglobulin chains are operably linked to control sequences in the expression vector(s) that ensure the expression of immunoglobulin polypeptides.

In other preferred embodiments the binding polypeptides of the invention of the instant invention may be expressed using polycistronic constructs. In these expression systems, multiple gene products of interest such as multiple binding polypeptides of multimer binding protein may be produced from a single polycistronic construct. These systems advantageously use an internal ribosome entry site (IRES) to provide relatively high levels of polypeptides of the invention in eukaryotic host cells. Compatible IRES sequences are disclosed in U.S. Pat. No. 6,193,980 which is also incorporated herein. Those skilled in the art will appreciate that such expression systems may be used to effectively produce the full range of polypeptides disclosed in the instant application.

More generally, once the vector or DNA sequence encoding a binding polypeptide has been prepared, the expression vector may be introduced into an appropriate host cell. That is, the host cells may be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Most preferably, plasmid introduction into the host is via electroporation. The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or flourescence-activated cell sorter analysis (FACS), immunohistochemistry and the like.

As used herein, the term “transformation” shall be used in a broad sense to refer to the introduction of DNA into a recipient host cell that changes the genotype and consequently results in a change in the recipient cell.

Along those same lines, “host cells” refers to cells that have been transformed with vectors constructed using recombinant DNA techniques and encoding at least one heterologous gene. In descriptions of processes for isolation of binding polypeptides from recombinant hosts, the terms “cell” and “cell culture” are used interchangeably to denote the source of binding polypeptide unless it is clearly specified otherwise. In other words, recovery of polypeptide from the “cells” may mean either from spun down whole cells, or from the cell culture containing both the medium and the suspended cells.

The host cell line used for protein expression is most preferably of mammalian origin; those skilled in the art are credited with ability to preferentially determine particular host cell lines which are best suited for the desired gene product to be expressed therein. Exemplary host cell lines include, but are not limited to, DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/O (mouse myeloma), P3.times.63-Ag3.653 (mouse myeloma), BFA-1c1BPT (bovine endothelial cells), RAJI (human lymphocyte) and 293 (human kidney). CHO cells are particularly preferred. Host cell lines are typically available from commercial services, the American Tissue Culture Collection or from published literature.

Genes encoding the polypeptides of the invention can also be expressed in non-mammalian cells such as bacteria or yeast or plant cells. In this regard it will be appreciated that various unicellular non-mammalian microorganisms such as bacteria can also be transformed; i.e., those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the polypeptides typically become part of inclusion bodies. The polypeptides must be isolated, purified and then assembled into functional molecules.

In addition to prokaryates, eukaryotic microbes may also be used. Saccharomyces cerevisiae, or common baker's yeast, is the most commonly used among eukaryotic microorganisms although a number of other strains are commonly available. For expression in Saccharomyces, the plasmid YRp7, for example, (Stinchcomb et al., Nature, 282:39 (1979); Kingsman et al., Gene, 7:141 (1979); Tschemper et al., Gene, 10:157 (1980)) is commonly used. This plasmid already contains the TRP1 gene which provides a selection marker for a mutant strain of yeast lacking the ability to grow in tryptophan, for example ATCC No. 44076 or PEP4-1 (Jones, Genetics, 85:12 (1977)). The presence of the trpl lesion as a characteristic of the yeast host cell genome then provides an effective environment for detecting transformation by growth in the absence of tryptophan. Other yeast hosts such Pichia may also be employed. Yeast expression vectors having expression control sequences (e.g., promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for methanol, maltose, and galactose utilization.

Alternatively, polypeptide-coding nucleotide sequences can be incorporated in transgenes for introduction into the genome of a transgenic animal and subsequent expression in the milk of the transgenic animal (see, e.g., Deboer et al., U.S. Pat. No. 5,741,957, Rosen, U.S. Pat. No. 5,304,489, and Meade et al., U.S. Pat. No. 5,849,992). Suitable transgenes include coding sequences for binding polypeptides in operable linkage with a promoter and enhancer from a mammary gland specific gene, such as casein or beta lactoglobulin.

In vitro production allows scale-up to give large amounts of the desired polypeptides. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose or (immuno-)affinity chromatography, e.g., after preferential biosynthesis of a synthetic hinge region polypeptide or prior to or subsequent to the HIC chromatography step described herein. An affinity tag sequence (e.g. a His(6) tag) may optionally be attached or included within the polypeptide sequence to facilitate downstream purification.

Wherein the binding polyeptides of the invention form multimeric proteins or multimers (e.g., dimeric binding polyeptides), the multimeric proteins can be expressed using a single vector or two vectors. When the binding polypeptides are cloned on separate expression vectors, the vectors are co-transfected to obtain expression and assembly of intact whole proteins. Once expressed, the whole proteins, their dimers, individual polypeptides (e.g. binding polypeptides), or other forms can be purified according to standard procedures of the art, including ammonium sulfate precipitation, affinity column chromatography, HPLC purification, gel electrophoresis and the like (see generally Scopes, Protein Purification (Springer-Verlag, N.Y., (1982)). Substantially pure proteins of at least about 90 to 95% homogeneity are preferred, and 98 to 99% or more homogeneity most preferred, for pharmaceutical uses.

### V. PURIFICATION OF BINDING MOLECULES

In one embodiment, the invention pertains to a method of purification of binding molecules of the invention which are expressed as double-chain (i.e., dimeric) scFc binding molecules comprising genetically-fused Fc regions (i.e., scFc regions) away from single-chain (i.e., monomeric) scFc binding molecules comprising genetically fused Fc regions. In other embodiments, the invention provides methods of purifying double-chain scFc binding molecules away from single-chain scFc binding molecules.

In one embodiment, a population comprising both single- and double-chain scFc proteins may be purified by size-exclusion chromatography. For example, single-chain scFc binding molecules may be separated from aggregates and double-chain scFc molecules, e.g., using a Superdex 200 gel filtration column. Gel filtration fractions may be analyzed, e.g., by reducing and non-reducing SDS-PAGE and appropriate fractions combined to obtain homogeneous pools of the single- and double-chain Fc populations. These pools may be further characterized to determine homogeneity and molecular mass of the molecules, e.g., by analytical SEC (TSK-Gel G3000 SW_{XL} column) with on-line light scattering analysis. The invention also pertains to purified populations of double-chain scFc binding molecules comprising genetically-fused Fc domains (i.e., dimeric scFc binding molecules) as well as purified populations of single-chain scFc binding molecules comprising genetically fused Fc domains (i.e., monomeric scFc binding molecules).

### VI. LABELING OR CONJUGATION OF FUNCTIONAL MOIETIES

The binding polypeptides of the present invention may be used in non-conjugated form or may be conjugated to at least one of a variety of functional moieties, e.g., to facilitate target detection or for imaging or therapy of the patient. The polypeptides of the invention can be labeled or conjugated either before or after purification, when purification is performed. In particular, the polypeptides of the present invention may be conjugated (e.g., via an engineered cysteine residue) to a functional moiety. Functional moieties are preferably attached to a portion of the binding polypeptide other than a binding site (e.g., a polypeptide linker or an Fc moiety of a genetically-fused Fc region (i.e., a scFc region)).

Exemplary functional moieties include affinity moieties, and effector moieties. Exemplary effector moieties include cytotoxins (such as radioisotopes, cytotoxic drugs, or toxins) therapeutic agents, cytostatic agents, biological toxins, prodrugs, peptides, proteins, enzymes, viruses, lipids, biological response modifiers, pharmaceutical agents, immunologically active ligands (e.g., lymphokines or other antibodies wherein the resulting molecule binds to both the neoplastic cell and an effector cell such as a T cell), PEG, or detectable molecules useful in imaging. In another embodiment, a polypeptide of the invention can be conjugated to a molecule that decreases vascularization of tumors. In other embodiments, the disclosed compositions may comprise polypeptides of the invention coupled to drugs or prodrugs. Still other embodiments of the present invention comprise the use of polypeptides of the invention conjugated to specific biotoxins or their cytotoxic fragments such as ricin, gelonin, Pseudomonas exotoxin or diphtheria toxin. The selection of which conjugated or unconjugated polypeptide to use will depend on the type and stage of cancer, use of adjunct treatment (e.g., chemotherapy or external radiation) and patient condition. It will be appreciated that one skilled in the art could readily make such a selection in view of the teachings herein.

It will be appreciated that, in previous studies, anti-tumor antibodies labeled with isotopes have been used successfully to destroy cells in solid tumors as well as lymphomas/leukemias in animal models, and in some cases in humans. Exemplary radioisotopes include: ⁹⁰Y, ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re. The radionuclides act by producing ionizing radiation which causes multiple strand breaks in nuclear DNA, leading to cell death. The isotopes used to produce therapeutic conjugates typically produce high energy α- or β-particles which have a short path length. Such radionuclides kill cells to which they are in close proximity, for example neoplastic cells to which the conjugate has attached or has entered. They have little or no effect on non-localized cells. Radionuclides are essentially non-immunogenic.

With respect to the use of radiolabeled conjugates in conjunction with the present invention, binding polypeptides of the invention may be directly labeled (such as through iodination) or may be labeled indirectly through the use of a chelating agent. As used herein, the phrases “indirect labeling” and “indirect labeling approach” both mean that a chelating agent is covalently attached to a binding polypeptide and at least one radionuclide is associated with the chelating agent. Such chelating agents are typically referred to as bifunctional chelating agents as they bind both the polypeptide and the radioisotope. Particularly preferred chelating agents comprise 1-isothiocycmatobenzyl-3-methyldiothelene triaminepentaacetic acid (“MX-DTPA”) and cyclohexyl diethylenetriamine pentaacetic acid (“CHX-DTPA”) derivatives. Other chelating agents comprise P-DOTA and EDTA derivatives. Particularly preferred radionuclides for indirect labeling include ¹¹¹In and ⁹⁰Y.

As used herein, the phrases “direct labeling” and “direct labeling approach” both mean that a radionuclide is covalently attached directly to a polypeptide (typically via an amino acid residue). More specifically, these linking technologies include random labeling and site-directed labeling. In the latter case, the labeling is directed at specific sites on the polypeptide, such as the N-linked sugar residues present only in an Fc domain of the conjugates. Further, various direct labeling techniques and protocols are compatible with the instant invention. For example, Technetium-99m labeled polypeptides may be prepared by ligand exchange processes, by reducing pertechnate (TcO₄⁻) with stannous ion solution, chelating the reduced technetium onto a Sephadex column and applying the polypeptides to this column, or by batch labeling techniques, e.g. by incubating pertechnate, a reducing agent such as SnCl₂, a buffer solution such as a sodium-potassium phthalate-solution, and the antibodies. In any event, preferred radionuclides for directly labeling antibodies are well known in the art and a particularly preferred radionuclide for direct labeling is 131, covalently attached via tyrosine residues. Polypeptides according to the invention may be derived, for example, with radioactive sodium or potassium iodide and a chemical oxidizing agent, such as sodium hypochlorite, chloramine T or the like, or an enzymatic oxidizing agent, such as lactoperoxidase, glucose oxidase and glucose. However, for the purposes of the present invention, the indirect labeling approach is particularly preferred.

Patents relating to chelators and chelator conjugates are known in the art. For instance, U.S. Pat. No. 4,831,175 of Gansow is directed to polysubstituted diethylenetriaminepentaacetic acid chelates and protein conjugates containing the same, and methods for their preparation. U.S. Pat. Nos. 5,099,069, 5,246,692, 5,286,850, 5,434,287 and 5,124,471 of Gansow also relate to polysubstituted DTPA chelates. These patents are incorporated herein in their entirety. Other examples of compatible metal chelators are ethylenediaminetetraacetic acid (EDTA), diethylenetriaminepentaacetic acid (DPTA), 1,4,8,11-tetraazatetradecane, 1,4,8,11-tetraazatetradecane-1,4,8,11-tetraacetic acid, 1-oxa-4,7,12,15-tetraazaheptadecane-4,7,12,15-tetraacetic acid, or the like. Cyclohexyl-DTPA or CHX-DTPA is particularly preferred and is exemplified extensively below. Still other compatible chelators, including those yet to be discovered, may easily be discerned by a skilled artisan and are clearly within the scope of the present invention.

Compatible chelators, including the specific bifunctional chelator used to facilitate chelation in co-pending application Ser. Nos. 08/475,813, 08/475,815 and 08/478,967, are preferably selected to provide high affinity for trivalent metals, exhibit increased tumor-to-non-tumor ratios and decreased bone uptake as well as greater in vivo retention of radionuclide at target sites, i.e., B-cell lymphoma tumor sites. However, other bifunctional chelators that may or may not possess all of these characteristics are known in the art and may also be beneficial in tumor therapy.

It will also be appreciated that, in accordance with the teachings herein, binding polypeptides may be conjugated to different radiolabels for diagnostic and therapeutic purposes. To this end the aforementioned co-pending applications, herein incorporated by reference in their entirety, disclose radiolabeled therapeutic conjugates for diagnostic “imaging” of tumors before administration of therapeutic antibody. “In2B8” conjugate comprises a murine monoclonal antibody, 2B8, specific to human CD20 antigen, that is attached to ¹¹¹In via a bifunctional chelator, i.e., MX-DTPA (diethylene-triaminepentaacetic acid), which comprises a 1:1 mixture of 1-isothiocyanatobenzyl-3-methyl-DTPA and 1-methyl-3-isothiocyanatobenzyl-DTPA. ¹¹¹In is particularly preferred as a diagnostic radionuclide because between about 1 to about 10 mCi can be safely administered without detectable toxicity; and the imaging data is generally predictive of subsequent ⁹⁰Y-labeled antibody distribution. Most imaging studies utilize 5 mCi ¹¹¹In-labeled antibody, because this dose is both safe and has increased imaging efficiency compared with lower doses, with optimal imaging occurring at three to six days after antibody administration. See, for example, Murray, J. Nuc. Med. 26: 3328 (1985) and Carraguillo et al., J. Nuc. Med. 26: 67 (1985).

As indicated above, a variety of radionuclides are applicable to the present invention and those skilled in the art can readily determine which radionuclide is most appropriate under various circumstances. For example, ¹³¹I is a well known radionuclide used for targeted immunotherapy. However, the clinical usefulness of ¹³¹I can be limited by several factors including: eight-day physical half-life; dehalogenation of iodinated antibody both in the blood and at tumor sites; and emission characteristics (e.g., large gamma component) which can be suboptimal for localized dose deposition in tumor. With the advent of superior chelating agents, the opportunity for attaching metal chelating groups to proteins has increased the opportunities to utilize other radionuclides such as ¹¹¹In and ⁹⁰Y. ⁹⁰Y provides several benefits for utilization in radioimmunotherapeutic applications: the 64 hour half-life of ⁹⁰Y is long enough to allow antibody accumulation by tumor and, unlike e.g., ¹³¹I, ⁹⁰Y is a pure beta emitter of high energy with no accompanying gamma irradiation in its decay, with a range in tissue of 100 to 1,000 cell diameters. Furthermore, the minimal amount of penetrating radiation allows for outpatient administration of ⁹⁰Y-labeled antibodies. Additionally, internalization of labeled antibody is not required for cell killing, and the local emission of ionizing radiation should be lethal for adjacent tumor cells lacking the target molecule.

Those skilled in the art will appreciate that these non-radioactive conjugates may also be assembled using a variety of techniques depending on the selected agent to be conjugated. For example, conjugates with biotin are prepared e.g. by reacting the polypeptides with an activated ester of biotin such as the biotin N-hydroxysuccinimide ester. Similarly, conjugates with a fluorescent marker may be prepared in the presence of a coupling agent, e.g. those listed above, or by reaction with an isothiocyanate, preferably fluorescein-isothiocyanate. Conjugates of the polypeptides of the invention with cytostatic/cytotoxic substances and metal chelates are prepared in an analogous manner.

Many effector molecules lack suitable functional groups to which binding polyeptides can be linked. In one embodiment, an effector molecule, e.g., a drug or prodrug is attached to the binding polypeptide through a linking molecule. In one embodiment, the linking molecule contains a chemical bond that allows for the activation of cytotoxicity at a particular site. Suitable chemical bonds are well known in the art and include disulfide bonds, acid labile bonds, photolabile bonds, peptidase labile bonds, thioether bonds formed between sulfhydryl and maleimide groups, and esterase labile bonds. Most preferably, the linking molecule comprises a disulfide bond or a thioether bond. In accordance with the invention, the linking molecule preferably comprises a reactive chemical group. Particularly preferred reactive chemical groups are N-succinimidyl esters and N-sulfosuccinimidyl esters. In a preferred embodiment, the reactive chemical group can be covalently bound to the effector via disulfide bonding between thiol groups. In one embodiment an effector molecule is modified to comprise a thiol group. One of ordinary skill in the art will appreciate that a thiol group contains a sulfur atom bonded to a hydrogen atom and is typically also referred to in the art as a sulfhydryl group, which can be denoted as “—SH” or “RSH.”

In one embodiment, a linking molecule may be used to join an effector molecule with a binding polypeptide of the invention. The linking molecule may be cleavable or non-cleavable. In one embodiment, the cleavable linking molecule is a redox-cleavable linking molecule, such that the linking molecule is cleavable in environments with a lower redox potential, such as the cytoplasm and other regions with higher concentrations of molecules with free sulfhydryl groups. Examples of linking molecules that may be cleaved due to a change in redox potential include those containing disulfides. The cleaving stimulus can be provided upon intracellular uptake of the binding protein of the invention where the lower redox potential of the cytoplasm facilitates cleavage of the linking molecule. In another embodiment, a decrease in pH triggers the release of the maytansinoid cargo into the target cell. The decrease in pH is implicated in many physiological and pathological processes, such as endosome trafficking, tumor growth, inflammation, and myocardial ischemia. The pH drops from a physiological 7.4 to 5-6 in endosomes or 4-5 in lysosomes. Examples of acid sensitive linking molecules which may be used to target lysosomes or endosomes of cancer cells, include those with acid-cleavable bonds such as those found in acetals, ketals, orthoesters, hydrazones, trityls, cis-aconityls, or thiocarbamoyls (see for example, Willner et al., (1993), Bioconj. Chem., 4: 521-7; U.S. Pat. Nos. 4,569,789, 4,631,190, 5,306,809, and 5,665,358). Other exemplary acid-sensitive linking molecules comprise dipeptide sequences Phe-Lys and Val-Lys (King et al., (2002), J. Med. Chem., 45: 4336-43). The cleaving stimulus can be provided upon intracellular uptake trafficking to low pH endosomal compartments (e.g. lysosomes). Other exemplary acid-cleavable linking molecules are the molecules that contain two or more acid cleavable bonds for attachment of two or more maytansinoids (King et al., (1999), Bioconj. Chem., 10: 279-88; WO 98/19705).

Cleavable linking molecules may be sensitive to biologically supplied cleaving agents that are associated with a particular target cell, for example, lysosomal or tumor-associated enzymes. Examples of linking molecules that can be cleaved enzymatically include, but are not limited to, peptides and esters. Exemplary enzyme cleavable linking molecules include those that are sensitive to tumor-associated proteases such as Cathepsin B or plasmin (Dubowchik et al., (1999), Pharm. Ther., 83: 67-123; Dubowchik et al., (1998), Bioorg. Med. Chem. Lett., 8: 3341-52; de Groot et al., (2000), J. Med. Chem., 43: 3093-102; de Groot et al., (1999)m 42: 5277-83). Cathepsin B-cleavable sites include the dipeptide sequences valine-citrulline and phenylalanine-lysine (Doronina et al., (2003), Nat. Biotech., 21(7): 778-84); Dubowchik et al., (2002), Bioconjug. Chem., 13: 855-69). Other exemplary enzyme-cleavable sites include those formed by oligopeptide sequences of 4 to 16 amino acids (e.g., Suc-β-Ala-Leu-Ala-Leu) which recognized by trouse proteases such as Thimet Oliogopeptidase (TOP), an enzyme that is preferentially released by neutrophils, macrophages, and other granulocytes.

In a further embodiment, a binding polypeptide of the invention is reacted with a linking molecule of the formula:

X-Y-Z

wherein:
X is an attachment molecule;
Y is a spacer molecule; and
Z is a effector attachment moiety.

The term “attachment molecule” includes molecules which allow for the covalent attachment of the linking molecule to a binding polypeptide of the invention. The attachment molecule may comprise, for example, a covalent chain of 1-60 carbon, oxygen, nitrogen, sulfur atoms, optionally substituted with hydrogen atoms and other substituents which allow the binding molecule to perform its intended function. The attachment molecule may comprise peptide, ester, alkyl, alkenyl, alkynyl, aryl, ether, thioether, etc. functional groups. Preferably, the attachment molecule is selected such that it is capable of reacting with a reactive functional group on a polypeptide comprising at least one antigen binding site, to form a binding molecule of the invention. Examples of attachment molecules include, for example, amino, carboxylate, and thiol attachment molecules.

Amino attachment molecules include molecules which react with amino groups on a binding polypeptide, such that a modified polypeptide is formed. Amino attachment molecules are known in the art. Examples of amino attachment molecules include, activated carbamides (e.g., which may react with an amino group on a binding molecule to form a linking molecule which comprises urea group), aldehydes (e.g., which may react with amino groups on a binding molecule), and activated isocyanates (which may react with an amino group on a binding polypeptide to from a linking molecule which comprises a urea group). Examples of amino attachment molecules include, but are not limited to, N-succinimidyl, N-sulfosuccinimidyl, N-phthalimidyl, N-sulfophthalimidyl, 2-nitrophenyl, 4-nitrophenyl, 2,4-dinitrophenyl, 3-sulfonyl-4-nitrophenyl, or 3-carboxy-4-nitrophenyl molecule.

Carboxylate attachment molecules include molecules which react with carboxylate groups on a binding polypeptide, such that a modified binding polypeptide of the invention is formed. Carboxylate attachment molecules are known in the art. Examples of carboxylate attachment molecules include, but are not limited to activated ester intermediates and activated carbonyl intermediates, which may react with a COOH group on a binding polypeptide to form a linking molecule which comprises a ester, thioester, or amide group.

Thiol attachment molecules include molecules which react with thiol groups present on a polypeptide, such that a binding molecule of the invention is formed. Thiol attachment molecules are known in the art. Examples of thiol attachment molecules include activated acyl groups (which may react with a sulfhydryl on a binding molecule to form a linking molecule which comprises a thioester), activated alkyl groups (which may react with a sulfhydryl on a binding molecule to form a linking molecule which comprises a thioester molecule), Michael acceptors such as maleimide or acrylic groups (which may react with a sulfhydryl on a binding molecule to form a Michael-type addition product), groups which react with sulfhydryl groups via redox reactions, activated di-sulfide groups (which may react with a sulfhydryl group on a binding molecule to form, for example, a linking molecule which comprises a disulfide molecule). Other thiol attachment molecules include acrylamides, alpha-iodoacetamides, and cyclopropan-1,1-dicarbonyl compounds. In addition, the thiol attachment molecule may comprise a molecule which modifies a thiol on the binding molecule to form another reactive species to which the linking molecule can be attached to form a binding molecule of the invention.

The spacer molecule, Y, is a covalent bond or a covalent chain of atoms which may contain one or more aminoacid residues. It may also comprise 0-60 carbon, oxygen, sulfur or nitrogen atoms optionally substituted with hydrogen or other substituents which allow the resulting binding molecule to perform its intended function.

In one embodiment, Y comprises an alkyl, alkenyl, alkynyl, ester, ether, carbonyl, or amide molecule.

In another embodiment, a thiol group on the binding polypeptide is converted into a reactive group, such as a reactive carbonyl group, such as a ketone or aldehyde. The attachment molecule is then reacted with the ketone or aldehyde to form a modified binding polypeptide. Examples of carbonyl reactive attachment molecules include, but are not limited to, hydrazines, hydrazides, O-substituted hydroxylamines, alpha-beta-unsaturated ketones, and H₂C═CH—CO—NH—NH₂. Other examples of attachment molecules and methods for modifying thiol molecules which can be used to form modified binding polypeptides are described Pratt, M. L. et al. J Am Chem. Soc. 2003 May 21; 125(20):6149-59; and Saxon, E. Science. 2000 Mar. 17; 287(5460):2007-10.

The linking molecule may be a molecule which is capable of reacting with an effector molecule or a derivative thereof to form a binding molecule of the invention. For example, the effector molecule may be linked to the remaining portions of the molecule through a disulfide bond. In such cases, the linking molecule is selected such that it is capable of reacting with an appropriate effector moiety derivative such that the effector molecule is attached to the binding polypeptide of the invention.

Preferred cytotoxic effector molecules for use in the present invention are cytotoxic drugs, particularly those which are used for cancer therapy. As used herein, “a cytotoxin or cytotoxic agent” means any agent that is detrimental to the growth and proliferation of cells and may act to reduce, inhibit or destroy a cell or malignancy. Exemplary cytotoxins include, but are not limited to, radionuclides, biotoxins, enzymatically active toxins, cytostatic or cytotoxic therapeutic agents, prodrugs, immunologically active ligands and biological response modifiers such as cytokines. Any cytotoxin that acts to retard or slow the growth of immunoreactive cells or malignant cells is within the scope of the present invention.

Exemplary cytotoxins include, in general, cytostatic agents, alkylating agents, antimetabolites, anti-proliferative agents, tubulin binding agents, hormones and hormone antagonists, and the like. Exemplary cytostatics that are compatible with the present invention include alkylating substances, such as mechlorethamine, triethylenephosphoramide, cyclophosphamide, ifosfamide, chlorambucil, busulfan, melphalan or triaziquone, also nitrosourea compounds, such as carmustine, lomustine, or semustine.

Exemplary molecules for conjugation are maytansinoids. Maytansinoids were originally isolated from the east African shrub belonging to the genus Maytenus, but were subsequently also discovered to be metabolites of soil bacteria, such as Actinosynnema pretiosum (see, e.g., U.S. Pat. No. 3,896,111). Maytansinoids are known in the art to include maytansine, maytansinol, C-3 esters of maytansinol, and other maytansinol analogues and derivatives (see, e.g., U.S. Pat. Nos. 5,208,020 and 6,441,163). C-3 esters of maytansinol can be naturally occurring or synthetically derived. Moreover, both naturally occurring and synthetic C-3 maytansinol esters can be classified as a C-3 ester with simple carboxylic acids, or a C-3 ester with derivatives of N-methyl-L-alanine, the latter being more cytotoxic than the former. Synthetic maytansinoid analogues also are known in the art and described in, for example, Kupchan et al., J. Med. Chem., 21, 31-37 (1978). Methods for generating maytansinol and analogues and derivatives thereof are described in, for example, U.S. Pat. No. 4,151,042.

Suitable maytansinoids for use as conjugates can be isolated from natural sources, synthetically produced, or semi-synthetically produced using methods known in the art. Moreover, the maytansinoid can be modified in any suitable manner, so long as sufficient cytotoxicity is preserved in the ultimate conjugate molecule.

Particularly preferred maytansinoids comprising a linking molecule that contains a reactive chemical group are C-3 esters of maytansinol and its analogs where the linking molecule contains a disulfide bond and the attachment molecule comprises a N-succinimidyl or N-sulfosuccinimidyl ester. Many positions on maytansinoids can serve as the position to chemically link the linking molecule, e.g., through an effector attachment molecule. For example, the C-3 position having a hydroxyl group, the C-14 position modified with hydroxymethyl, the C-15 position modified with hydroxy and the C-20 position having a hydroxy group are all useful. The linking molecule most preferably is linked to the C-3 position of maytansinol. Most preferably, the maytansinoid used in connection with the inventive composition is N.sup.2′-deacetyl-N.sup.2′-(-3-mercapto-1-oxopropyl)-maytansine (DM1) or N.sup.2′-deacetyl-N.sup.2′-(4-mercapto-4-methyl-1-oxopentyl)-maytansine (DM4).

Linking molecules with other chemical bonds also can be used in the context of the invention, as can other maytansinoids. Specific examples of other chemical bonds which may be incorporated in the linking molecules include those described above, such as, for example acid labile bonds, thioether bonds, photolabile bonds, peptidase labile bonds and esterase labile bonds. Methods for producing maytansinoids with linking molecules and/or effector attachment molecules are described in, for example, U.S. Pat. Nos. 5,208,020, 5,416,064, and 6,333,410.

The linking molecule (and/or the effector attachment molecule) of a maytansinoid typically and preferably is part of a larger peptide molecule that is used to join the binding polypeptide to the maytansinoid. Any suitable peptide molecule can be used in connection with the invention, so long as the linking molecule provides for retention of the cytotoxicity and targeting characteristics of the maytansinoid and the antibody, respectively. The linking molecule joins the maytansinoid to the binding polypeptide through chemical bonds (as described above), such that the maytansinoid and the binding polypeptide are chemically coupled (e.g., covalently bonded) to each other. Desirably, the linking molecule chemically couples the maytansinoid to the binding polypeptide through disulfide bonds or thioether bonds. Most preferably, the binding polypeptide is chemically coupled to the maytansinoid via disulfide bonds.

Other preferred classes of cytotoxic agents include, for example, the anthracycline family of drugs, the vinca drugs, the mitomycins, the bleomycins, the cytotoxic nucleosides, the pteridine family of drugs, diynenes, and the podophyllotoxins. Particularly useful members of those classes include, for example, adriamycin, caminomycin, daunorubicin (daunomycin), doxorubicin, aminopterin, methotrexate, methopterin, mithramycin, streptonigrin, dichloromethotrexate, mitomycin C, actinomycin-D, porfiromycin, 5-fluorouracil, floxuridine, ftorafur, 6-mercaptopurine, cytarabine, cytosine arabinoside, podophyllotoxin, or podophyllotoxin derivatives such as etoposide or etoposide phosphate, melphalan, vinblastine, vincristine, leurosidine, vindesine, leurosine and the like. Still other cytotoxins that are compatible with the teachings herein include taxol, taxane, cytochalasin B, gramicidin D, ethidium bromide, emetine, tenoposide, colchicin, dihydroxy anthracin dione, mitoxantrone, procaine, tetracaine, lidocaine, propranolol, and puromycin and analogs or homologs thereof. Hormones and hormone antagonists, such as corticosteroids, e.g. prednisone, progestins, e.g. hydroxyprogesterone or medroprogesterone, estrogens, e.g. diethylstilbestrol, antiestrogens, e.g. tamoxifen, androgens, e.g. testosterone, and aromatase inhibitors, e.g. aminogluthetimide are also compatible with the teachings herein. As noted previously, one skilled in the art may make chemical modifications to the desired compound in order to make reactions of that compound more convenient for purposes of preparing conjugates of the invention.

Other exemplary cytotoxins comprise members or derivatives of the enediyne family of anti-tumor antibiotics, including calicheamicin, esperamicins or dynemicins. These toxins are extremely potent and act by cleaving nuclear DNA, leading to cell death. Unlike protein toxins which can be cleaved in vivo to give many inactive but immunogenic polypeptide fragments, toxins such as calicheamicin, esperamicins and other enediynes are small molecules which are essentially non-immunogenic. These non-peptide toxins are chemically-linked to the dimers or tetramers by techniques which have been previously used to label monoclonal antibodies and other molecules. These linking technologies include site-specific linkage via the N-linked sugar residues present on an Fc moiety of a binding polypeptide of the invention. Such site-directed linking methods have the advantage of reducing the possible effects of linkage on the binding properties of the constructs.

Among other cytotoxins, it will be appreciated that polypeptides can also be associated with a biotoxin such as ricin subunit A, abrin, diptheria toxin, botulinum, cyanginosins, saxitoxin, shigatoxin, tetanus, tetrodotoxin, trichothecene, verrucologen or a toxic enzyme. Preferably, such constructs will be made using genetic engineering techniques that allow for direct expression of the antibody-toxin construct. Other biological response modifiers that may be associated with the polypeptides of the invention include cytokines such as lymphokines and interferons. In view of the instant disclosure it is submitted that one skilled in the art could readily form such constructs using conventional techniques.

Another class of compatible cytotoxins that may be used in conjunction with the disclosed polypeptides are radiosensitizing drugs that may be effectively directed to tumor or immunoreactive cells. Such drugs enhance the sensitivity to ionizing radiation, thereby increasing the efficacy of radiotherapy. A binding polypeptide conjugate internalized by the tumor cell would deliver the radiosensitizer nearer the nucleus where radiosensitization would be maximal. The unbound radiosensitizer-linked polypeptides of the invention would be cleared quickly from the blood, localizing the remaining radiosensitization agent in the target tumor and providing minimal uptake in normal tissues. After rapid clearance from the blood, adjunct radiotherapy would be administered in one of three ways: 1.) external beam radiation directed specifically to the tumor, 2.) radioactivity directly implanted in the tumor or 3.) systemic radioimmunotherapy with the same targeting antibody. A potentially attractive variation of this approach would be the attachment of a therapeutic radioisotope to the radiosensitized immunoconjugate, thereby providing the convenience of administering to the patient a single drug.

In one embodiment, a molecule that enhances the stability or efficacy of the polypeptide can be conjugated. For example, in one embodiment, PEG can be conjugated to the polypeptides of the invention to increase their half-life in vivo. Leong, S. R., et al. 2001. Cytokine 16:106; 2002; Adv. in Drug Deliv. Rev. 54:531; or Weir et al. 2002. Biochem. Soc. Transactions 30:512.

As previously alluded to, compatible cytotoxins may comprise a prodrug. As used herein, the term “prodrug” refers to a precursor or derivative form of a pharmaceutically active substance that is less cytotoxic to tumor cells compared to the parent drug and is capable of being enzymatically activated or converted into the more active parent form. Prodrugs compatible with the invention include, but are not limited to, phosphate-containing prodrugs, thiophosphate-containing prodrugs, sulfate containing prodrugs, peptide containing prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs that can be converted to the more active cytotoxic free drug. In one embodiment, a cytotoxic agent, such as a maytansinoid, is administered as a prodrug which is released by the hydrolysis of disulfide bonds. Further examples of cytotoxic drugs that can be derivatized into a prodrug form for use in the present invention comprise those chemotherapeutic agents described above.

### VI. METHODS OF USE OF THE POLYPEPTIDES OF THE INVENTION

The polypeptides of the invention can be used in a number of applications, for example in screening assays as well as for diagnostic or therapeutic purposes. Preferred embodiments of the present invention provide kits and methods for the diagnosis and/or treatment of disorders, e.g., neoplastic disorders in a mammalian subject in need of such treatment. Preferably, the subject is a human.

A. Screening Methods

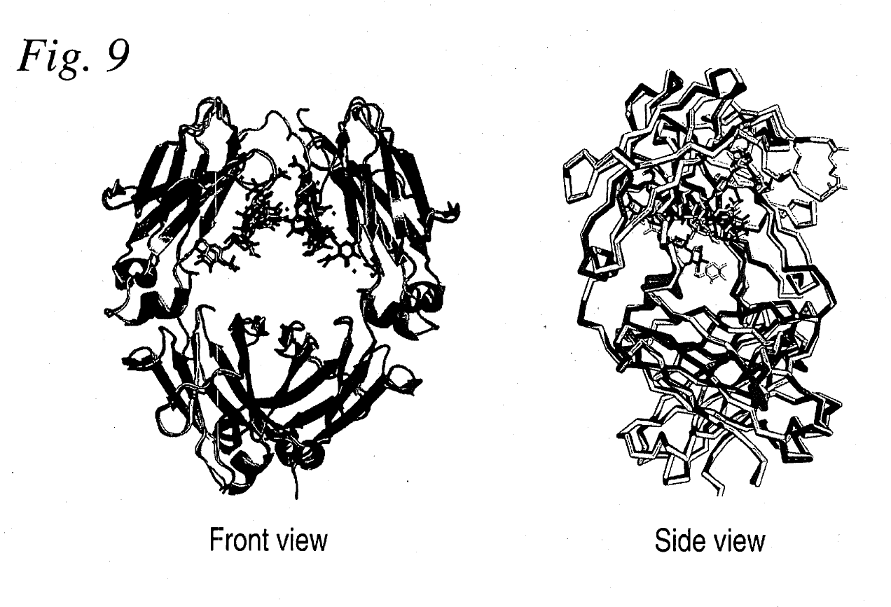

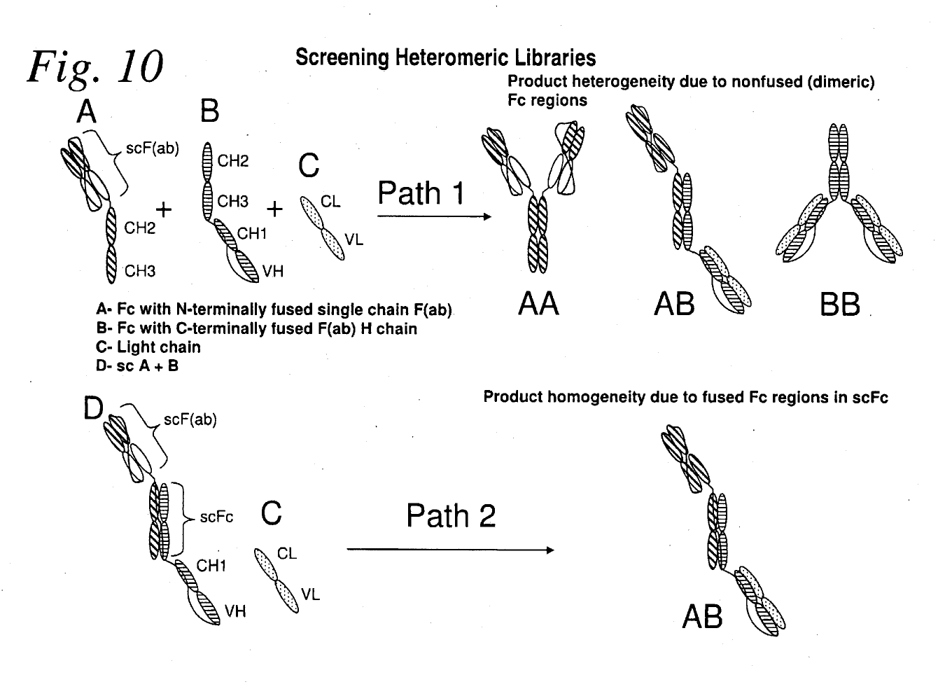

The subject binding molecules are also useful in screening methods. When synthesized as bispecific molecules, the subject binding molecules have numerous advantages over prior art bispecific molecules, including as agents for use in screening assays. FIG. 9 depicts the advantages of using a scFc binding molecule of the invention in screening for bispecific antibody function as compared to use of a conventional bispecific antibody. The use of the scFc region prevents unwanted heterogeneity of the binding domains. Such heterogeneity would result in complicating assays designed to screen for activities unique to bispecific antibodies. “Path 1” is an example of the heterogeneous binding protein combinations that would typically occur when three genes are coexpressed in a eukaryotic system to form a bispecific antibody: (A) a scF(ab) fused to the N-terminus of an Fc; (B) a F(ab) heavy chain fused to the C-terminus of the CH3 domain of an Fc; and (C) the light chain comprising the CL and VL domains. “Path 2” is an example of how fusing two genes ((A) and (B)) into a single genetic construct, results in the Fc moieties being linked by a polypeptide linker, an scFc (D). Coexpression of (C) and (D) results in the homogeneous expression of a single bispecific mAb. Accordingly, binding molecules of the invention present an advantage when screening, e.g., for the ability of a bispecific antibody to bind to one or more of its targets, for example, using methods known in the art.

In another aspect, the invention provides a process for screening for multispecific binding proteins (e.g. bispecific binding proteins such as bispecific antibodies) which activate or inhibit activation of a target protein. In particular, binding polypeptides of the invention having a first binding specificity may be co-expressed or covalently linked with one or more polypeptides of different specificity to form a multi-specific binding protein. In a particularly preferred embodiment, the binding polypeptides of the invention may be expressed as a single genetic construct comprising a genetically-fused Fc region (i.e., a scFc region) with binding sites (e.g., scFv or Fabs) of different specificities at the N- and C-terminus thereof. The binding proteins may be screened in an assay (e.g., a cell-based assay) which measures the relative activity against one or more target proteins of interest. In general, such screening procedures involve contacting the multi-specific binding polypeptide of the invention with the target protein to observe binding, stimulation or inhibition of a functional response. A multi-specific binding protein may be selected if it exhibits stimulation or inhibition relative to a corresponding mono-specific binding polypeptide.

Art-recognized assay which are appropriate for measuring the activity (e.g., biochemical or biological activity) of a target protein may be employed. For example, where the target protein is a kinase, an appropriate assay may comprise measuring the inhibition or activation of the phosphorylation state of the substrate of the kinase. In other exemplary embodiment, where the target protein is G-protein coupled receptor (GPCR) an appropriate assay may measure extracellular pH changes to determine whether the multispecific binding polypeptide activates or inhibits the receptor. Where the target protein is a receptor, a screening assay may involve determining relative binding of labeled ligand to cells which have the receptor on the surface thereof. The ligand can be labeled, e.g., by radioactivity. The amount of labeled ligand bound to the receptors is measured, e.g., by measuring radioactivity of the receptors. If the compound binds to the receptor as determined by a reduction of labeled ligand which binds to the receptors, the binding of labeled ligand to the receptor is inhibited.

In one exemplary embodiment, the assay comprises the inhibition of an LTβR protein (e.g, a LTα1β2 protein). Since the LTβR molecule is a trimer comprised of three, non-identical subunits separated by three clefts, it is desirable to block more than one of the three clefts to optimally inactivate LTβR activity. For example, it is desirable to obtain a multi-specific binding polypeptide having a first specificity for a first cleft of LTβR and a second specificity for a second cleft of LTβR. Accordingly, a multi-specific binding protein having more than one binding specificity for LTβR may be screened in an assay of LTβR activity to determine if it exhibits improved activity relative to a corresponding monospecific antibody.

B. Anti-Tumor Therapy

The polypeptides of the instant invention will be useful in a number of different applications. For example, in one embodiment, the subject binding polypeptides should be useful for reducing or eliminating cells bearing an epitope recognized by the binding polypeptide. In another embodiment, the subject binding polypeptides are effective in reducing the concentration of or eliminating soluble antigen in the circulation

In one embodiment, the binding polypeptides of the invention which recognize tumor-associated antigens may reduce tumor size, inhibit tumor growth and/or prolong the survival time of tumor-bearing animals. Accordingly, this invention also relates to a method of treating tumors in a human or other animal by administering to such human or animal an effective, non-toxic amount of said binding polypeptides. One skilled in the art would be able, by routine experimentation, to determine what an effective, non-toxic amount of modified binding polypeptide would be for the purpose of treating malignancies. For example, a therapeutically active amount of a binding polypeptide may vary according to factors such as the disease stage (e.g., stage I versus stage IV), age, sex, medical complications (e.g., immunosuppressed conditions or diseases) and weight of the subject, and the ability of the binding polypeptide to elicit a desired response in the subject. The dosage regimen may be adjusted to provide the optimum therapeutic response. For example, several divided doses may be administered daily, or the dose may be proportionally reduced as indicated by the exigencies of the therapeutic situation. Generally, however, an effective dosage is expected to be in the range of about 0.05 to 100 milligrams per kilogram body weight per day and more preferably from about 0.5 to 10, milligrams per kilogram body weight per day.

In general, the polypeptides of the invention may be used to prophylactically or therapeutically treat any neoplasm comprising an antigenic marker that allows for the targeting of the cancerous cells by the modified antibody. Exemplary cancers that may be treated include, but are not limited to, prostate, gastric carcinomas such as colon, skin, breast, ovarian, lung and pancreatic. More particularly, the binding polypeptides of the instant invention may be used to treat Kaposi's sarcoma, CNS neoplasias (capillary hemangioblastomas, meningiomas and cerebral metastases), melanoma, gastrointestinal and renal sarcomas, rhabdomyosarcoma, glioblastoma (preferably glioblastoma multi forme), leiomyo sarcoma, retinoblastoma, papillary cystadenocarcinoma of the ovary, Wilm's tumor or small cell lung carcinoma. It will be appreciated that appropriate target binding polypeptides may be derived for tumor associated antigens related to each of the forgoing neoplasias without undue experimentation in view of the instant disclosure.

Exemplary hematologic malignancies that are amenable to treatment with the disclosed invention include Hodgkins and non-Hodgkins lymphoma as well as leukemias, including ALL-L3 (Burkitt's type leukemia), chronic lymphocytic leukemia (CLL) and monocytic cell leukemias. It will be appreciated that the compounds and methods of the present invention are particularly effective in treating a variety of B-cell lymphomas, including low grade/follicular non-Hodgkin's lymphoma (NHL), cell lymphoma (FCC), mantle cell lymphoma (MCL), diffuse large cell lymphoma (DLCL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL and Waldenstrom's Macroglobulinemia. It should be clear to those of skill in the art that these lymphomas will often have different names due to changing systems of classification, and that patients having lymphomas classified under different names may also benefit from the combined therapeutic regimens of the present invention. In addition to the aforementioned neoplastic disorders, it will be appreciated that the polypeptides of the invention may advantageously be used to treat additional malignancies bearing compatible tumor associated antigens.

C. Immune Disorder Therapies

Besides neoplastic disorders, the polypeptides of the instant invention are particularly effective in the treatment of autoimmune disorders or abnormal immune responses. In this regard, it will be appreciated that the polypeptides of the present invention may be used to control, suppress, modulate or eliminate unwanted immune responses to both external and autoantigens. For example, in one embodiment, the antigen is an autoantigen. In another embodiment, the antigen is an allergen. In yet other embodiments, the antigen is an alloantigen or xenoantigen. Use of the binding polypeptides of the invention to reduce an immune response to alloantigens and xenoantigens is of particular use in transplantation, for example to inhibit rejection by a transplant recipient of a donor graft, e.g. a tissue or organ graft or bone marrow transplant. Additionally, suppression or elimination of donor T cells within a bone marrow graft is useful for inhibiting graft versus host disease.

In yet other embodiments the polypeptides of the present invention may be used to treat immune disorders that include, but are not limited to, allergic bronchopulmonary aspergillosis; Allergic rhinitis Autoimmune hemolytic anemia; Acanthosis nigricans; Allergic contact dermatitis; Addison's disease; Atopic dermatitis; Alopecia greata; Alopecia universalis; Amyloidosis; Anaphylactoid purpura; Anaphylactoid reaction; Aplastic anemia; Angioedema, hereditary; Angioedema, idiopathic; Ankylosing spondylitis; Arteritis, cranial; Arteritis, giant cell; Arteritis, Takayasu's; Arteritis, temporal; Asthma; Ataxia-telangiectasia; Autoimmune oophoritis; Autoimmune orchitis; Autoimmune polyendocrine failure; Behcet's disease; Berger's disease; Buerger's disease; bronchitis; Bullous pemphigus; Candidiasis, chronic mucocutaneous; Caplan's syndrome; Post-myocardial infarction syndrome; Post-pericardiotomy syndrome; Carditis; Celiac sprue; Chagas's disease; Chediak-Higashi syndrome; Churg-Strauss disease; Cogan's syndrome; Cold agglutinin disease; CREST syndrome; Crohn's disease; Cryoglobulinemia; Cryptogenic fibrosing alveolitis; Dermatitis herpetifomis; Dermatomyositis; Diabetes mellitus; Diamond-Blackfan syndrome; DiGeorge syndrome; Discoid lupus erythematosus; Eosinophilic fasciitis; Episcleritis; Drythema elevatum diutinum; Erythema marginatum; Erythema multiforme; Erythema nodosum; Familial Mediterranean fever; Felty's syndrome; Fibrosis pulmonary; Glomerulonephritis, anaphylactoid; Glomerulonephritis, autoimmune; Glomerulonephritis, post-streptococcal; Glomerulonephritis, post-trans-plantation; Glomerulopathy, membranous; Goodpasture's syndrome; Granulocytopenia, immune-mediated; Granuloma annulare; Granulomatosis, allergic; Granulomatous myositis; Grave's disease; Hashimoto's thyroiditis; Hemolytic disease of the newborn; Hemochromatosis, idiopathic; Henoch-Schoenlein purpura; Hepatitis, chronic active and chronic progressive; Histiocytosis X; Hypereosinophilic syndrome; Idiopathic thrombocytopenic purpura; Job's syndrome; Juvenile dermatomyositis; Juvenile rheumatoid arthritis (Juvenile chronic arthritis); Kawasaki's disease; Keratitis; Keratoconjunctivitis sicca; Landry-Guillain-Barre-Strohl syndrome; Leprosy, lepromatous; Loeffler's syndrome; lupus; Lyell's syndrome; Lyme disease; Lymphomatoid granulomatosis; Mastocytosis, systemic; Mixed connective tissue disease; Mononeuritis multiplex; Muckle-Wells syndrome; Mucocutaneous lymph node syndrome; Mucocutaneous lymph node syndrome; Multicentric reticulohistiocytosis; Multiple sclerosis; Myasthenia gravis; Mycosis fungoides; Necrotizing vasculitis, systemic; Nephrotic syndrome; Overlap syndrome; Panniculitis; Paroxysmal cold hemoglobinuria; Paroxysmal nocturnal hemoglobinuria; Pemphigoid; Pemphigus; Pemphigus erythema-tosus; Pemphigus foliaceus; Pemphigus vulgaris; Pigeon breeder's disease; Pneumonitis, hypersensitivity; Polyarteritis nodosa; Polymyalgia rheumatic; Polymyositis; Polyneuritis, idiopathic; Portuguese familial polyneuropathies; Pre-eclampsia/eclampsia; Primary biliary cirrhosis; Progressive systemic sclerosis (Scleroderma); Psoriasis; Psoriatic arthritis; Pulmonary alveolar proteinosis; Pulmonary fibrosis, Raynaud's phenomenon/syndrome; Reidel's thyroiditis; Reiter's syndrome, Relapsing polychrondritis; Rheumatic fever; Rheumatoid arthritis; Sarcoidosis; Scleritis; Sclerosing cholangitis; Serum sickness; Sezary syndrome; Sjogren's syndrome; Stevens-Johnson syndrome; Still's disease; Subacute sclerosing panencephalitis; Sympathetic ophthalmia; Systemic lupus erythematosus; Transplant rejection; Ulcerative colitis; Undifferentiated connective tissue disease; Urticaria, chronic; Urticaria, cold; Uveitis; Vitiligo; Weber-Christian disease; Wegener's granulomatosis and Wiskott-Aldrich syndrome.

D. Anti-Inflammatory Therapy

In yet other embodiments, the polypeptides of the present invention may be used to treat inflammatory disorders that are caused, at least in part, or exacerbated by inflammation, e.g., increased blood flow, edema, activation of immune cells (e.g., proliferation, cytokine production, or enhanced phagocytosis). Exemplary inflammatory disorders include those in which inflammation or inflammatory factors (e.g., matrix metalloproteinases (MMPs), nitric oxide (NO), TNF, interleukins, plasma proteins, cellular defense systems, cytokines, lipid metabolites, proteases, toxic radicals, mitochondria, apoptosis, adhesion molecules, etc.) are involved or are present in an area in aberrant amounts, e.g., in amounts which may be advantageous to alter, e.g., to benefit the subject. The inflammatory process is the response of living tissue to damage. The cause of inflammation may be due to physical damage, chemical substances, micro-organisms, tissue necrosis, cancer or other agents. Acute inflammation is short-lasting, lasting only a few days. If it is longer lasting however, then it may be referred to as chronic inflammation.

Inflammatory disorders include acute inflammatory disorders, chronic inflammatory disorders, and recurrent inflammatory disorders. Acute inflammatory disorders are generally of relatively short duration, and last for from about a few minutes to about one to two days, although they may last several weeks. The main characteristics of acute inflammatory disorders include increased blood flow, exudation of fluid and plasma proteins (edema) and emigration of leukocytes, such as neutrophils. Chronic inflammatory disorders, generally, are of longer duration, e.g., weeks to months to years or even longer, and are associated histologically with the presence of lymphocytes and macrophages and with proliferation of blood vessels and connective tissue. Recurrent inflammatory disorders include disorders which recur after a period of time or which have periodic episodes. Examples of recurrent inflammatory disorders include asthma and multiple sclerosis. Some disorders may fall within one or more categories.

Inflammatory disorders are generally characterized by heat, redness, swelling, pain and loss of function. Examples of causes of inflammatory disorders include, but are not limited to, microbial infections (e.g., bacterial, viral and fungal infections), physical agents (e.g., burns, radiation, and trauma), chemical agents (e.g. toxins and caustic substances), tissue necrosis and various types of immunologic reactions. Examples of inflammatory disorders include, but are not limited to, osteoarthritis, rheumatoid arthritis, acute and chronic infections (bacterial, viral and fungal); acute and chronic bronchitis, sinusitis, and other respiratory infections, including the common cold; acute and chronic gastroenteritis and colitis; acute and chronic cystitis and urethritis; acute respiratory distress syndrome; cystic fibrosis; acute and chronic dermatitis; acute and chronic conjunctivitis; acute and chronic serositis (pericarditis, peritonitis, synovitis, pleuritis and tendinitis); uremic pericarditis; acute and chronic cholecystis; acute and chronic vaginitis; acute and chronic uveitis; drug reactions; and burns (thermal, chemical, and electrical).

E. Neurological Disorders

In yet other embodiments, a binding polypeptide of the invention is useful in treating a neurological disease or disorder. For example, as set forth above, a binding polypeptide may bind to an antigen present on a neural cell (e.g., a neuron or a glial cell). In certain embodiments, the antigen associated with a neurological disorder may be an autoimmune or inflammatory disorder described supra. As used herein, the term “neurological disease or disorder” includes disorders or conditions in a subject wherein the nervous system either degenerates (e.g., neurodegenerative disorders, as well as disorders where the nervous system fails to develop properly or fails to regenerate following injury, e.g., spinal cord injury. Examples of neurological disorders that can be diagnosed, prevented or treated by the methods and compositions of the present invention include, but are not limited to, Multiple Sclerosis, Huntington's Disease, Alzheimer's Disease, Parkinson's Disease, neuropathic pain, traumatic brain injury, Guillain-Barré syndrome and chronic inflammatory demyelinating polyneuropathy (CIDP), cerebrovascular disease, and encephalitis.

### VIII. METHODS OF ADMINISTERING POLYPEPTIDES OF THE INVENTION

Methods of preparing and administering polypeptides of the invention to a subject are well known to or are readily determined by those skilled in the art. The route of administration of the polypeptides of the invention may be oral, parenteral, by inhalation or topical. The term parenteral as used herein includes intravenous, intraarterial, intraperitoneal, intramuscular, subcutaneous, rectal or vaginal administration. The intravenous, intraarterial, subcutaneous and intramuscular forms of parenteral administration are generally preferred. While all these forms of administration are clearly contemplated as being within the scope of the invention, a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc. However, in other methods compatible with the teachings herein, the polypeptides can be delivered directly to the site of the adverse cellular population thereby increasing the exposure of the diseased tissue to the therapeutic agent.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a binding polypeptide by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in co-pending U.S. Ser. No. 09/259,337 and U.S. Ser. No. 09/259,338 each of which is incorporated herein by reference. Such articles of manufacture will preferably have labels or package inserts indicating that the associated compositions are useful for treating a subject suffering from, or predisposed to autoimmune or neoplastic disorders.

Effective doses of the compositions of the present invention, for the treatment of the above described conditions vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages may be titrated using routine methods known to those of skill in the art to optimize safety and efficacy.

For passive immunization with a binding polypeptide, the dosage can range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example dosages can be 1 mg/kg body weight or 10 mg/kg body weight or within the range of 1-10 mg/kg, preferably at least 1 mg/kg. Doses intermediate in the above ranges are also intended to be within the scope of the invention. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two or more binding polypeptides with different binding specificities are administered simultaneously, in which case the dosage of each binding polypeptide administered falls within the ranges indicated.

Polypeptides of the invention can be administered on multiple occasions. Intervals between single dosages can be weekly, monthly or yearly. Intervals can also be irregular as indicated by measuring blood levels of modified binding polypeptide or antigen in the patient. In some methods, dosage is adjusted to achieve a plasma modified binding polypeptide concentration of 1-1000 μg/ml and in some methods 25-300 μg/ml. Alternatively, polypeptides can be administered as a sustained release formulation, in which case less frequent administration is required. Dosage and frequency vary depending on the half-life of the polypeptide in the patient.

The dosage and frequency of administration can vary depending on whether the treatment is prophylactic or therapeutic. In prophylactic applications, compositions containing the polypeptides of the invention or a cocktail thereof are administered to a patient not already in the disease state to enhance the patient's resistance. Such an amount is defined to be a “prophylactic effective dose.” In this use, the precise amounts again depend upon the patient's state of health and general immunity, but generally range from 0.1 to 25 mg per dose, especially 0.5 to 2.5 mg per dose. A relatively low dosage is administered at relatively infrequent intervals over a long period of time. Some patients continue to receive treatment for the rest of their lives.

In therapeutic applications, a relatively high dosage (e.g., from about 1 to 400 mg/kg of binding polypeptide per dose, with dosages of from 5 to 25 mg being more commonly used for radioimmunoconjugates and higher doses for cytotoxin-drug modified binding polypeptides) at relatively short intervals is sometimes required until progression of the disease is reduced or terminated, and preferably until the patient shows partial or complete amelioration of symptoms of disease. Thereafter, the patient can be administered a prophylactic regime.

Polypeptides of the invention can optionally be administered in combination with other agents that are effective in treating the disorder or condition in need of treatment (e.g., prophylactic or therapeutic).

Effective single treatment dosages (i.e., therapeutically effective amounts) of ⁹⁰Y-labeled modified binding polypeptides of the invention range from between about 5 and about 75 mCi, more preferably between about 10 and about 40 mCi. Effective single treatment non-marrow ablative dosages of ¹³¹I-modified antibodies range from between about 5 and about 70 mCi, more preferably between about 5 and about 40 mCi. Effective single treatment ablative dosages (i.e., may require autologous bone marrow transplantation) of ¹³¹I-labeled antibodies range from between about 30 and about 600 mCi, more preferably between about 50 and less than about 500 mCi. In conjunction with a chimeric antibody, owing to the longer circulating half life vis-á-vis murine antibodies, an effective single treatment non-marrow ablative dosages of iodine-131 labeled chimeric antibodies range from between about 5 and about 40 mCi, more preferably less than about 30 mCi. Imaging criteria for, e.g., the ¹¹¹In label, are typically less than about 5 mCi.

While the polypeptides of the invention may be administered as described immediately above, it must be emphasized that, in other embodiments, polypeptides may be administered to otherwise healthy patients as a first line therapy. In such embodiments the polypeptides may be administered to patients having normal or average red marrow reserves and/or to patients that have not, and are not, undergoing. As used herein, the administration of polypeptides of the invention in conjunction or combination with an adjunct therapy means the sequential, simultaneous, coextensive, concurrent, concomitant or contemporaneous administration or application of the therapy and the disclosed binding polypeptides. Those skilled in the art will appreciate that the administration or application of the various components of the combined therapeutic regimen may be timed to enhance the overall effectiveness of the treatment. For example, chemotherapeutic or biologic agents could be administered in standard, well known courses of treatment in conjunction with the subject binding molecules. A skilled artisan (e.g. a physician) would be readily be able to discern effective combined therapeutic regimens without undue experimentation based on the selected adjunct therapy and the teachings of the instant specification.

In this regard it will be appreciated that the combination of the polypeptide and the agent may be administered in any order and within any time frame that provides a therapeutic benefit to the patient. That is, the agent and binding polypeptide may be administered in any order or concurrently. In selected embodiments the polypeptides of the present invention will be administered to patients that have previously undergone chemotherapy. In yet other embodiments, the binding polypeptides and the chemotherapeutic treatment will be administered substantially simultaneously or concurrently. For example, the patient may be given the binding polypeptide while undergoing a course of chemotherapy. In preferred embodiments the binding polypeptide will be administered within 1 year of any agent or treatment. In other preferred embodiments the binding polypeptide will be administered within 10, 8, 6, 4, or 2 months of any agent or treatment. In still other preferred embodiments the binding polypeptide will be administered within 4, 3, 2 or 1 week of any agent or treatment. In yet other embodiments the binding polypeptide will be administered within 5, 4, 3, 2 or 1 days of the selected agent or treatment. It will further be appreciated that the two agents or treatments may be administered to the patient within a matter of hours or minutes (i.e. substantially simultaneously).

It will further be appreciated that the polypeptides of the instant invention may be used in conjunction or combination with any agent or agents (e.g. to provide a combined therapeutic regimen) that eliminates, reduces, inhibits or controls the growth of neoplastic cells in vivo. Exemplary chemotherapeutic agents that are compatible with the instant invention include alkylating agents, vinca alkaloids (e.g., vincristine and vinblastine), procarbazine, methotrexate and prednisone. The four-drug combination MOPP (mechlethamine (nitrogen mustard), vincristine (Oncovin), procarbazine and prednisone) is very effective in treating various types of lymphoma and comprises a preferred embodiment of the present invention. In MOPP-resistant patients, ABVD (e.g., adriamycin, bleomycin, vinblastine and dacarbazine), ChlVPP (chlorambucil, vinblastine, procarbazine and prednisone), CABS (lomustine, doxorubicin, bleomycin and streptozotocin), MOPP plus ABVD, MOPP plus ABV (doxorubicin, bleomycin and vinblastine) or BCVPP (carmustine, cyclophosphamide, vinblastine, procarbazine and prednisone) combinations can be used. Arnold S. Freedman and Lee M. Nadler, Malignant Lymphomas, in HARRISON'S PRINCIPLES OF INTERNAL MEDICINE 1774-1788 (Kurt J. Isselbacher et al., eds., 13^{th} ed. 1994) and V. T. DeVita et al., (1997) and the references cited therein for standard dosing and scheduling. These therapies can be used unchanged, or altered as needed for a particular patient, in combination with one or more polypeptides of the invention as described herein.

Additional regimens that are useful in the context of the present invention include use of single alkylating agents such as cyclophosphamide or chlorambucil, or combinations such as CVP (cyclophosphamide, vincristine and prednisone), CHOP (CVP and doxorubicin), C-MOPP (cyclophosphamide, vincristine, prednisone and procarbazine), CAP-BOP(CHOP plus procarbazine and bleomycin), m-BACOD (CHOP plus methotrexate, bleomycin and leucovorin), ProMACE-MOPP (prednisone, methotrexate, doxorubicin, cyclophosphamide, etoposide and leucovorin plus standard MOPP), ProMACE-CytaBOM (prednisone, doxorubicin, cyclophosphamide, etoposide, cytarabine, bleomycin, vincristine, methotrexate and leucovorin) and MACOP-B (methotrexate, doxorubicin, cyclophosphamide, vincristine, fixed dose prednisone, bleomycin and leucovorin). Those skilled in the art will readily be able to determine standard dosages and scheduling for each of these regimens. CHOP has also been combined with bleomycin, methotrexate, procarbazine, nitrogen mustard, cytosine arabinoside and etoposide. Other compatible chemotherapeutic agents include, but are not limited to, 2-chlorodeoxyadenosine (2-CDA), 2′-deoxycoformycin and fludarabine.

For patients with intermediate- and high-grade NHL, who fail to achieve remission or relapse, salvage therapy is used. Salvage therapies employ drugs such as cytosine arabinoside, carboplatin, cisplatin, etoposide and ifosfamide given alone or in combination. In relapsed or aggressive forms of certain neoplastic disorders the following protocols are often used: IMVP-16 (ifosfamide, methotrexate and etoposide), MIME (methyl-gag, ifosfamide, methotrexate and etoposide), DHAP (dexamethasone, high dose cytarabine and cisplatin), ESHAP (etoposide, methylpredisolone, HD cytarabine, cisplatin), CEPP(B) (cyclophosphamide, etoposide, procarbazine, prednisone and bleomycin) and CAMP (lomustine, mitoxantrone, cytarabine and prednisone) each with well known dosing rates and schedules.

In one embodiment, a binding polypeptide of the invention may be administered in combination with a biologic. The term “biologic” or “biologic agent” refers to any pharmaceutically active agent made from living organisms and/or their products which is intended for use as a therapeutic. In one embodiment of the invention, biologic agents which can be used in combination with a binding molecule comprising an scFc include, but are not limited to e.g., antibodies, nucleic acid molecules, e.g., antisense nucleic acid molecules, polypeptides or proteins. Such biologics can be administered in combination with a binding molecule by administration of the biologic agent, e.g., prior to the administration of the binding molecule, concomitantly with the binding molecule, or after the binding molecule.

The amount of agent to be used in combination with the polypeptides of the instant invention may vary by subject or may be administered according to what is known in the art. See for example, Bruce A Chabner et al., Antineoplastic Agents, in GOODMAN & GILMAN's THE PHARMACOLOGICAL BASIS OF THERAPEUTICS 1233-1287 ((Joel G. Hardman et al., eds., 9^{th} ed. 1996). In another embodiment, an amount of such an agent consistent with the standard of care is administered.

As previously discussed, the polypeptides of the present invention, may be administered in a pharmaceutically effective amount for the in vivo treatment of mammalian disorders. In this regard, it will be appreciated that the polypeptides of the invention can be formulated to facilitate administration and promote stability of the active agent. Preferably, pharmaceutical compositions in accordance with the present invention comprise a pharmaceutically acceptable, non-toxic, sterile carrier such as physiological saline, non-toxic buffers, preservatives and the like. For the purposes of the instant application, a pharmaceutically effective amount of a polypeptide of the invention, conjugated or unconjugated to a therapeutic agent, shall be held to mean an amount sufficient to achieve effective binding to an antigen and to achieve a benefit, e.g., to ameliorate symptoms of a disease or disorder or to detect a substance or a cell. In the case of tumor cells, the polypeptide will be preferably be capable of interacting with selected immunoreactive antigens on neoplastic or immunoreactive cells and provide for an increase in the death of those cells. Of course, the pharmaceutical compositions of the present invention may be administered in single or multiple doses to provide for a pharmaceutically effective amount of the polypeptide.

In keeping with the scope of the present disclosure, the polypeptides of the invention may be administered to a human or other animal in accordance with the aforementioned methods of treatment in an amount sufficient to produce a therapeutic or prophylactic effect. A polypeptide of the invention can be administered to such human or other animal in a conventional dosage form prepared by combining the polypeptide with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of polypeptides of the invention may prove to be particularly effective.

This invention is further illustrated by the following examples which should not be construed as limiting. The contents of all references, patents and published patent applications cited throughout this application are incorporated herein by reference.

### EXAMPLES

Throughout the examples, the following materials and methods were used unless otherwise stated.

### General Materials and Methods

In general, the practice of the present invention employs, unless otherwise indicated, conventional techniques of chemistry, biophysics, molecular biology, recombinant DNA technology, immunology (especially, e.g., antibody technology), and standard techniques in electrophoresis. See, e.g., Sambrook, Fritsch and Maniatis, Molecular Cloning Cold Spring Harbor Laboratory Press (1989); Antibody Engineering Protocols (Methods in Molecular Biology), 510, Paul, S., Humana Pr (1996); Antibody Engineering: A Practical Approach (Practical Approach Series, 169), McCafferty, Ed., Irl Pr (1996); Antibodies: A Laboratory Manual, Harlow et al., C.S.H.L. Press, Pub. (1999); and Current Protocols in Molecular Biology, eds. Ausubel et al., John Wiley & Sons (1992).

### Example 1

### Expression and Purification of scFc

A human 5C8 IgG1 antibody comprising a genetically-fused Fc region (i.e., a single-chain (scFc) region) was expressed in DG44 CHO cells according to previously described methods. To affinity purify the recombinantly-expressed single- and double-chain scFc proteins that resulted (see FIG. 1 for schematic), the CHO cell fermentation medium (1 L) was adjusted to pH 7.0 and the protein was affinity captured on a 5 ml HiTrap rProteinA FF column (GE Healthcare) that had been previously equilibrated in binding buffer (100 mM NaPO₄, pH 7, 150 mM NaCl). The column was washed in binding buffer until the A280 trace reached baseline and the bound protein was eluted in 25 mM glycine pH 2.8, 100 mM sodium chloride. Fractions were immediately neutralized by addition of 0.1 volumes 1M Tris buffer, pH 8. Protein in A280 absorbing fractions were analyzed by reducing and non-reducing SDS-PAGE, pooled and concentrated for further purification by size-exclusion chromatography. Single-chain (i.e., monomeric) scFc polypeptides were separated from aggregates and double-chain (i.e., dimeric) scFc polypeptides on a Superdex 200 gel filtration column in PBS, pH7. Gel filtration fractions were analyzed by reducing and non-reducing SDS-PAGE and appropriate fractions were combined to obtain homogeneous pools of the single- and double-chain scFc antibody populations. These pools were further characterized to determine homogeneity and molecular mass of the proteins by analytical SEC (TSK-Gel G3000 SW_{XL} column) with on-line light scattering analysis. Intact mass measurements and mapping of interchain and intrachain disulfide bonds were made by mass spectroscopy on non-reduced, deglycosylated sc- and dc-Fc pools.

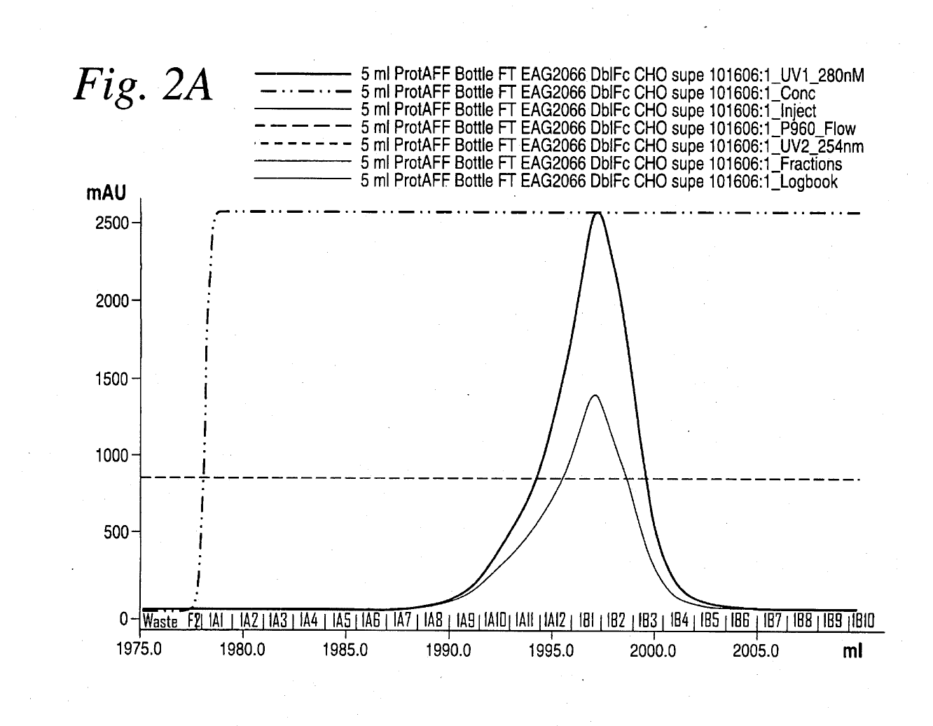

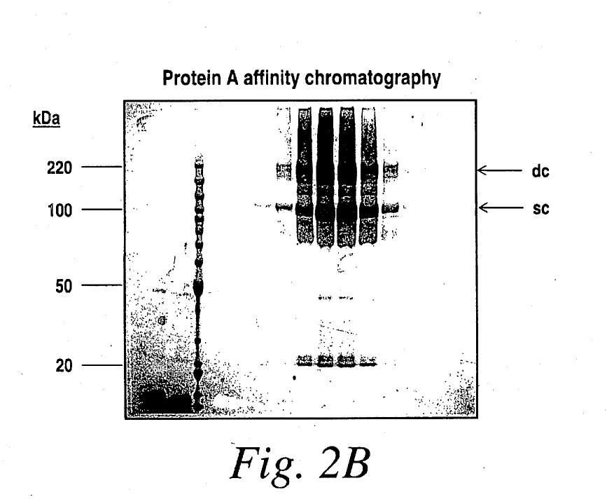

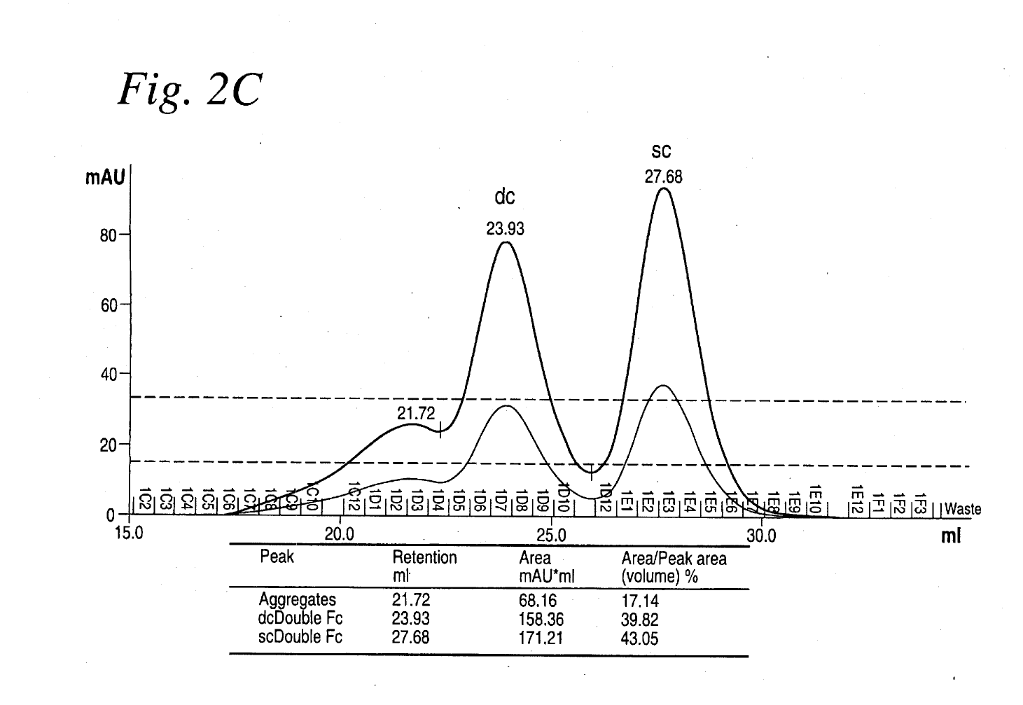

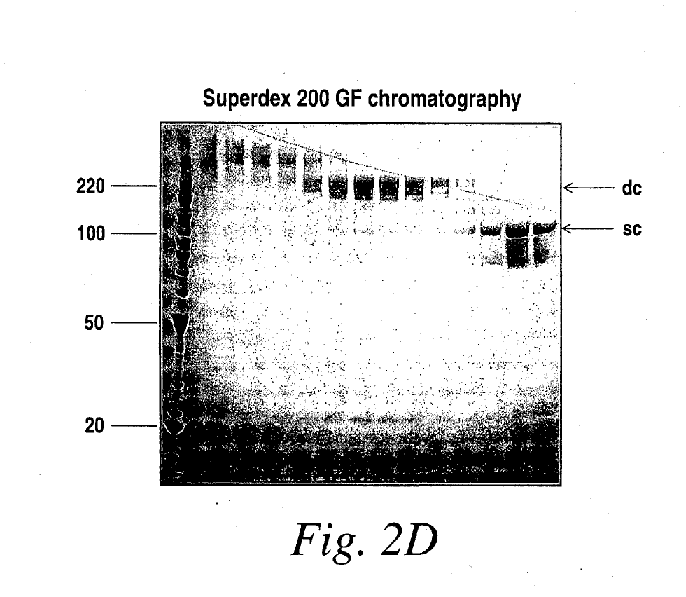

FIGS. 2A-D show the results of the two-step purification process for separating monomeric (“sc”) and dimeric (“dc”) scfc proteins. The purification process employed affinity chromatography followed by gel filtration chromatography. FIG. 2A shows the absorbance profile of column fractions eluted at low pH from the Protein A affinity column. FIG. 2B shows the corresponding SD-PAGE analysis of those eluted fractions which contain both dimeric (“dc”) and monomeric (“sc”) forms of the scFc binding polypeptide. Both the monomeric and dimeric forms eluted essentially as a single peak from the protein A column. FIG. 2C shows that the Superdex 200 gel filtration column elutant can separate this mixture into two distinct peaks. FIG. 2D shows corresponding SDS-PAGE analysis of the gel filtration eluates. The peaks represent the purified monomeric (“sc”) and dimeric (“dc”) forms of the human 5C8 scFc IgG1 antibody, respectively.

FIG. 3 shows an SDS-PAGE of the dimeric (“dc”) and monomeric (“sc”) forms of the scFc binding polypeptide at a preparative scale under non-reducing (Panel A) and reducing (Panel B) conditions. For each panel, Lanes 1 and 2 contain the respective dimeric form (“dc”; 205 kDa) and monomeric (“sc”; 105 kDa) form respectively. Lane 3 contains the control human 5C8 IgG1 antibody (5C8; 150 kDa).

### Example 2

### Assays for Determining Functional Interaction of Monomeric and Dimeric scFc Antibodies

(a) shCD40L Binding Assays

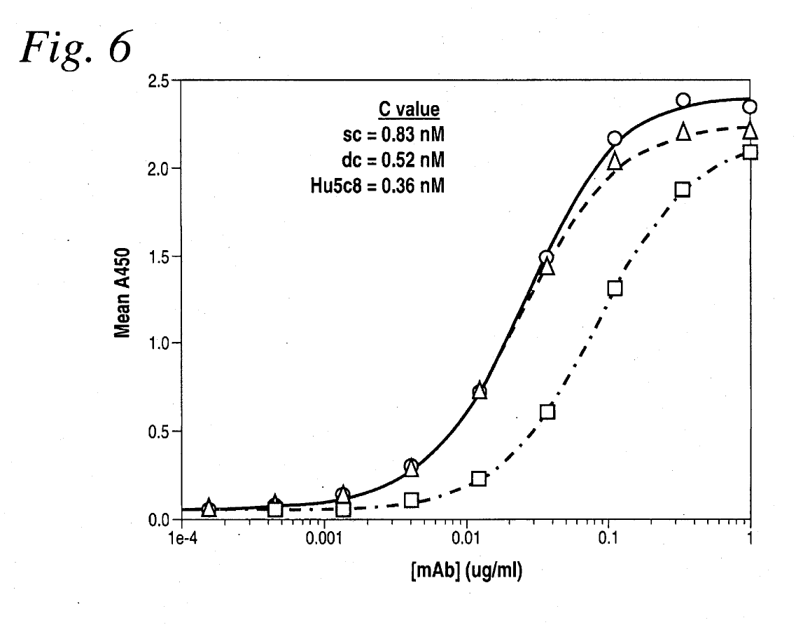

To detect direct antigen binding of monomeric (“sc”) and dimeric (“dc”) scFc antibodies, soluble human CD40L (CD154) was coated on Nunc MaxiSorp 96-well plates at 2 μg/ml in PBS, pH7, ON at 4° C., 100 μL per well. The IgG solution was shaken out of the plates and the wells were blocked for 2 hr at room temperature in blocking buffer (300 μL per well) containing 10 mM NaPi, 0.362M NaCl, 0.05% Tween-20, 0.1% Casein, 5% FBS, pH7. The plates were emptied and biotinylated WT 5c8 hIgG1, sc or dc scFcs were titrated in from 1 μg/ml diluted 1:3 across the plate in blocking buffer 100 μL per well. After incubation for 2 hr at room temperature, the plates were washed four times in PBS, 0.05% Tween-20. Horse-radish peroxidase conjugated streptavidin was diluted 1:10,000 in blocking buffer and 100 μL per well, was added to the plates for 1 hr at room temperature to detect the bound biotinylated scFcs. The plates were washed, and color development was allowed to proceed for approximately 5 min. in the presence of the substrate tetramethyl benzidine (TMB, 100 μL per well). The reaction was quenched by the addition of 0.5M H₂SO₄, 100 μL per well, and absorbance at 450 nm was read. FIG. 6 shows the results of an ELISA binding assay comparing the antigen binding affinity of the monomeric (“sc”) scFc antibody, the dimeric (“dc”) scFc antibody, and a conventional IgG1 antibody (Hu 5C8).

Biacore analysis was also performed to determine the affinities and the off-rates measured by Biacore for the binding of a 5c8 IgG1 bivalent mAb, monovalent Fab and monovalent 3×G4S-linked hemiglycosylated scFc to the antigen CD40L (see Table 2). The affinity of the scFc for its target antigen was found to be weaker than the mAb but comparable to that of the Fab.

TABLE 2
Biacore Analysis

| Molecule | KD (pM) | kd (×10-4. s-1) |
|---|---|---|
| 5C8 mAb | <46 | 0.8 |
| 5C8 F(ab) | 560 | 4.5 |
| 5C8 scFc | 200 | 3.6 |

(b) FcRn Binding ELISA

To detect direct binding to human and rat FcRn, wild-type (WT) 5C8 hIgG1 and monomeric (“sc”) and dimeric (“dc”) scFc antibodies were coated on Nunc MaxiSorp 96-well plates at 5 μg/ml in PBS, pH6, ON at 4° C. The IgG solution was shaken out of the plates and the wells were blocked for 2 hr at room temperature in blocking buffer containing 0.1 M sodium phosphate, 0.1 M sodium chloride, 0.05% Tween20 and 0.1% gelatin, pH 6. The plates were washed in PBS pH 6 and biotinylated human or rat FcRn-Fc in blocking buffer was titrated in at a starting concentration of 1 μg/ml and diluted serially 1:3 down the plate. After incubation for 2 hr at room temperature, the plates were washed in PBS, pH 6, and horse-radish peroxidase conjugated streptavidin diluted 1:10,000 in blocking buffer was added to the wells for 1.5 hr at room temperature to detect bound biotinylated FcRn-Fc. The plates were washed, and color development was allowed to proceed for approximately 15 min. in the presence of the substrate tetramethyl benzidine (TMB). The reaction was quenched by the addition of 0.5M H₂SO₄ and the plates were read at 450 nm.

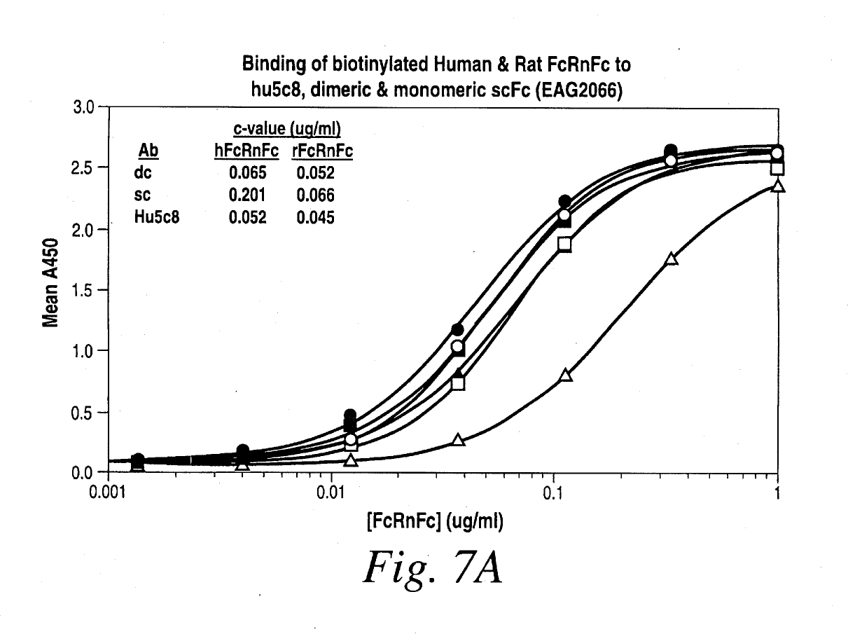

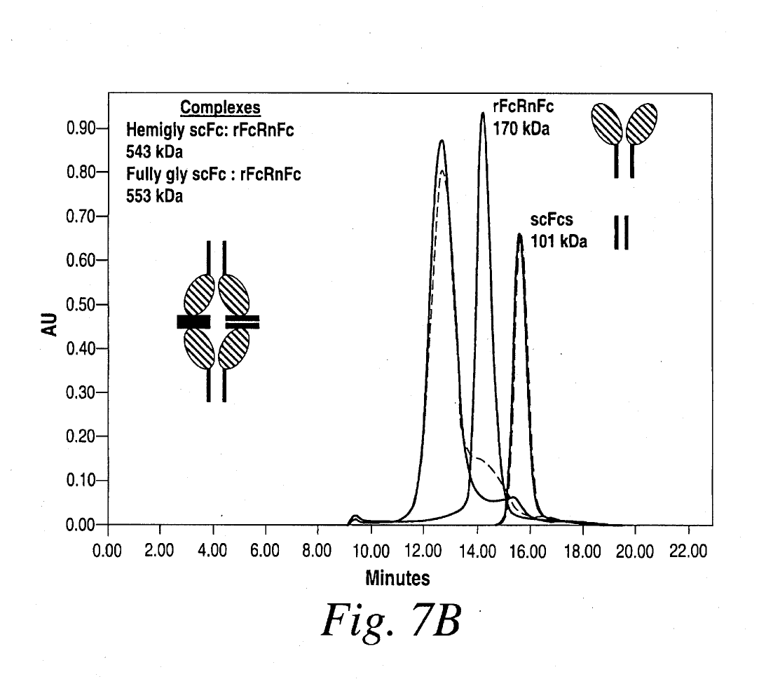

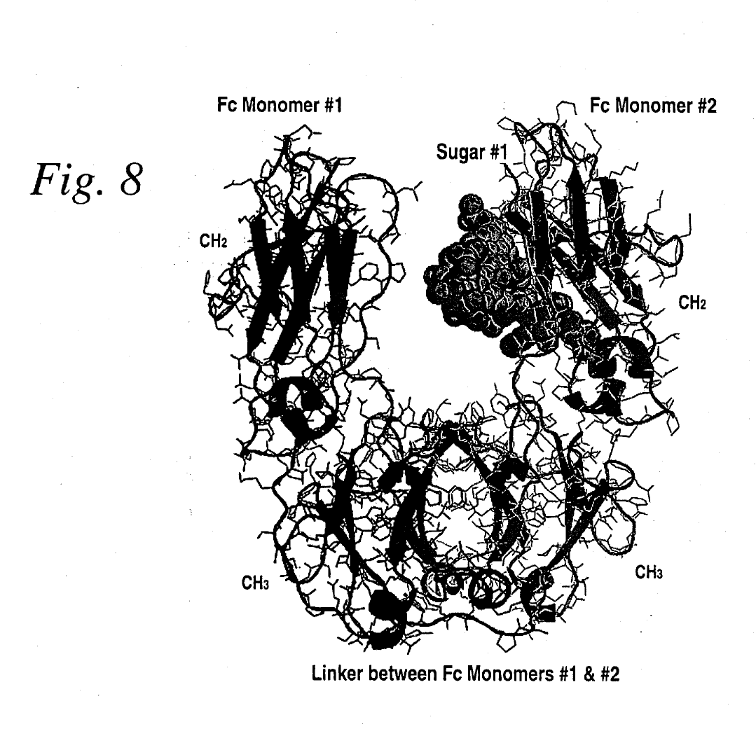

FIG. 7 shows the results of an FcRn binding assay comparing the FcRn binding affinity of the dimeric and monomeric forms of the scFc binding polyeptide, with that of the conventional IgG1 antibody (Hu 5C8). FcRn binding was determined using biotinylated forms of both a human and a rat FcRn-Fc construct. In this assay each Fc containing construct was coated on the plate and binding of a biotinylated rat or human FcRn-Fc construct was detected with streptavidin HRP. The determined c-value for binding of the monomeric scFc to human FcRn (but not to rat FcRn) was approximately four-fold lower than that of the Hu5C8 or the dimeric scFc antibody.

(c) FcγR Binding Assays

Binding of the WT 5C8 hIgG1 antibody and the monomeric (“sc”) and dimeric (“dc”) scFc antibodies to FcγRI (CD64) was measured in a cell-based bridging assay that has been previously described (Ferrant J L et al., International Immunology, 2004). The FcγRI (CD64) bridging assays were performed on 96-well Maxisorb ELISA plates (Nalge-Nunc, Rochester, N.Y., USA) coated with human recombinant soluble CD40L (CD154) at 10 μg/ml to capture the test antibodies. Test antibodies were titrated into the wells starting at 1 μg/ml and serially diluted 1:3 down the plate. Antibody-dependent binding of fluorescently labeled CD64⁺CD32⁺U937 cells (ATCC) was measured at ex. 485 nm/em. 530 nm.

The ability of scFc antibodies comprising GGGGS (“1×-G4S”) or (GGGS)3 (“3×-G4S”) linkers to engage Fcγ receptors was also evaluated in an Amplified Luminescent Proximity Homogeneous Assay (Alphascreen®; Perkin Elmer). Laser excitation (680 nm) of a donor bead generates singlet oxygen which, if in close proximity to an acceptor bead, initiates a cascase of events ultimately leading to fluorescence emission at 520-620 nm. Donor and acceptor beads decorated with ligand and receptor proteins, respectively, are brought into desired proximity only when the receptor and ligand become functionally engaged.

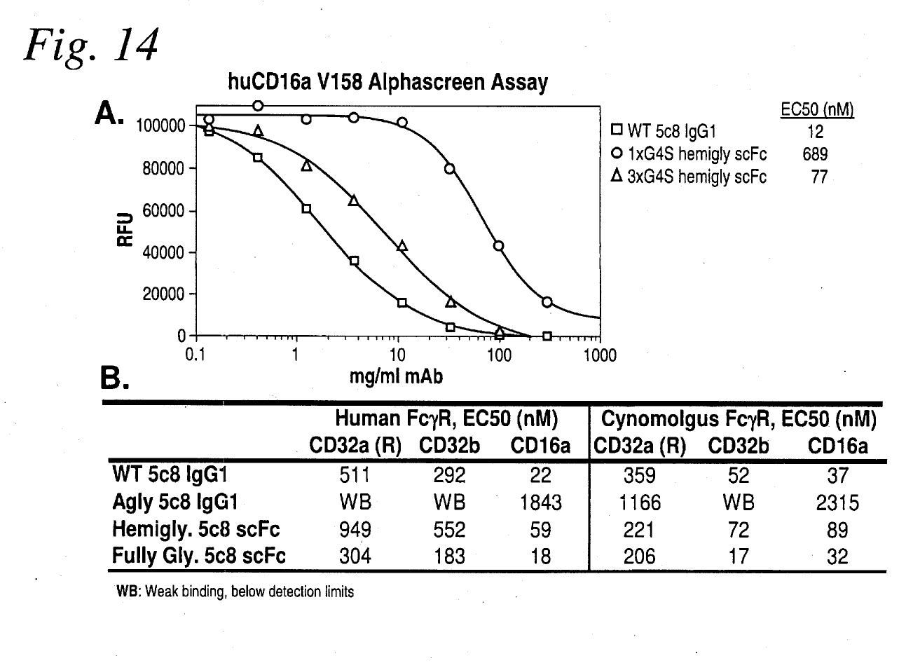

The Alphascreen® assay was performed in a competitive format in which serial dilutions of test antibodies (WT IgG1 or scFc) were incubated with human FcRIII-GST (CD16a, V158) and anti-GST acceptor beads overnight at 4° C. in a 96-well white plate. Streptavidin donor beads and biotinylated wild-type IgG1 were also incubated overnight at 4° C. in a separate tube and then added to the assay plate the next day. After a two-hour incubation at room temperature with gentle shaking the plates were read in an Envision® plate reader (Perkin Elmer). Compared to the fully glycosylated WT human IgG1, the hemi-glycosylated 3×- and 1×-G4S linked scFcs were shown to have approximately 4-fold and 57-fold lower affinity for the high affinity variant (V158) of the low affinity human FcγRIII, respectively (see FIG. 14A).

An Alphascreen assay was performed to evaluate FcγR-binding activity of hemiglycosylated or fully glycosylated scFc polypeptides relative to that of wild-type human IgG1 antibody (5c8) and an engineered a glycosylated variant of the human IgG1 (“Agly 5c8 scFc”) which is expected to exhibit significantly attenuated binding to the Fcγ receptors. Binding to human and cynomolgus FcγR IIa (CD32a), IIb (CD32b) and III (CD16a) was determined in this assay. Both the hemiglycosylated as well as the fully glycosylated 3×G4S linked scFc variants bound to the receptors with apparent affinities comparable to WT IgG1 whereas binding of the 1×G4S linked scFc to FcγRIII was approximately 60-fold weaker than that measured for WT IgG1 (see FIG. 14B).

(d) SEC-LS of scFc Antibody Complexes with Human CD40L

Non-equilibrium analytical gel filtration with on-line light scattering experiments were set up in order to determine the size and stoichiometry of the complexes formed when single-chain (“sc”; i.e., monomeric) or double-chain (“dc”; i.e., dimeric) scFc antibodies and the trimeric ligand, CD40L were mixed at various molar ratios. The WT human IgG1 anti-human CD40L mAb (5C8) was used as a control molecule in these studies. Each scFc antibody was mixed with ligand to obtain near equimolar ratios of binding sites (1:1) as well as at ratios where the trimeric ligand was present in 3-fold excess (1:3). The proteins were mixed, made up to the final volume in PBS and allowed to equilibrate for ≧4 hr. Soluble human CD40L concentration was held constant at 3 μM for the mixes representing equimolar binding sites, and added at 6 μM into mixes representing 3-fold excess of ligand binding sites. Complexes were injected onto a TSK-GEL G4000 SW_{XL} column (7.8 mm-x 30 cm, Tosoh) in a 40 μl volume at 0.6 ml/min. The HPLC system (Waters 2690) used for these studies was outfitted with on-line UV, light scattering (PD2000 DLS, Precision Detectors) and R1 detectors so that the molecular weight and stoichiometry of the complexes could be readily determined.

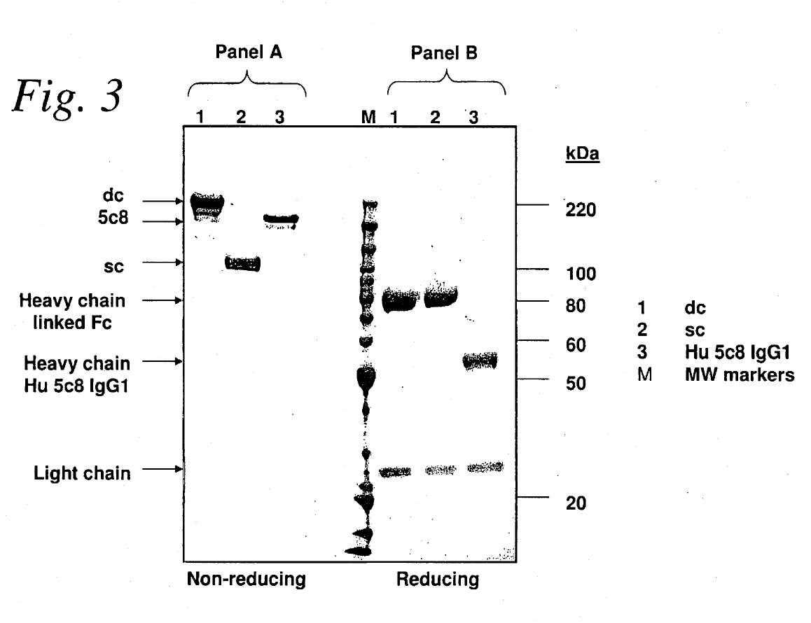

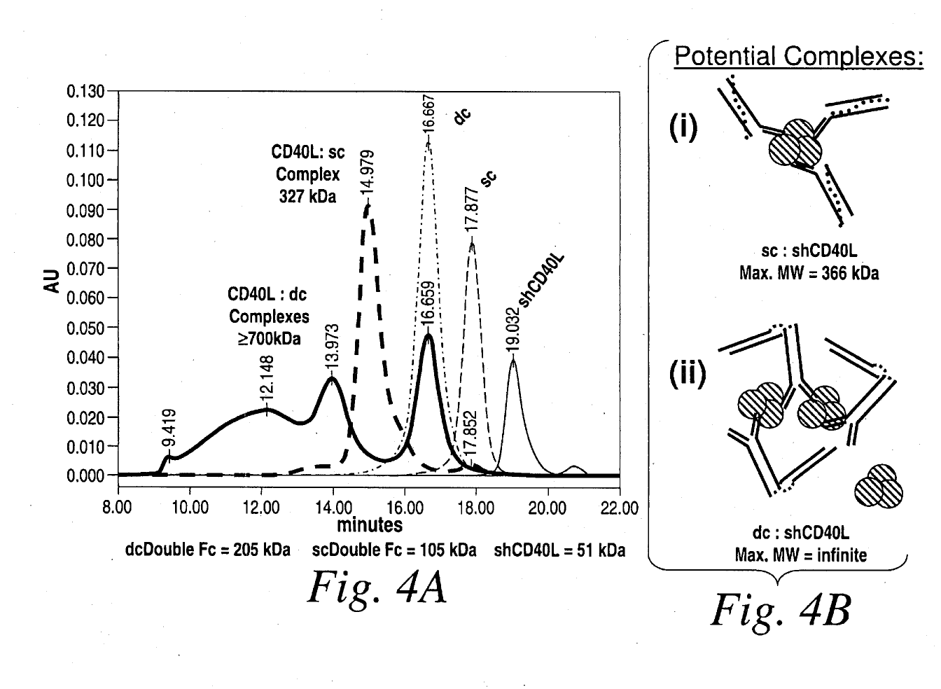

FIGS. 4A-B show the characterization of complexes of single-chain (“sc”) or double-chain (“dc”) scFc antibodies bound to the homotrimeric shCD40L antigen.

FIG. 4A shows a composite of the size exclusion chromatograms obtained for the SEC-LS experiments that were performed in order to determine the molecular weight of each complex. FIG. 4B shows a schematic of the predicted complexes formed upon binding of the (i) single-chain (“sc”) and (ii) double-chain (“dc”) scFc antibodies to shCD40L, respectively, as well as the theoretical molecular weight of those complexes.

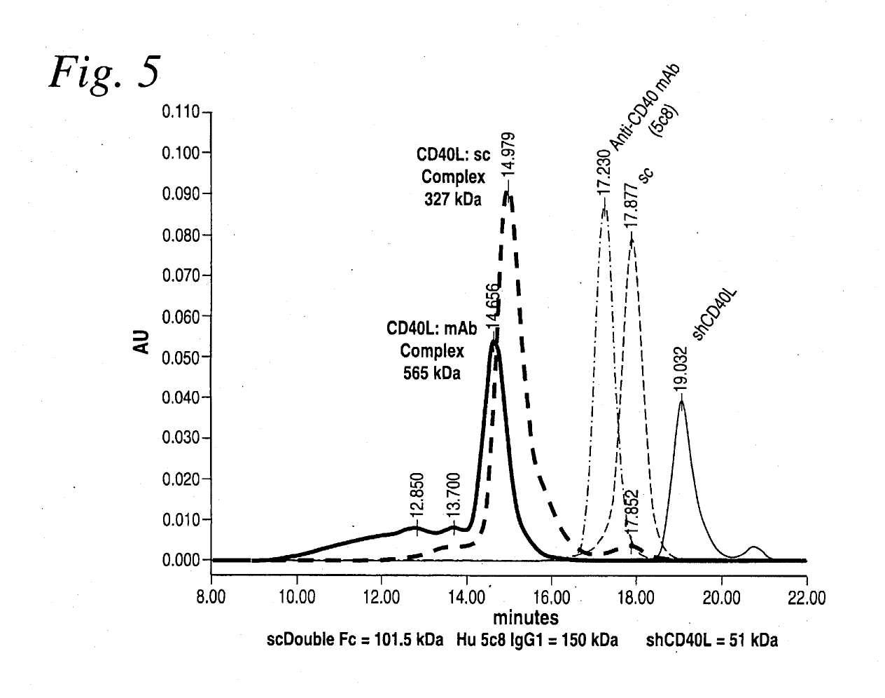

FIG. 5 shows a comparison of the shCD40L-containing complexes formed in the presence of either the single-chain (“sc”) scFc antibody or the conventional human IgG1 anti-CD40L mAb (5C8). The determined molecular weight of each complex is denoted above each peak. The predicted molecular weights of the single-chain (“sc”) scFc, 5C8 and shCD40L, are 105 kDa, 150 kDa and 51 kDa, respectively.

### Example 3

### Enhanced Expression of scFc Polypeptides with Specific Polypeptide Linkers

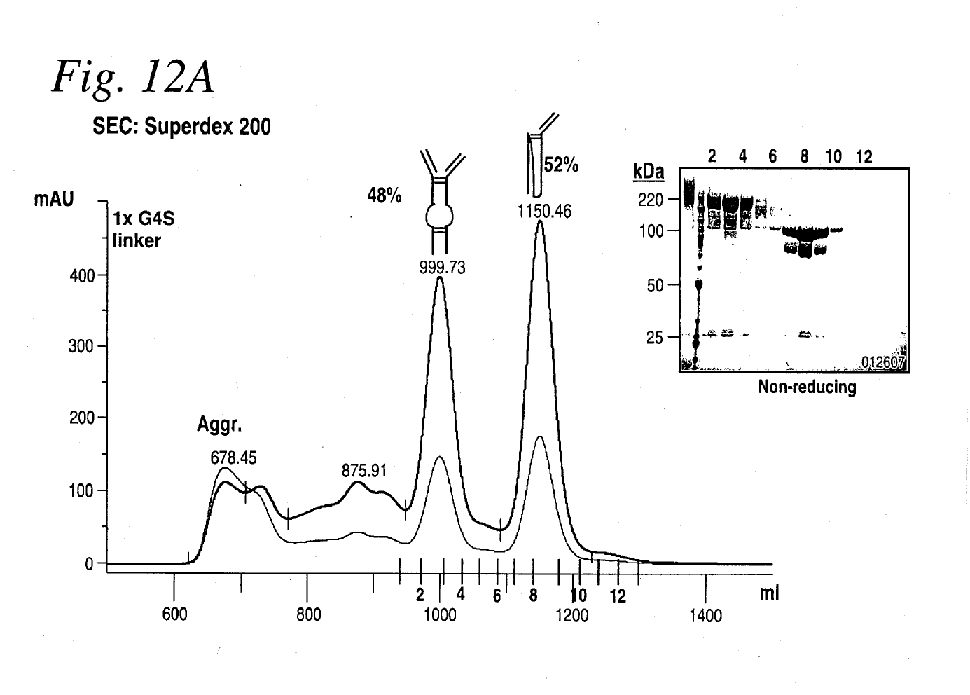

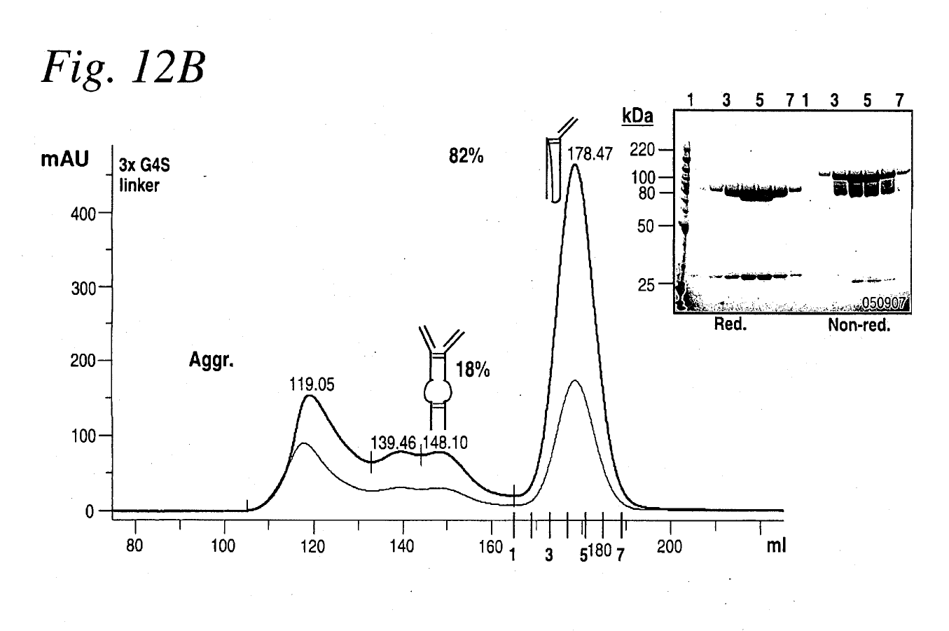

The use of specific polypeptide linkers can be used to select for preferential expression of either of the single- (i.e., monomeric) or double-chain (i.e., dimeric) scFc constructs. FIG. 12 shows the characterization Protein-A affinity purified scFc constructs containing either a 1×G4S or a 3×G4S linker interposed between the constituent Fc moieties of their scFc region. Preparative scale size exclusion chromatography of the proteinA pools obtained for the 1×G4S (FIG. 12A) and 3×G4S (FIG. 12B) scFc show a clear correlation between increased linker length and percent protein expressed as a monomeric (“sc”) vs. dimeric (“dc”) scFc. The sc- & dc-scFc populations contained within the eluted material were analyzed by SDS-PAGE of the indicated fractions. An overlay of the analytical size exclusion chromatography traces obtained for ProteinA affinity purified scFc constructs comprising either a 1×G4S or a 3×G4S linker also shows that the 1×G4S linked scFc construct comprises a mixed population with appreciable amounts of dimeric scFc polypeptide (“dc”) in addition to the desired monomeric scFc (“sc”), whereas the 3×G4S linker construct is significantly enriched for the monomeric scFc population (FIGS. 12C i & ii). Analytical SEC-LS analysis of the 1× and 3×G4S linked scFc proteins obtained following the 2-step purification protocol shows that both scFc proteins can be prepared to homogeneity and the preparations contain material of the expected molecular mass (100 kDa). Thus, scFc polypeptides comprising Fc moieties genetically-fused by the 1×G4S linker comprise a mixed population of molecules with appreciable amounts of double-chain (“dc”) scFc polypeptide in addition to the desired single-chain (“sc”) scFc polypeptide. In contrast, the 3×-G4S linked scFc construct is significantly enriched for the single-chain (“sc”) scFc polypeptide population.

### Example 4

### Determination of the Pharmacokinetic (PK) Characteristics of scFc Polypeptides

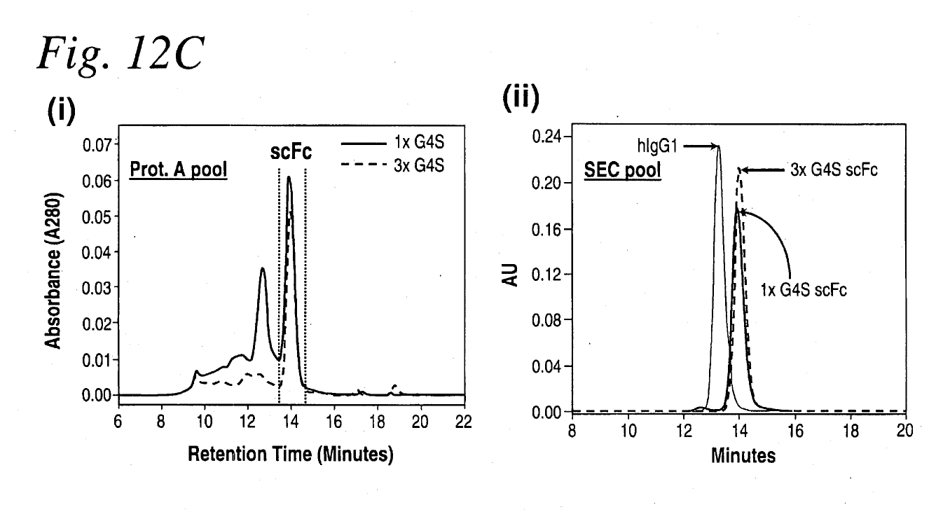

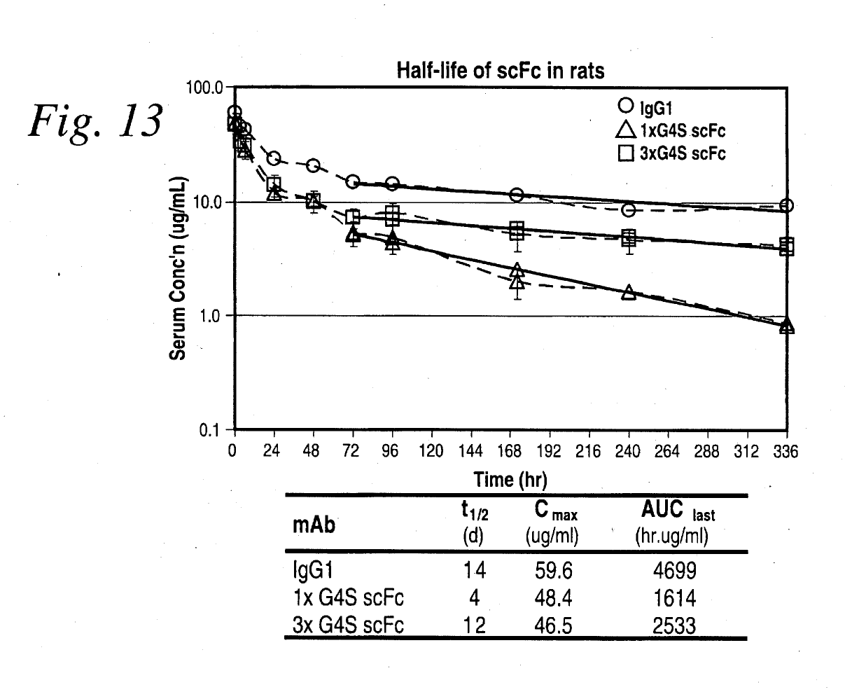

The terminal half-life of a WT human IgG1 (5C8) and the scFc constructs containing the 1×- and 3×-G4S linkers was determined in rats. Each construct was administered to three animals intravenously at 2.5 mg/kg in PBS pH 7. Serum samples were collected at predetermined timepoints and stored frozen at −80° C. for analysis. A time course for the serum concentrations of each construct was determined by ELISA. Briefly, recombinant, soluble human CD40L (CD154) was coated at 5 μg/ml in PBS pH7 on Nunc MaxiSorp® 96-well plates at ON at 4° C. Plates were blocked with blocking buffer (1% Casein Hydrolysate in 10 mM PBS pH7, 300 μl/well for 2 h at RT). Serial dilutions of serum samples obtained at various time points were prepared in 10 mM PBS pH7, 0.362M NaCl, 0.05% Tween-20, 0.1% Casein, 5% FBS and applied to the appropriate wells at RT for 2 hr. The plates were washed 4× in PBS with 0.05% Tween-20 and the captured IgG and scFc polypeptides were detected at 450 nm following incubation for 2 hr with a horseradish peroxidase conjugated donkey anti-human IgG secondary antibody (Jackson Immunoresearch 709-035-149). The time-dependent change in serum concentrations of the WT and scFc polypeptides was analysed using the WinNonLin® software package. The WT human IgG1 and the 3×-G4S linked scFc polypeptide exhibited similar beta-phase half-life of 14 days and 12 days, respectively. The half-life of the 1×-G4S linked scFc was 4.2 days which is—significantly shorter than the WT mAb (see FIG. 13)

### Example 5

### Preparation of Hemiglycosylated scFc Polypeptides

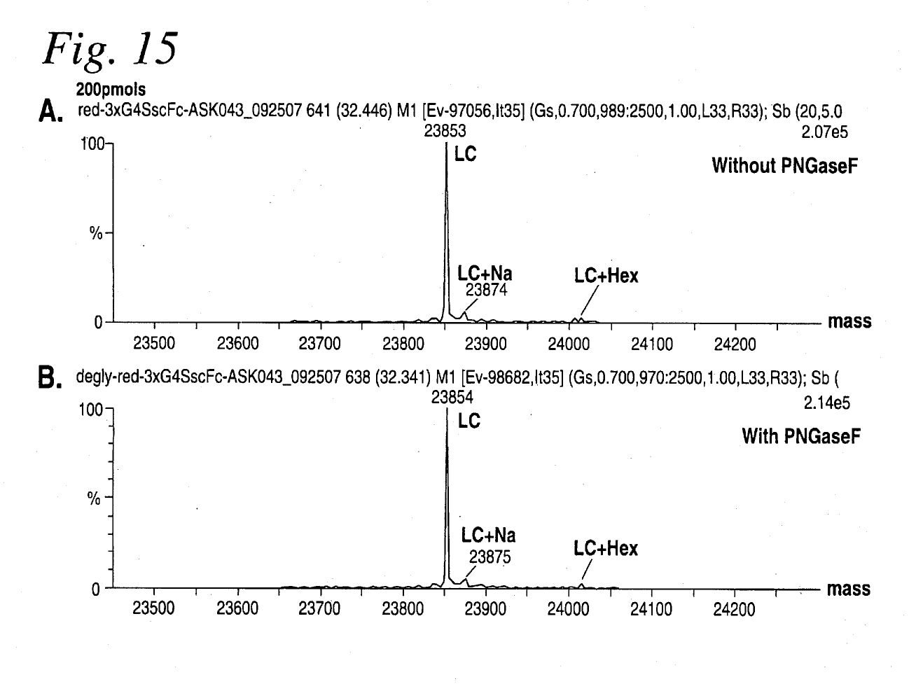

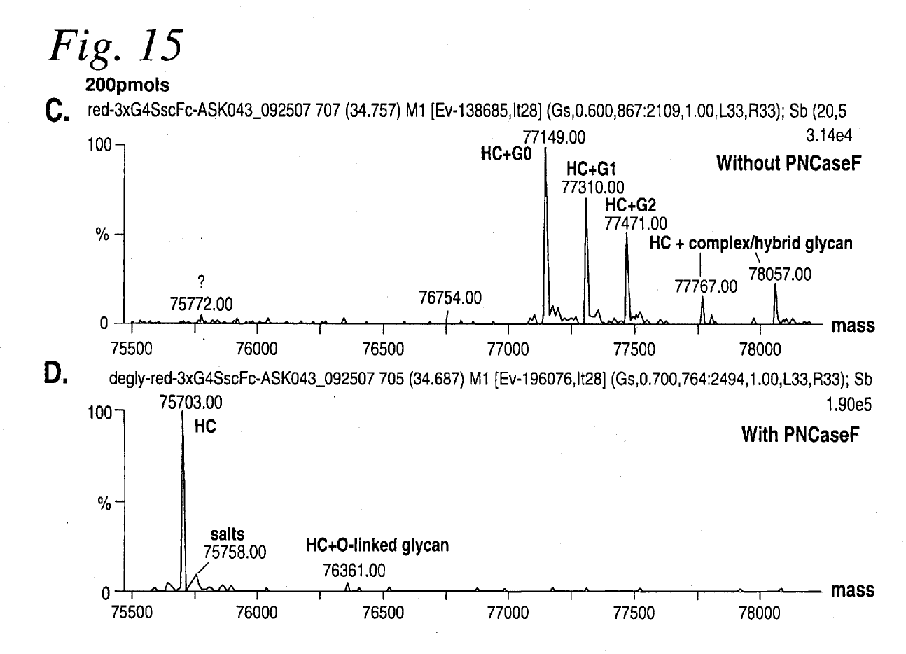

An exemplary scFc polypeptide of the invention is a 3×G4S-linked hemiglycosylated 5c8 scFc polypeptide having a single glycan in one of two Fc moieties of its scFc region. To confirm that hemiglycosylation occurred, the scFc polypeptide was subjected to deconvoluted mass spectrometry (MS) both prior to and following enzymatic removal of the hemiglycan. FIG. 15 shows the deconvoluted mass spectra obtained pre- and post PNGaseF deglycosylation of the protein. The masses determined for the deglycosylated (and reduced) heavy and light chains of the scFc polypeptide are 75,703 Da and 23,854 Da, respectively. The intact, deglycosylated molecule should therefore have a mass of 99,557 Da which is in good agreement with the calculated mass of 100 kDa. Accordingly, the molecular weight of the deglycosylated scFc polypeptide is consistent with hemiglycosylation.

### Example 6

### Biophysical Analysis of scFc Polypeptides

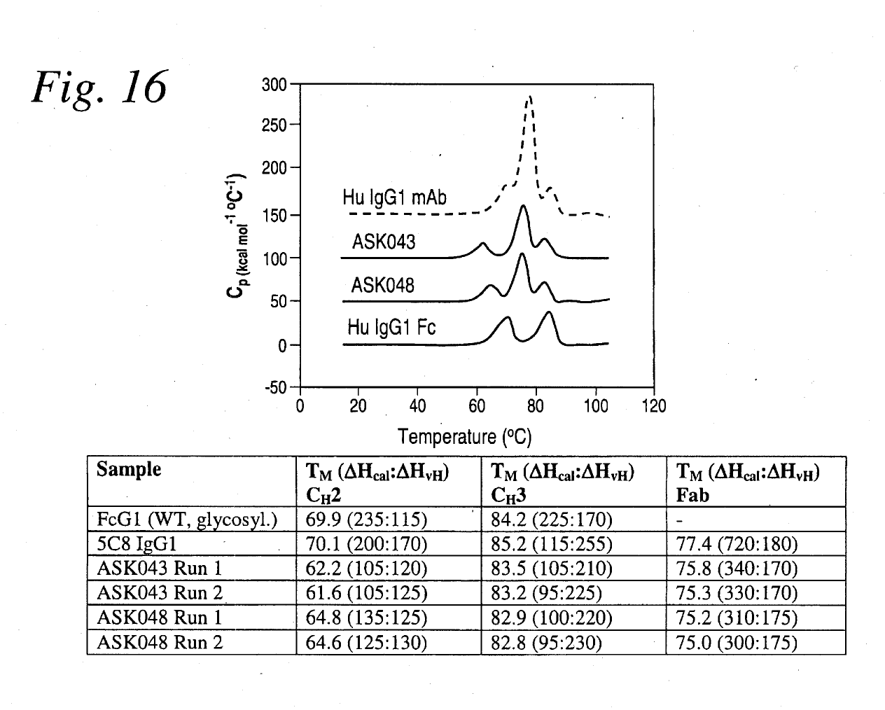

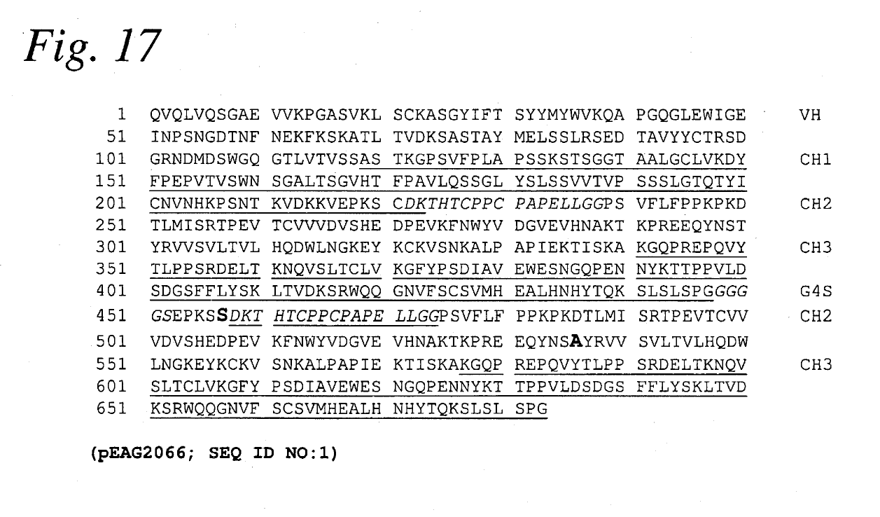

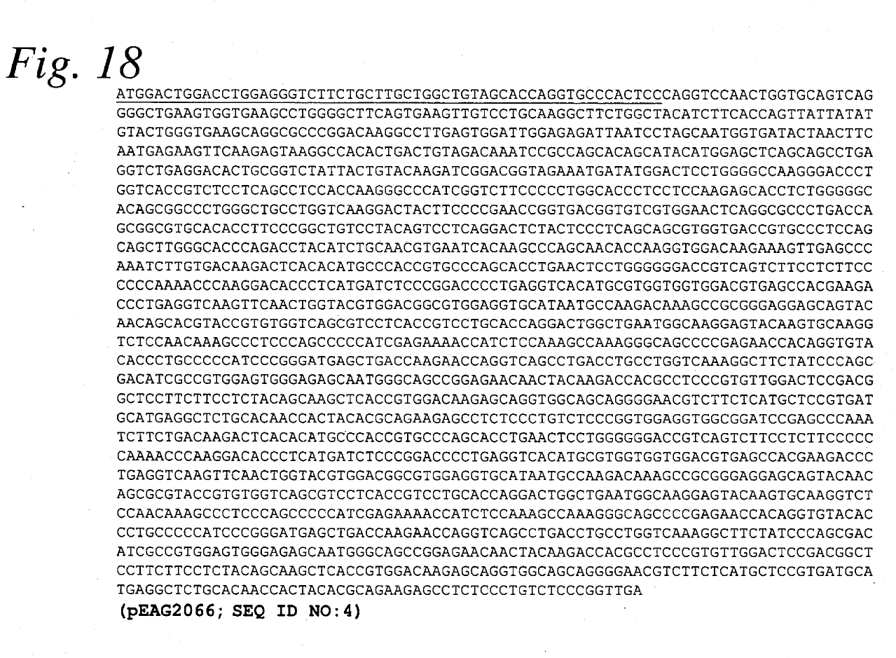

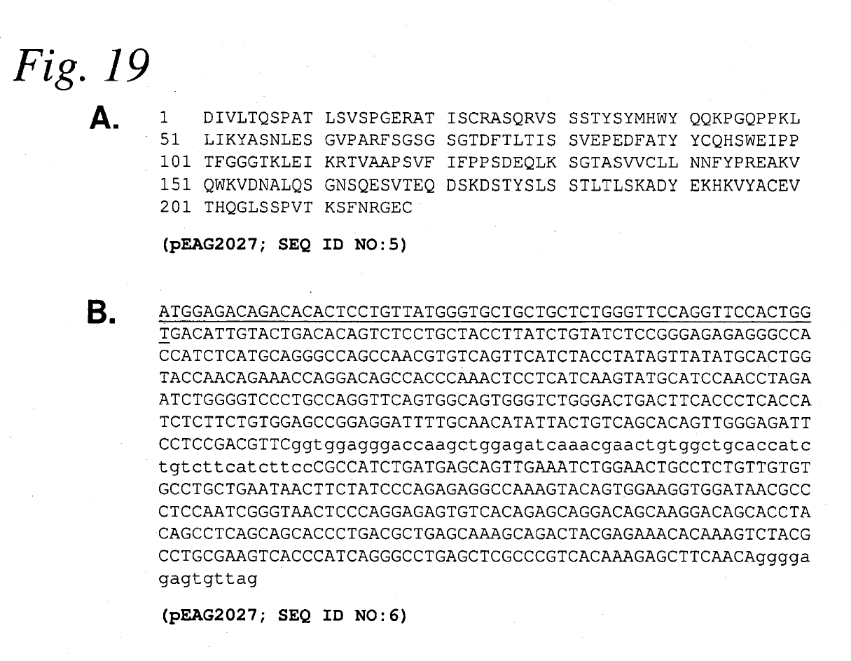

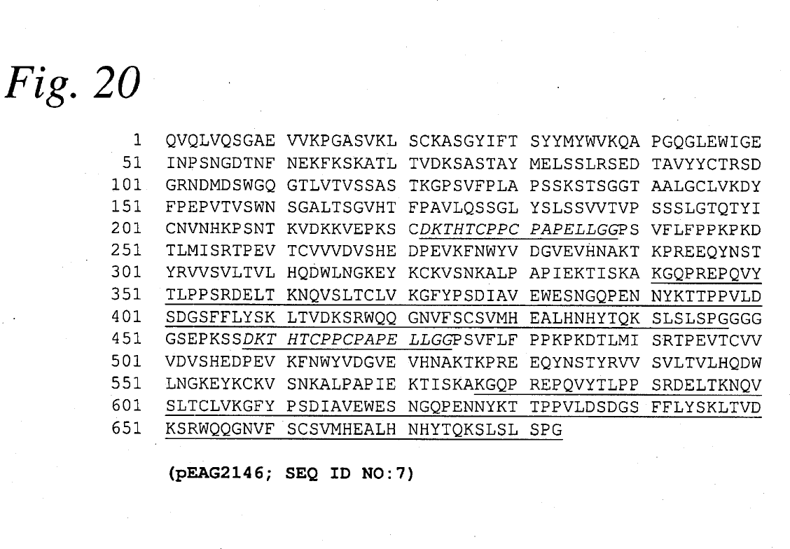

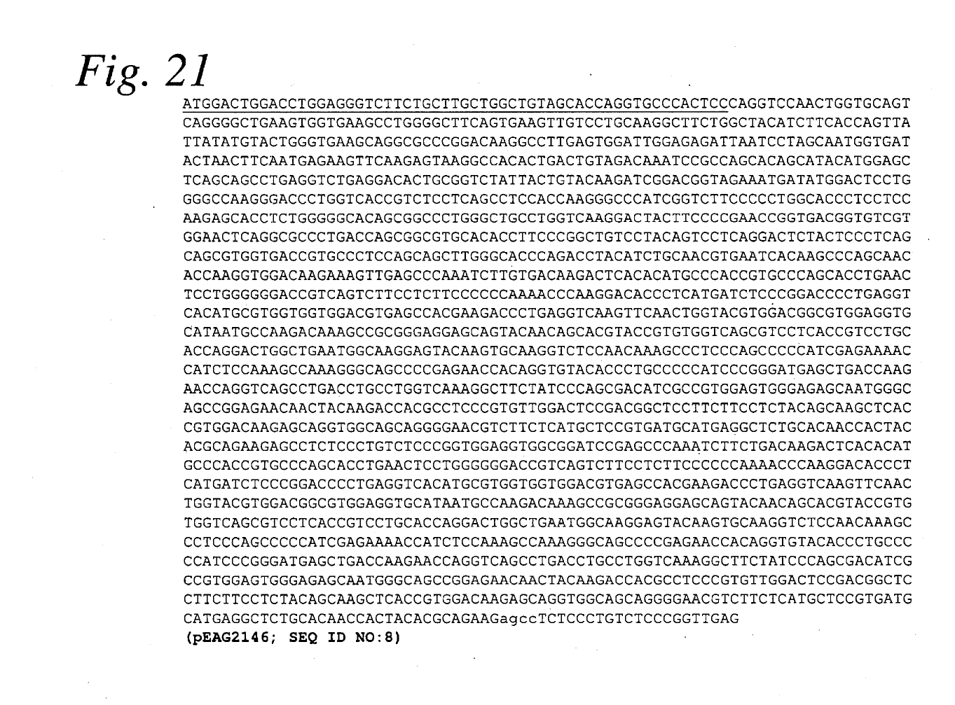

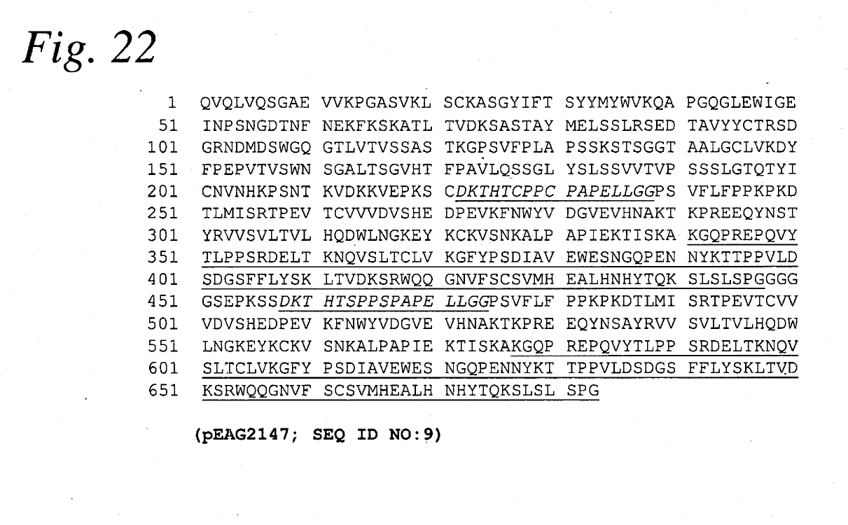

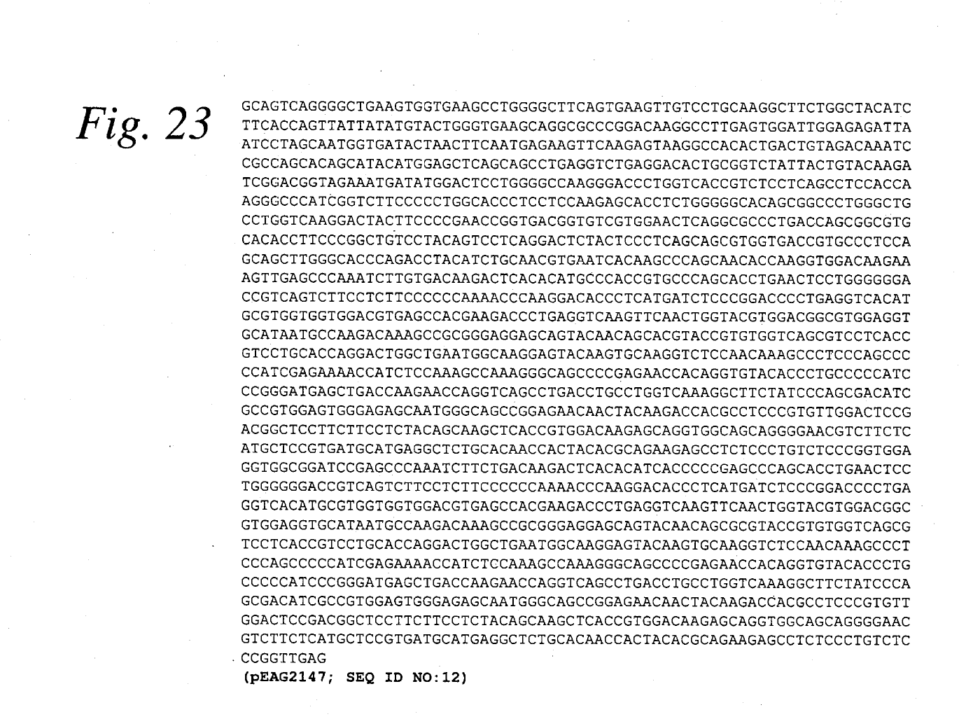

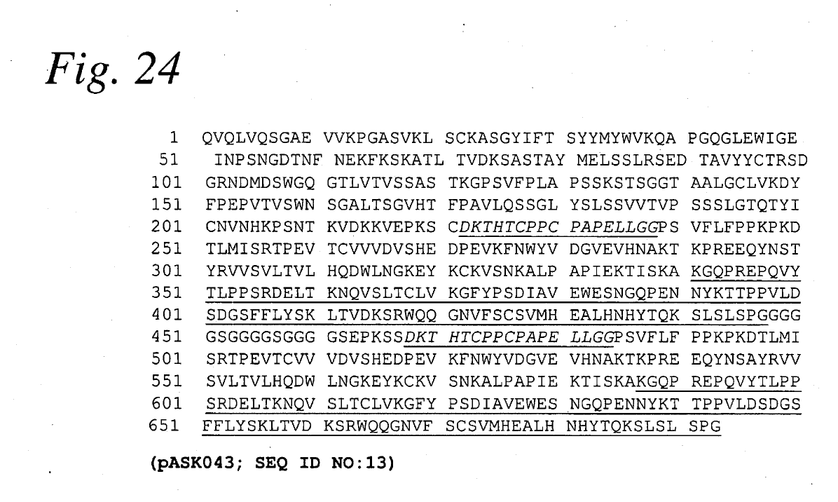

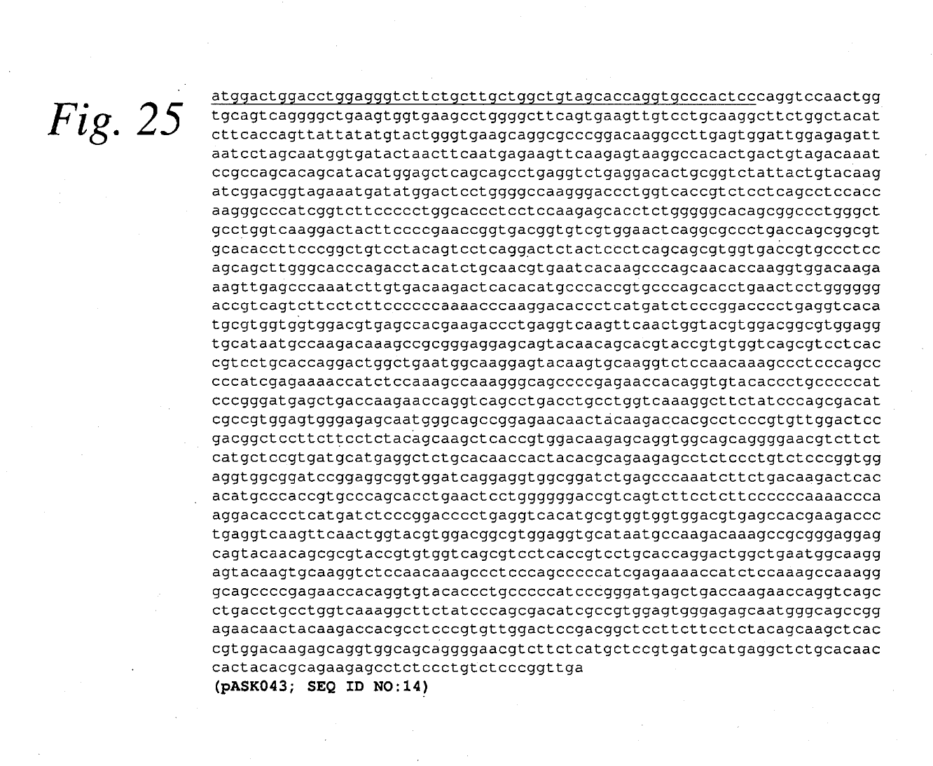

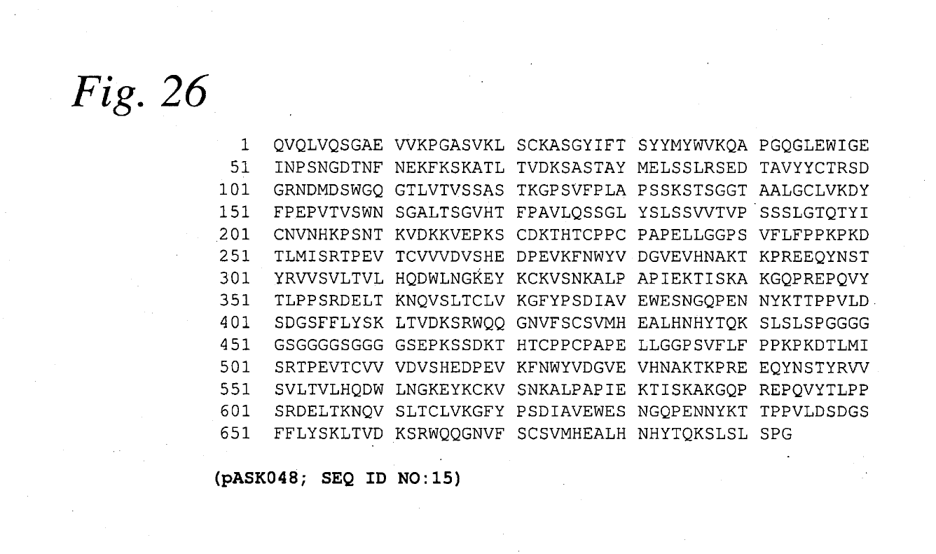

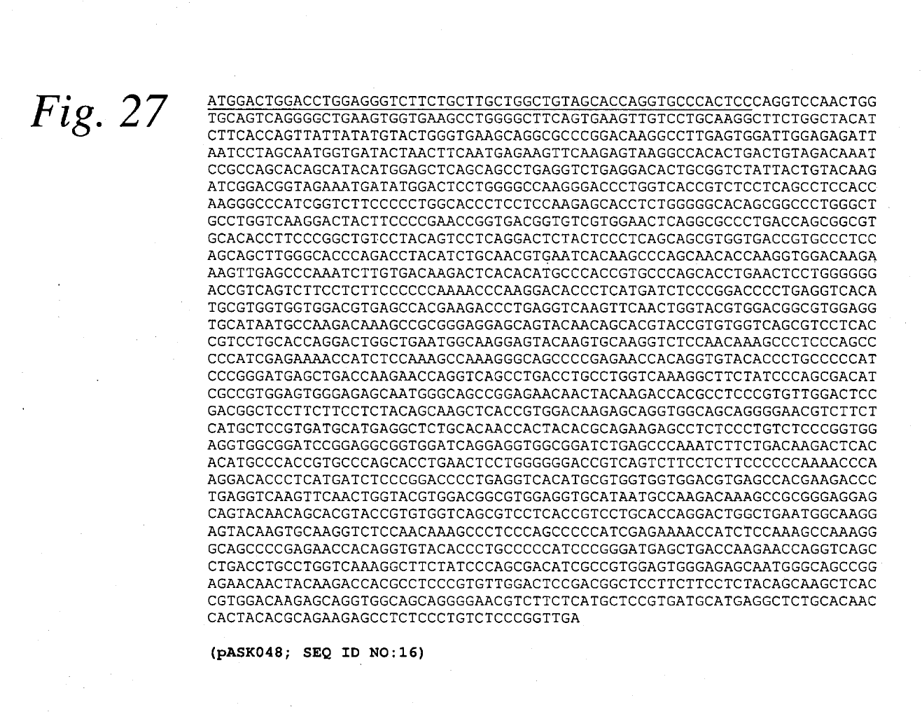

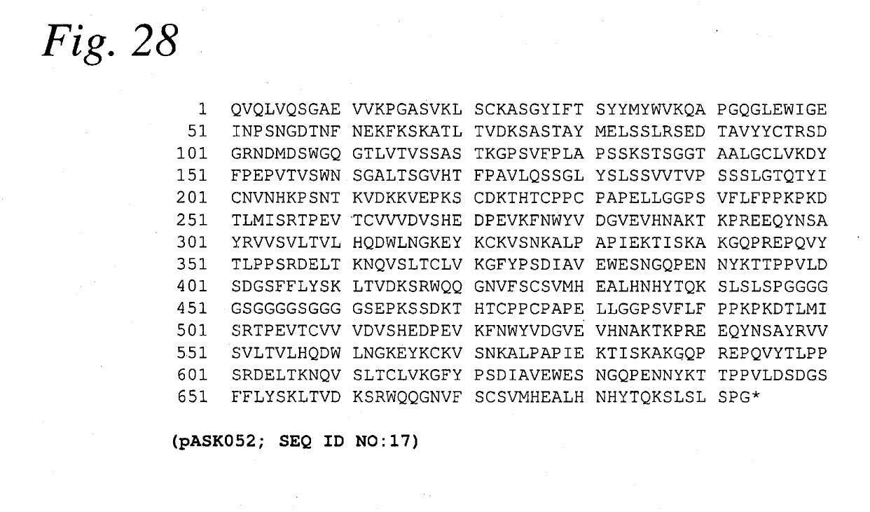

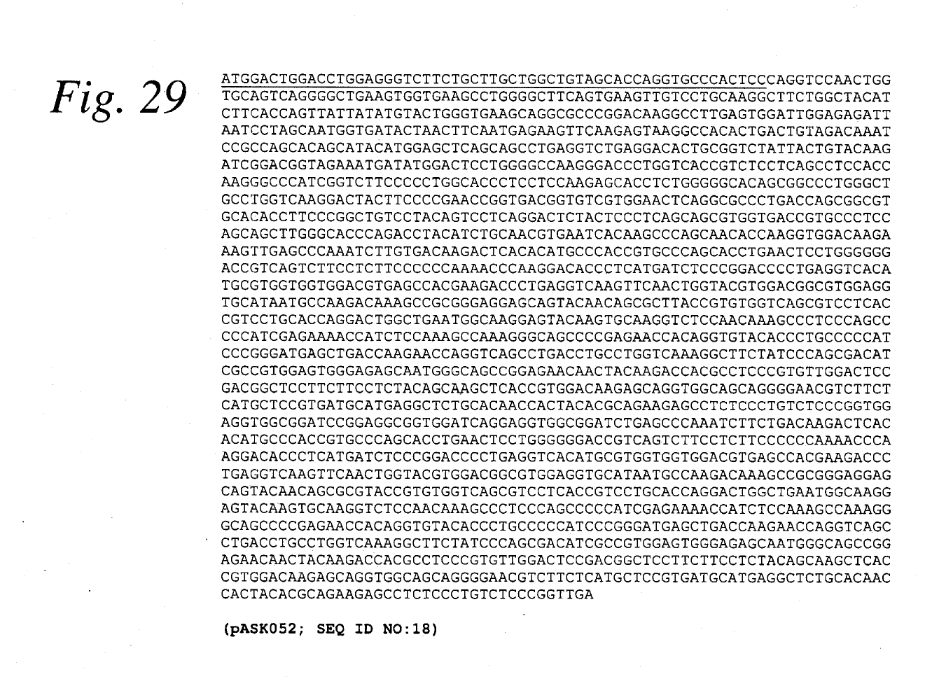

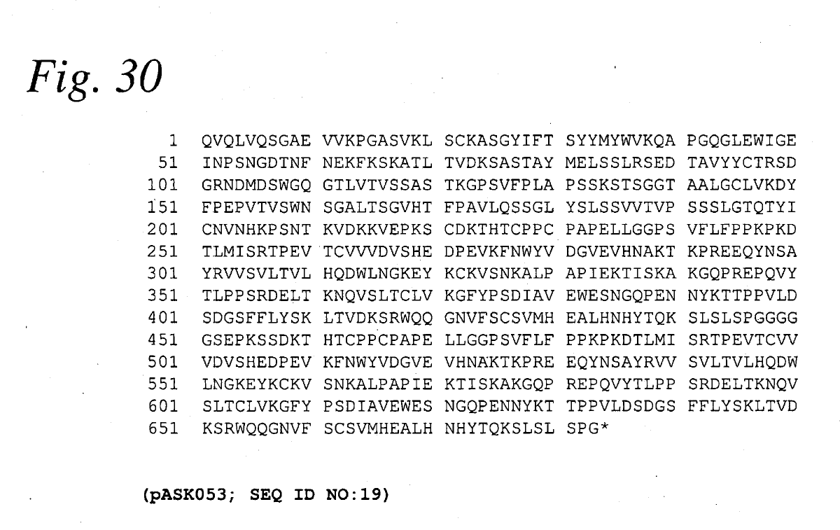

The scFc polypeptides of the invention preferably have biophysical properties (e.g., thermal stability) which are comparable to conventional polypeptides. Thermal stability of a 5c8 scFc polypeptide was compared with the thermal stability of a WT huIgG1 mAb and Fc by differential scanning calorimetry (DSC). The melting profiles for the Fab and CH3 domains of the scFc were found to be in good agreement with those obtained for the mAb and the Fc fragment of IgG1 (see FIG. 16). In particular, the CH2 domain of the hemiglycosylated scFc (ASK043) has a Tm similar to that of aglycosylated IgG1 (61.9° C. vs. 61° C., respectively), whereas the fully glycosylated scFc (ASK048) has improved stability comparable to that determined for the CH2 domain of WT IgG1.

### Example 7

### Anti-LINGO scFc Antibodies with Improved Solubility

LINGO-1 is a CNS-specific and membrane associated glycoprotein that, together with NgR1/p75 and NgR1/TAJ (TROY), form a signaling complex that binds myelin inhibitors and mediates axonal outgrowth. Soluble LINGO-1 (LINGO-1-Fc), which antagonizes LINGO-1 binding, can significantly improve functional recovery in spinal tract injury models.

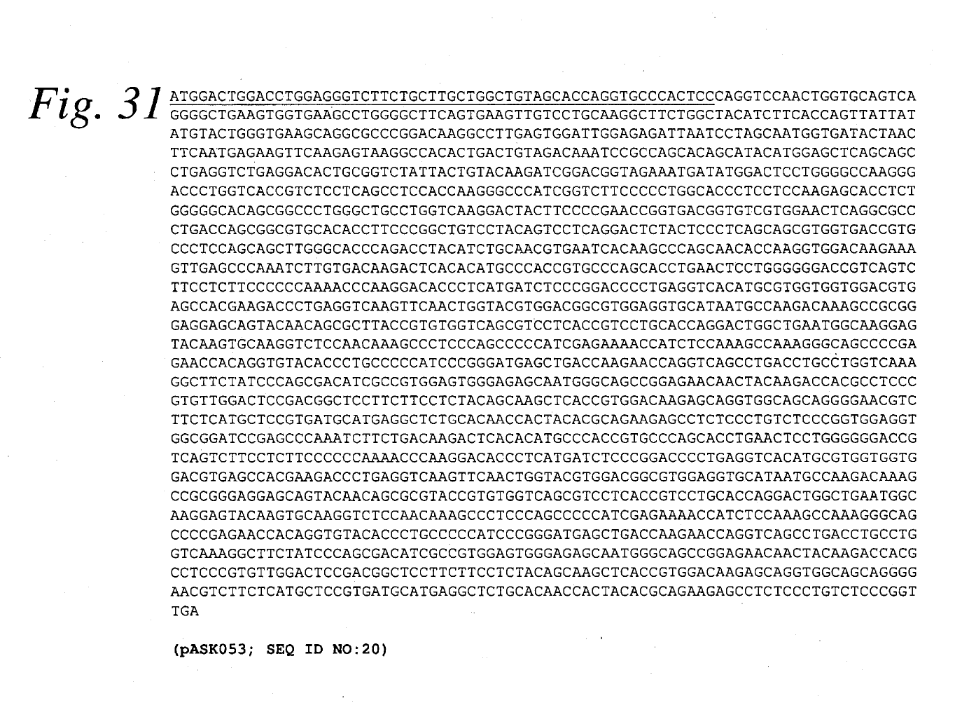

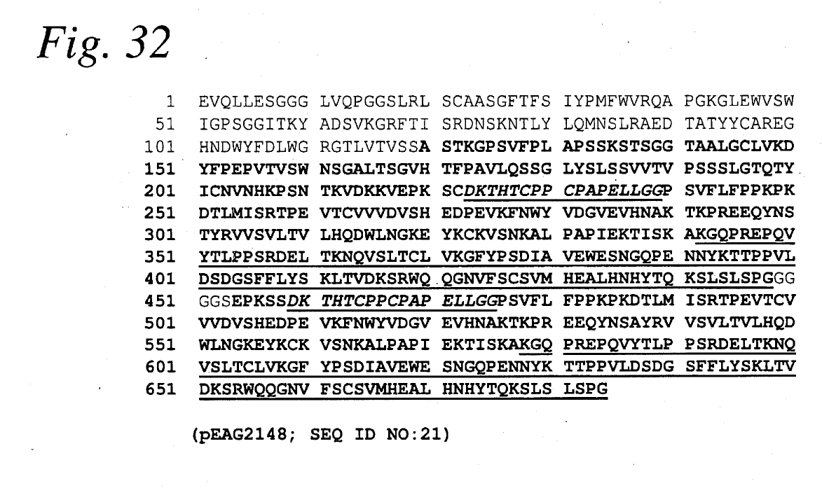

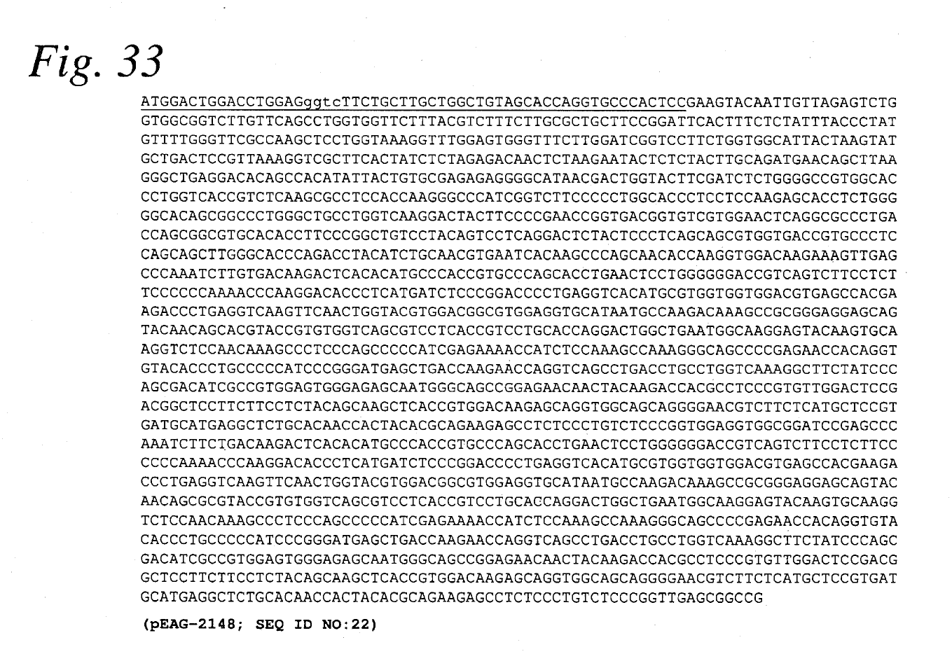

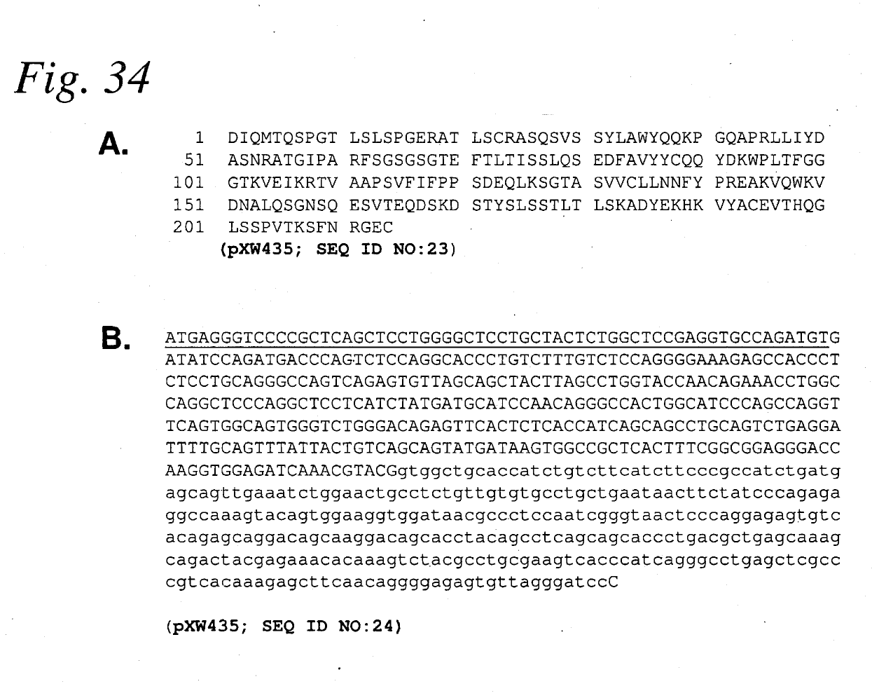

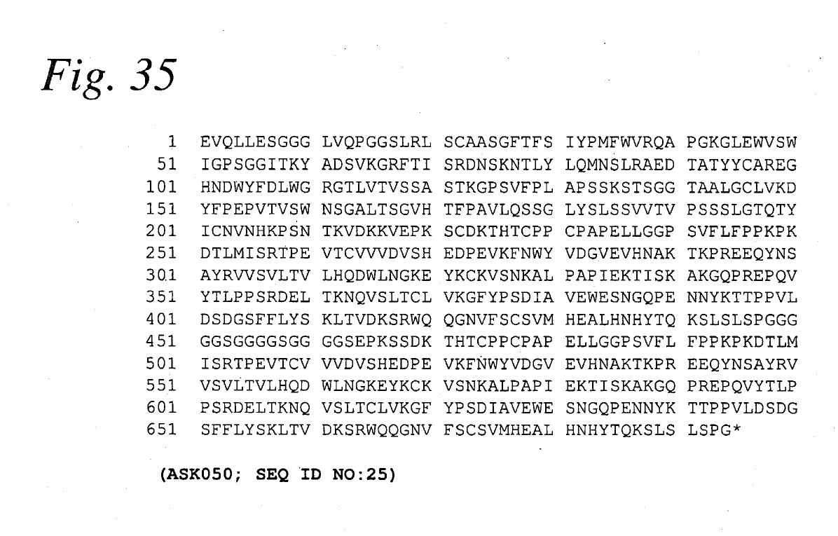

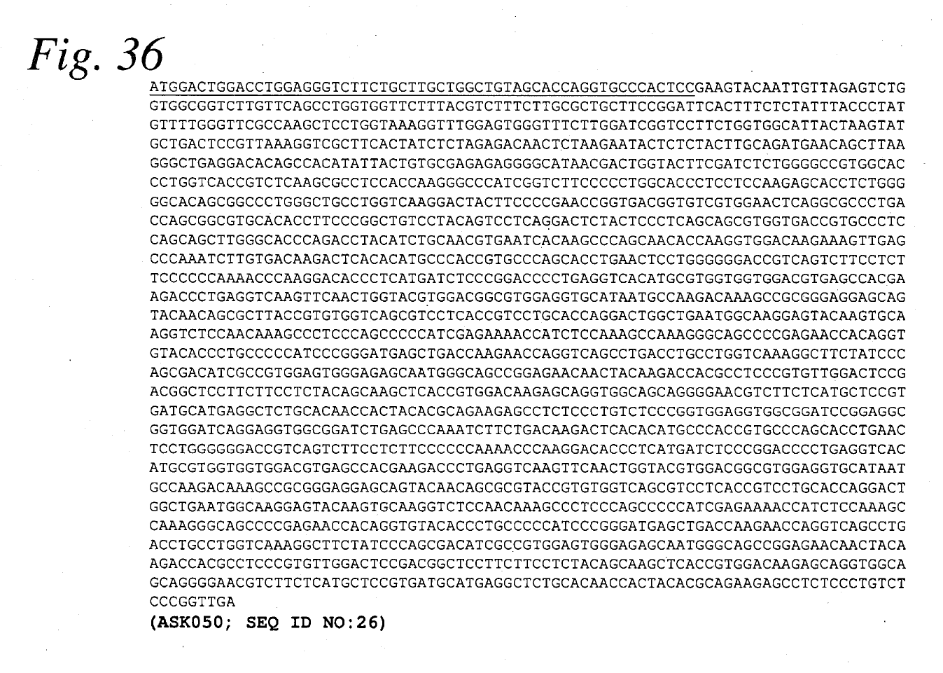

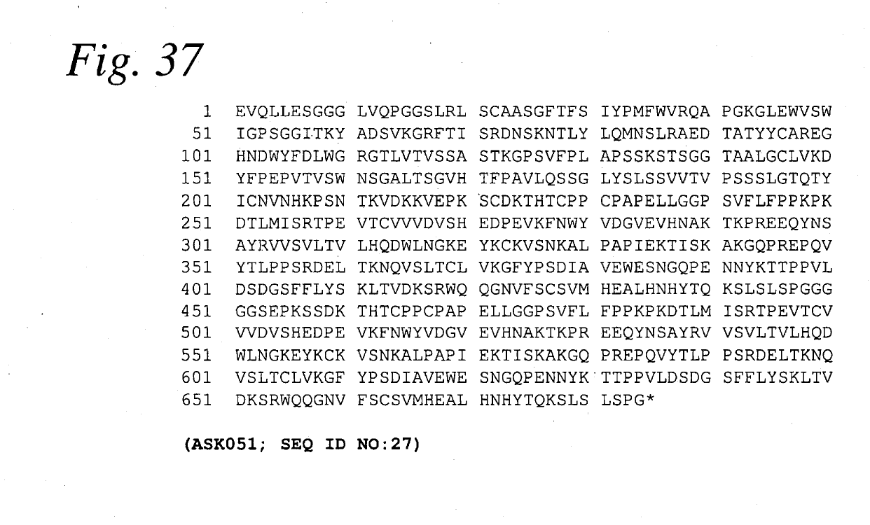

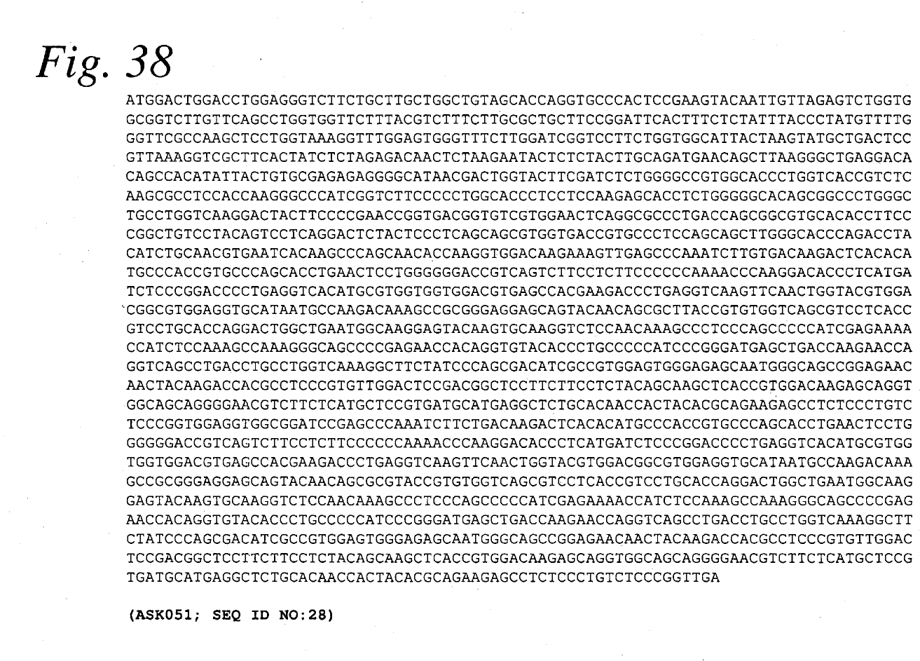

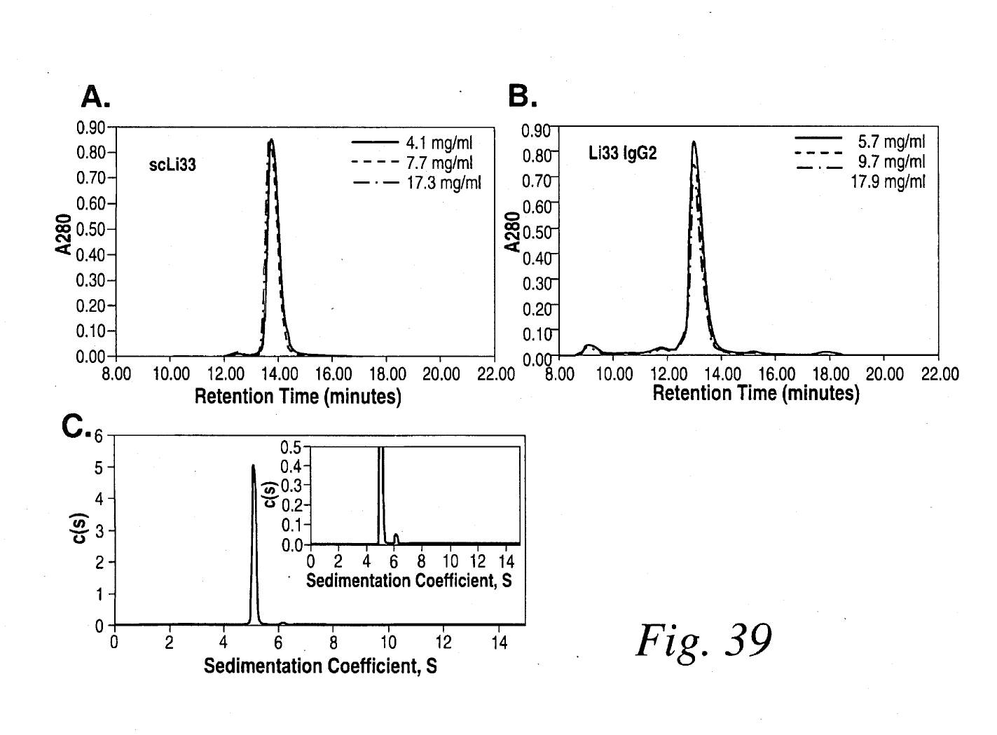

Several anti-LINGO scFc antibodies was synthesized according to the methods of the invention. The amino acid and nucleotide sequences of the heavy chains of an exemplary anti-LINGO antibody (EAG2148) are shown in FIGS. 32, 33 and FIGS. 35-38. The amino acid and nucleotide sequences of the light chain of each antibody are shown in FIG. 34. FIG. 39 shows an analysis of the protein concentration dependent solubility characteristics of the anti-Lingo, scFc antibody molecule as determined by analytical SEC (A & B) and ultracentrifugation (C). Li33, an anti-LINGO human IgG1 antibody is prone to aggregation and therefore the more soluble human IgG2 version was generated. However, even in comparison to the more soluble IgG2 construct (B), 1×G4S linked, hemiglycosylated Li33 scFc (A) exhibits significantly better solubility characteristics since with increasing protein concentration the area under the curve decreases for IgG2 monomer peak but not for the scFc. (C) An assessment of the homogeneity of anti-LINGO Li33 scFc in 20 mM Tris, 150 mM NaCl, pH 8 at 0.7 mg/ml was made by analytical ultracentrifugation using sedimentation velocity measurements. Whereas no more that 1% aggregated material was detectable in the Li33 scFc preparation at this concentration, the Li33 IgG2 mAb preparations in PBS, pH 7 at concentrations as low as 0.3 mg/ml comprised 4%-16% aggregated protein.

### Example 8

### Anti-CD2 scFc Antibodies

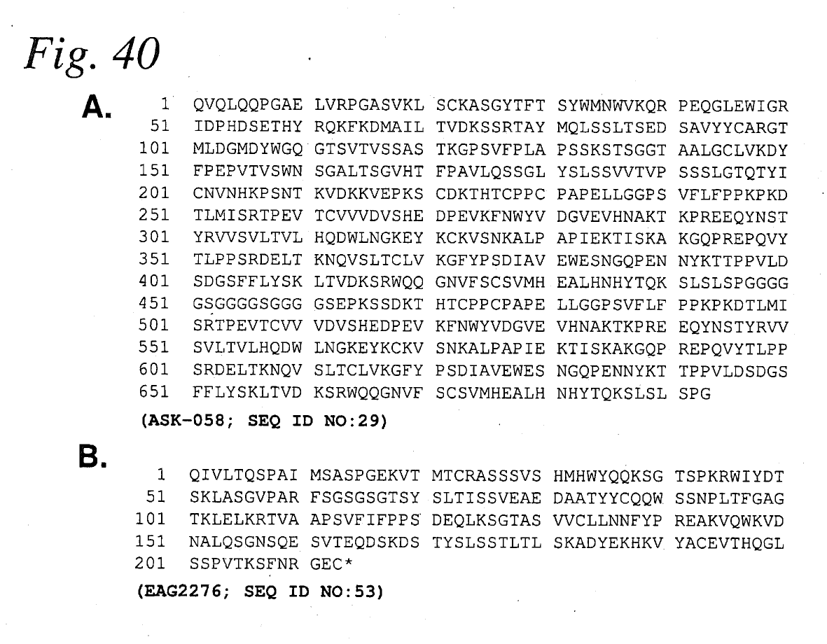

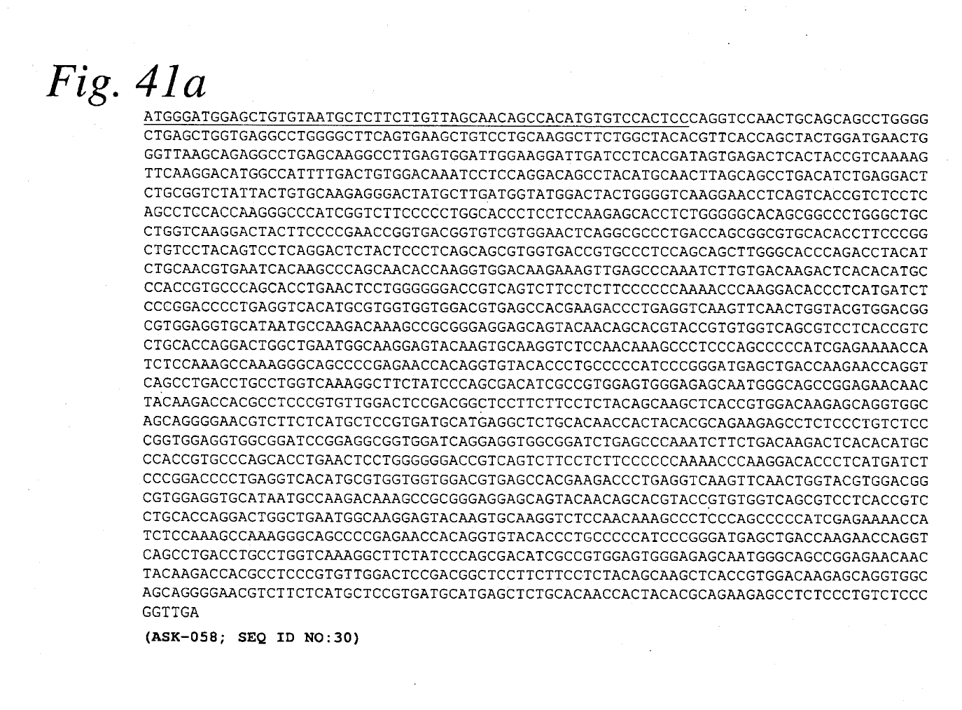

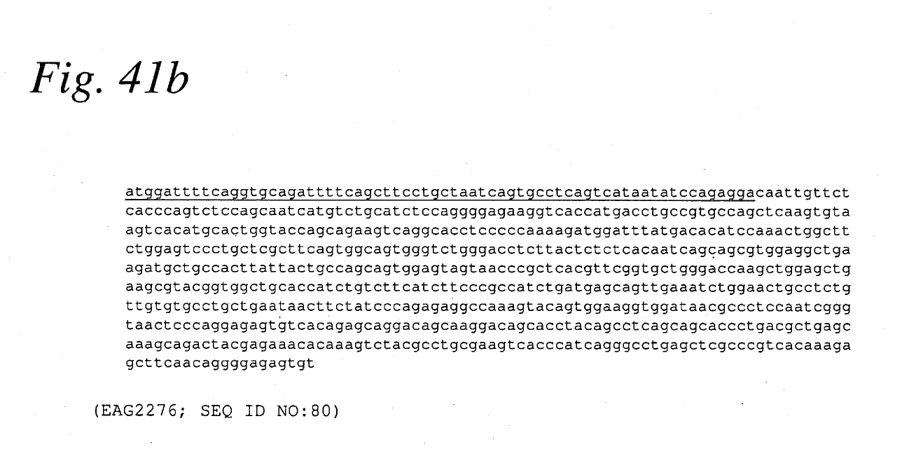

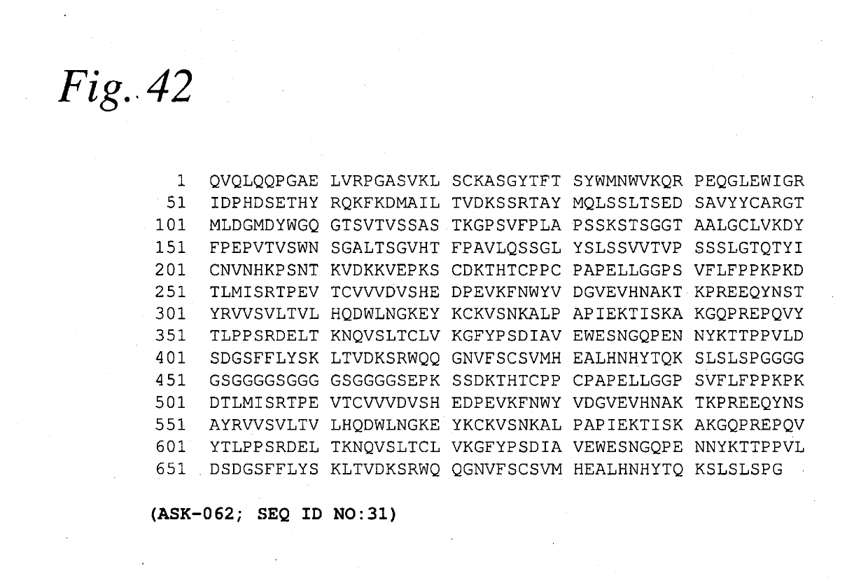

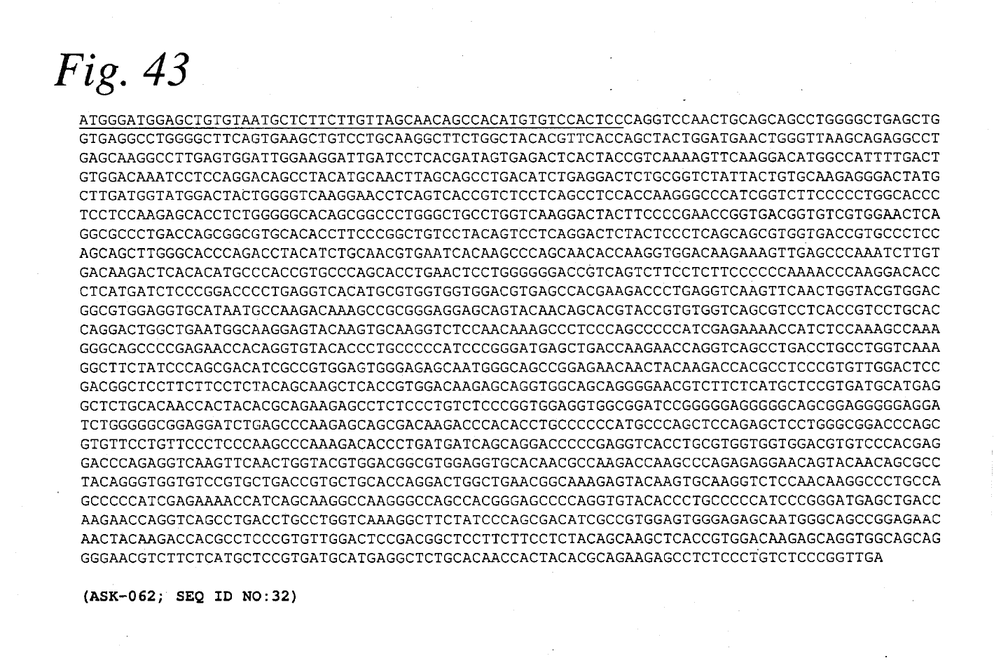

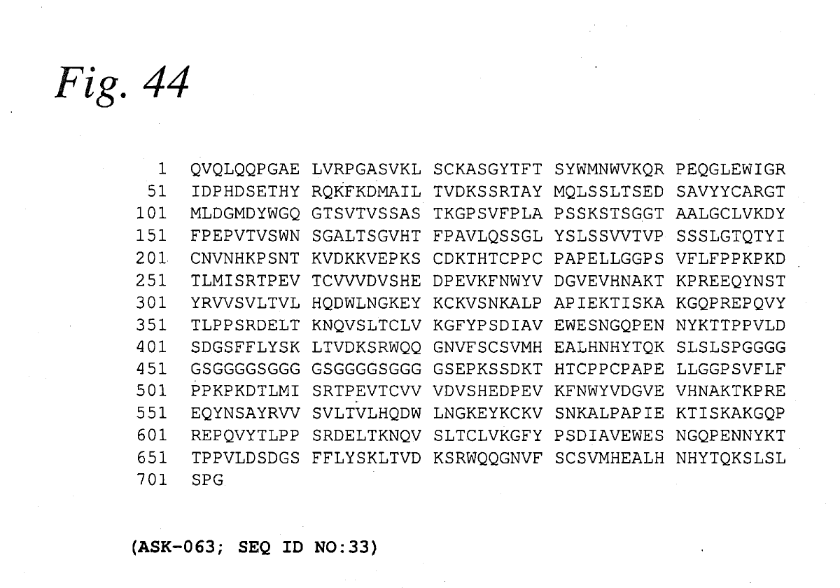

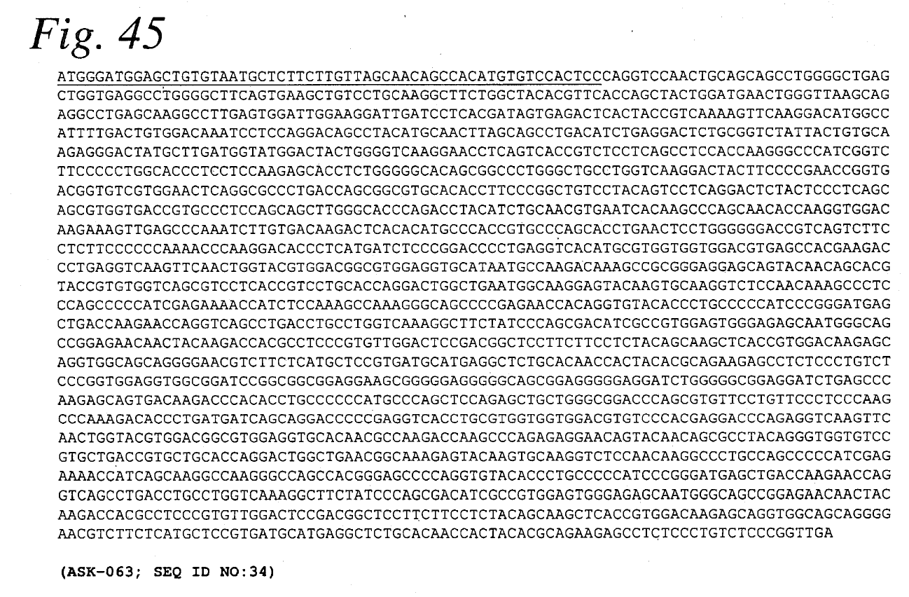

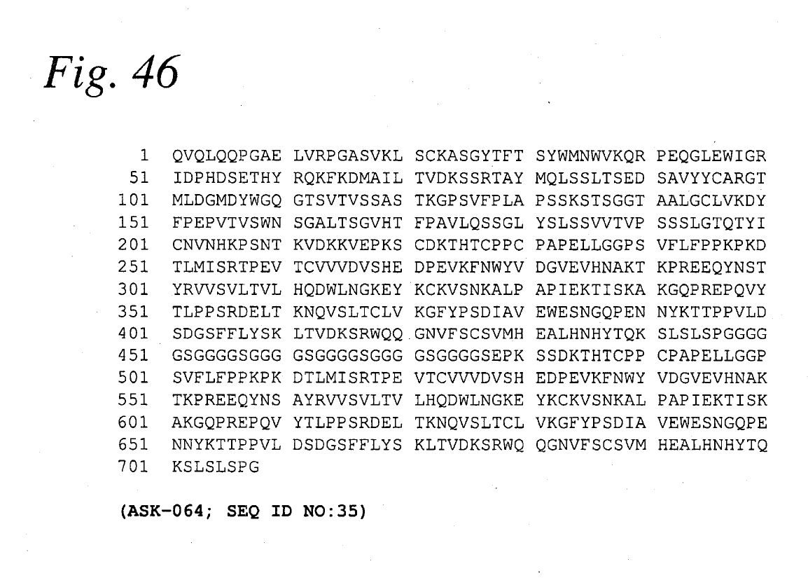

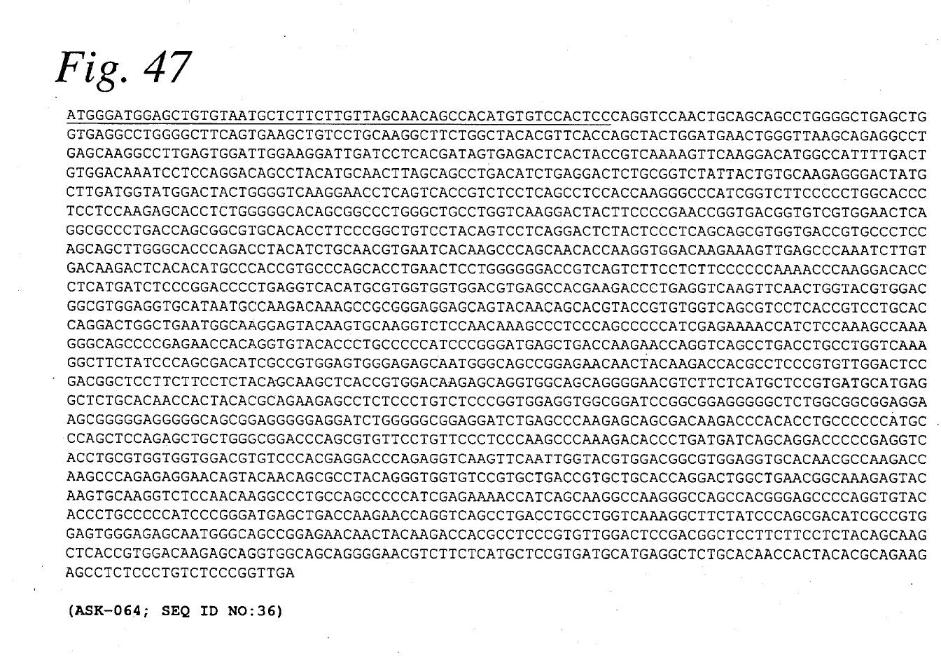

CD2 is a tumor-associated pan T cell antigen found on T-cells and natural killer (NK) cells that is associated with a number of T-cell associated disorders including certain autoimmune disorders (e.g., Graft versus Host Disease, psoriasis, renal transplantation) and T-cell cancers (e.g., Non-Hodgkin's Lymphoma). Exemplary anti-CD2 scFc antibodies may be synthesized according to the methods of the invention. chCB6 is a human CD2-specific chimeric monoclonal antibody (IgG1, kappa). Fully glycosylated chCB6 chimeric scFc antibodies comprising 3×G4S, 4×G4S, 5×G4S, or 6×G4S linkers were synthesized according to the invention. Exemplary heavy chain amino acid and nucleotide sequences are shown in FIGS. 40-47.

### Example 9

### Anti-LTβR scFc Antibodies

Lymphotoxin β Receptor (LTβR) is a member of the tumor necrosis factor (TNF) family of receptors and has been implicated in apotosis and cancer. Exemplary anti-LTβR scFc antibodies may be synthesized according to the methods of the invention. For example, the binding site of BDA8 may be fused to an scFc region of the invention. Exemplary heavy chain amino acid and nucleotide sequences of a BDA8 antibody are shown in FIG. 64.

### Example 10

### GFRα3: scFc Fusion Polypeptides

GFRα3 is a member a family of glial-derived neurotrophic factor (GDNF) receptors. Because of their physiological role, soluble neurotrophic factors may be useful in treating the degeneration of nerve cells and loss of differentiated function that occurs in a variety of neurodegenerative diseases. For example, soluble GDNFRα that retains both ligand binding, preferably GDNF binding, and receptor signaling function (via Ret receptor tyrosine kinase) can be used to impart, restore, or enhance GDNFRα-ligand (preferably GDNF) responsiveness to neurons or other cells.

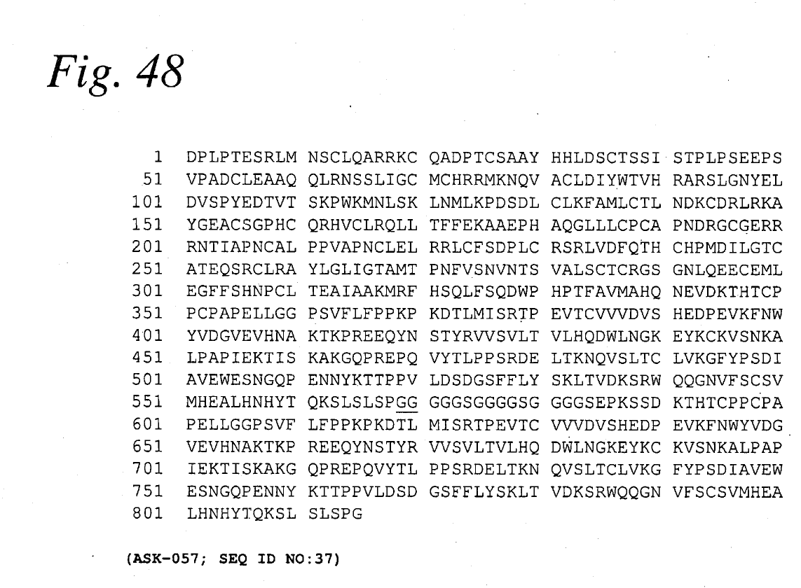

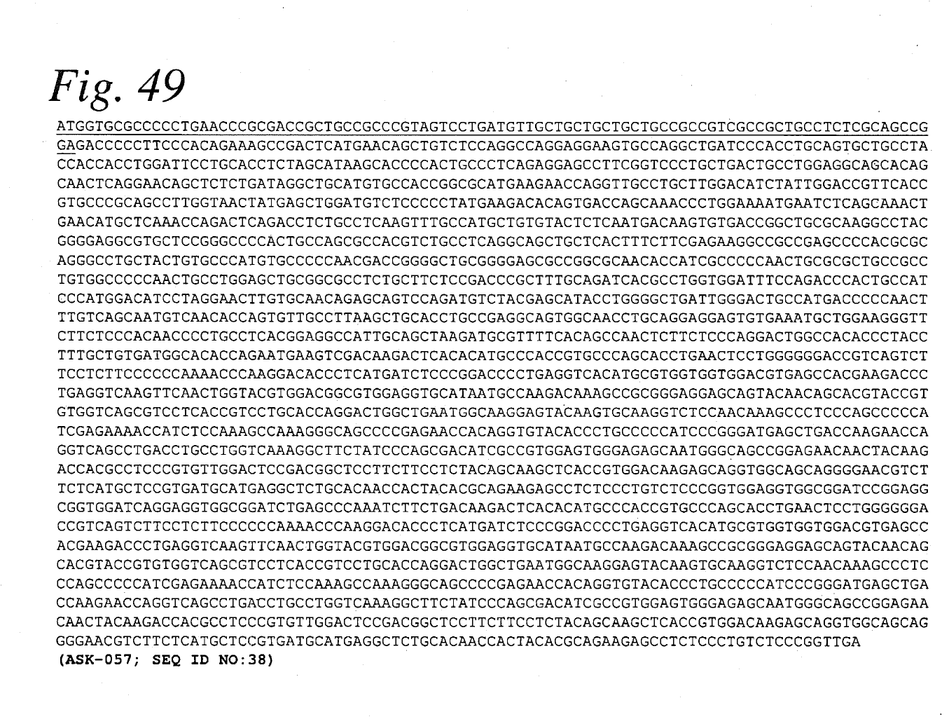

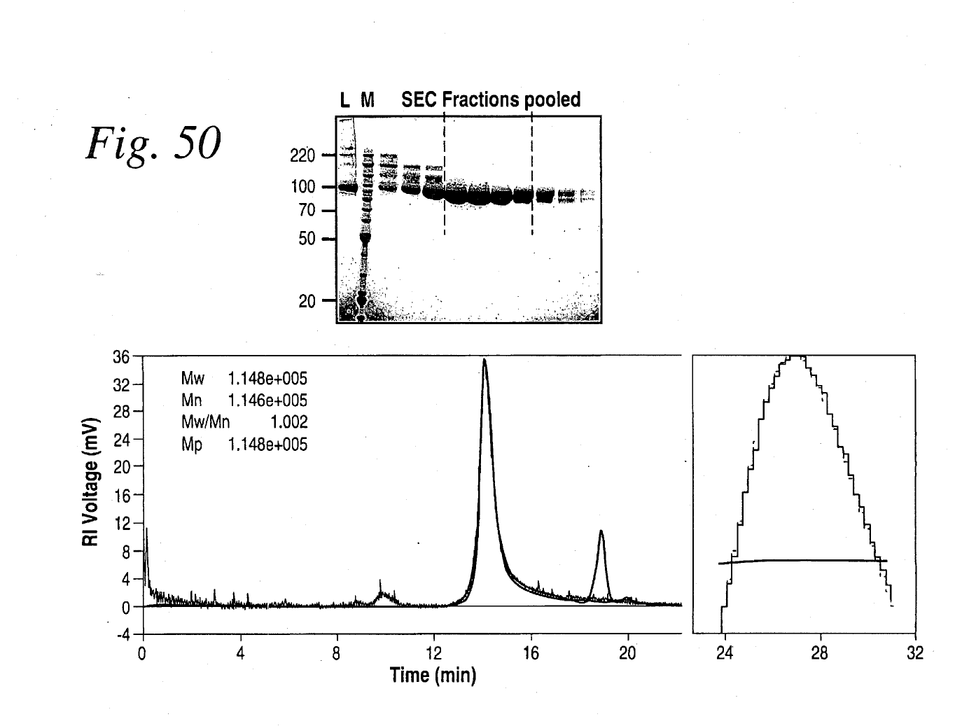

An exemplary GFRα3:scFc fusion protein (ASK057) was synthesized according to the methods of the invention. The amino acid and nucleotide sequences of the ASK057 are shown in FIG. 48 and FIG. 49. FIG. 50 shows the non-reducing SDS-PAGE and analytical SEC-LS characterization following expression of the fusion protein. The material eluted from the ProteinA column was pooled (L) and loaded on a Superdex 200 gel filtration column for separation of the monomeric (sc) and dimeric (dc) scFc populations (lane 1 of the gel) followed by the molecular weight standards in lane 2 (M). The SEC fractions pooled for the GFRα3:scFc are bracketed between two dashed lines. SEC-LS analysis of GFRα3:scFc obtained after the 2-step purification indicated a homogeneous preparation with a molecular mass of 114.8 kDa. The stoichiometry of GFRα3:scFc binding to homodimeric neublastin was determined by solution phase Biacore experiments to be 2 GFRα3: scFc: 1 neublastin dimer.

### Example 11

### IFN-β: scFc Fusion Polypeptides

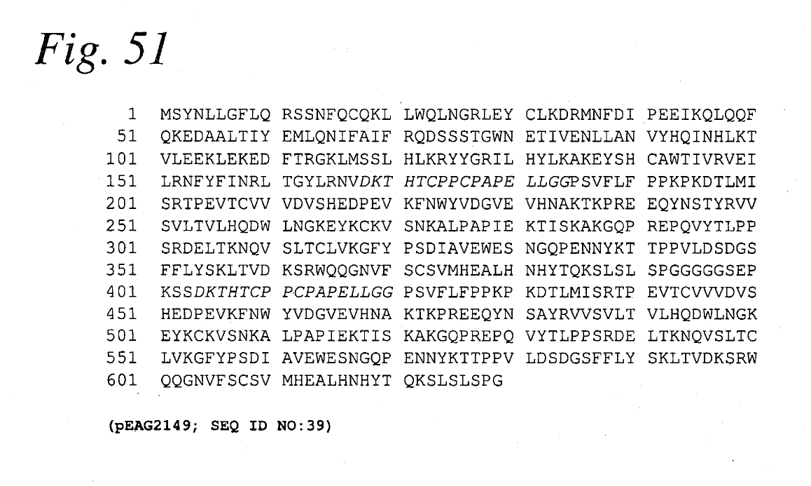

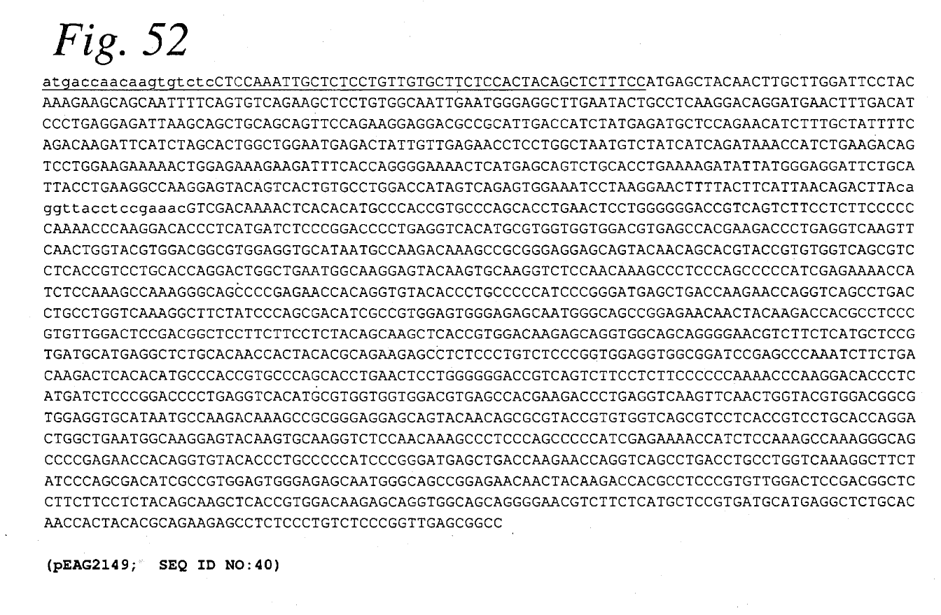

Interferon beta-1a (e.g., AVONEX®) is useful for the treatment of multiple scleros. An exemplary IFN-β:scFc immunoadhesin (EAG2149) was synthesized according to the methods of the invention. The EAG2149 molecule contained a 1×G4S linker and was modified to facilitate hemiglycosylation. The amino acid and nucleotides sequences of ASK057 are shown in FIG. 51 and FIG. 52.

### Example 12

### LTβR: scFc Fusion Polypeptides

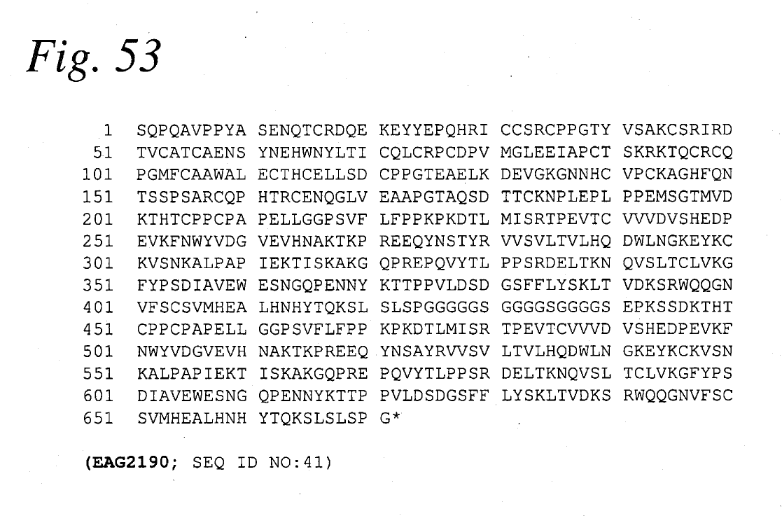

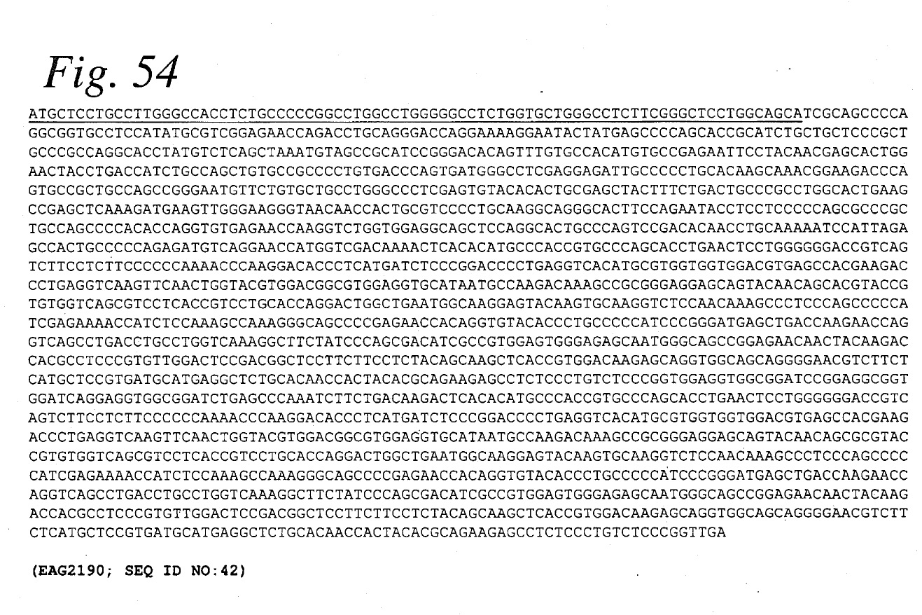

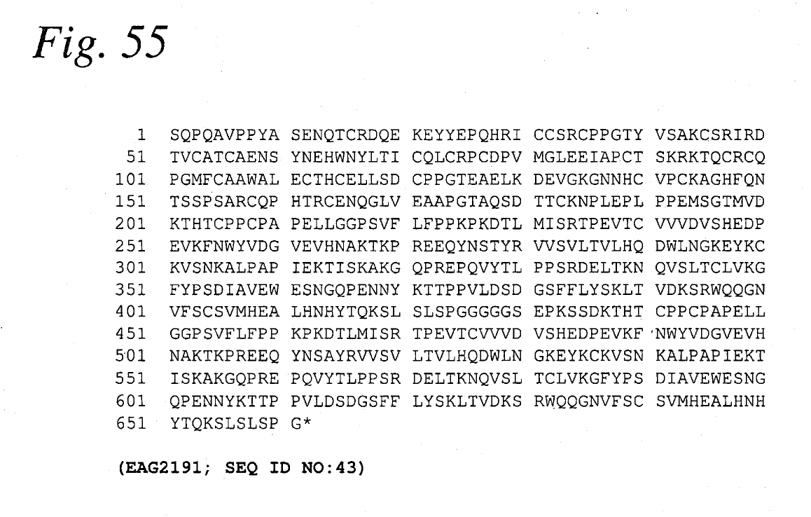

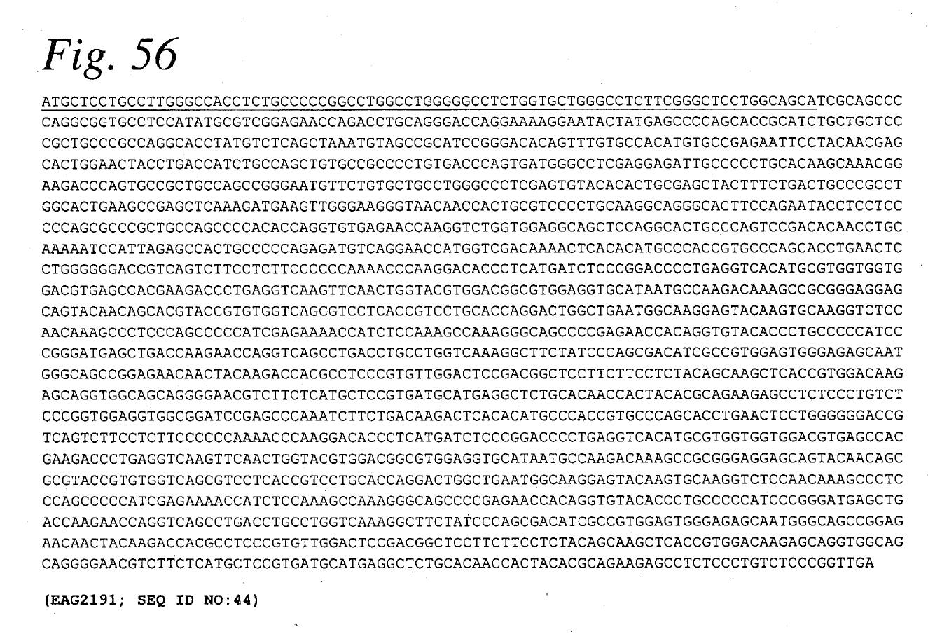

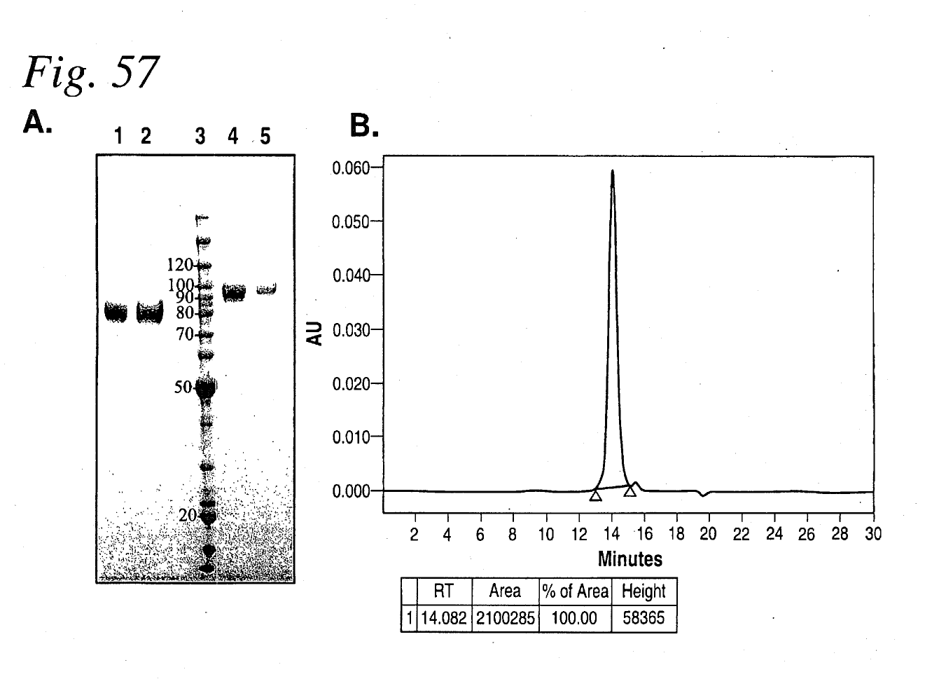

Exemplary LTβR:scFc immunoadhesins (EAG2190 and EAG2191) were synthesized according to the methods of the invention. The EAG2190 molecule contained a 3×G4S linker and was modified to facilitate hemiglycosylation. The amino acid and nucleotides sequences of these molecules are shown in FIGS. 53-56. A 4-12% gradient SDS-PAGE of the purified LTβR:scFc fusion protein was performed revealing a single band corresponding to the expected molecular weight of 75 kD for the DLI33scFc. Analytical gel filtration of the purified protein was performed on a Phenomonex Biosep-S-3000 column in 20 mM sodium phosphate pH 7.2, 150 mM NaCl (PBS) at 0.5 mL/min. Eluant was monitored at 280 nM. The purified LTβR:scFc was then characterized for homogeneity and binding activity to a known ligand LTα1β2. Reducing and nonreducing SDS PAGE of the purified LTβRscFc is shown in FIG. 57A. Analytical size exclusion chromatography shows a single peak of high homogeneity for the purified LTβR:scFc (FIG. 57B).

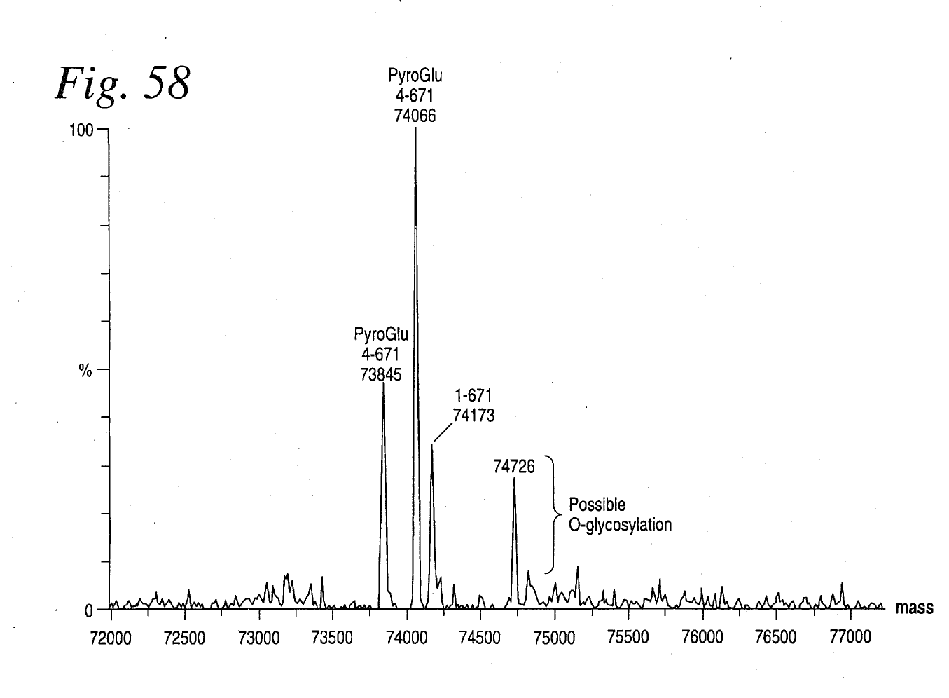

The LTβR:scFc was analyzed by mass spectrometry on a LCZ mass spectrometer after deglycosylation with PNGaseF by reduction with DTT and incubation for 12 h at room temperature (see FIG. 58). Mass spectrometry of the reduced and N-deglycosylated molecule showed that the theoretical molecular mass of 73,844.5 was in agreement with found value of 73,846. N-terminal proteolytic heterogeneity was observed in the first four amino acids of the expressed LTβR:scFc and the first residue of both the N−1 and N−3 components is pyroglutamate. Low levels of O-glycosylation were observed in the mass spectrometry reflected in the peaks at 74726 and 74820.

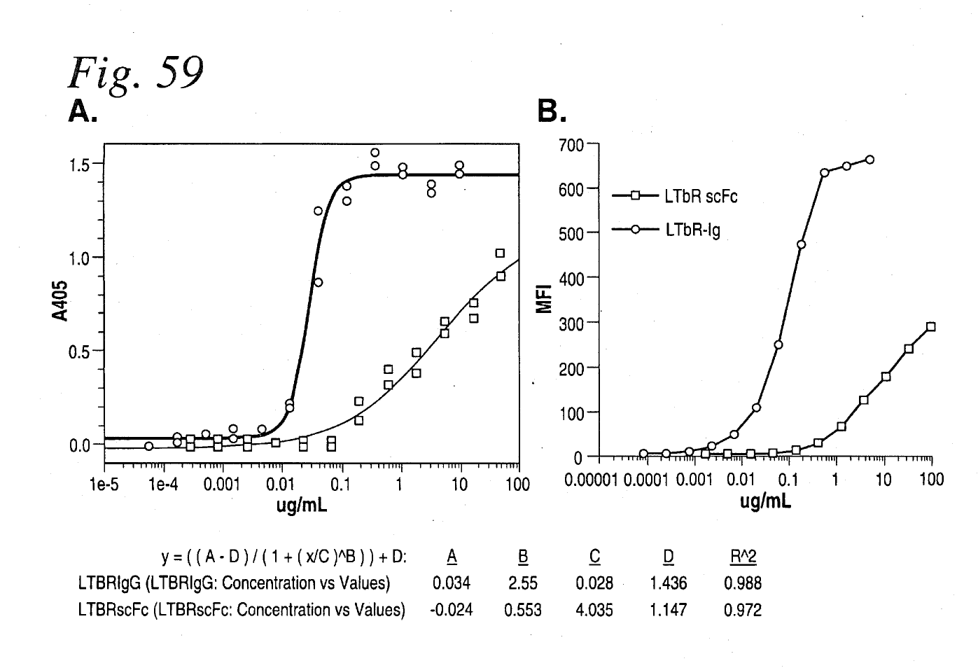

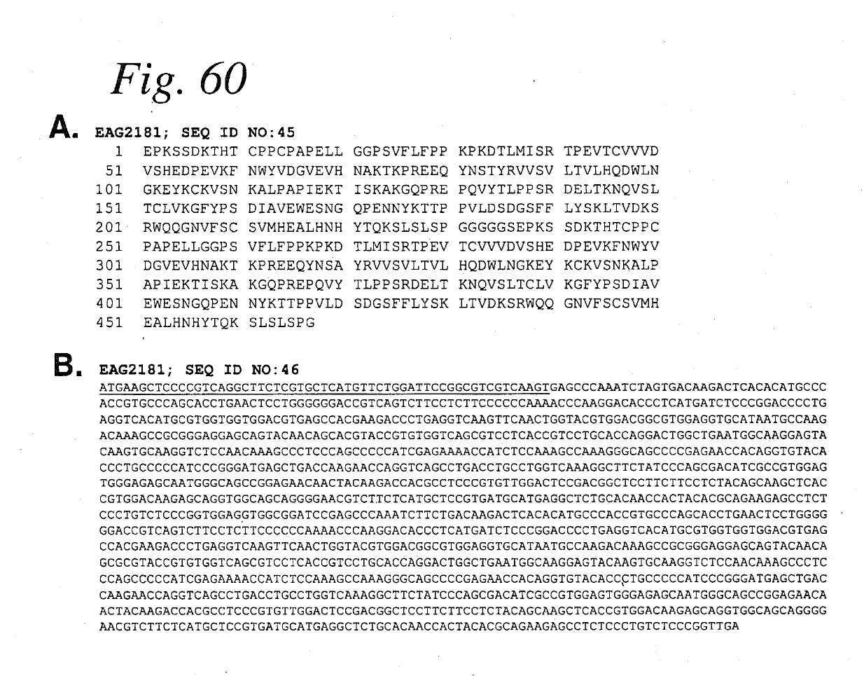

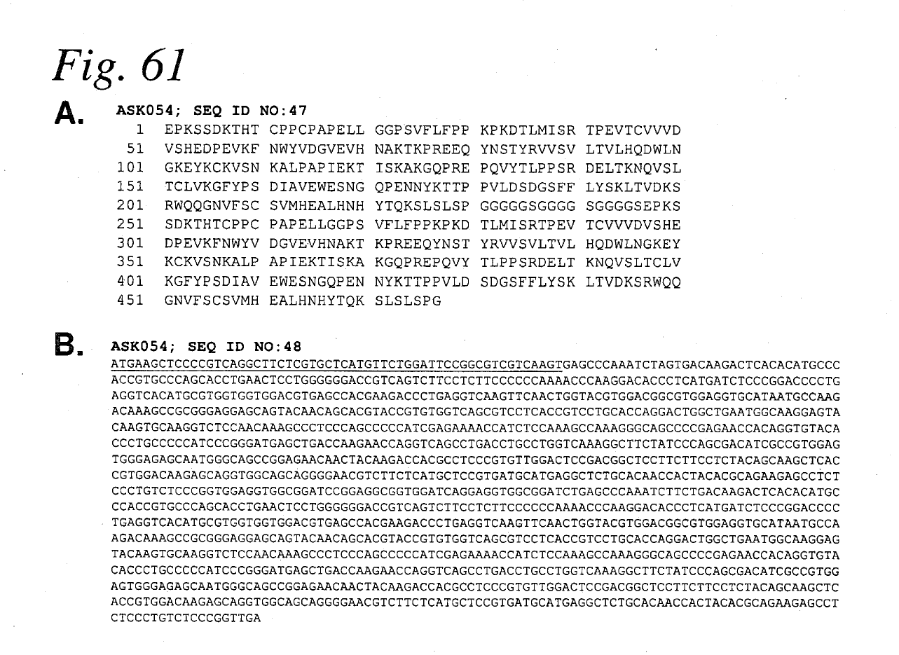

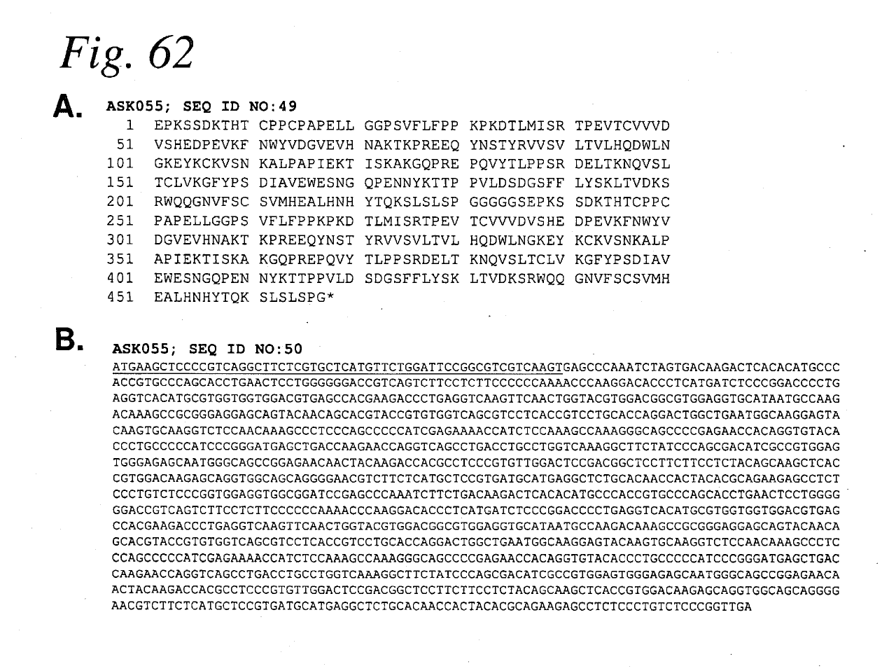

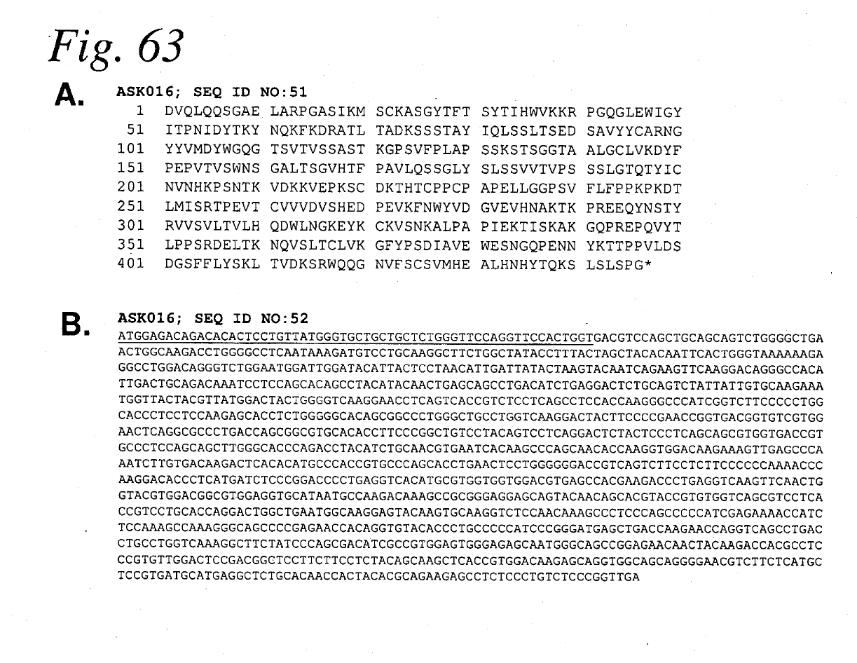

FIG. 59A depicts the results of an ELISA evaluating the binding affinity of the monomeric LTβR:scFc to LTα1β2. ELISA plates were coated overnight at 4° C. with 5 ug/ml LTα1β2 in PBS. Plates were then blocked with 1% Casein in 10 mM PBS, pH 7.0, then washed with 10 mM PBS, pH 7.0, 0.1% Tween 20 (wash buffer) three times. LTβR:IgG or LTBβ:scFc serially diluted into 10 mM PBS, 362 mM NaCl, 0.055 Tween-20, 0.1% Casein, 5% FBS pH 7.0 (assay diluent) and incubated for 1 h, then washed with wash buffer. To each well was added Donkey anti-human heavy and light specific HRP (Jackson Labs) conjugated secondary antibody diluted 1:5000 in assay diluents for 60 min then washed with PBS. The HRP was developed using tetramethybenzidine and hydrogen peroxide in 100 mM NaAcetate pH 4.0 after several minutes, the assay was stopped by the addition of 100 uL of 1N sulfuric acid and sample absorbance read at 450 nM. The ELISA analysis shows that the LTβR:scFc has a reduced binding affinity compared with LTβR:IgG. This result was reflects the reduced avidity of the monomeric, LTβR:scFc relative to dimeric LTβR:IgG. The Kd values of dimeric LTβRIgG (<0.10 nM) and monomeric LTβR (40 nM) were previously shown to differ by over 400 fold (Eldredge, Berkowitz et al. 2006)

The binding affinity of the monomeric LTβR:scFc was also evaluated by FACS analysis (FIG. 59B). FACS was done on II-23 cells according to the methods of Eldredge et al. (Eldredge, Berkowitz et al. 2006). FACS binding assays using flow cytometry were done according to the method previously described (Force, Walter et al. 1995). Briefly, in direct binding assays human LTα1β2 was detected on phorbol myristate acetate activated II-23 cells (American Type Culture Collection (ATCC) Manassas, Va.) in FACS buffer with varied concentrations of either the LTβRIgG or biotinylated LTβRIgG (bLTβRIgG). Cells were incubated on ice for 1-2 h then washed with PBS and centrifuged. The appropriate steptavidin phycoerythrin or anti-hFc phycoerythrin labeled secondary (Molecular Probes, Eugene, Oreg.) in FACS buffer was added and incubated with the cells for an additional 1 h and washed again. The fluorescent staining of the cellular bound LTβRIgG on the II-23 cells was quantified by determining the mean channel fluorescence by FACS. Both the FACS and ELISA show the LTβRscFc has a reduced binding affinity compared with LTβRIgG. This result was expected since the monomeric protein lacks the avidity of LTβRIgG. The Kd values of dimeric LTβRIgG (<0.10 nM) and monomeric LTPR (40 nM) were previously shown to differ by over 400 fold (Eldredge, Berkowitz et al. 2006)

What is claimed:

1. A nucleic acid molecule comprising a nucleotide sequence encoding an isolated binding polypeptide comprising (i) a first binding site, and (ii) a first Fc region encoded in a single contiguous genetic sequence wherein:
a. said Fc region is a heteromeric Fc region and comprises at least two Fc moieties,
b. said Fc region is fused via a polypeptide linker sequence interposed between said Fc moieties; and said Fc region imparts at least one effector function to said binding polypeptide 1.

2. The nucleic acid molecule of claim 1, which is in an expression vector.

3. A host cell comprising the expression vector of claim 2.

4. A method for producing a binding polypeptide comprising culturing the host cell of claim 3 in culture such that the binding polypeptide is produced.

5. A single chain Fc polypeptide comprising two CH2 domains and two CH3 domains characterized in that said CH₂ and CH3 domains form a functional Fc region within the polypeptide chain.

6. A single chain Fc polypeptide according to claim 5 wherein the Fc region is capable of folding intramolecularly such that a first CH2 domain is dimerized with a second CH2 domain and a first CH3 domain is dimerized with a second CH3 domain within the polypeptide chain.

7. A polypeptide according to claim 6 in which, in N- to C-terminal sequence a first CH2 domain is linked at its C-terminus to the N-terminus of a first CH3 domain, optionally via a linker, and said first CH3 domain is linked at its C-terminus via a linker to the N-terminus of a second CH2 domain which is linked at its C-terminus to the N-terminus of said second CH3 domain, optionally via a linker.

8. A polypeptide according to claim 6 which further comprises two CH4 domains.

9. A polypeptide according to claim 1 in which each CH2 domain is from a human IgG1, IgG2, IgG3, or IgG4 molecule.

10. A polypeptide according to claim 1 in which each CH3 domain is from a human IgG1, IgG2, IgG3, or IgG4 molecule.

11. A polypeptide according to claim 1 in which said polypeptide comprises a polypeptide having least 80% identity or similarity to a human IgG1, IgG2, IgG3, or IgG4 molecule.

12. A single chain Fc polypeptide according to claim 1 which is linked to at least one binding site specific for a target molecule which mediates a biological effect.

13. A single chain Fc polypeptide according to claim 12 wherein the binding site is a ligand binding portion of a receptor or an antibody fragment.

14. A single chain Fc polypeptide according to claim 12 wherein a binding site is linked to the N-terminus of the first CH2 domain of the single chain Fc polypeptide.

15. A single chain Fc polypeptide according to claim 12 in which the binding site and the single chain Fc polypeptide are linked by a peptide linker of between 1 and 50 amino acids in length.

16. A single chain Fc polypeptide according to claim 13 wherein the linker comprises a cysteine residue.

17. A single chain Fc polypeptide according to claim 13 or claim 100 in which the linker comprises an antibody hinge selected from: (i) a polypeptide linker comprising an IgG1 upper hinge domain (SEQ NO:15) and IgG1 middle hinge domain (SEQ ID NO:16): (ii) polypeptide linker comprising an IgG2 upper hinge domain (SEQ NO:82) and an IgG2 middle hinge domain (SEQ ID NO:16); (iii) a polypeptide linker comprising an IgG3 upper hinge domain (SEQ ID NO:18) and an IgG3 middle hinge domain (SEQ NO:19); (iv) a polypeptide linker comprising an IgG4 upper hinge domain (SEQ ID NO:21.) and an IgG4 middle hinge domain (SEQ ID NO:22): (v) a modified variant of linker (i), wherein the variant comprises fewer cysteines; (vi) a modified variant of linker (ii),wherein the variant comprises fewer cysteines; (vii) a modified variant of linker (iii), wherein the variant comprises fewer cysteines; and (viii) a modified variant of linker (iv), wherein the variant comprises fewer cysteines.

18. A single chain Fc polypeptide according to claim 12 wherein the binding site comprises an antibody fragment.

19. A single chain Fc polypeptide, according to claim 18 wherein the antibody fragment is selected from VHH, VH, VL, VH-CH1, VL-CL, Fab, Fab′ or a scFv.

20. A single chain Fc polypeptide according to claim 19 wherein the antibody fragment is a Fab and the C-terminus of the VH-CH1 chain of the Fab is genetically fused to the N-terminus of the single chain Fc polypeptide, and the VL-CL chain of the Fab is linked to the VH-CH1 chain by a disulphide bond.

21. A single chain Fc polypeptide according to claim 12 to which one or more effector molecules is attached.

22. An isolated DNA sequence encoding the single chain Fc polypeptide according to claim 12.

23. An expression vector comprising one or more DNA sequences according to claim 22.

24. A host cell comprising one or more expression vectors according to claim 23.

25. A process for the production of a single chain Fc polypeptide comprising culturing the host cell of claim 24 and isolating the single chain Fc polypeptide.

26. A pharmaceutical composition comprising a single chain Fc polypeptide according to claim 12, in combination with one or more of a pharmaceutically acceptable excipient, diluent or carrier.

27. A pharmaceutical composition according to claim 26, additionally comprising other active ingredients.

28. An scFc polypeptide comprising at least two Fc moieties and at least one polypeptide linker

29. The scFc polypeptide of claim 28, wherein said scFc polypeptide comprises a first Fc moiety comprising a CH2 domain and a CH₃ domain and a second Fc moiety comprising a CH2 domain and a CH3 domain.

30. The scFc polypeptide of claim 28, wherein said scFc polypeptide further comprises one or more binding sites.

31. The scFc polypeptide of claim 30, wherein said binding entity is a soluble receptor or a ligand-binding fragment thereof.

32. The scFc polypeptide of claim 30, wherein said one or more binding entities are connected to the scFc by one or more polypeptide linkers.

33. The scFc polypeptide of claim 32, wherein said scFc polypeptide comprises two Fc monomers, a linker and further comprises at least one binding entity

34. A polynucleotide molecule comprising a polynucleotide sequence encoding the scFc polypeptide of claim 28.

35. The polynucleotide molecule of claim 34, further comprising a polynucleotide sequence encoding at least one binding moiety.

36. A cultured cell comprising the scFc polypeptide expression vector according claim 34.

37. The cultured cell of claim 36, wherein said cell is a yeast cell

38. The cultured cell of claim 36, wherein said cell is a mammalian cell

39. A method of producing an scFc polypeptide comprising:culturing a cell according to claim 38 under conditions wherein an scFc polynucleotide is expressed from said scFc polypeptide expression vector; and recovering said expressed scFc polypeptide

40. The polynucleotide molecule of claim 35, wherein said polynucleotide molecule is in an expression vector.

41. The polynucleotide molecule of claim 35, wherein a binding entity binds an antigen selected from the group consisting of: PDGFR, and HER2.


SEQUENCE LISTING (81 sequences)

SEQ ID NO: 1 - protein, 683 aa, Artificial Sequence
  Synthetic construct
QVQLVQSGAEVVKPGASVKLSCKASGYIFTSYYMYWVKQAPGQGLEWIGEINPSNGDTNFNEKFKSKATLTVDKSASTAYMELSSLRSEDTAVYYCTRSDGRNDMDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEPKSSDKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 226 aa, Artificial Sequence
  Synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 226 aa, Artificial Sequence
  Synthetic construct
DKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - DNA, 2109 bp, Artificial Sequence
  Synthetic construct
atggactggacctggagggtcttctgcttgctggctgtagcaccaggtgcccactcccaggtccaactggtgcagtcaggggctgaagtggtgaagcctggggcttcagtgaagttgtcctgcaaggcttctggctacatcttcaccagttattatatgtactgggtgaagcaggcgcccggacaaggccttgagtggattggagagattaatcctagcaatggtgatactaacttcaatgagaagttcaagagtaaggccacactgactgtagacaaatccgccagcacagcatacatggagctcagcagcctgaggtctgaggacactgcggtctattactgtacaagatcggacggtagaaatgatatggactcctggggccaagggaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtggaggtggcggatccgagcccaaatcttctgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcgcgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 5 - protein, 218 aa, Artificial Sequence
  Synthetic construct
DIVLTQSPATLSVSPGERATISCRASQRVSSSTYSYMHWYQQKPGQPPKLLIKYASNLESGVPARFSGSGSGTDFTLTISSVEPEDFATYYCQHSWEIPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - DNA, 717 bp, Artificial Sequence
  Synthetic construct
atggagacagacacactcctgttatgggtgctgctgctctgggttccaggttccactggtgacattgtactgacacagtctcctgctaccttatctgtatctccgggagagagggccaccatctcatgcagggccagccaacgtgtcagttcatctacctatagttatatgcactggtaccaacagaaaccaggacagccacccaaactcctcatcaagtatgcatccaacctagaatctggggtccctgccaggttcagtggcagtgggtctgggactgacttcaccctcaccatctcttctgtggagccggaggattttgcaacatattactgtcagcacagttgggagattcctccgacgttcggtggagggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 7 - protein, 683 aa, Artificial Sequence
  Synthetic construct
QVQLVQSGAEVVKPGASVKLSCKASGYIFTSYYMYWVKQAPGQGLEWIGEINPSNGDTNFNEKFKSKATLTVDKSASTAYMELSSLRSEDTAVYYCTRSDGRNDMDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - DNA, 2110 bp, Artificial Sequence
  Synthetic construct
atggactggacctggagggtcttctgcttgctggctgtagcaccaggtgcccactcccaggtccaactggtgcagtcaggggctgaagtggtgaagcctggggcttcagtgaagttgtcctgcaaggcttctggctacatcttcaccagttattatatgtactgggtgaagcaggcgcccggacaaggccttgagtggattggagagattaatcctagcaatggtgatactaacttcaatgagaagttcaagagtaaggccacactgactgtagacaaatccgccagcacagcatacatggagctcagcagcctgaggtctgaggacactgcggtctattactgtacaagatcggacggtagaaatgatatggactcctggggccaagggaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtggaggtggcggatccgagcccaaatcttctgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttgag

SEQ ID NO: 9 - protein, 683 aa, Artificial Sequence
  Synthetic construct
QVQLVQSGAEVVKPGASVKLSCKASGYIFTSYYMYWVKQAPGQGLEWIGEINPSNGDTNFNEKFKSKATLTVDKSASTAYMELSSLRSEDTAVYYCTRSDGRNDMDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEPKSSDKTHTSPPSPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 226 aa, Artificial Sequence
  Synthetic construct
DKTHTSPPSPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 30 aa, Artificial Sequence
  Synthetic peptide
GSEPKSSDKTHTSPPSPAPELLGGPSVFLF

SEQ ID NO: 12 - DNA, 2039 bp, Artificial Sequence
  Synthetic construct
gcagtcaggggctgaagtggtgaagcctggggcttcagtgaagttgtcctgcaaggcttctggctacatcttcaccagttattatatgtactgggtgaagcaggcgcccggacaaggccttgagtggattggagagattaatcctagcaatggtgatactaacttcaatgagaagttcaagagtaaggccacactgactgtagacaaatccgccagcacagcatacatggagctcagcagcctgaggtctgaggacactgcggtctattactgtacaagatcggacggtagaaatgatatggactcctggggccaagggaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtggaggtggcggatccgagcccaaatcttctgacaagactcacacatcacccccgagcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcgcgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttgag

SEQ ID NO: 13 - protein, 693 aa, Artificial Sequence
  Synthetic construct
QVQLVQSGAEVVKPGASVKLSCKASGYIFTSYYMYWVKQAPGQGLEWIGEINPSNGDTNFNEKFKSKATLTVDKSASTAYMELSSLRSEDTAVYYCTRSDGRNDMDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - DNA, 2139 bp, Artificial Sequence
  Synthetic construct
atggactggacctggagggtcttctgcttgctggctgtagcaccaggtgcccactcccaggtccaactggtgcagtcaggggctgaagtggtgaagcctggggcttcagtgaagttgtcctgcaaggcttctggctacatcttcaccagttattatatgtactgggtgaagcaggcgcccggacaaggccttgagtggattggagagattaatcctagcaatggtgatactaacttcaatgagaagttcaagagtaaggccacactgactgtagacaaatccgccagcacagcatacatggagctcagcagcctgaggtctgaggacactgcggtctattactgtacaagatcggacggtagaaatgatatggactcctggggccaagggaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtggaggtggcggatccggaggcggtggatcaggaggtggcggatctgagcccaaatcttctgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcgcgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 15 - protein, 693 aa, Artificial Sequence
  Synthetic construct
QVQLVQSGAEVVKPGASVKLSCKASGYIFTSYYMYWVKQAPGQGLEWIGEINPSNGDTNFNEKFKSKATLTVDKSASTAYMELSSLRSEDTAVYYCTRSDGRNDMDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - DNA, 2139 bp, Artificial Sequence
  Synthetic construct
atggactggacctggagggtcttctgcttgctggctgtagcaccaggtgcccactcccaggtccaactggtgcagtcaggggctgaagtggtgaagcctggggcttcagtgaagttgtcctgcaaggcttctggctacatcttcaccagttattatatgtactgggtgaagcaggcgcccggacaaggccttgagtggattggagagattaatcctagcaatggtgatactaacttcaatgagaagttcaagagtaaggccacactgactgtagacaaatccgccagcacagcatacatggagctcagcagcctgaggtctgaggacactgcggtctattactgtacaagatcggacggtagaaatgatatggactcctggggccaagggaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtggaggtggcggatccggaggcggtggatcaggaggtggcggatctgagcccaaatcttctgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 17 - protein, 693 aa, Artificial Sequence
  Synthetic construct
QVQLVQSGAEVVKPGASVKLSCKASGYIFTSYYMYWVKQAPGQGLEWIGEINPSNGDTNFNEKFKSKATLTVDKSASTAYMELSSLRSEDTAVYYCTRSDGRNDMDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - DNA, 2139 bp, Artificial Sequence
  Synthetic construct
atggactggacctggagggtcttctgcttgctggctgtagcaccaggtgcccactcccaggtccaactggtgcagtcaggggctgaagtggtgaagcctggggcttcagtgaagttgtcctgcaaggcttctggctacatcttcaccagttattatatgtactgggtgaagcaggcgcccggacaaggccttgagtggattggagagattaatcctagcaatggtgatactaacttcaatgagaagttcaagagtaaggccacactgactgtagacaaatccgccagcacagcatacatggagctcagcagcctgaggtctgaggacactgcggtctattactgtacaagatcggacggtagaaatgatatggactcctggggccaagggaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcgcttaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtggaggtggcggatccggaggcggtggatcaggaggtggcggatctgagcccaaatcttctgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcgcgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 19 - protein, 683 aa, Artificial Sequence
  Synthetic construct
QVQLVQSGAEVVKPGASVKLSCKASGYIFTSYYMYWVKQAPGQGLEWIGEINPSNGDTNFNEKFKSKATLTVDKSASTAYMELSSLRSEDTAVYYCTRSDGRNDMDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEPKSSDKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - DNA, 2109 bp, Artificial Sequence
  Synthetic construct
atggactggacctggagggtcttctgcttgctggctgtagcaccaggtgcccactcccaggtccaactggtgcagtcaggggctgaagtggtgaagcctggggcttcagtgaagttgtcctgcaaggcttctggctacatcttcaccagttattatatgtactgggtgaagcaggcgcccggacaaggccttgagtggattggagagattaatcctagcaatggtgatactaacttcaatgagaagttcaagagtaaggccacactgactgtagacaaatccgccagcacagcatacatggagctcagcagcctgaggtctgaggacactgcggtctattactgtacaagatcggacggtagaaatgatatggactcctggggccaagggaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcgcttaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtggaggtggcggatccgagcccaaatcttctgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcgcgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 21 - protein, 684 aa, Artificial Sequence
  Synthetic construct
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGHNDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEPKSSDKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - DNA, 2119 bp, Artificial Sequence
  Synthetic construct
atggactggacctggagggtcttctgcttgctggctgtagcaccaggtgcccactccgaagtacaattgttagagtctggtggcggtcttgttcagcctggtggttctttacgtctttcttgcgctgcttccggattcactttctctatttaccctatgttttgggttcgccaagctcctggtaaaggtttggagtgggtttcttggatcggtccttctggtggcattactaagtatgctgactccgttaaaggtcgcttcactatctctagagacaactctaagaatactctctacttgcagatgaacagcttaagggctgaggacacagccacatattactgtgcgagagaggggcataacgactggtacttcgatctctggggccgtggcaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtggaggtggcggatccgagcccaaatcttctgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcgcgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttgagcggccg

SEQ ID NO: 23 - protein, 214 aa, Artificial Sequence
  Synthetic construct
DIQMTQSPGTLSLSPGERATLSCRASQSVSSYLAWYQQKPGQAPRLLIYDASNRATGIPARFSGSGSGTEFTLTISSLQSEDFAVYYCQQYDKWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - DNA, 712 bp, Artificial Sequence
  Synthetic construct
atgagggtccccgctcagctcctggggctcctgctactctggctccgaggtgccagatgtgatatccagatgacccagtctccaggcaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagagttcactctcaccatcagcagcctgcagtctgaggattttgcagtttattactgtcagcagtatgataagtggccgctcactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagggatccc

SEQ ID NO: 25 - protein, 694 aa, Artificial Sequence [sequence omitted]

SEQ ID NO: 26 - DNA, 2142 bp, Artificial Sequence
  Synthetic construct
atggactggacctggagggtcttctgcttgctggctgtagcaccaggtgcccactccgaagtacaattgttagagtctggtggcggtcttgttcagcctggtggttctttacgtctttcttgcgctgcttccggattcactttctctatttaccctatgttttgggttcgccaagctcctggtaaaggtttggagtgggtttcttggatcggtccttctggtggcattactaagtatgctgactccgttaaaggtcgcttcactatctctagagacaactctaagaatactctctacttgcagatgaacagcttaagggctgaggacacagccacatattactgtgcgagagaggggcataacgactggtacttcgatctctggggccgtggcaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcgcttaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtggaggtggcggatccggaggcggtggatcaggaggtggcggatctgagcccaaatcttctgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcgcgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 27 - protein, 684 aa, Artificial Sequence
  Synthetic construct
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGHNDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEPKSSDKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - DNA, 2112 bp, Artificial Sequence
  Synthetic construct
atggactggacctggagggtcttctgcttgctggctgtagcaccaggtgcccactccgaagtacaattgttagagtctggtggcggtcttgttcagcctggtggttctttacgtctttcttgcgctgcttccggattcactttctctatttaccctatgttttgggttcgccaagctcctggtaaaggtttggagtgggtttcttggatcggtccttctggtggcattactaagtatgctgactccgttaaaggtcgcttcactatctctagagacaactctaagaatactctctacttgcagatgaacagcttaagggctgaggacacagccacatattactgtgcgagagaggggcataacgactggtacttcgatctctggggccgtggcaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcgcttaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtggaggtggcggatccgagcccaaatcttctgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcgcgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 29 - protein, 693 aa, Artificial Sequence
  Synthetic construct
QVQLQQPGAELVRPGASVKLSCKASGYTFTSYWMNWVKQRPEQGLEWIGRIDPHDSETHYRQKFKDMAILTVDKSSRTAYMQLSSLTSEDSAVYYCARGTMLDGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - DNA, 2138 bp, Artificial Sequence
  Synthetic construct
atgggatggagctgtgtaatgctcttcttgttagcaacagccacatgtgtccactcccaggtccaactgcagcagcctggggctgagctggtgaggcctggggcttcagtgaagctgtcctgcaaggcttctggctacacgttcaccagctactggatgaactgggttaagcagaggcctgagcaaggccttgagtggattggaaggattgatcctcacgatagtgagactcactaccgtcaaaagttcaaggacatggccattttgactgtggacaaatcctccaggacagcctacatgcaacttagcagcctgacatctgaggactctgcggtctattactgtgcaagagggactatgcttgatggtatggactactggggtcaaggaacctcagtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtggaggtggcggatccggaggcggtggatcaggaggtggcggatctgagcccaaatcttctgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgagctctgcacaaccactacacgcagaagagcctctccctgtctcccggttga

SEQ ID NO: 31 - protein, 698 aa, Artificial Sequence
  Synthetic construct
QVQLQQPGAELVRPGASVKLSCKASGYTFTSYWMNWVKQRPEQGLEWIGRIDPHDSETHYRQKFKDMAILTVDKSSRTAYMQLSSLTSEDSAVYYCARGTMLDGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - DNA, 2154 bp, Artificial Sequence
  Synthetic construct
atgggatggagctgtgtaatgctcttcttgttagcaacagccacatgtgtccactcccaggtccaactgcagcagcctggggctgagctggtgaggcctggggcttcagtgaagctgtcctgcaaggcttctggctacacgttcaccagctactggatgaactgggttaagcagaggcctgagcaaggccttgagtggattggaaggattgatcctcacgatagtgagactcactaccgtcaaaagttcaaggacatggccattttgactgtggacaaatcctccaggacagcctacatgcaacttagcagcctgacatctgaggactctgcggtctattactgtgcaagagggactatgcttgatggtatggactactggggtcaaggaacctcagtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtggaggtggcggatccgggggagggggcagcggagggggaggatctgggggcggaggatctgagcccaagagcagcgacaagacccacacctgccccccatgcccagctccagagctcctgggcggacccagcgtgttcctgttccctcccaagcccaaagacaccctgatgatcagcaggacccccgaggtcacctgcgtggtggtggacgtgtcccacgaggacccagaggtcaagttcaactggtacgtggacggcgtggaggtgcacaacgccaagaccaagcccagagaggaacagtacaacagcgcctacagggtggtgtccgtgctgaccgtgctgcaccaggactggctgaacggcaaagagtacaagtgcaaggtctccaacaaggccctgccagcccccatcgagaaaaccatcagcaaggccaagggccagccacgggagccccaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 33 - protein, 703 aa, Artificial Sequence
  Synthetic construct
QVQLQQPGAELVRPGASVKLSCKASGYTFTSYWMNWVKQRPEQGLEWIGRIDPHDSETHYRQKFKDMAILTVDKSSRTAYMQLSSLTSEDSAVYYCARGTMLDGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - DNA, 2169 bp, Artificial Sequence
  Synthetic construct
atgggatggagctgtgtaatgctcttcttgttagcaacagccacatgtgtccactcccaggtccaactgcagcagcctggggctgagctggtgaggcctggggcttcagtgaagctgtcctgcaaggcttctggctacacgttcaccagctactggatgaactgggttaagcagaggcctgagcaaggccttgagtggattggaaggattgatcctcacgatagtgagactcactaccgtcaaaagttcaaggacatggccattttgactgtggacaaatcctccaggacagcctacatgcaacttagcagcctgacatctgaggactctgcggtctattactgtgcaagagggactatgcttgatggtatggactactggggtcaaggaacctcagtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtggaggtggcggatccggcggcggaggaagcgggggagggggcagcggagggggaggatctgggggcggaggatctgagcccaagagcagtgacaagacccacacctgccccccatgcccagctccagagctgctgggcggacccagcgtgttcctgttccctcccaagcccaaagacaccctgatgatcagcaggacccccgaggtcacctgcgtggtggtggacgtgtcccacgaggacccagaggtcaagttcaactggtacgtggacggcgtggaggtgcacaacgccaagaccaagcccagagaggaacagtacaacagcgcctacagggtggtgtccgtgctgaccgtgctgcaccaggactggctgaacggcaaagagtacaagtgcaaggtctccaacaaggccctgccagcccccatcgagaaaaccatcagcaaggccaagggccagccacgggagccccaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 35 - protein, 708 aa, Artificial Sequence
  Synthetic construct
QVQLQQPGAELVRPGASVKLSCKASGYTFTSYWMNWVKQRPEQGLEWIGRIDPHDSETHYRQKFKDMAILTVDKSSRTAYMQLSSLTSEDSAVYYCARGTMLDGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - DNA, 2184 bp, Artificial Sequence
  Synthetic construct
atgggatggagctgtgtaatgctcttcttgttagcaacagccacatgtgtccactcccaggtccaactgcagcagcctggggctgagctggtgaggcctggggcttcagtgaagctgtcctgcaaggcttctggctacacgttcaccagctactggatgaactgggttaagcagaggcctgagcaaggccttgagtggattggaaggattgatcctcacgatagtgagactcactaccgtcaaaagttcaaggacatggccattttgactgtggacaaatcctccaggacagcctacatgcaacttagcagcctgacatctgaggactctgcggtctattactgtgcaagagggactatgcttgatggtatggactactggggtcaaggaacctcagtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtggaggtggcggatccggcggagggggctctggcggcggaggaagcgggggagggggcagcggagggggaggatctgggggcggaggatctgagcccaagagcagcgacaagacccacacctgccccccatgcccagctccagagctgctgggcggacccagcgtgttcctgttccctcccaagcccaaagacaccctgatgatcagcaggacccccgaggtcacctgcgtggtggtggacgtgtcccacgaggacccagaggtcaagttcaattggtacgtggacggcgtggaggtgcacaacgccaagaccaagcccagagaggaacagtacaacagcgcctacagggtggtgtccgtgctgaccgtgctgcaccaggactggctgaacggcaaagagtacaagtgcaaggtctccaacaaggccctgccagcccccatcgagaaaaccatcagcaaggccaagggccagccacgggagccccaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 37 - protein, 815 aa, Artificial Sequence [sequence omitted]

SEQ ID NO: 38 - DNA, 2541 bp, Artificial Sequence
  Synthetic construct
atggtgcgccccctgaacccgcgaccgctgccgcccgtagtcctgatgttgctgctgctgctgccgccgtcgccgctgcctctcgcagccggagacccccttcccacagaaagccgactcatgaacagctgtctccaggccaggaggaagtgccaggctgatcccacctgcagtgctgcctaccaccacctggattcctgcacctctagcataagcaccccactgccctcagaggagccttcggtccctgctgactgcctggaggcagcacagcaactcaggaacagctctctgataggctgcatgtgccaccggcgcatgaagaaccaggttgcctgcttggacatctattggaccgttcaccgtgcccgcagccttggtaactatgagctggatgtctccccctatgaagacacagtgaccagcaaaccctggaaaatgaatctcagcaaactgaacatgctcaaaccagactcagacctctgcctcaagtttgccatgctgtgtactctcaatgacaagtgtgaccggctgcgcaaggcctacggggaggcgtgctccgggccccactgccagcgccacgtctgcctcaggcagctgctcactttcttcgagaaggccgccgagccccacgcgcagggcctgctactgtgcccatgtgcccccaacgaccggggctgcggggagcgccggcgcaacaccatcgcccccaactgcgcgctgccgcctgtggcccccaactgcctggagctgcggcgcctctgcttctccgacccgctttgcagatcacgcctggtggatttccagacccactgccatcccatggacatcctaggaacttgtgcaacagagcagtccagatgtctacgagcatacctggggctgattgggactgccatgacccccaactttgtcagcaatgtcaacaccagtgttgccttaagctgcacctgccgaggcagtggcaacctgcaggaggagtgtgaaatgctggaagggttcttctcccacaacccctgcctcacggaggccattgcagctaagatgcgttttcacagccaactcttctcccaggactggccacaccctacctttgctgtgatggcacaccagaatgaagtcgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtggaggtggcggatccggaggcggtggatcaggaggtggcggatctgagcccaaatcttctgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 39 - protein, 629 aa, Artificial Sequence
  Synthetic construct
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEPKSSDKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - DNA, 1959 bp, Artificial Sequence
  Synthetic construct
atgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaacgtcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtggaggtggcggatccgagcccaaatcttctgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcgcgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttgagcggcc

SEQ ID NO: 41 - protein, 671 aa, Artificial Sequence
  Synthetic construct
SQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 42 - DNA, 2097 bp, Artificial Sequence
  Synthetic construct
atgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggcagggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatggtcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtggaggtggcggatccggaggcggtggatcaggaggtggcggatctgagcccaaatcttctgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcgcgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 43 - protein, 661 aa, Artificial Sequence
  Synthetic construct
SQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEPKSSDKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - DNA, 2067 bp, Artificial Sequence
  Synthetic construct
atgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggcagggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatggtcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtggaggtggcggatccgagcccaaatcttctgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcgcgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 45 - protein, 467 aa, Artificial Sequence
  Synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEPKSSDKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 46 - DNA, 1461 bp, Artificial Sequence
  Synthetic construct
atgaagctccccgtcaggcttctcgtgctcatgttctggattccggcgtcgtcaagtgagcccaaatctagtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtggaggtggcggatccgagcccaaatcttctgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcgcgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 47 - protein, 477 aa, Artificial Sequence
  Synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - DNA, 1491 bp, Artificial Sequence
  Synthetic construct
atgaagctccccgtcaggcttctcgtgctcatgttctggattccggcgtcgtcaagtgagcccaaatctagtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtggaggtggcggatccggaggcggtggatcaggaggtggcggatctgagcccaaatcttctgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 49 - protein, 467 aa, Artificial Sequence
  Synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 50 - DNA, 1461 bp, Artificial Sequence
  Synthetic construct
atgaagctccccgtcaggcttctcgtgctcatgttctggattccggcgtcgtcaagtgagcccaaatctagtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtggaggtggcggatccgagcccaaatcttctgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 51 - protein, 446 aa, Artificial Sequence
  Synthetic construct
DVQLQQSGAELARPGASIKMSCKASGYTFTSYTIHWVKKRPGQGLEWIGYITPNIDYTKYNQKFKDRATLTADKSSSTAYIQLSSLTSEDSAVYYCARNGYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 52 - DNA, 1401 bp, Artificial Sequence
  Synthetic construct
atggagacagacacactcctgttatgggtgctgctgctctgggttccaggttccactggtgacgtccagctgcagcagtctggggctgaactggcaagacctggggcctcaataaagatgtcctgcaaggcttctggctatacctttactagctacacaattcactgggtaaaaaagaggcctggacagggtctggaatggattggatacattactcctaacattgattatactaagtacaatcagaagttcaaggacagggccacattgactgcagacaaatcctccagcacagcctacatacaactgagcagcctgacatctgaggactctgcagtctattattgtgcaagaaatggttactacgttatggactactggggtcaaggaacctcagtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 53 - protein, 213 aa, Artificial Sequence
  Synthetic construct
QIVLTQSPAIMSASPGEKVTMTCRASSSVSHMHWYQQKSGTSPKRWIYDTSKLASGVPARFSGSGSGTSYSLTISSVEAEDAATYYCQQWSSNPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 17 aa, Homo sapiens
DKTHTCPPCPAPELLGG

SEQ ID NO: 55 - protein, 7 aa, Artificial Sequence
  Synthetic peptide
ERKCCVE

SEQ ID NO: 56 - protein, 7 aa, Artificial Sequence
  Synthetic peptide
APPVAGP

SEQ ID NO: 57 - protein, 10 aa, Artificial Sequence
  Synthetic peptide
EPKSCDKTHT

SEQ ID NO: 58 - protein, 5 aa, Artificial Sequence
  Synthetic peptide
CPPCP

SEQ ID NO: 59 - protein, 8 aa, Artificial Sequence
  Synthetic peptide
APELLGGP

SEQ ID NO: 60 - protein, 12 aa, Artificial Sequence
  Synthetic peptide
ELKTPLGDTTHT

SEQ ID NO: 61 - protein, 50 aa, Artificial Sequence
  Synthetic peptide
CPRCPEPKSCDTPPPCPRCPEPKSCDTPPPCPRCPEPKSCDTPPPCPRCP

SEQ ID NO: 62 - protein, 8 aa, Artificial Sequence
  Synthetic peptide
APELLGGP

SEQ ID NO: 63 - protein, 7 aa, Artificial Sequence
  Synthetic peptide
ESKYGPP

SEQ ID NO: 64 - protein, 5 aa, Artificial Sequence
  Synthetic peptide
CPSCP

SEQ ID NO: 65 - protein, 8 aa, Artificial Sequence
  Synthetic peptide
APEFLGGP

SEQ ID NO: 66 - protein, 50 aa, Artificial Sequence
  Synthetic peptide
GGGGSGGGGSGGGGSGGGGSGGGGSGGGGSGGGGSGGGGSGGGGSGGGGS

SEQ ID NO: 67 - protein, 20 aa, Artificial Sequence
  Synthetic peptide
GGGGSGGGGSGGGGSGGGGS

SEQ ID NO: 68 - protein, 15 aa, Artificial Sequence
  Synthetic peptide
GGGGSGGGGSGGGGS

SEQ ID NO: 69 - protein, 5 aa, Artificial Sequence
  Synthetic peptide
GGGGS

SEQ ID NO: 70 - protein, 25 aa, Artificial Sequence
  Synthetic peptide
GGGGSGGGGSGGGGSGGGGSGGGGS

SEQ ID NO: 71 - protein, 30 aa, Artificial Sequence
  Synthetic peptide
GGGGSGGGGSGGGGSGGGGSGGGGSGGGGS

SEQ ID NO: 72 - protein, 31 aa, Artificial Sequence
  Synthetic peptide
SGGGGSGGGGSGGGGSGGGGSGGGGSGGGGS

SEQ ID NO: 73 - protein, 15 aa, Artificial Sequence
  Synthetic peptide
EPKSCDTPPPCPRCP

SEQ ID NO: 74 - protein, 25 aa, Artificial Sequence
  Synthetic peptide
GGGGSGGGGSGGGGSGGGGSGGGGS

SEQ ID NO: 75 - protein, 10 aa, Artificial Sequence
  Synthetic peptide
GGGSSGGGSG

SEQ ID NO: 76 - protein, 10 aa, Artificial Sequence
  Synthetic peptide
IGKTISKKAK

SEQ ID NO: 77 - protein, 25 aa, Artificial Sequence
  Synthetic peptide
GGGGSGGGGSGGGGSGGGGSGGGAS

SEQ ID NO: 78 - protein, 17 aa, Artificial Sequence
  Synthetic peptide
SLSLSPGGGGGSEPKSS

SEQ ID NO: 79 - protein, 30 aa, Artificial Sequence; identical to SEQ ID NO: 71
  Synthetic peptide

SEQ ID NO: 80 - DNA, 704 bp, Artificial Sequence
  Synthetic construct
atggattttcaggtgcagattttcagcttcctgctaatcagtgcctcagtcataatatccagaggacaattgttctcacccagtctccagcaatcatgtctgcatctccaggggagaaggtcaccatgacctgccgtgccagctcaagtgtaagtcacatgcactggtaccagcagaagtcaggcacctcccccaaaagatggatttatgacacatccaaactggcttctggagtccctgctcgcttcagtggcagtgggtctgggacctcttactctctcacaatcagcagcgtggaggctgaagatgctgccacttattactgccagcagtggagtagtaacccgctcacgttcggtgctgggaccaagctggagctgaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 81 - protein, 12 aa, Artificial Sequence
  Synthetic peptide
GGGSGGGSGGGS